(19) United States
(12) Patent Application Publication
(10) Pub. No.: US 2025/0034579 A1
(43) Pub. Date: Jan. 30, 2025

(54) METHODS AND COMPOSITIONS FOR GENETICALLY MODIFYING HUMAN GUT MICROBES

(71) Applicant: Cornell University, Ithaca, NY (US)

(72) Inventors: Chun-Jun GUO, New York, NY (US); Wenbing JIN, New York, NY (US); Tingting LI, New York, NY (US)

(73) Assignee: Cornell University, Ithaca, NY (US)

(21) Appl. No.: 18/716,858

(22) PCT Filed: Dec. 6, 2022
(86) PCT No.: PCT/US2022/051979

§ 371 Date: Jun. 5, 2024

Related U.S. Application Data
(60) Provisional application No. 63/286,736, filed on Dec. 7, 2021.

(51) Int. Cl. C12N 15/74; C12N 1/20; C12N 15/11

(52) U.S. Cl. CPC C12N 15/74; C12N 1/20; C12N 15/11; C12N 2510/00

(57) ABSTRACT

Dysbiosis has been linked to diseases such as inflammatory bowel disease and obesity. Multi-omics studies have uncovered significant associations between microbiota genes and diseases. Many of these genes are exclusively expressed in non-model microbes such as Firmicutes/Clostridia. A pipeline for building microbial genetic manipulation systems would be a first step to manipulating these genes in vivo and causally connecting them with host diseases. The present technology relates generally to compositions and the methods of preparations thereof for genetically engineering gut-microbiota in vitro. The present technology further relates to uses of compositions in vivo.

### CROSS-REFERENCE TO RELATED APPLICATIONS

This application claims the benefit of and priority to U.S. Provisional Patent Application No. 63/286,736, filed Dec. 7, 2021, the entire contents of which are incorporated herein by reference.

### GOVERNMENT SUPPORT

This invention was made with government support under DK126871, AI151599, AI095466, AI095608, AI142213 and 1DP2HD101401-01 awarded by the National Institutes of Health. The government has certain rights in the invention.

### TECHNICAL FIELD

The present technology relates generally to compositions and the methods of preparations thereof for genetically engineering gut-microbiota in vitro. The present technology further relates to uses of compositions in vivo.

### BACKGROUND

The following description of the background of the present technology is provided simply as an aid in understanding the present technology and is not admitted to describe or constitute prior art to the present technology.

Dysbiosis, or perturbation of the microbiome, has been linked to diseases such as inflammatory bowel disease and obesity. Multi-omics studies uncover many microbiota genes that are associated with host biology. However, it remains challenging to unravel the causal mechanisms underlying microbiota gene-host biology interactions, mainly because many are encoded by non-model gut microbes like Firmicutes/Clostridia. While genetic toolsets are readily available for model bacteria like E. coli or B. thetaiotaomicron, the limitation lies in that the optimal condition identified in one study is not readily applicable to the other. Most of the gut commensals, especially those that are dominant in the gut, are non-model gut bacteria (e.g., Bacteroides, Prevotella, and Clostridium) are still resistant to genetic modifications. In addition, engineering therapeutic functions into the microbiome requires targeted genomic edits, which presents a further challenge because many non-model gut bacteria (e.g., Lachnospiraceae, Prevotella) are not genome sequenced, and it is unknown how to introduce exogenous DNA or which gene manipulation tool to select (Waller et al., 2017a).

There is an urgent need for efficient, standardized, and in vitro pipeline to identify their gene transfer methods and build their genetic manipulation systems without prior knowledge of their genome information. Such pipelines are important for three reasons: 1) Multi-omics studies have uncovered significant associations between microbiota genes and diseases. Many of these genes are exclusively expressed in non-model microbes such as Firmicutes/Clostridia (Lloyd-Price et al., 2019; Thomas et al., 2019; Wang et al., 2012; Wirbel et al., 2019; Yachida et al., 2019; Zhou et al., 2019). A pipeline addressing this need would be a first step to manipulating these genes in vivo and causally connecting them with host diseases. 2) The gut microbiota plays an essential role in regulating host biology, but little is known about which bacteria and genes are responsible. A desirable pipeline would enable gene toggling in previously non-targetable microbes and boost in-depth mechanistic studies of microbiota-host physiology interactions. 3) The microbiota impacts multiple therapies such as fecal microbiota transplantation and cancer immunotherapy (Helmink et al., 2019; Roy and Trinchieri, 2017), but the molecular mechanisms behind them largely remain elusive.

### SUMMARY OF THE PRESENT TECHNOLOGY

In one aspect, the present disclosure provides a bacterial expression vector comprising (a) a nucleic acid encoding a target gene that is conserved in a plurality of human gut commensal gram-negative bacterial species and (b) a heterologous nucleic acid encoding a selectable marker, wherein the selectable marker is an antibiotic resistance gene or an auxotrophic marker, and optionally wherein the target gene is selected from the group consisting of 16s rRNA, 23s rRNA, mmdA, RokA (Clucokinase gene), and an ABC transporter gene. Additionally or alternatively, in some embodiments, the bacterial expression vector further comprises at least one open reading frame encoding a bioluminescent protein, a chemiluminescent protein, a fluorescent protein, a CRISPR enzyme, a Group II intron-encoded protein, at least one sgRNA, at least one Group II intron, or any combination thereof. In some embodiments, the 16s rRNA comprises the nucleic acid sequence of SEQ ID NO: 11. Additionally or alternatively, in some embodiments, the bacterial expression vector comprises the nucleic acid sequence of SEQ ID NO: 310.

In one aspect, the present disclosure provides a bacterial expression vector comprising (a) a gram-positive bacteria replication origin comprising a sequence selected from the group consisting of SEQ ID NOs: 1-9 or 311-319, (b) a heterologous nucleic acid encoding a selectable marker that is an antibiotic resistance gene or an auxotrophic marker, and (c) at least one open reading frame, wherein the at least one open reading frame encodes a bioluminescent protein, a chemiluminescent protein, a fluorescent protein, a CRISPR enzyme, a Group II intron-encoded protein, at least one sgRNA, at least one Group II intron, or any combination thereof. The bacterial expression vector of the present technology may further comprise one or more bacterial conjugation transfer genes and/or an E. coli replication origin. Examples of bacterial conjugation transfer genes include traJ and oriT, and examples of E. coli replication origin include colE1, pBR, and R6K. Additionally or alternatively, in some embodiments, the one or more bacterial conjugation transfer genes, the gram-positive bacteria replication origin, and the heterologous nucleic acid encoding the selectable marker are codon optimized. Additionally or alternatively, in some embodiments, the at least one sgRNA or the at least one Group II intron targets one or more genes selected from among 16S rRNA, porA, bcat, croA, baiA2, baiCD, baiF, baiH, baiB, baiE, baiG and bail.

In any and all embodiments of the bacterial expression vectors disclosed herein, the antibiotic resistance gene is selected from the group consisting of catP, ermB, aad9, tetA, and ampR, or the auxotrophic marker is pyrG, or pyrF.

In any of the preceding embodiments of the bacterial expression vectors disclosed herein, the CRISPR enzyme is selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

Examples of fluorescent proteins include, but are not limited to, GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOκ, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa. Examples of chemiluminescent proteins include, but are not limited to, β-galactosidase, horseradish peroxidase (RP), or alkaline phosphatase. Examples of bioluminescent protein include, but are not limited to, Aequorin, firefly luciferase, Renilla luciferase, red luciferase, luxAB, or nanoluciferase.

In any and all embodiments of the bacterial expression vectors disclosed herein, the at least one sgRNA specifically hybridizes with a heterologous or endogenous target gene expressed in a gut bacterial host cell, and/or wherein the at least one sgRNA and/or the CRISPR enzyme is operably linked to a constitutive promoter or a conditional promoter. Additionally or alternatively, in some embodiments of the bacterial expression vectors disclosed herein, the at least one Group II intron specifically targets a heterologous or endogenous gene expressed in a gut bacterial host cell, and/or wherein the at least one Group II intron and/or the Group II intron-encoded protein is operably linked to a constitutive promoter or a conditional promoter.

In another aspect, the present disclosure provides an engineered gram-negative human gut bacterial cell comprising any and all embodiments of the gram-negative specific bacterial expression vector described herein, wherein the engineered gram-negative human gut bacterial cell is derived from a family selected from the group consisting of Enterobacteriaceae, Bacteroidaceae, Tannerellaceae, and Prevotellaceae. In some embodiments, the engineered gram-negative human gut bacterial cell is derived from Bacteroides cellulosilyticus, Bacteroides cellulosilyticus, Bacteroides dorei, Bacteroides eggerthii, Bacteroides finegoldii, Bacteroides fragilis, Bacteroides intestinalis, Bacteroides nordii, Bacteroides oleiciplenus, Bacteroides ovatus, Bacteroides salyersiae, Bacteroides sp., Bacteroides thetaiotaomicron, Bacteroides unformis, Bacteroides vulgatus, Bacteroides xylanisolvens, Parabacteroides faecis, Parabacteroides merdae, or Prevotella bivia.

In one aspect, the present disclosure provides an engineered gram-positive human gut bacterial cell comprising any and all embodiments of the gram-positive specific bacterial expression vector disclosed herein, wherein the engineered gram-positive human gut bacterial cell is derived from a family selected from the group consisting of Clostridiaceae, Lachnospiraceae, Eubacteriaceae, Erysipelotrichaceae, Enterococcaceae, and Bifidobacteriaceae. In some embodiments, the engineered gram-positive human gut bacterial cell is derived from Blautia hydrogenotrophica, Blautia luti, Blautia sp., Blautia wexlerae, Clostridium bolteae, Clostridium innocuum, Clostridium paraputrificum, Clostridium saccharolyticum, Clostridium senegalense, Clostridium sp., Clostridium sporogenes, Clostridium symbiosum, Eubacterium limosum, Eubacterium maltosivorans, Eubacterium ramulus, Eubacterium sp., Roseburia inulinivorans, Bifidobacterium catenulatum, Enterococcus faecium, Escherichia fergusonii, Roseburia inulinivorans, or Bifidobacterium catenulatum.

In one aspect, the present disclosure provides a method for modifying a gram-negative human gut bacteria cell genome comprising transferring at least one gram-negative specific bacterial expression vector described herein into a gram-negative human gut bacteria cell via conjugation. In some embodiments, the at least one bacterial expression vector is integrated into the genome of the gram-negative human gut bacteria cell.

In another aspect, the present disclosure provides a method for genetically modifying a gram-positive human gut bacteria cell comprising transferring two or more distinct bacterial expression vectors into a gram-positive human gut bacteria cell simultaneously via conjugation, wherein each of the two or more distinct bacterial expression vectors comprise: (a) a gram-positive bacteria replication origin comprising a sequence selected from the group consisting of SEQ ID NOs: 1-9 or 311-319, (b) a heterologous nucleic acid encoding a selectable marker that is an antibiotic resistance gene or an auxotrophic marker, and (c) at least one open reading frame, wherein the at least one open reading frame encodes a bioluminescent protein, a chemiluminescent protein, a fluorescent protein, a CRISPR enzyme, a Group II intron-encoded protein, at least one sgRNA, at least one Group II intron, or any combination thereof. The antibiotic resistance gene or the auxotrophic marker of each of the two or more distinct bacterial expression vectors may be independently selected from the group consisting of catP, ermB, aad9, tetA, ampR, pyrG, and pyrF.

In some embodiments, each of the two or more distinct bacterial expression vectors further comprise one or more bacterial conjugation transfer genes and/or an E. coli replication origin, optionally wherein the one or more bacterial conjugation transfer genes are selected from the group consisting of traJ, and oriT and/or the E. coli replication origin is selected from the group consisting of colE1, pBR, and R6K.

Additionally or alternatively, in some embodiments, the CRISPR enzyme of each of the two or more distinct bacterial expression vectors is independently selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

Additionally or alternatively, in some embodiments of the methods disclosed herein, the fluorescent protein of each of the two or more distinct bacterial expression vectors is independently selected from the group consisting of GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOκ, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, and Dronpa. Additionally or alternatively, in certain embodiments of the methods disclosed herein, the chemiluminescent protein of each of the two or more distinct bacterial expression vectors is independently β-galactosidase, horseradish peroxidase (HRP), or alkaline phosphatase. Additionally or alternatively, in some embodiments of the methods of the present technology, the bioluminescent protein of each of the two or more distinct bacterial expression vectors is independently Aequorin, firefly luciferase, Renilla luciferase, red luciferase, luxAB, or nanoluciferase.

In any and all embodiments of the methods disclosed herein, the at least one sgRNA sequence of the two or more distinct bacterial expression vectors specifically hybridizes with a heterologous or endogenous target gene expressed in a gut bacterial host cell, and/or wherein the at least one sgRNA and/or the CRISPR enzyme is operably linked to a constitutive promoter or a conditional promoter. Additionally or alternatively, in some embodiments of the methods disclosed herein, the at least one Group II intron of the two or more distinct bacterial expression vectors specifically targets a heterologous or endogenous gene expressed in a gut bacterial host cell, and/or wherein the at least one Group II intron and/or the Group II intron-encoded protein is operably linked to a constitutive promoter or a conditional promoter. In some embodiments, three or four distinct bacterial expression vectors are simultaneously transferred into a gram-positive human gut bacteria cell simultaneously via conjugation.

In any and all embodiments of the methods disclosed herein, the gram-negative or gram-positive human gut bacteria cell is isolated from a colonic mucosa-enriched lavage sample, a fecal sample, a rectal swab, or an intestinal sample obtained from a human subject.

Also disclosed herein are engineered human gut bacterial cells generated by any and all embodiments of the methods of the present technology.

Also provided herein are kits comprising any and all embodiments of the bacterial expression vectors of the present technology and instructions for using the bacterial expression vectors to genetically modify human gut bacteria. The kits may further comprise one or more primers and/or gRNAs comprising the sequence of any one of SEQ ID NOs: 23-287.

### BRIEF DESCRIPTION OF THE DRAWINGS

FIGS. 1A-IC. Overview of the genetic manipulation (GM) pipeline for non-model gut commensals. FIG. 1A: A total of 201 human gut isolates from >140 species and 5 phyla were subject to the GM pipeline. The pipeline identifies gene transfer methods for 91 non-model gut microbes (of 72 species) and build gene manipulation tools for 72 of them. For Gram-negative gut microbes, identifying their gene transfer methods and building their gene insertion tools are achieved in one step via the chimeric-16s rRNA strategy. FIG. 1B: Phylogenetic tree (colored by Family) of the 16s rRNA sequences from the 91 genetically targetable microbes identified via the GM pipeline. FIG. 1C: Detailed phylogenetic information of the 91 genetically targetable microbes identified in this study. These microbes are from 72 bacterial species in 16 families.

FIGS. 2A-2D. Developing a genetic manipulation pipeline for non-model gut commensals. FIG. 2A: Schematic view of a multifactorial optimization of the conjugation/transformation parameters to identify gene transfer conditions for 38 non-model gut Firmicutes/Clostridia that are mostly untransformed. FIG. 2B: Establishment of a dCpf1-lacZα platform for non-model gut Firmicutes/Clostridia. (left) Schematic view of a dCpf1-lacZα system. The promoter and CDS region of lacZα are targeted by a duplex gRNA G1 and G2. (right) The dCpf1-lacZα system efficiently suppresses lacZα expression in 25 Clostridia microbes. The panel shows the mean gene expression of three biological replicates as determined by qPCR. The dCpf-1-only and gRNA-only vectors are used as negative controls. Three out of 25 qPCR results are shown. The numbering of the strains corresponds to the strain information shown in FIG. 22. Error bar: standard deviation. DR: direct repeat, G1: guide RNA-coding sequence 1, G2: guide RNA-coding sequence 2, Ter: terminator. FIG. 2C: Schematic view of the 16s-tron strategy for non-model Clostridia. The Clostridia 16s rRNA sequences were aligned to identify a conserved target site of Group II intron. The 16s targeting Group II intron (16s-tron) was introduced into 19 Clostridia commensals due to RAM (retrotransposition-activated marker) availability. We identified 16 Clostridia whose chromosomes have been integrated by the 16s-tron. FIG. 2D: Schematic view of a Bacteroidia/Prevotella GM pipeline. The Prevotella 16s rRNA sequences were aligned to generate a ˜1 kb chimeric 16s (chi-16s) fragment. The chi-16 was assembled to get a suicide vector pGM-NAC₂P. The pGM-NAC₂P (NAC₂B for Bacteroides) was conjugated to 21 Prevotella, 39 Bacteroides, and 6 Parabacteroides commensals targeting their chromosomal 16s rRNA genes. We identified 31 targetable Bacteroidia whose 16s rRNA genes have been integrated by pGM-NAC₂P (or NAC₂B).

FIGS. 3A-3D. Modulating Clostridia gene expression and microbiome-derived metabolites using gene manipulation tools developed via the GM pipeline. FIG. 3A (top): Schematic view of a duplex gRNA targeting the branched-chain amino acid aminotransferase bcat in the Clostridia commensals. FIG. 3A (bottom): The bcat gene of 12 Clostridia microbes was efficiently repressed using dCpf1. The panel shows the mean gene expression of three biological replicates as determined by qPCR. Only three representative results (S54, S74, and S110, FIG. 22) are shown. FIG. 3B (top): Schematic view of knocking out the Bacteroides mmdA genes using pGM vectors. The pMG vector was assembled with ˜1 kb fragment of the mmdA genes, and the mmdA genes of three Bacteroides microbes were knocked out via single crossover integration. FIG. 3B (bottom): Three Bacteroides ΔmmdA mutants (S25, S27, and S31, FIG. 22) deplete propionate in vitro. The bacterial culture supernatant was derivatized and propionate production was examined using LC-MS (EIC: 216.1137). The Bacteroides ΔmmdA mutant depletes propionate in vivo. Germ-free Swiss Webster mice (n=3 or 4 per group) were mono-colonized with the B. sp. 1_1_6 control strain (Con, 16s integrated by pGM-NAC₂B) and ΔmmdA mutant (Mut). Propionate was depleted in the host by mmdA deletion. Student's T-test was performed, and the asterisk indicates p-value <0.05 (*) or <0.01 (**). Error bar: standard deviation. FIG. 3C (top): Schematic view of modulating butyrate production in the Clostridia commensals using dCpf1 or Group II intron. FIG. 3C (bottom): The butyrate production (quantified by LC-MS) was significantly reduced in three Clostridia microbes S110 C. symbiosum (by dCpf1), S115 E. limosum (by Group II intron), and S117 E. maltosivorans (by dCpf1) (FIG. 22). The cecal butyrate (quantified by LC-MS) in the germ-free Swiss Webster mice mono-colonized with S117 E. maltosivorans mutant (Mut, dCpf1+gRNAs) is significantly lower compared to the control (Con, dCpf1 only). Student's T-test was performed, and the asterisk indicates p-value <0.05 (*) or <0.01 (**). Error bar: standard deviation. FIG. 3D: In vitro and in vivo depletion of branched short-chain fatty acids (BSCFAs) by S107 C. sporogenes using CRISPR-dCpf1. FIG. 3D (top): Schematic view of targeting the BSCFAs gene porA using CRISPR-dCpf1. The dCpf1 gRNA (G1) targets the porA promoter region. FIG. 3D (bottom): The porA expression (by qPCR) is significantly reduced in the mutant (Mut, dCpf1 with gRNA) compared to the control (Con, dCpf1 only) in vitro. Germ-free Swiss Webster mice (n=4 per group) mono-associated with porA repression mutant have much less isovalerate (quantified by LC-MS) in their feces than the control (dCpf1 only). Student's T-test was performed, and the asterisk indicates p-value <0.05 (*) or <0.01 (**). Error bar: standard deviation. For (A), (C), and (D), DR: direct repeat, G1: guide RNA-coding sequence 1, G2: guide RNA-coding sequence 2, Ter: terminator. The numbering of the strains corresponds to the strain information shown in FIG. 22.

FIGS. 4A-4D. Knocking out baiH in gnotobiotic mice. FIG. 4A: The orientation of the S122 bai operon for bile acid 7α-dehydroxylation. The mutated gene baiH (by Group II intron) is highlighted in red. The S122 bai operon is actively transcribed under host colonization, and three representative results of metatranscriptomic analyses of the S122 bai operon are shown.

FIG. 4B: The biosynthetic scheme of bile acid 7α-dehydroxylation. The baiH encodes an oxidoreductase that reduces the 6,7-olefinic bond of the intermediate 3-oxo-4,5-6,7-didehydro-DCA (2, EIC: 385.2384). The S122 ΩbaiH mutant accumulates the predicted intermediate (2, EIC: 385.2384) and no longer converts CA (1, EIC: 407.2803) to DCA (3, EIC: 391.2854) in vitro. The structure of the intermediate (2) was determined by comparing its retention time and exact mass to the published literature. The asterisk indicates a residual amount of DCA that is a contaminant from the CA chemical standard. EIC: extracted ion chromatogram. FIG. 4C: Germ-free C57BL/6J mice (n=3 or 4 per group) were co-colonized with S25 plus the S122 control (Con) or ΩbaiH mutant (Mut) (by Group II intron) strain. The relative abundances of S122 in the control and mutant group were assessed by 16s rRNA sequencing and were comparable. FIG. 4D: Depleting baiH using Group II intron abolishes gut 7α-dehydroxylating activity and modifies gut bile acid pool in gnotobiotic mice. CA, DCA, and 7-oxo CA (see FIG. 20 for their structures) were quantified using LCMS. Data in FIG. 4C and FIG. 4D were analyzed using unpaired two-tailed Student's T-test. The asterisk indicates p-value <0.05 (*) or <0.01 (**). The numbering of the strains corresponds to the strain information shown in FIG. 22.

FIGS. 5A-5H. Knocking out baiH in the context of a complex microbiota impacts the host bile acid pool and the gut microbiome. FIG. 5A: SPF C57BL/6J mice (n=5 per group) given low dose antibiotic water (15 μg/ml thiamphenicol and 10 μg/ml erythromycin) were colonized with genetically tagged S122 control (Con) or ΩbaiH mutant (Mut) (by Group II intron) strain. FIG. 5B: The relative abundances of S122 in the control and mutant group were assessed by 16s rRNA sequencing and were comparable. The SPF mice are stably colonized with S122 control (Con) and the ΩbaiH mutant (Mut) at about the same level with a comparable total bacterial load. FIG. 5C: Principal coordinates analysis (PCoA) of the fecal microbiome of the control and ΩbaiH mutant mice. FIG. 5D: Targeted metabolomics analyses (quantified by LC-MS) of the stool bile acid (BA) compositions of the control (Con) and ΩbaiH mutant (Mut) colonized SPF mice. FIG. 5E: The relative abundance of taxonomic phyla in the gut microbiota of the control and ΩbaiH mutant mice. FIG. 5F: Relative abundances of inflammation-associated gut microbial taxa in the stool microbiome of the control and ΩbaiH mutant mice. FIG. 5G: Volcano plot of differential bacterial OTU abundances calculated from 16S rRNA gene sequencing. Significantly different OTUs (n=56, FDR <0.05) are colored and plotted. The Bacteroidia OTU and Erysipelotrichaceae OTU with high relative abundances (>10%) are marked with an upward pointing arrow. FIG. 5H: Gut 7α-dehydroxylating activity is positively associated with fecal calprotectin level in nonIBD people. In FIG. 5B, FIG. 5D, and FIG. 5F data were analyzed using unpaired two-tailed Student's T-test, and the asterisk indicates p-value <0.01 (**). The data in FIG. 5C, FIG. 5E, FIG. 5F, and FIG. 5G are representative of two independent experiments with n=4 or 5 per group, and only the changes in taxonomic groups that are consistent between the two experiments are shown. Data are shown as mean±SEM. The numbering of the strains corresponds to the strain information shown in FIG. 22.

FIGS. 6A-6J. baiH modulates intestinal inflammation in the context of complex gut microbiota. FIGS. 6A, 6F: DSS-induced murine colitis model was applied to the SPF or gnotobiotic mice colonized with the genetically tagged S122 control (Con) and ΩbaiH mutant (Mut). Mice were colonized with the control or mutant strain for at least two weeks before giving DSS, SPF mice were given 2.5% DSS (in water supplemented with 15 μg/ml thiamphenicol and 10 μg/ml erythromycin) for 8 days, and gnotobiotic mice were given 2.0% DSS (in water supplemented with 15 μg/ml thiamphenicol) for 7 days. The disease state was monitored by weight loss (FIGS. 6B, 6G), hematoxylin and eosin (H&E) staining of the distal colon (FIGS. 6C, 6H), colon shortening, and histopathologic score (FIGS. 6D, 6I), and fecal lipocalin-2 and daily hematochezia score (FIGS. 6E, 6J). Data shown in FIGS. 6B-6E, 6G-6J are representations of n=4 to 5 mice per group replicated in two or more independent experiments.

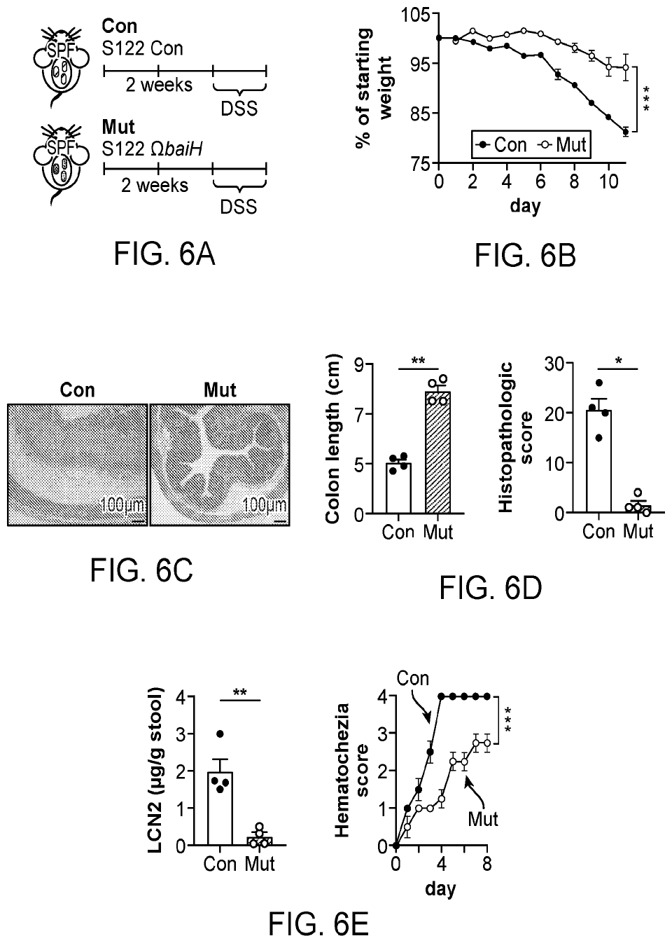

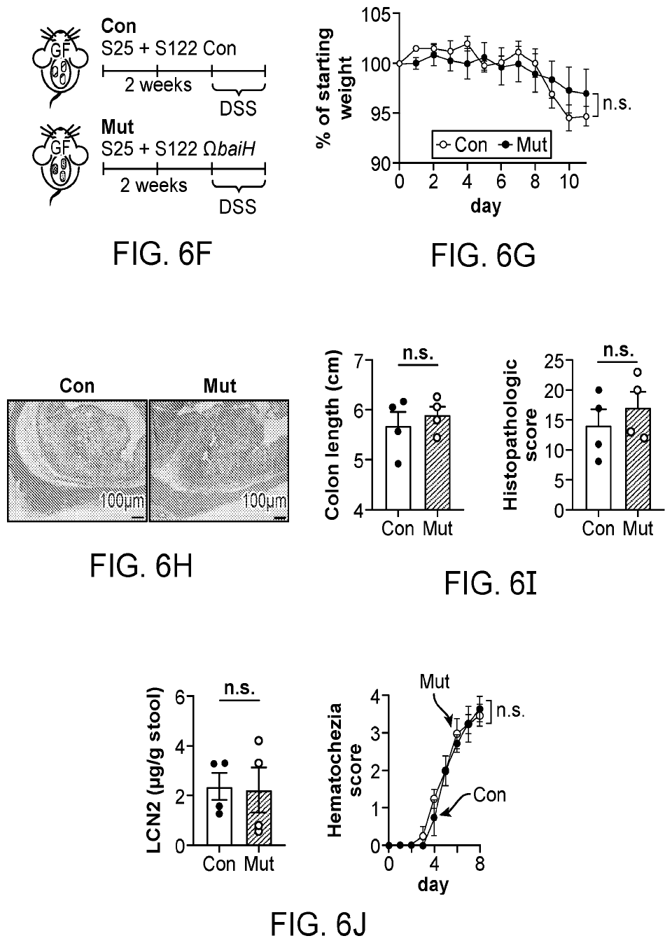

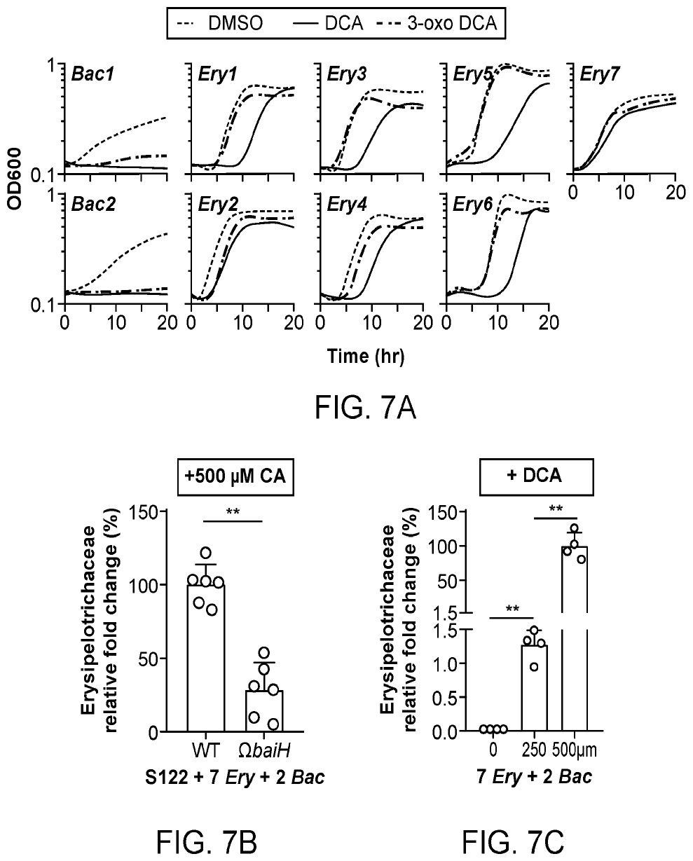

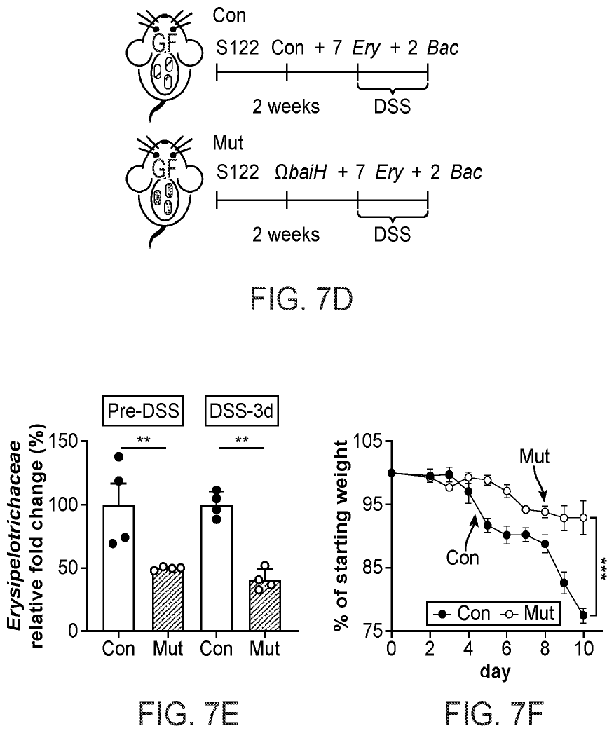

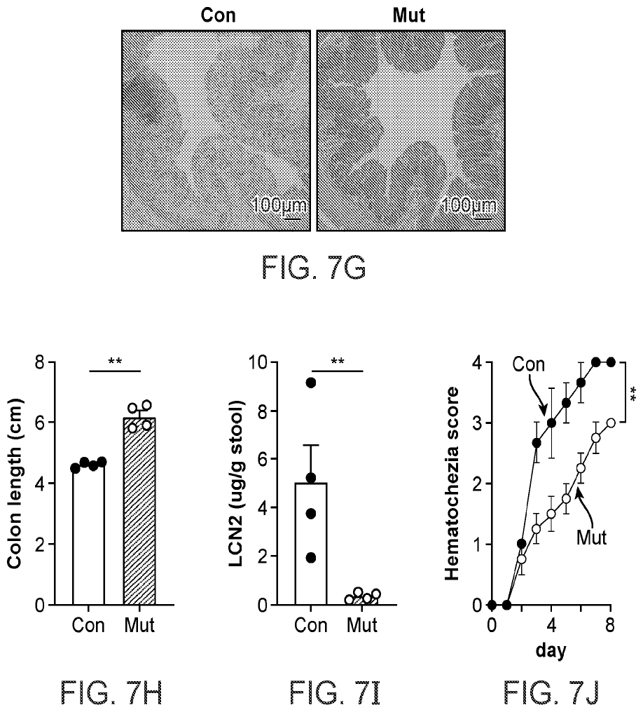

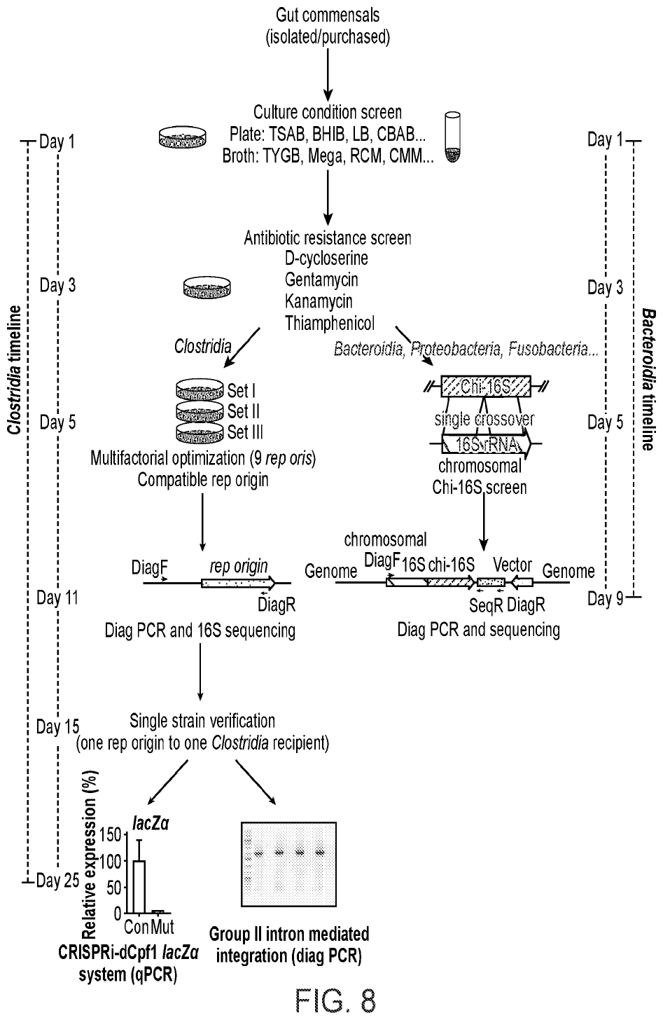

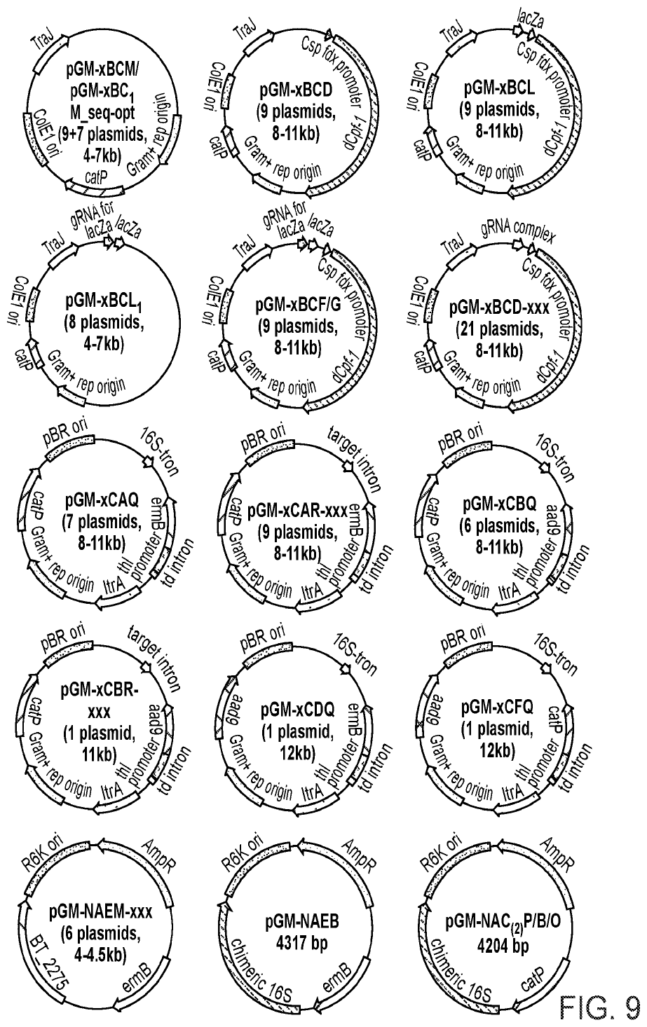

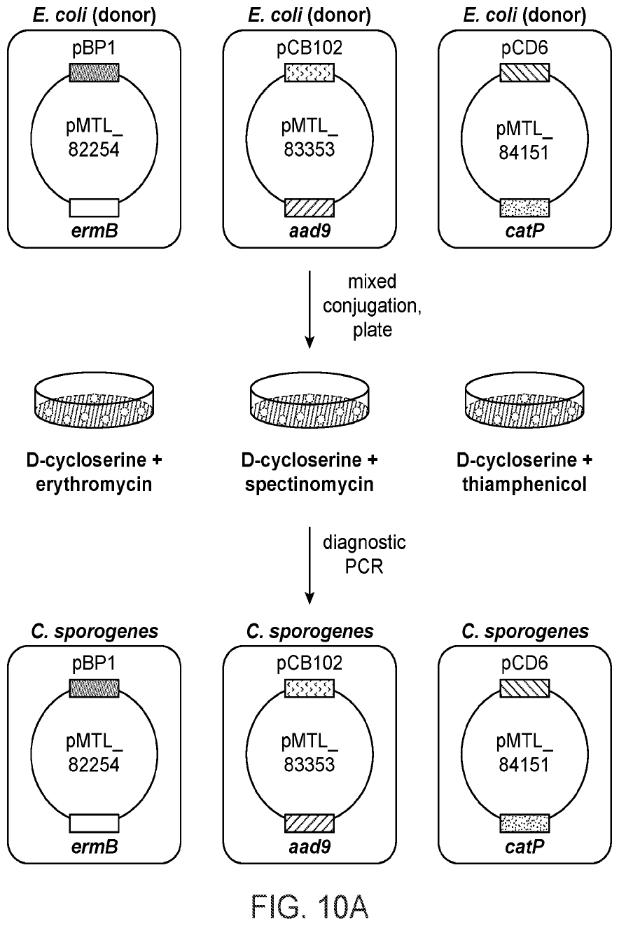

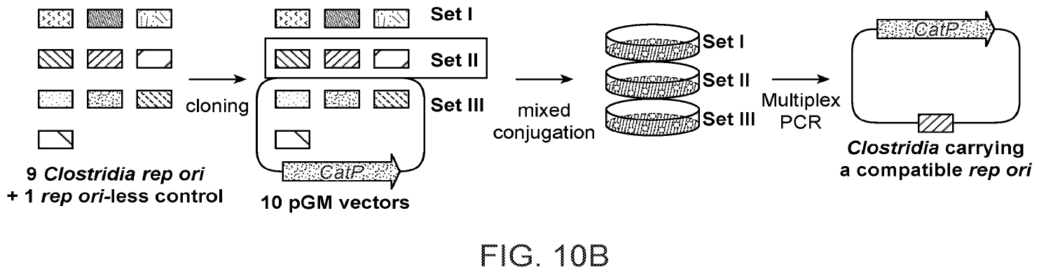

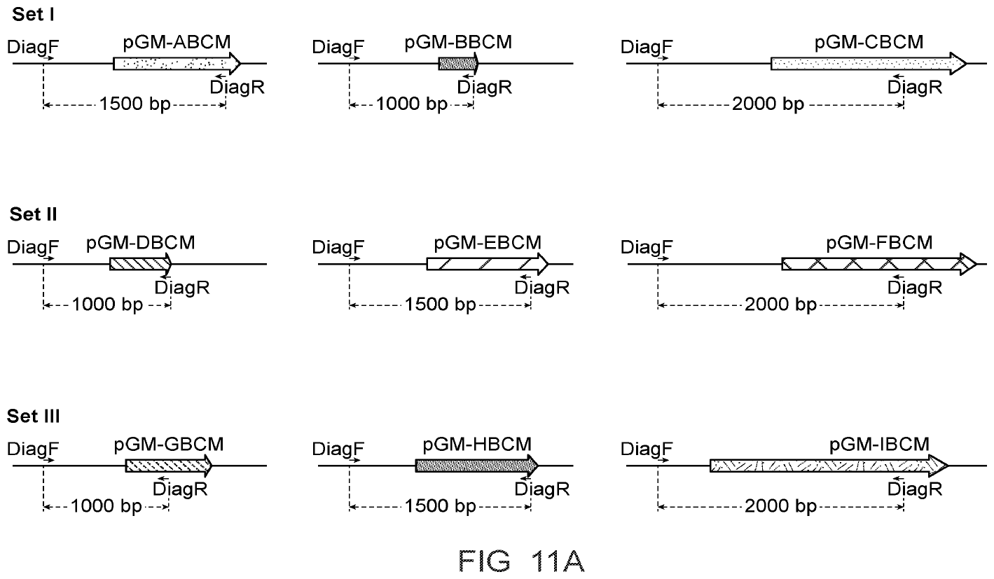

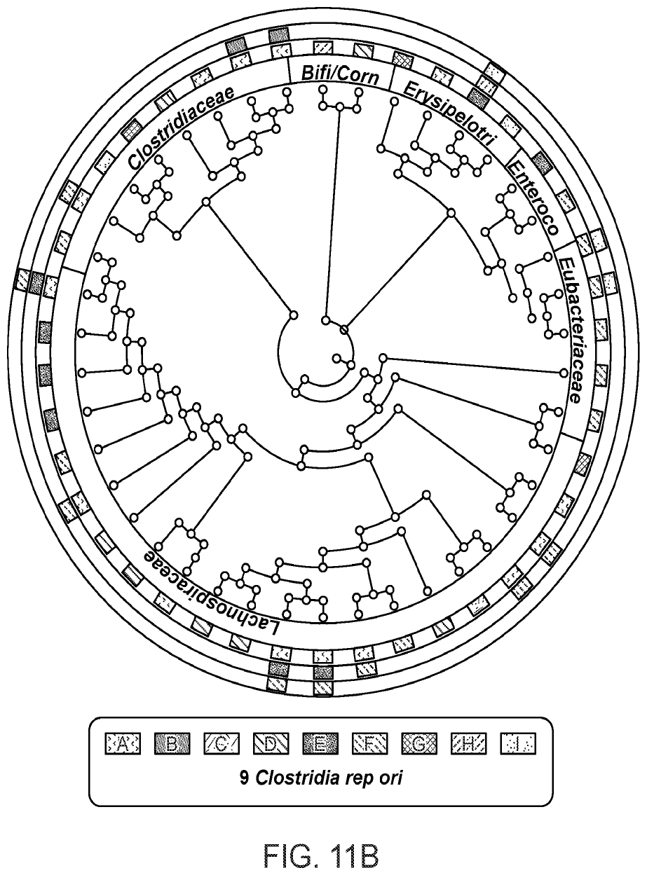

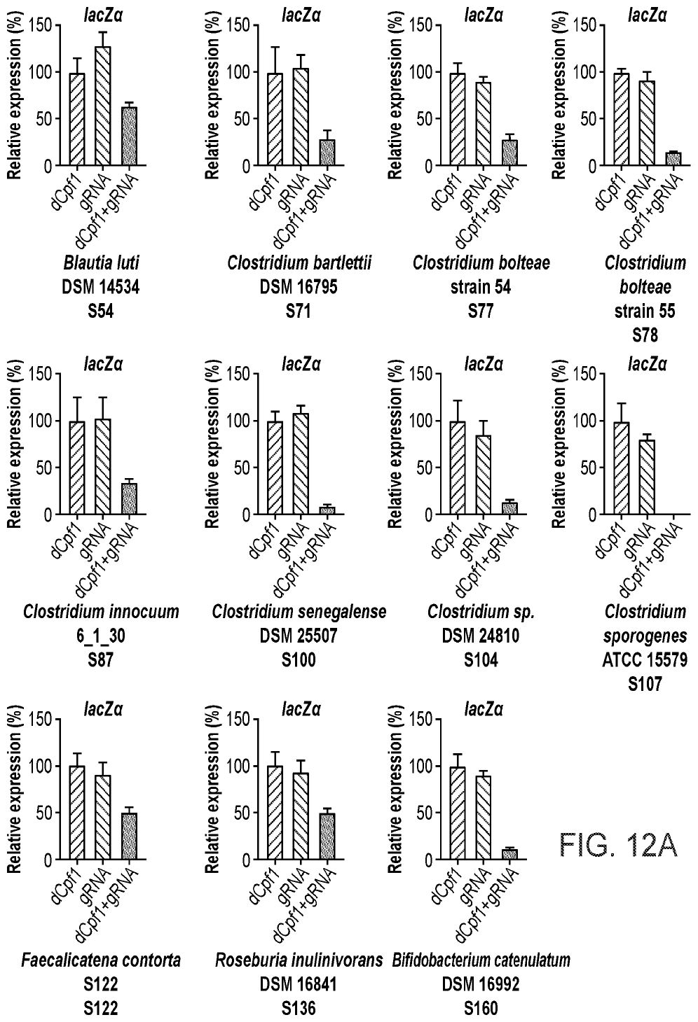

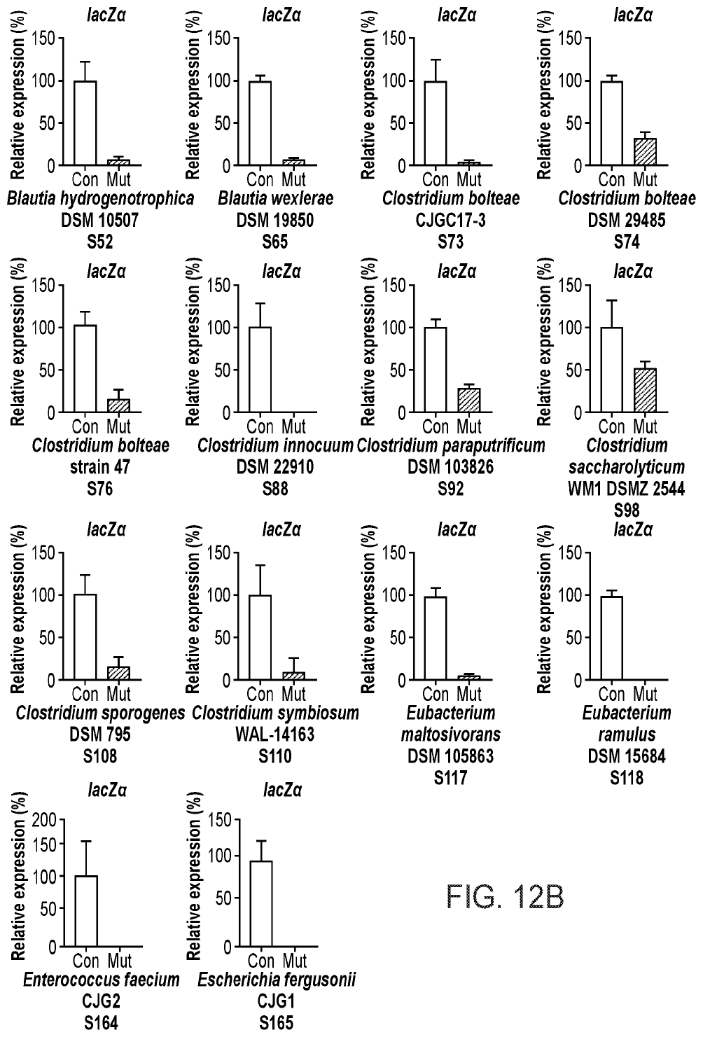

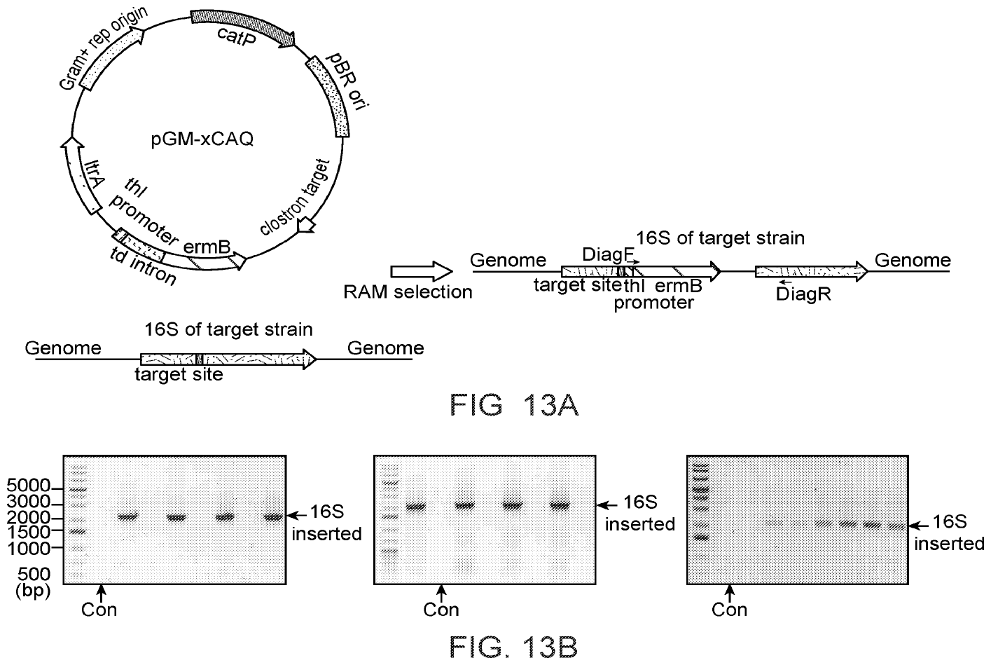

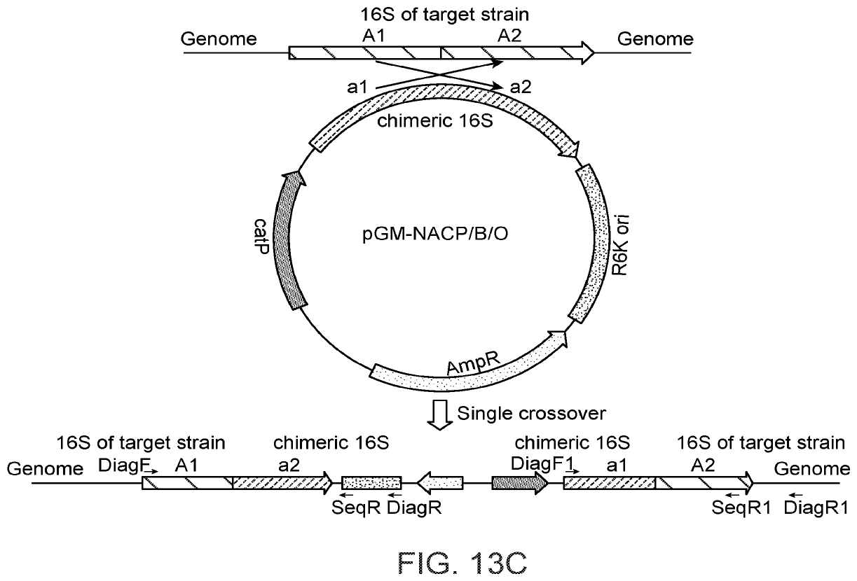

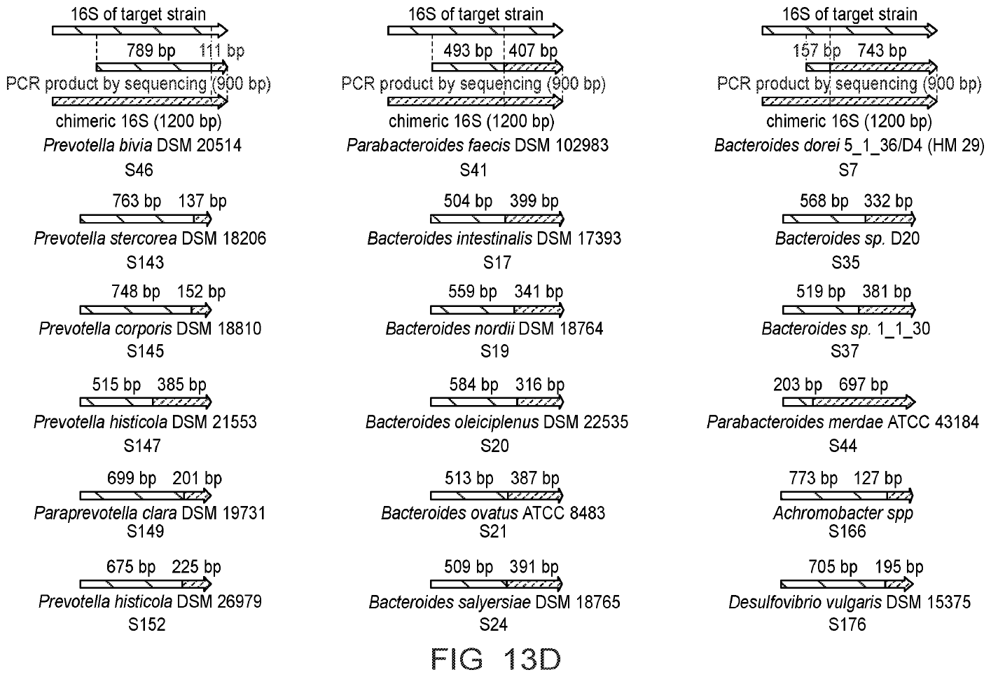

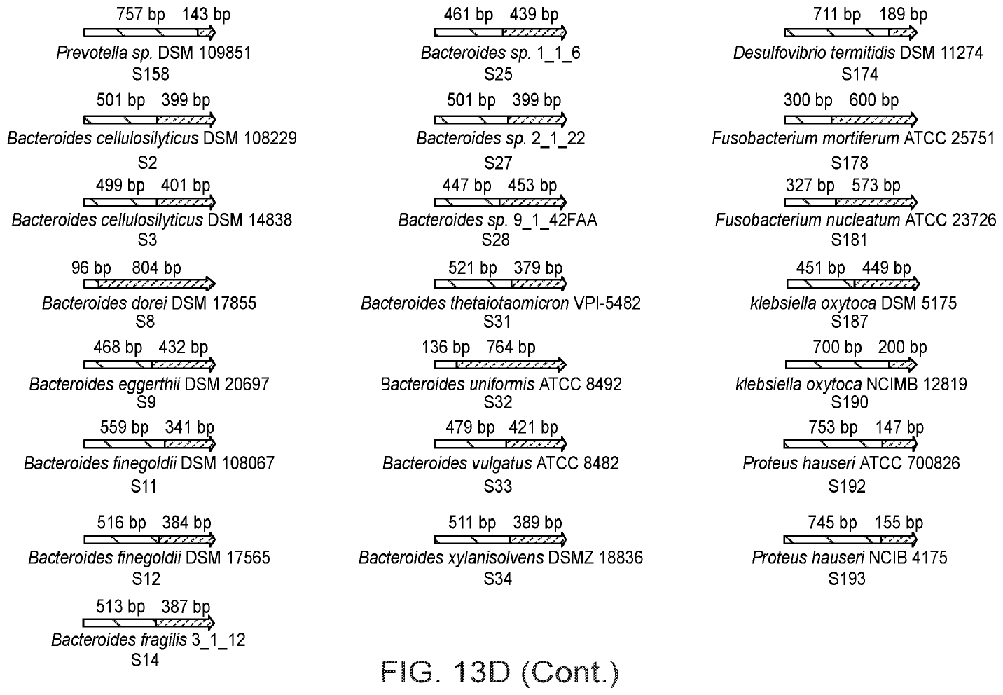

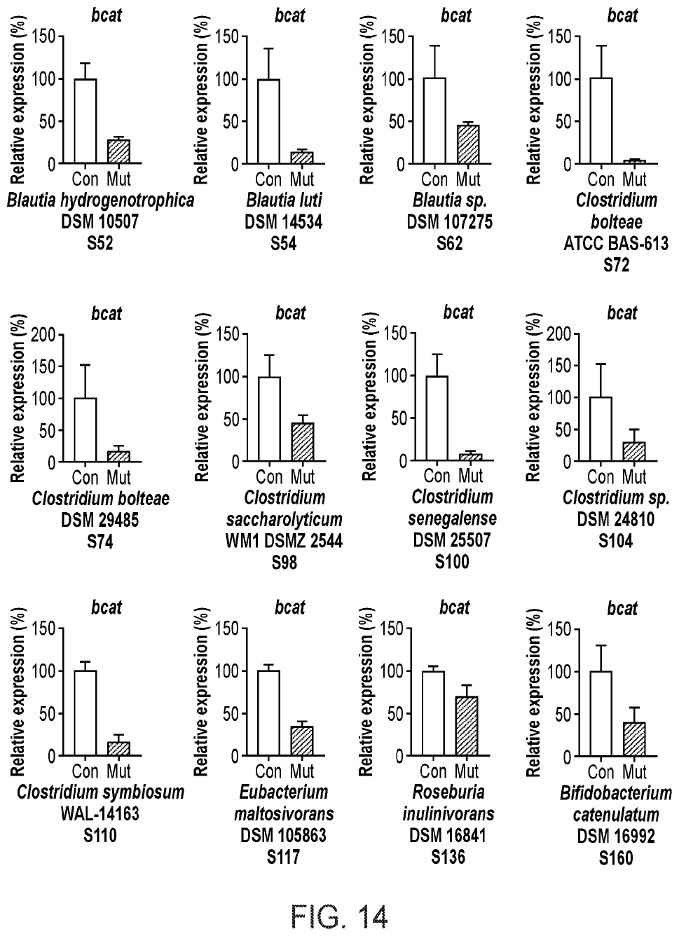

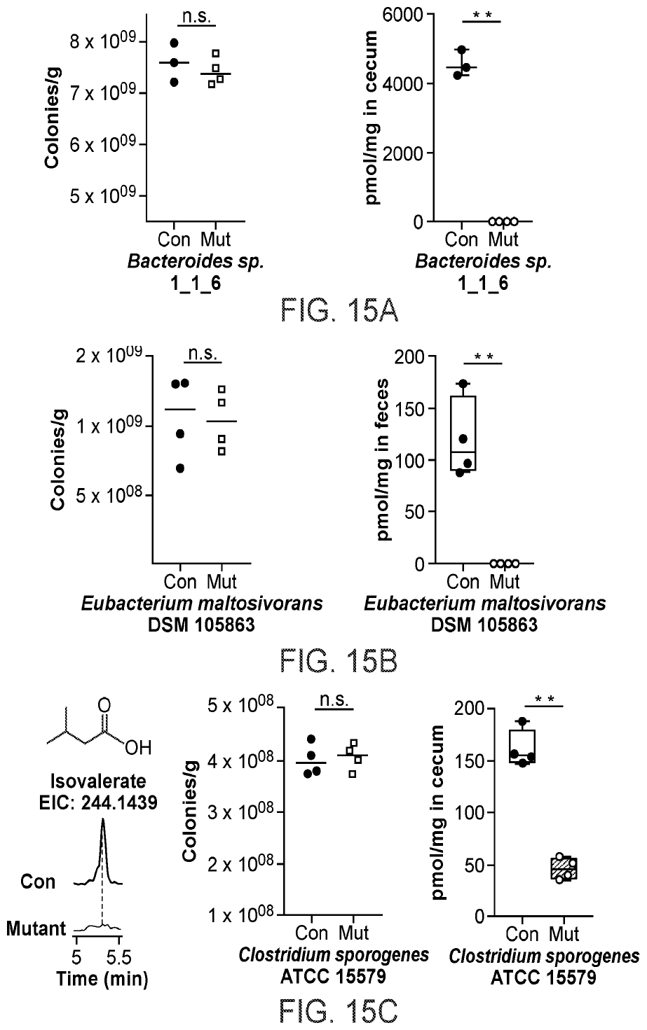

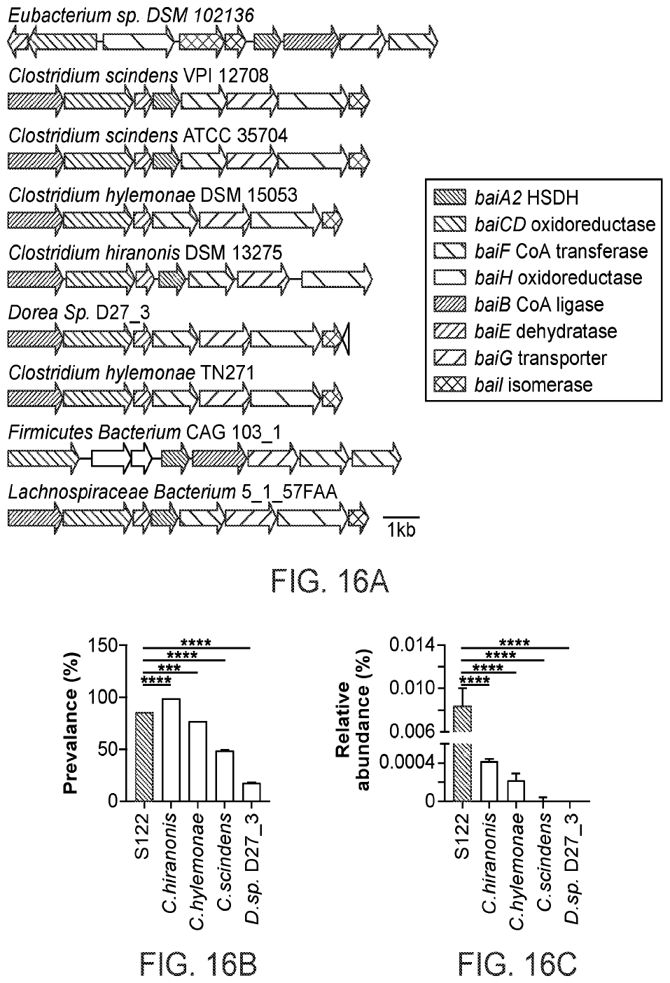

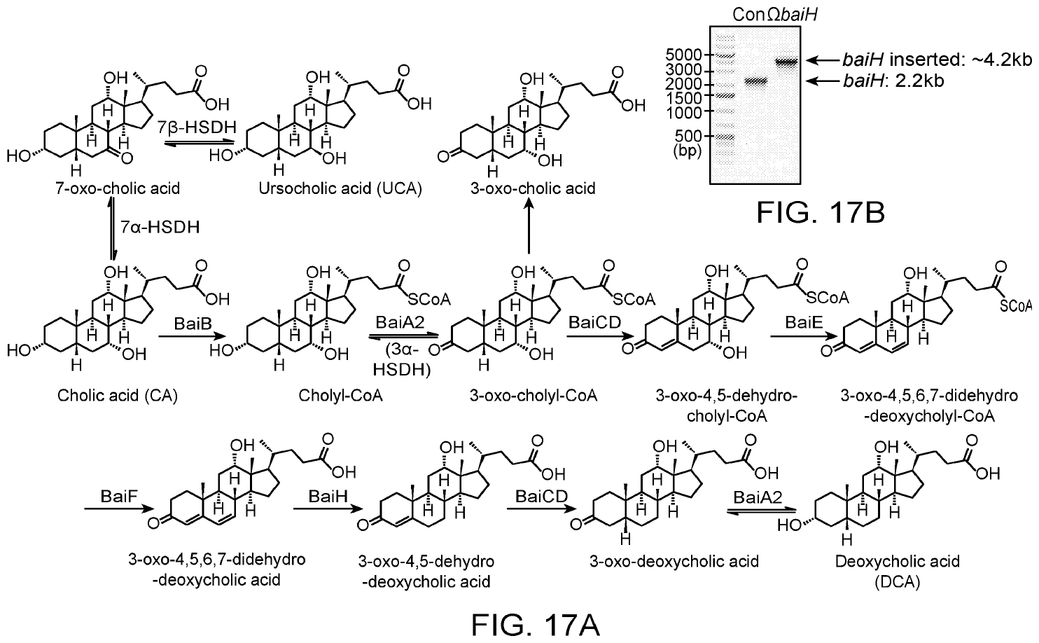

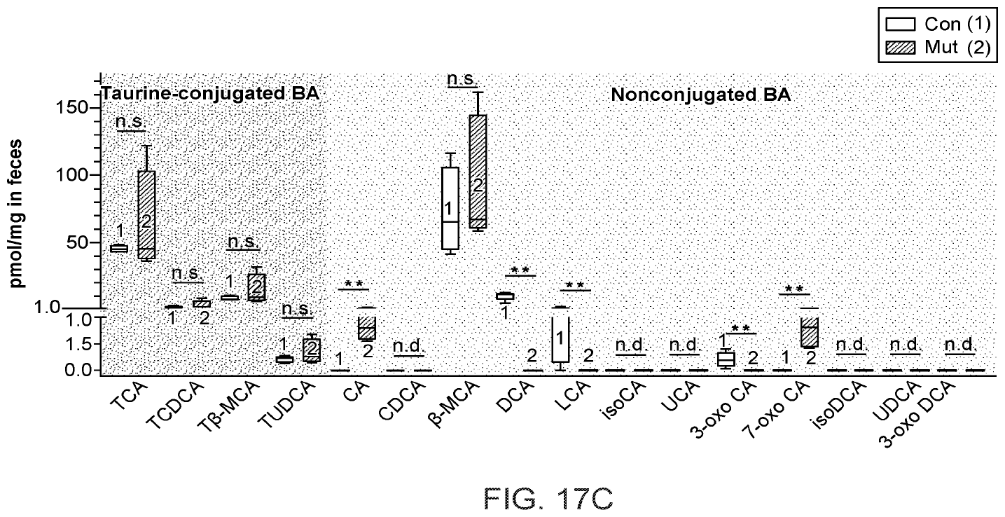

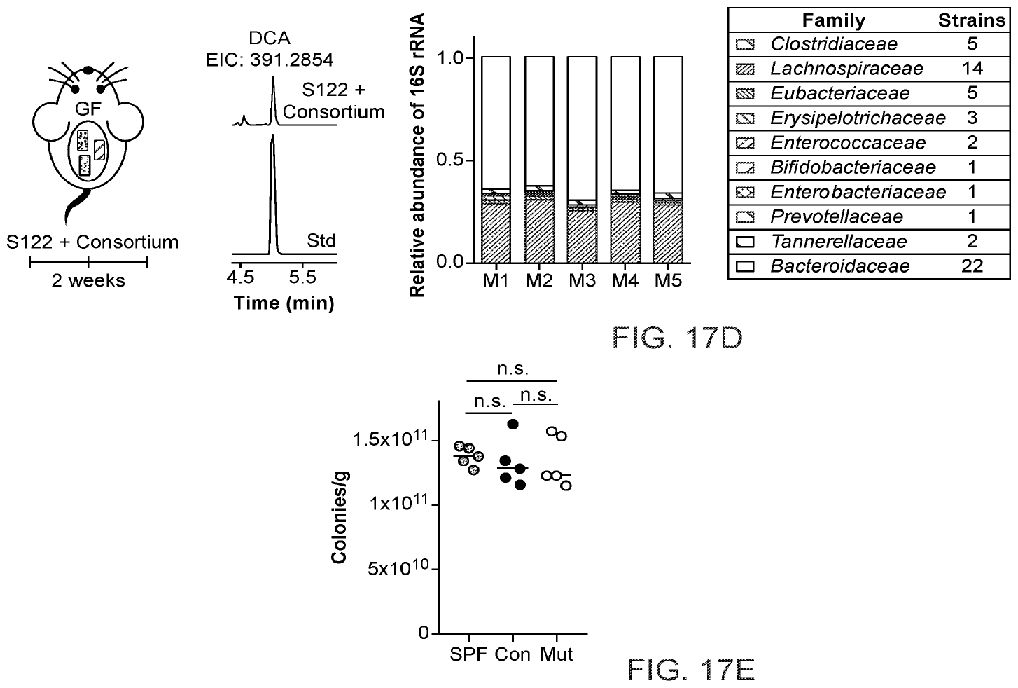

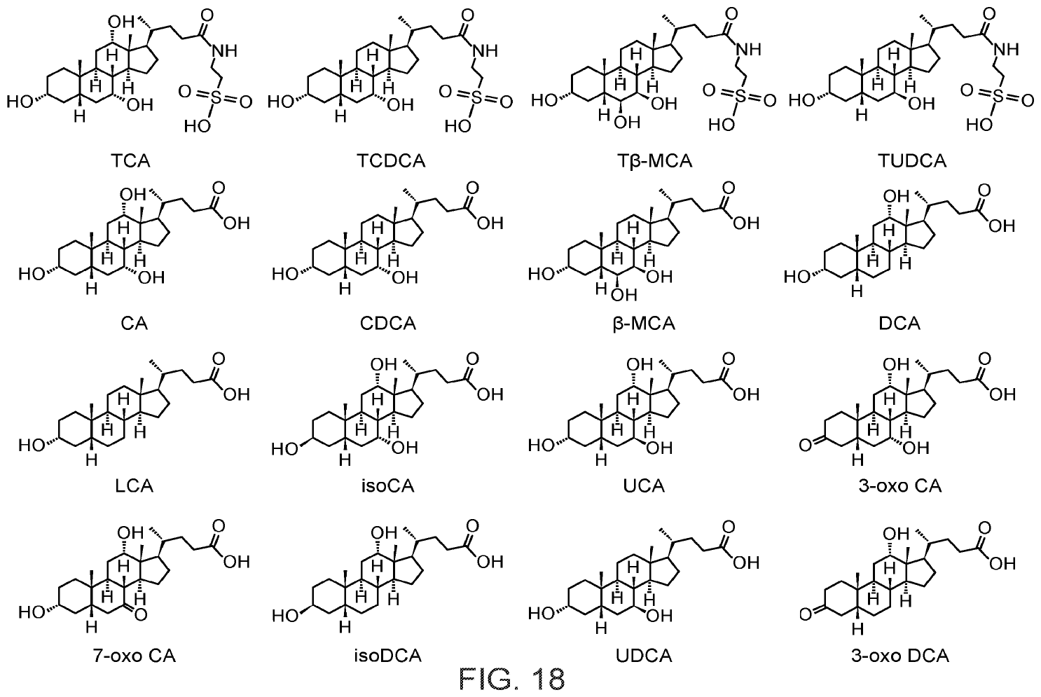

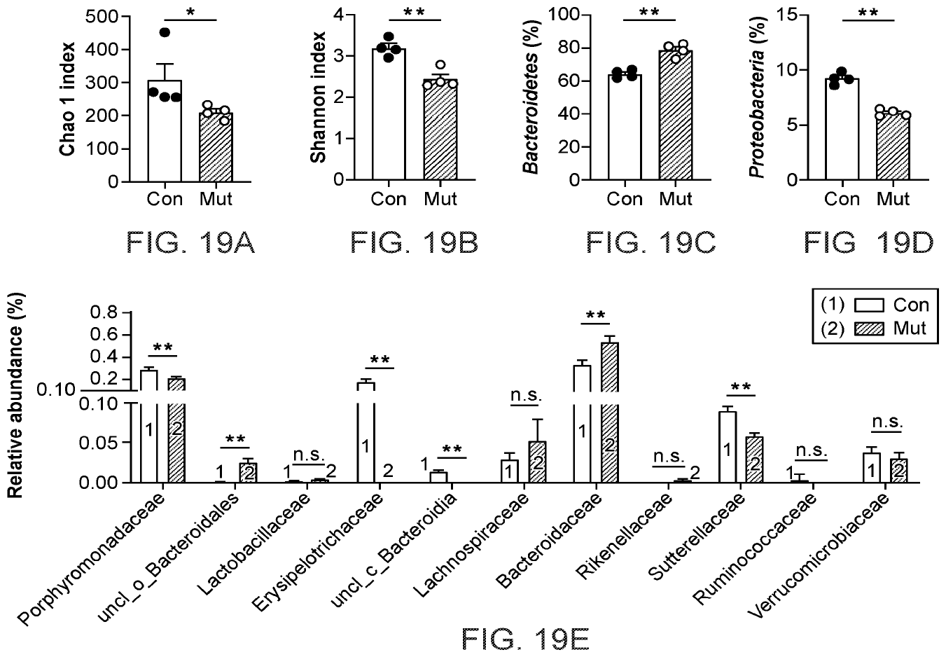

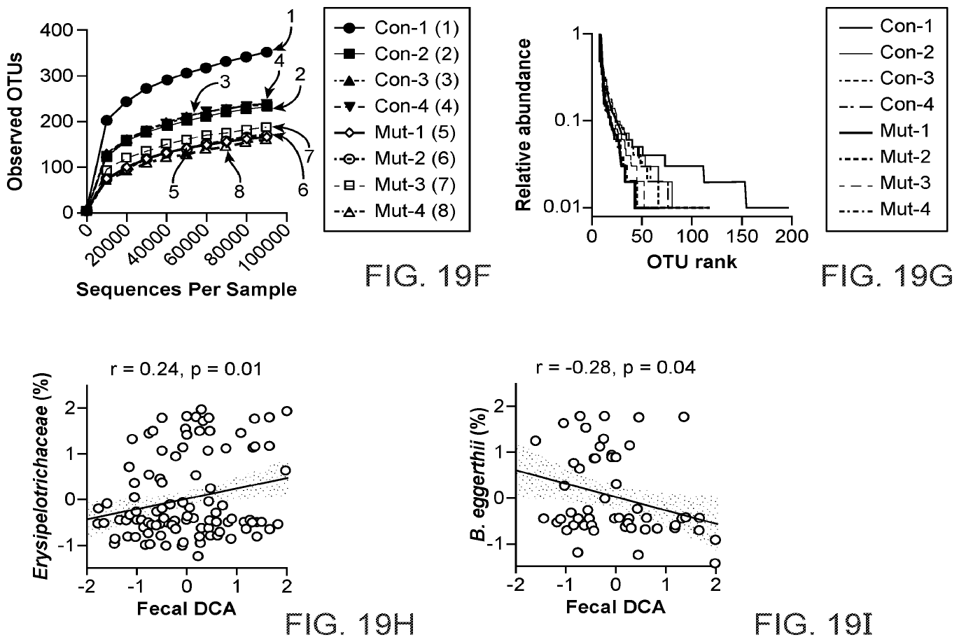

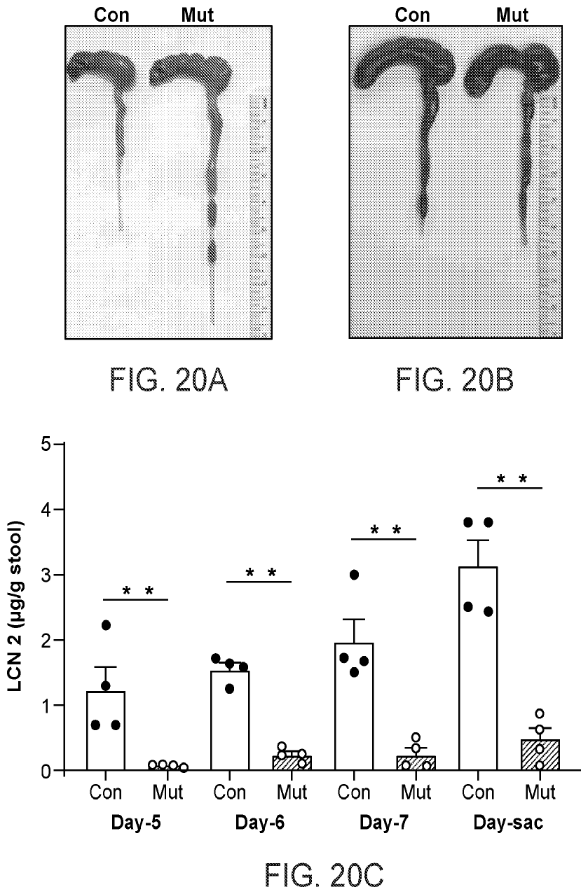

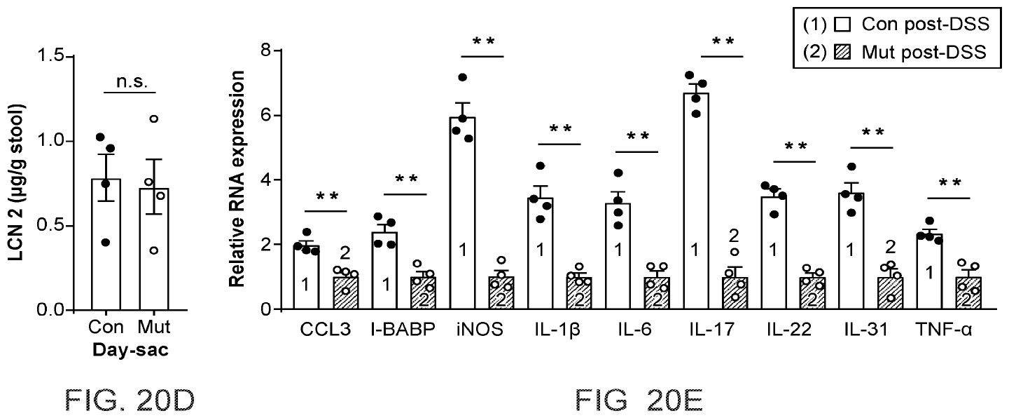

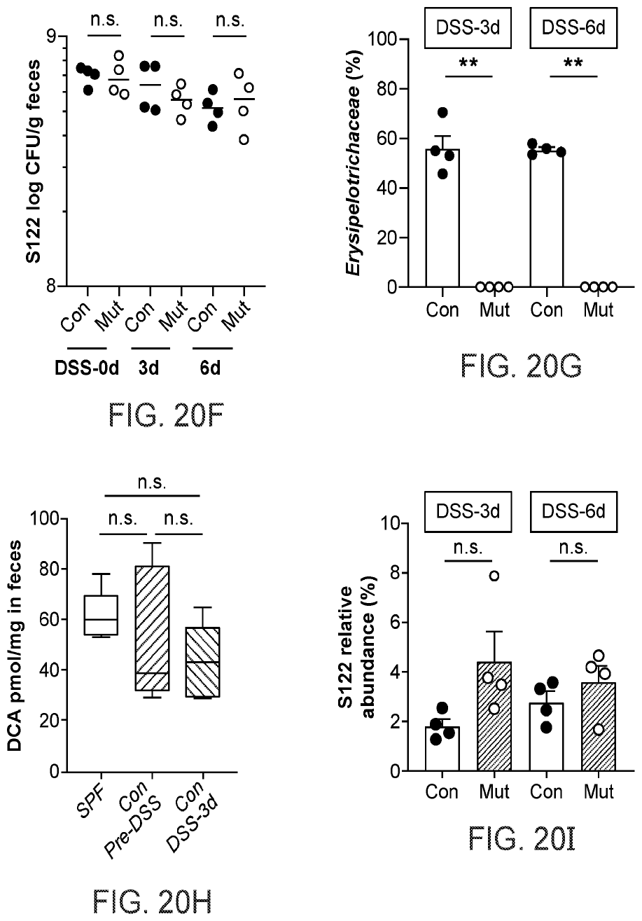

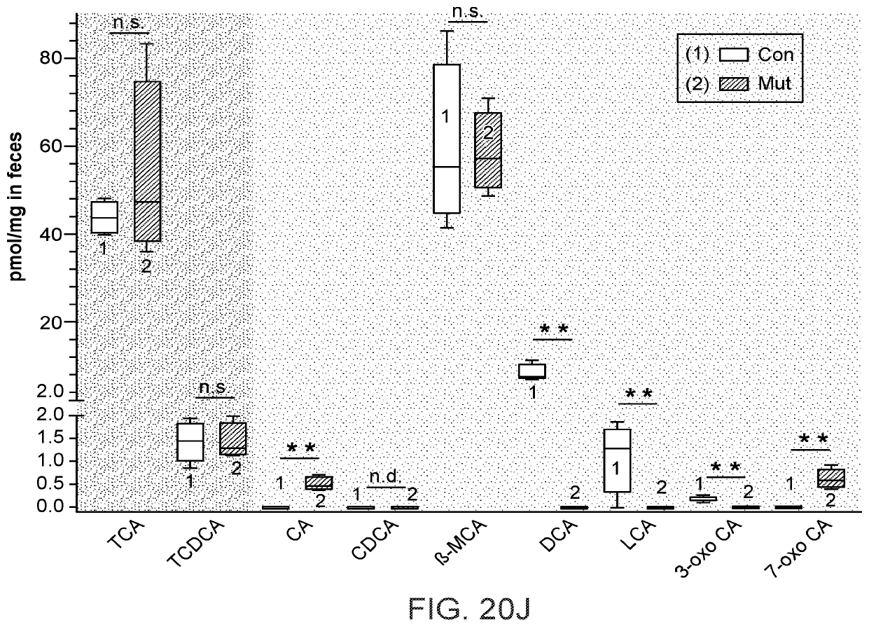

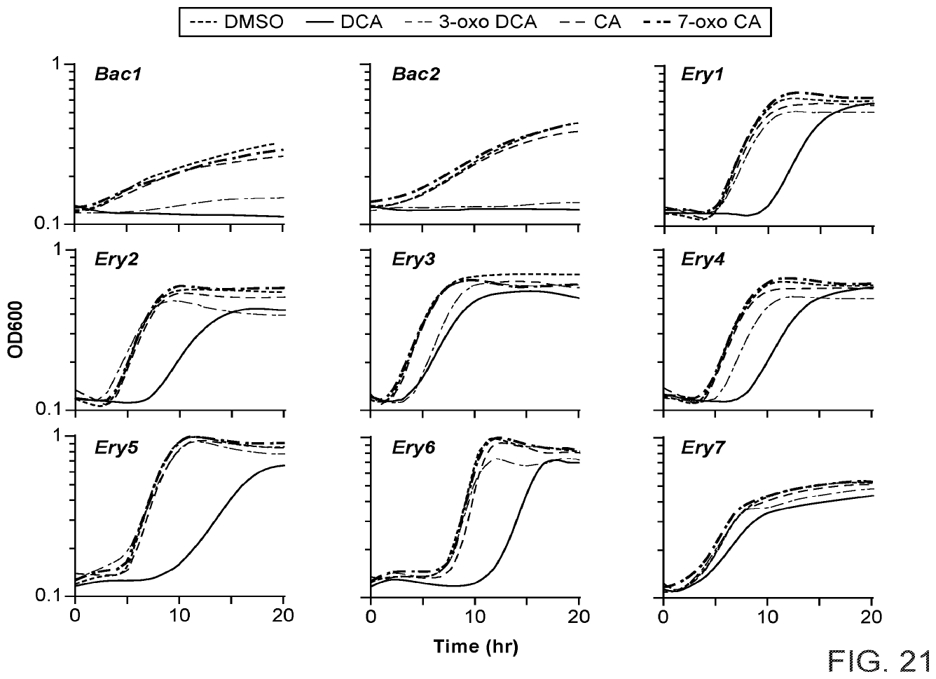

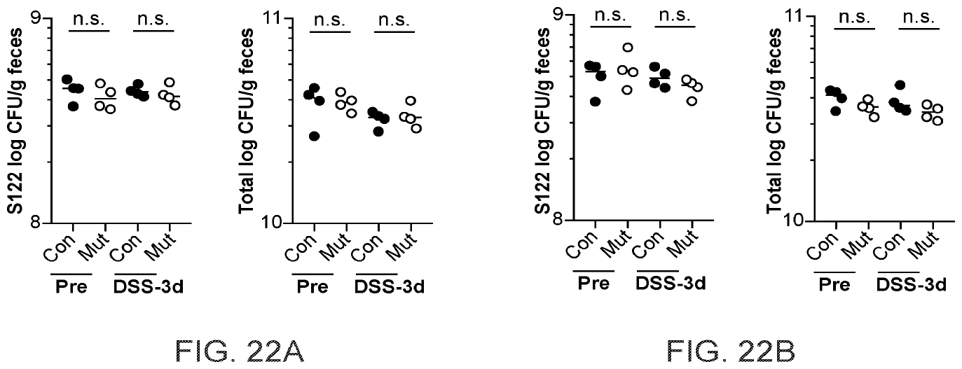

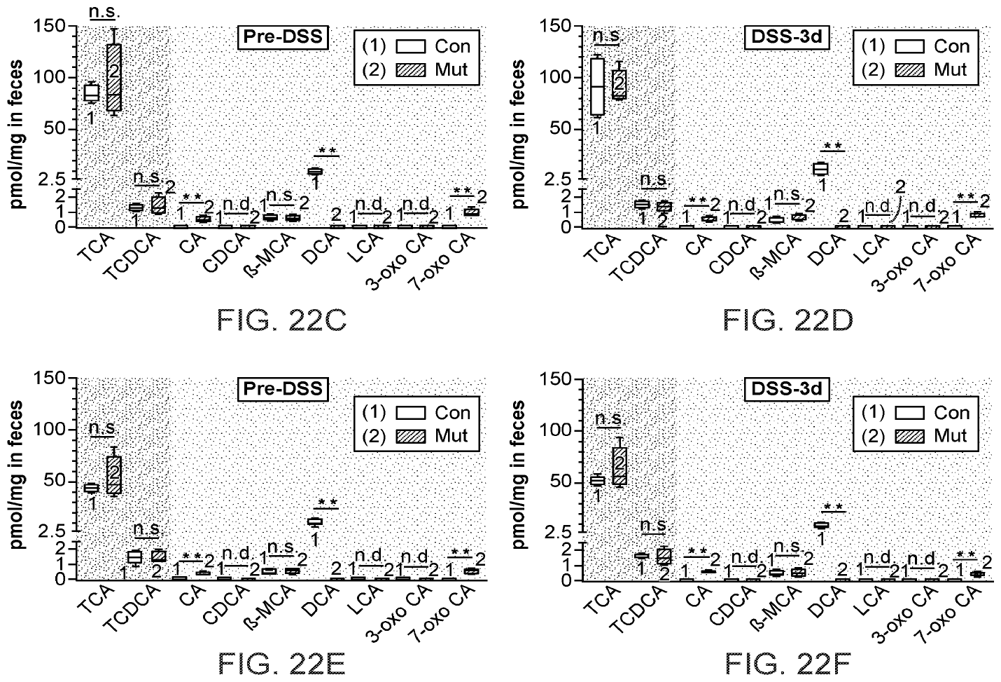

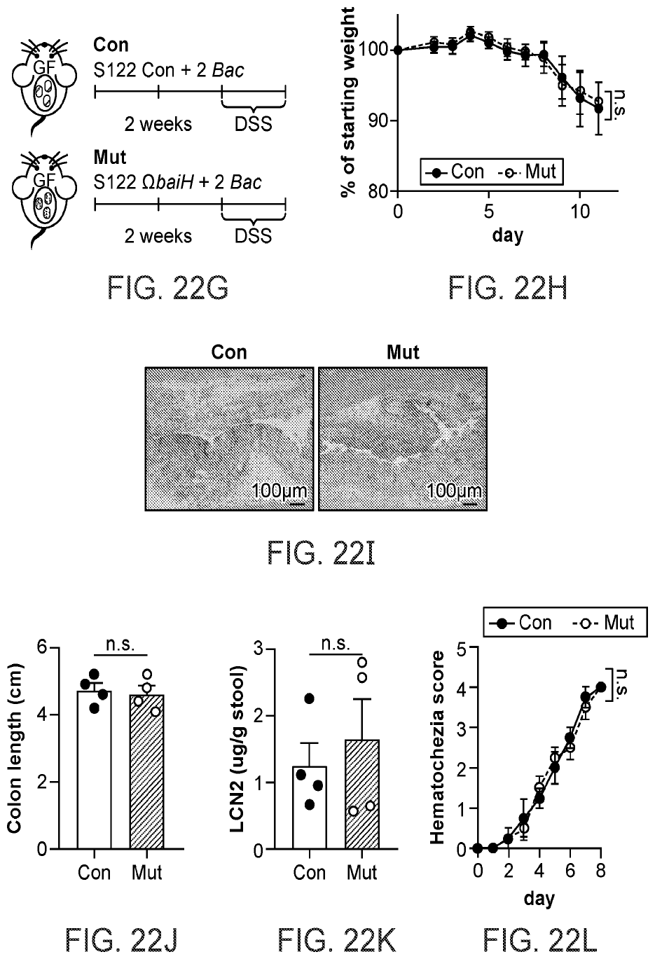

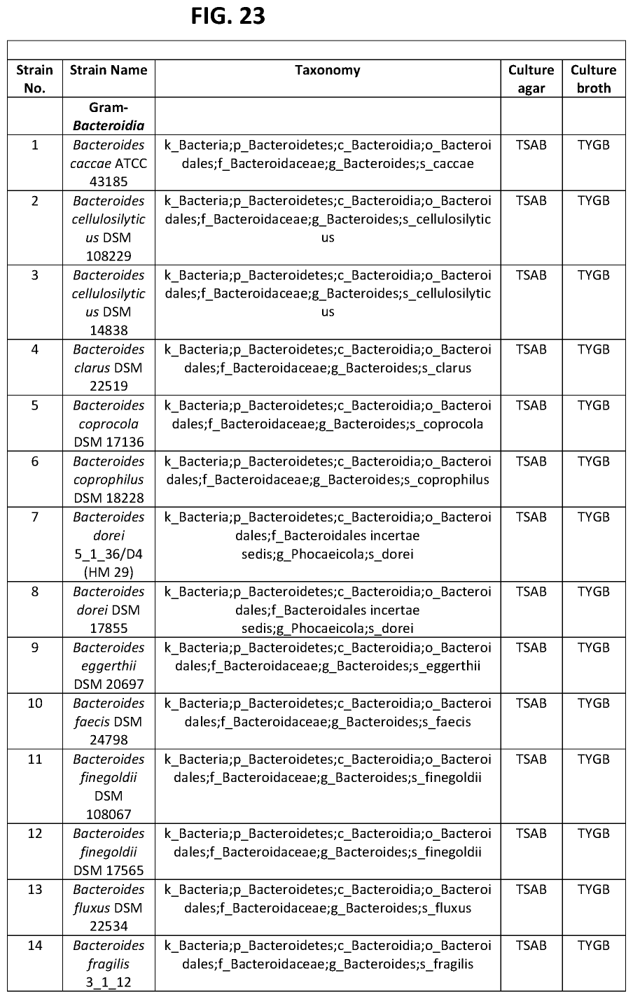

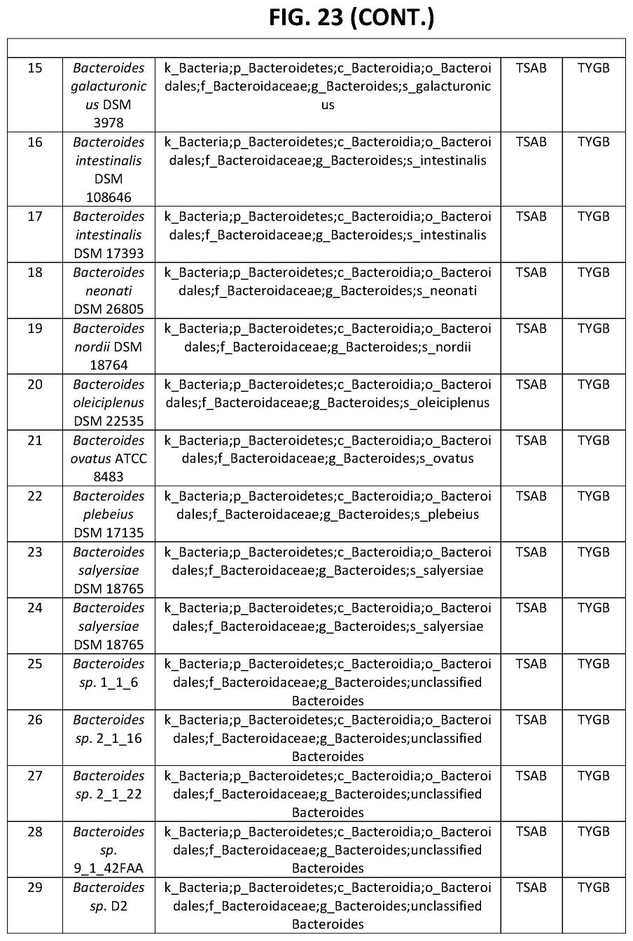

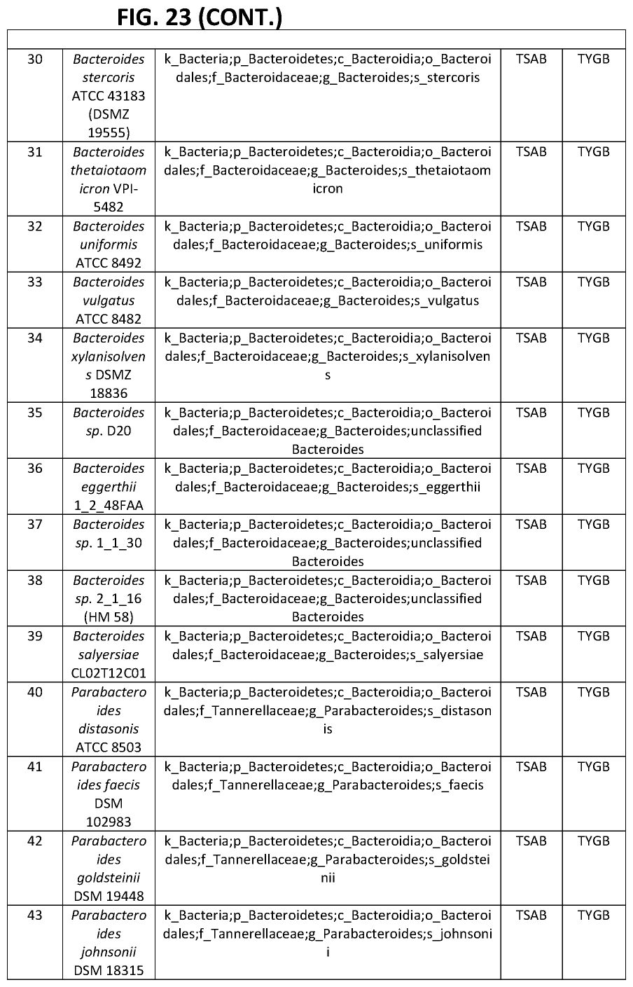

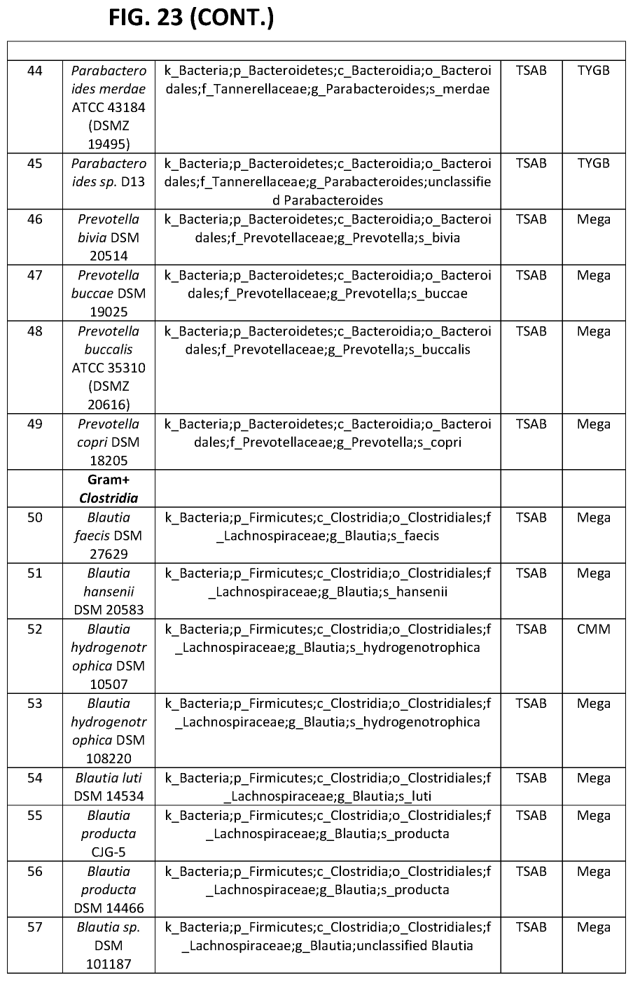

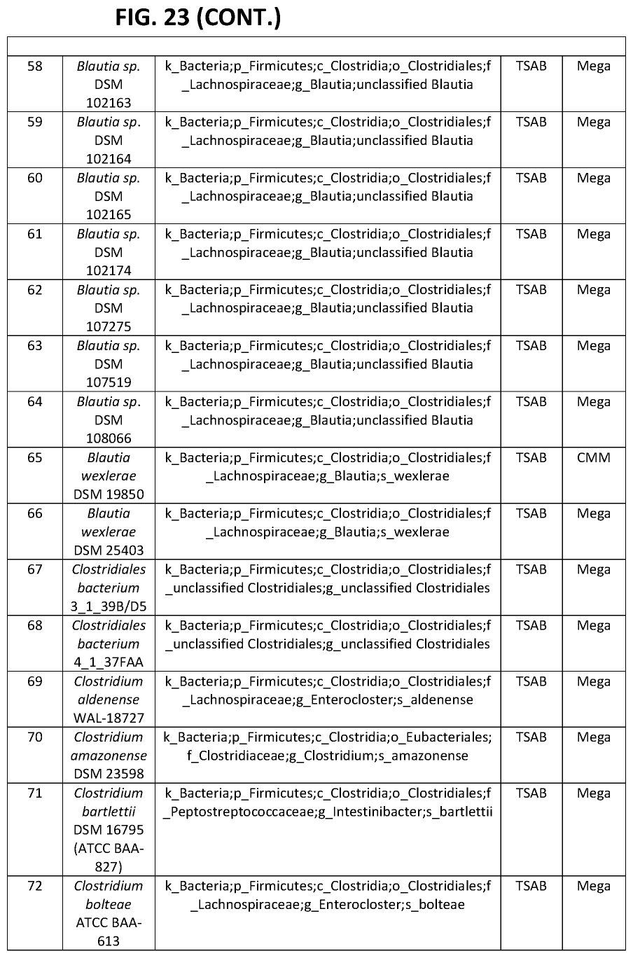

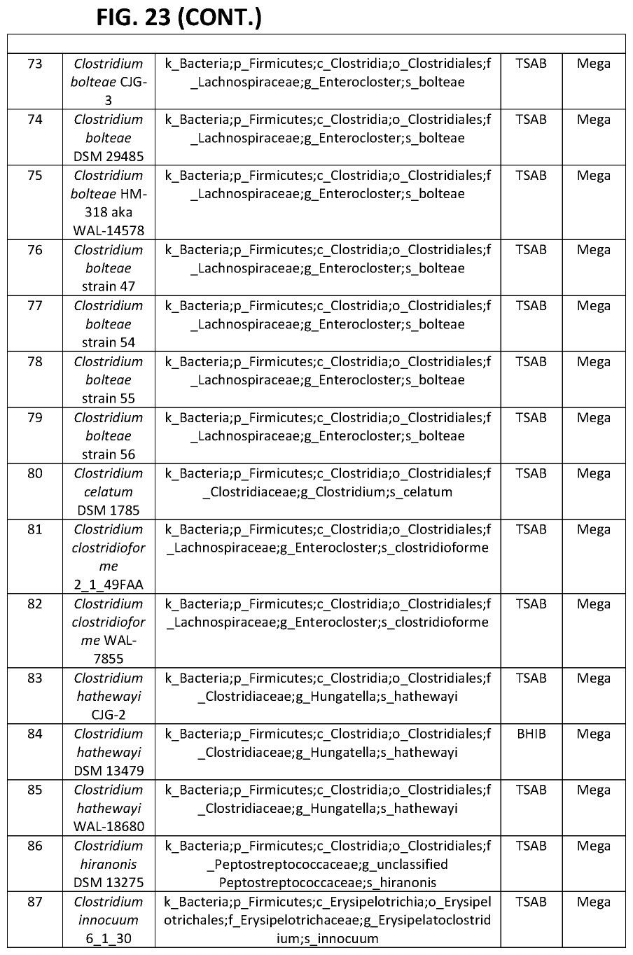

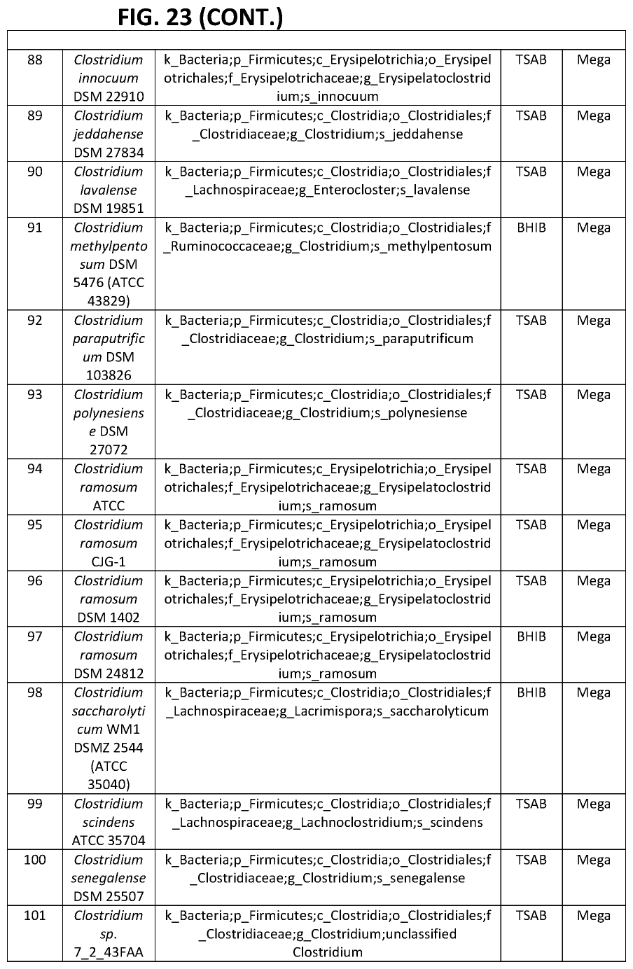

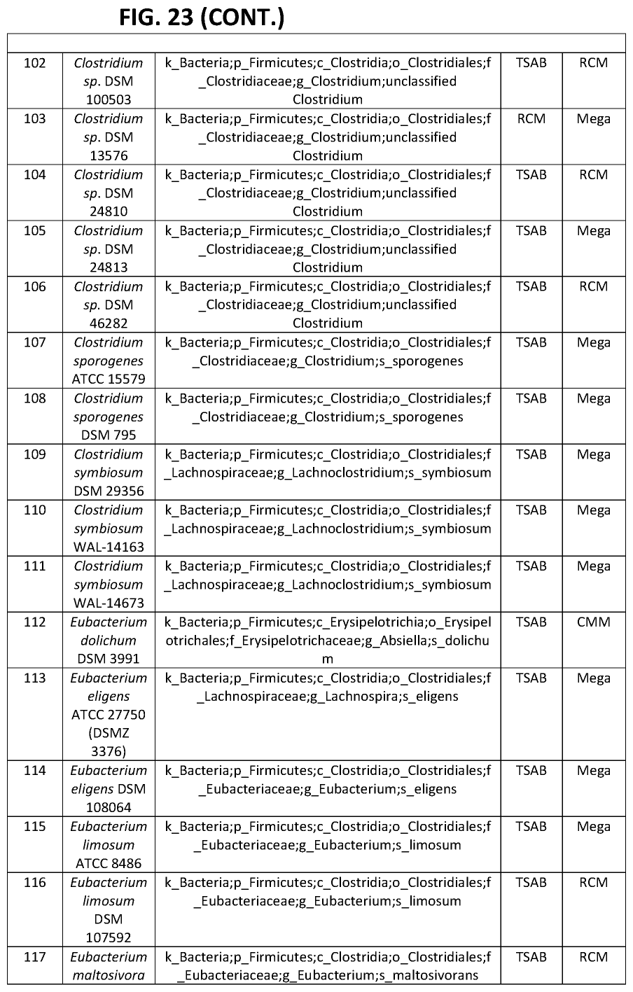

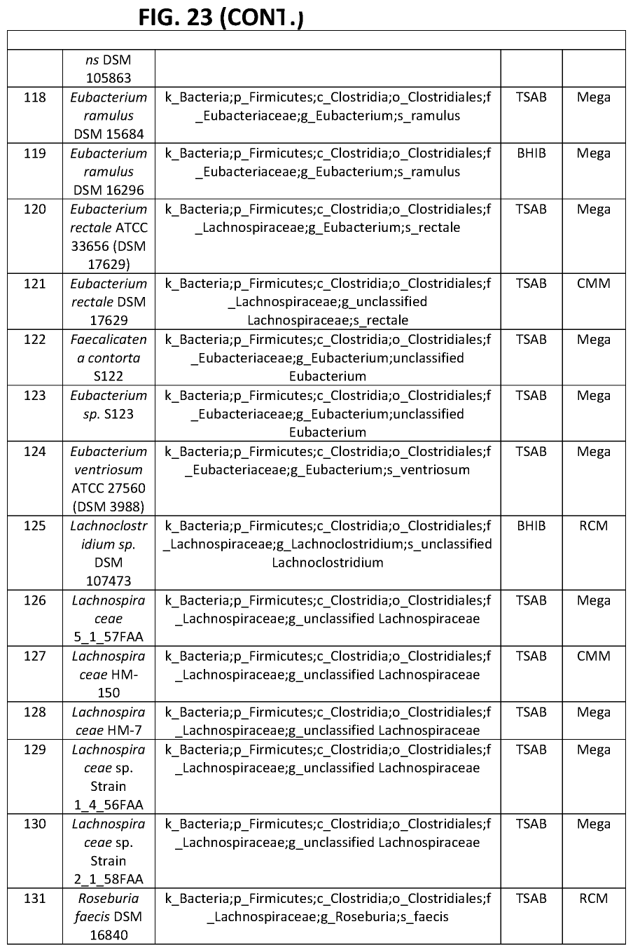

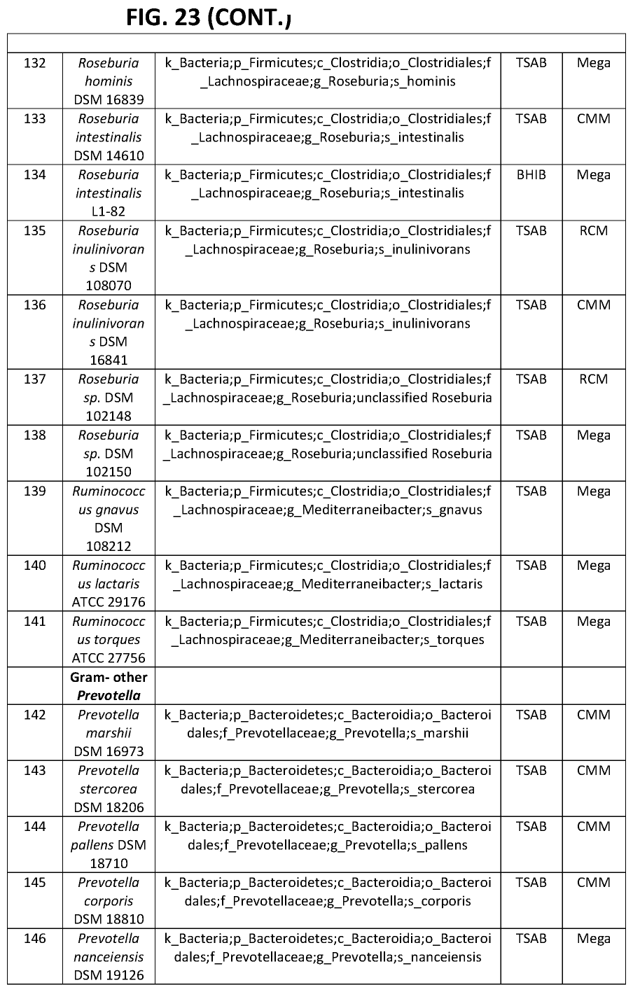

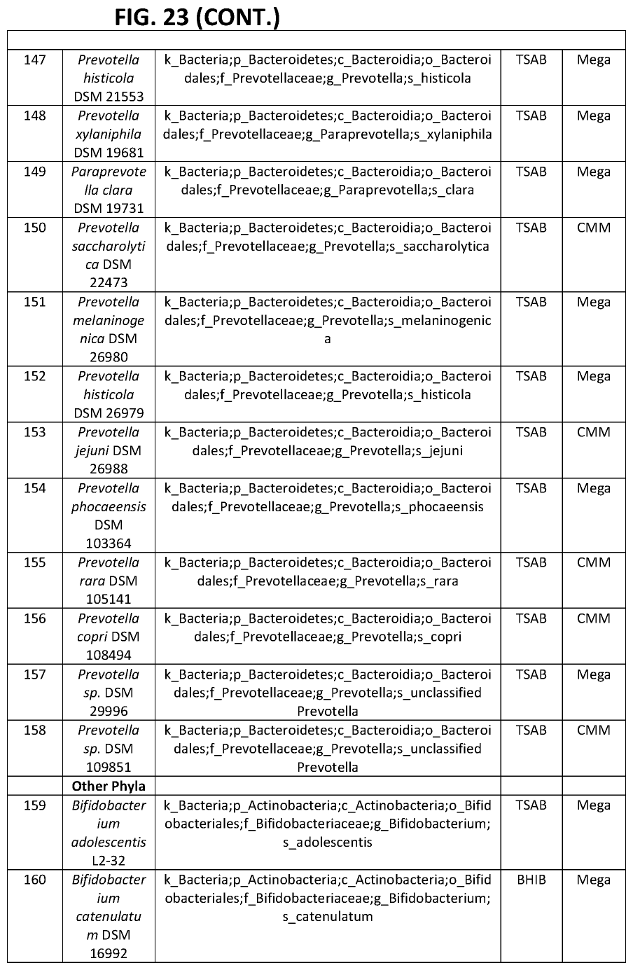

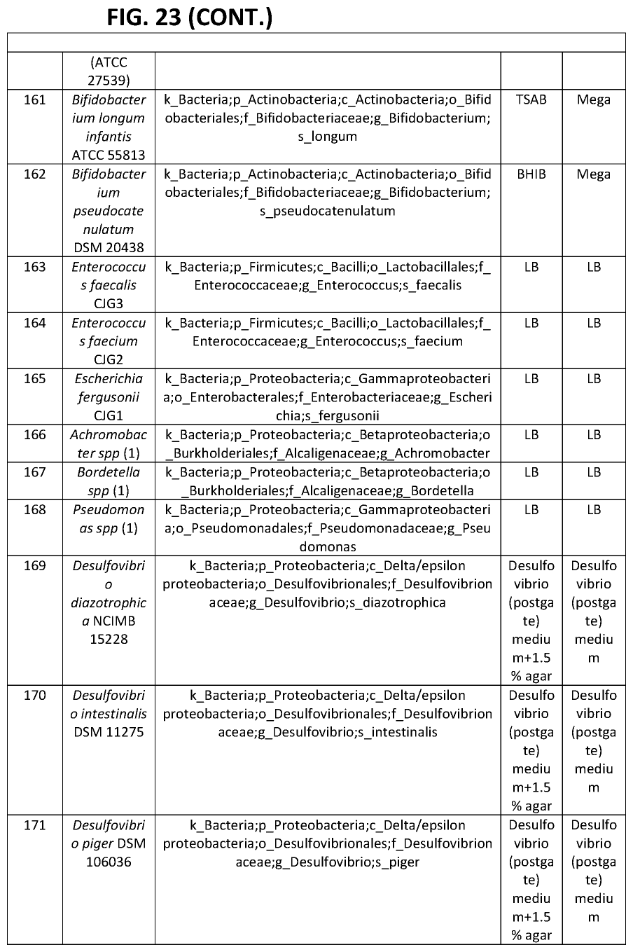

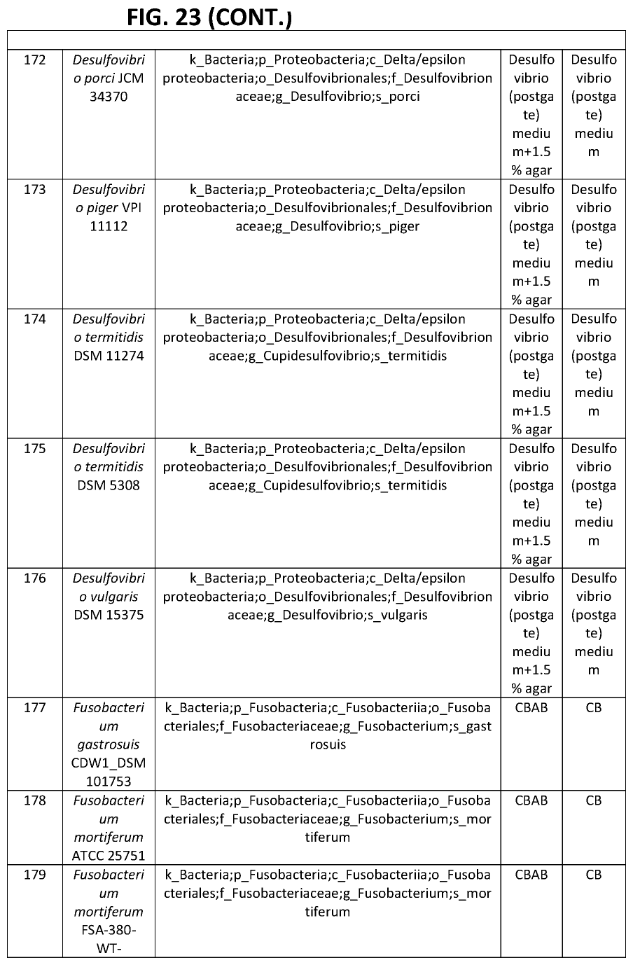

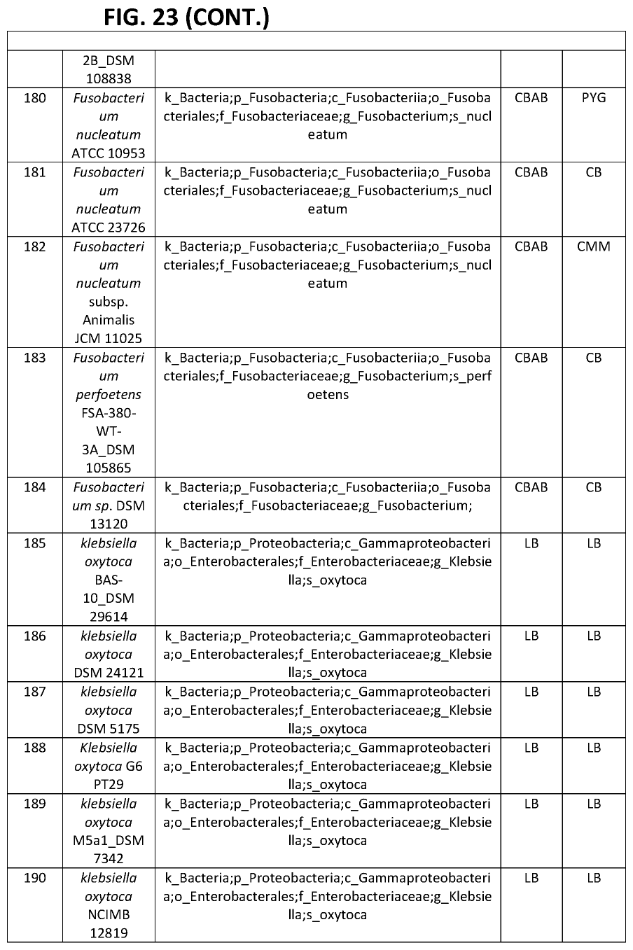

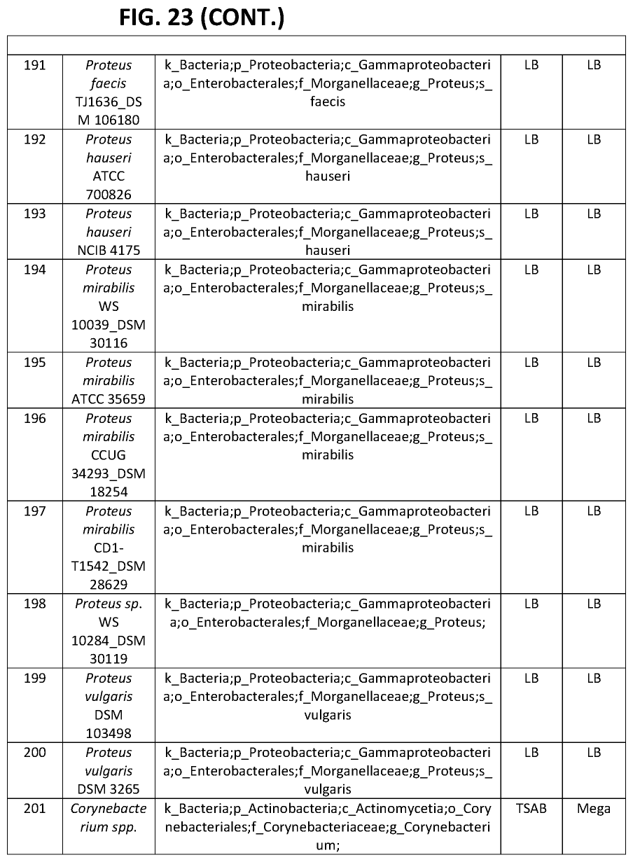

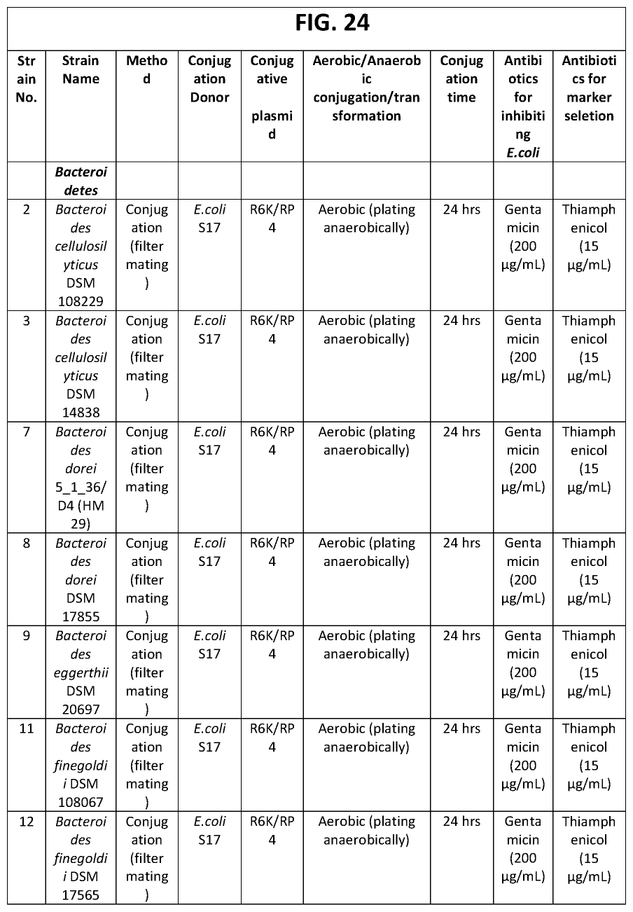

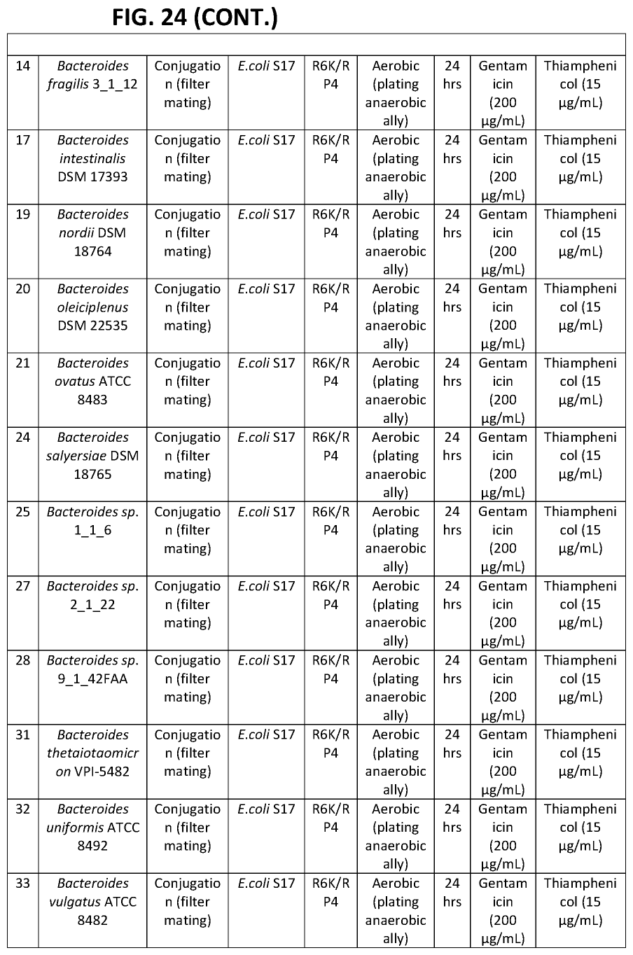

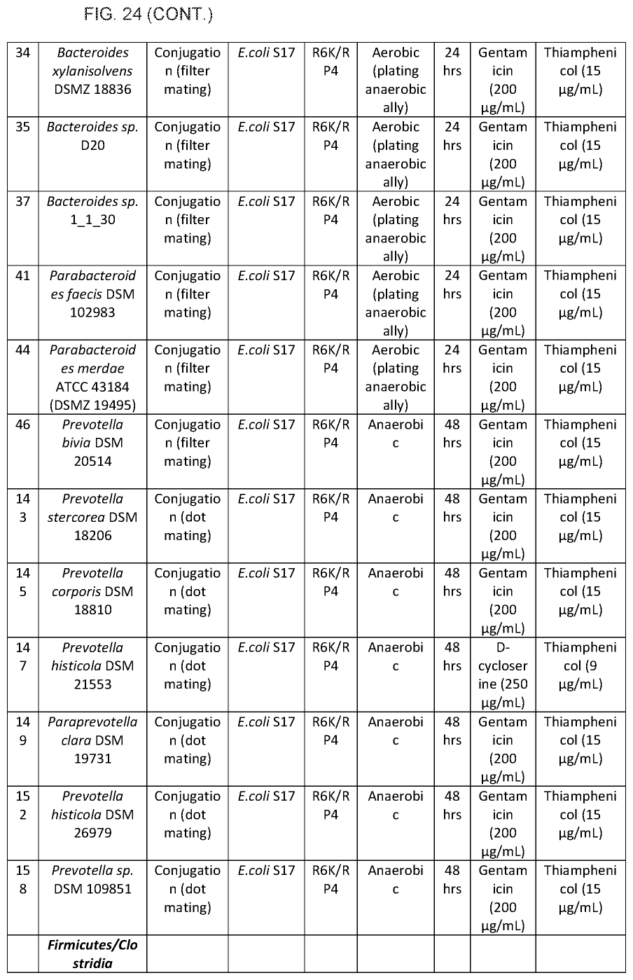

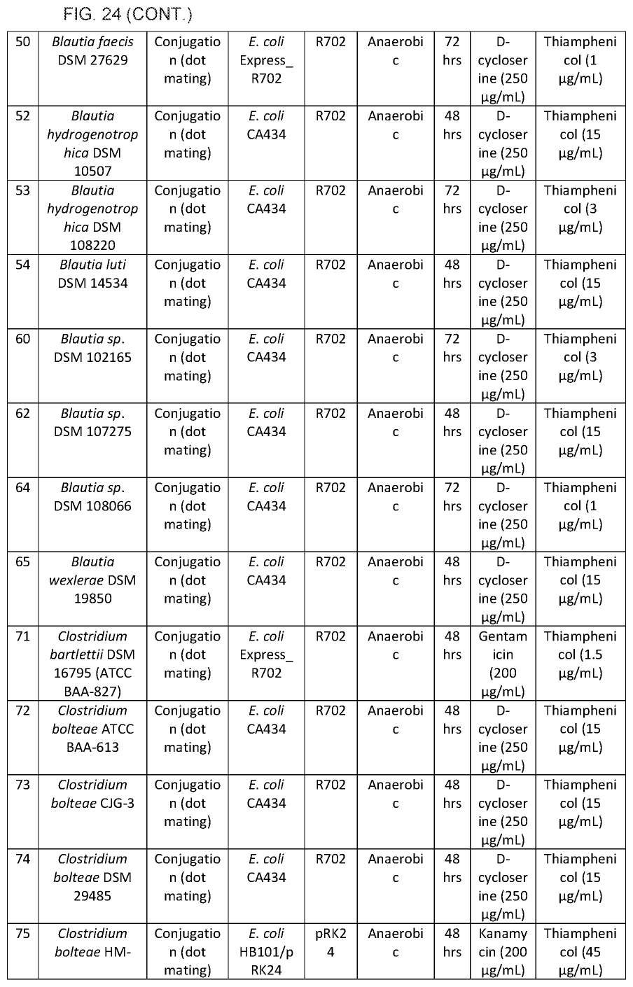

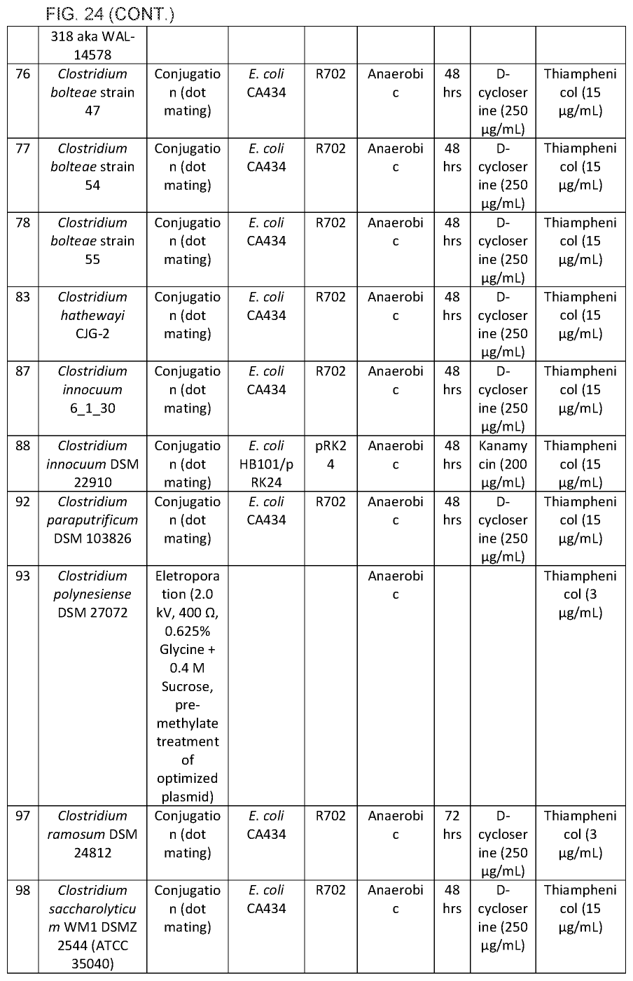

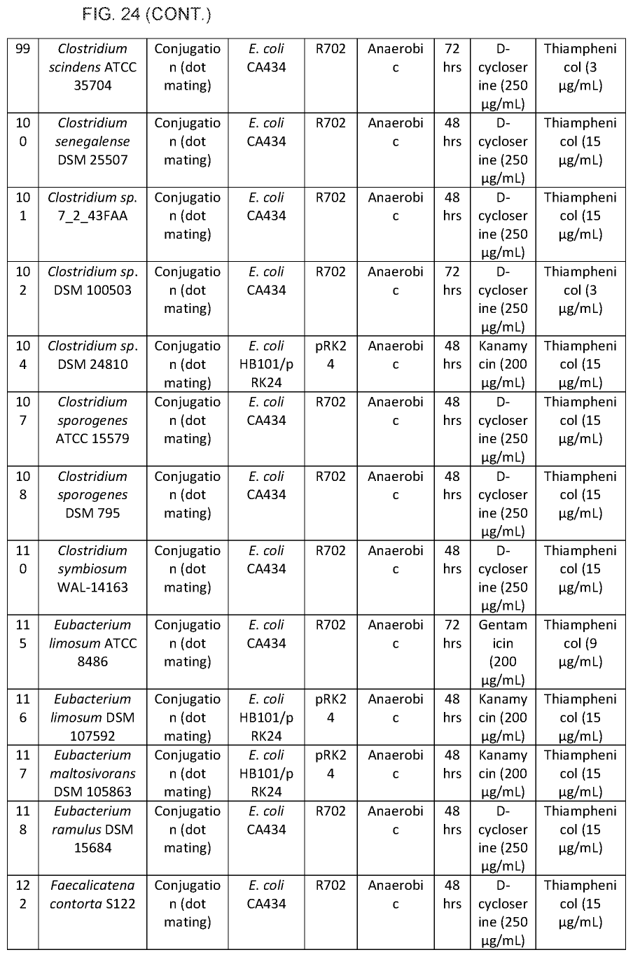

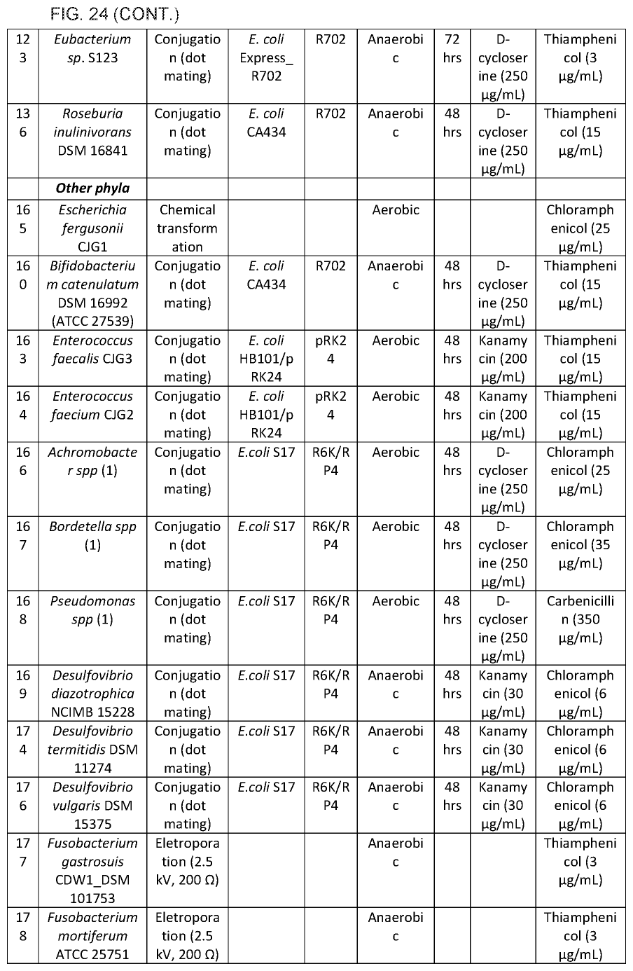

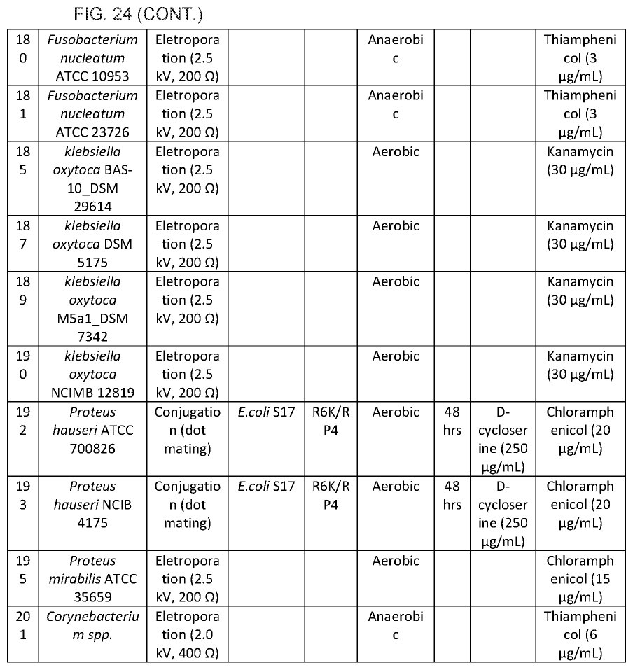

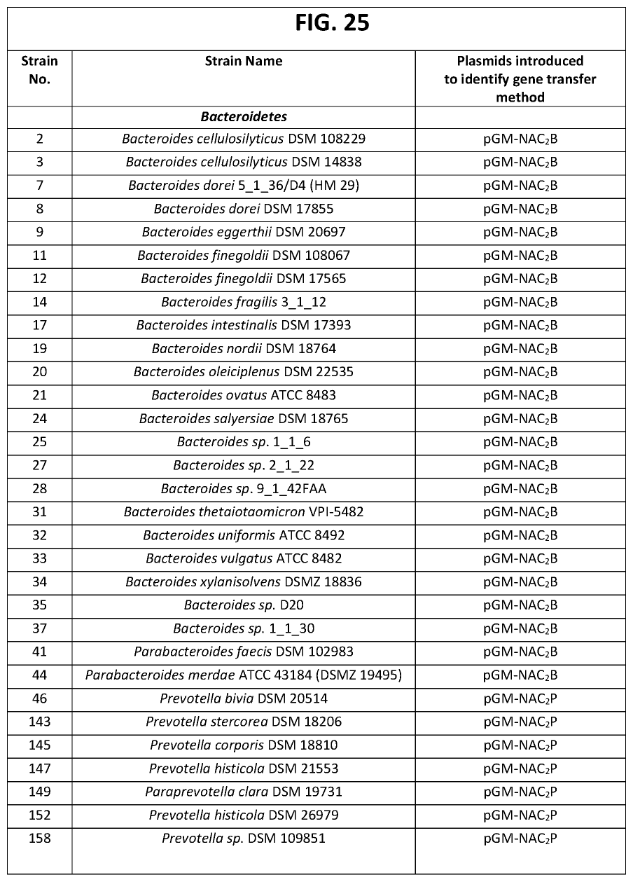

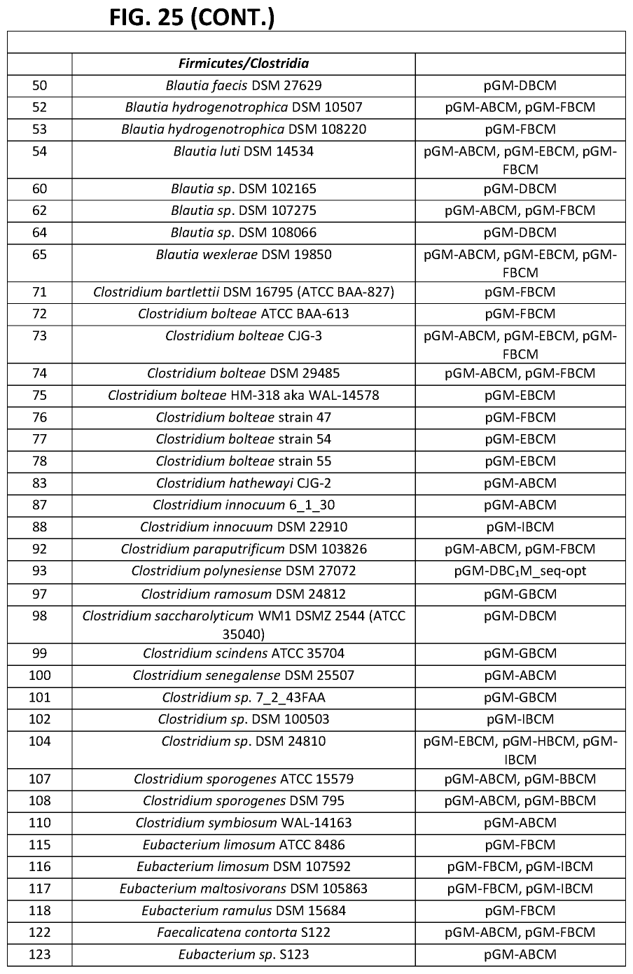

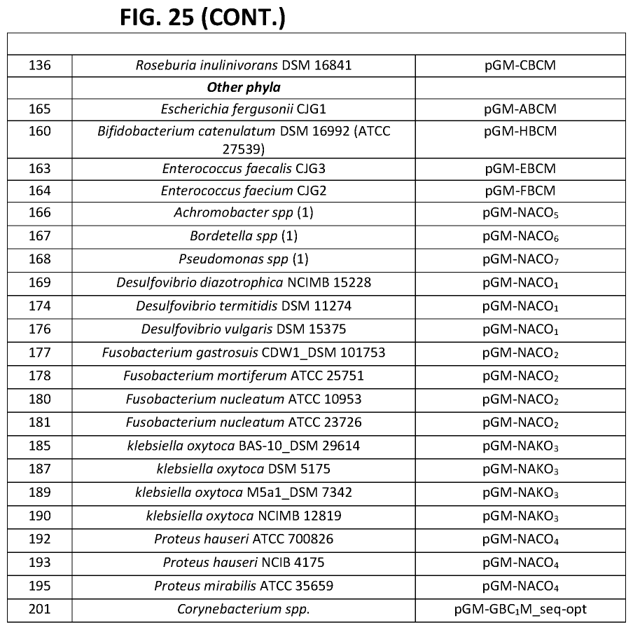

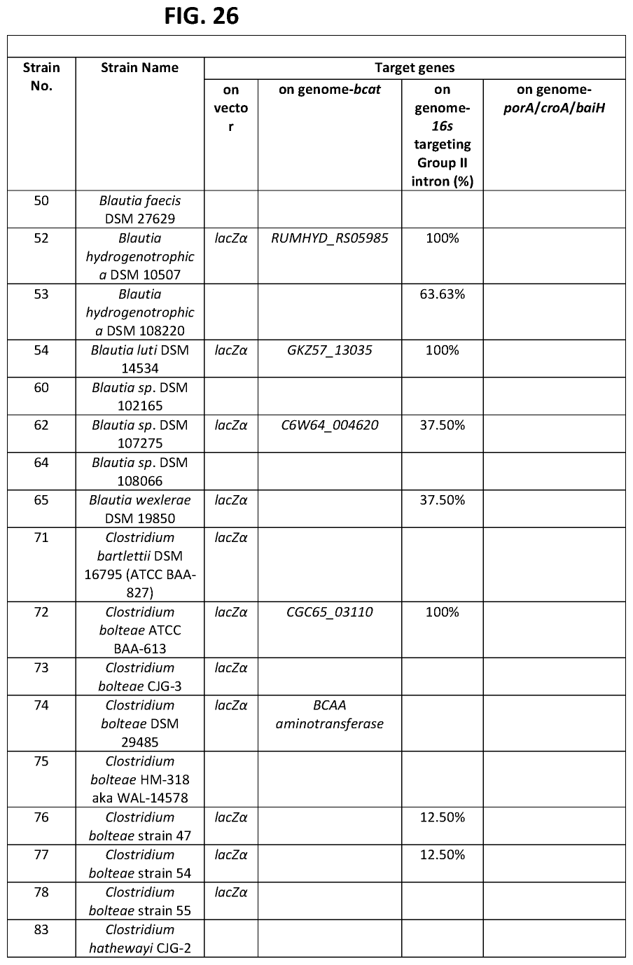

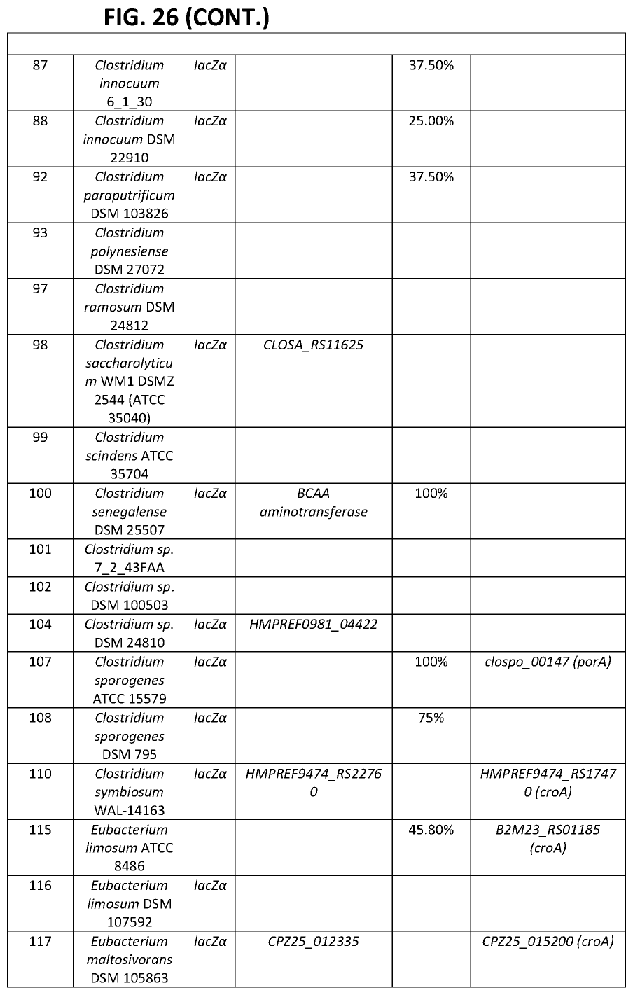

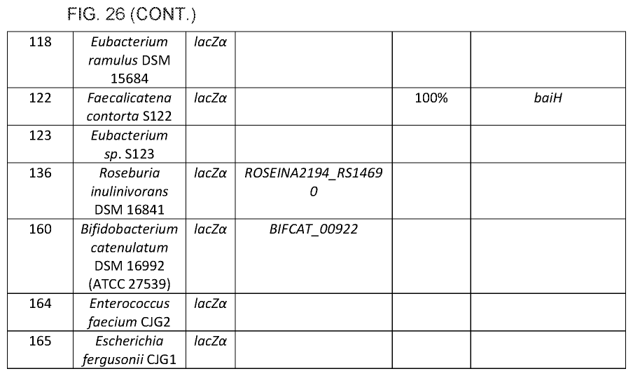

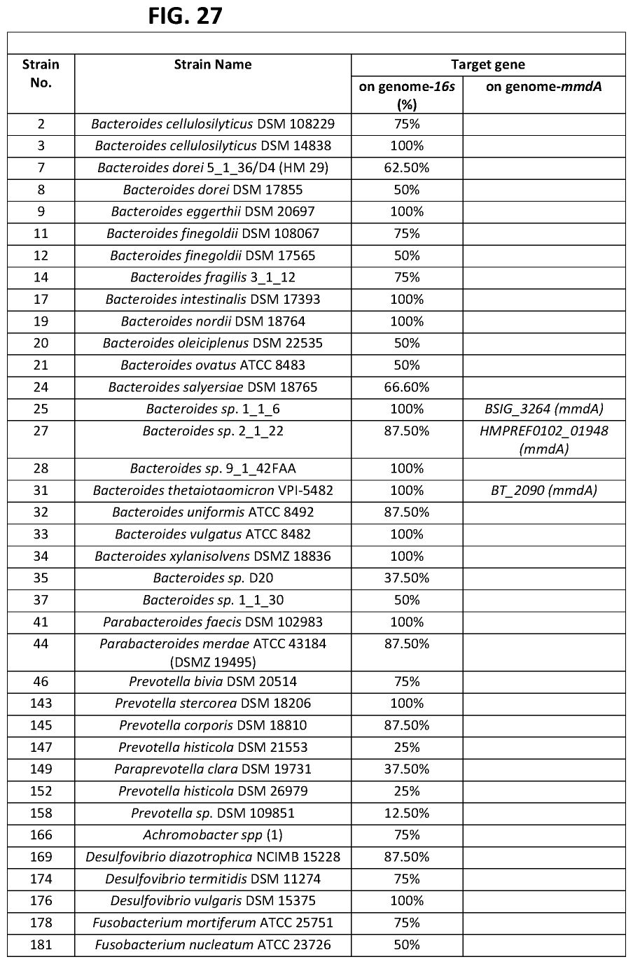

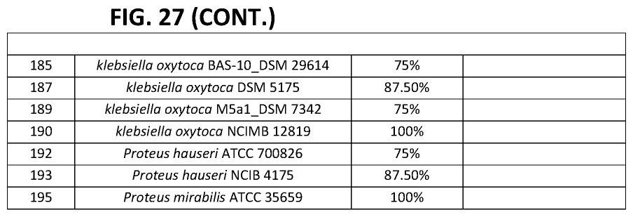

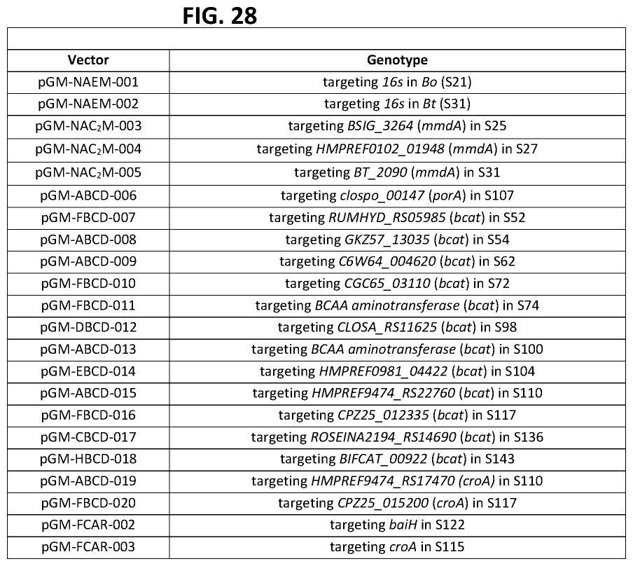

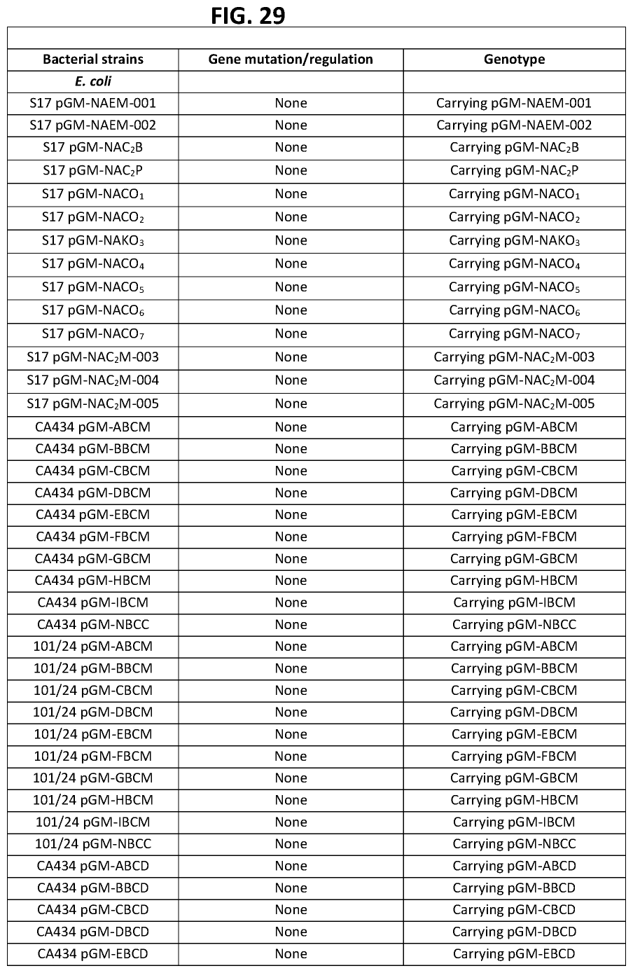

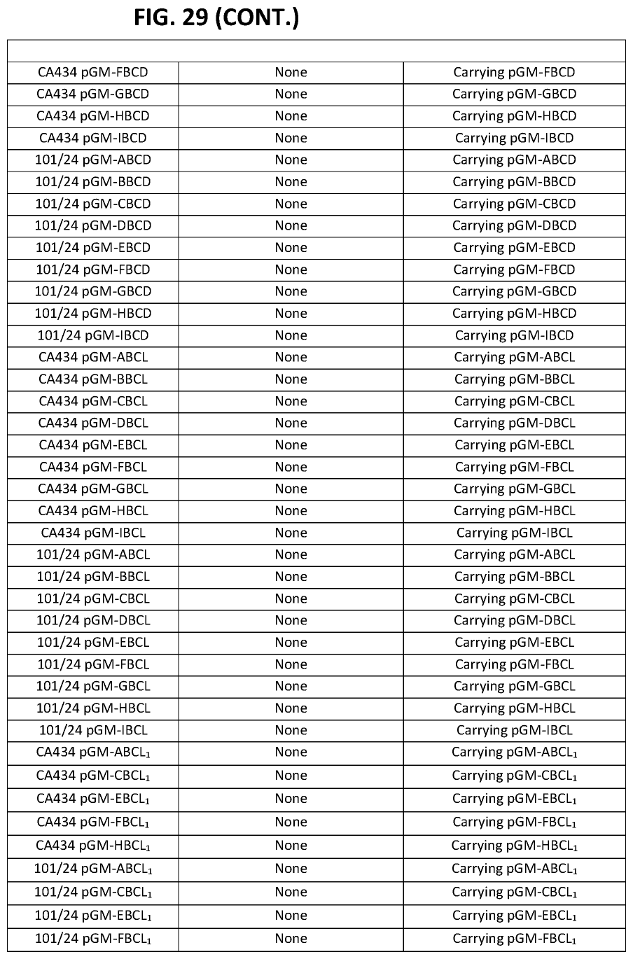

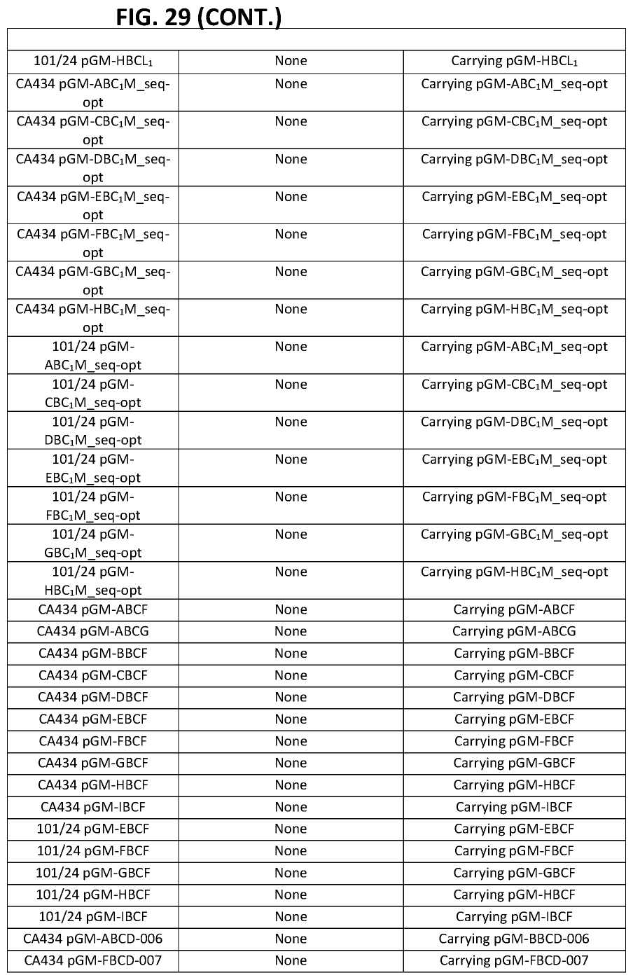

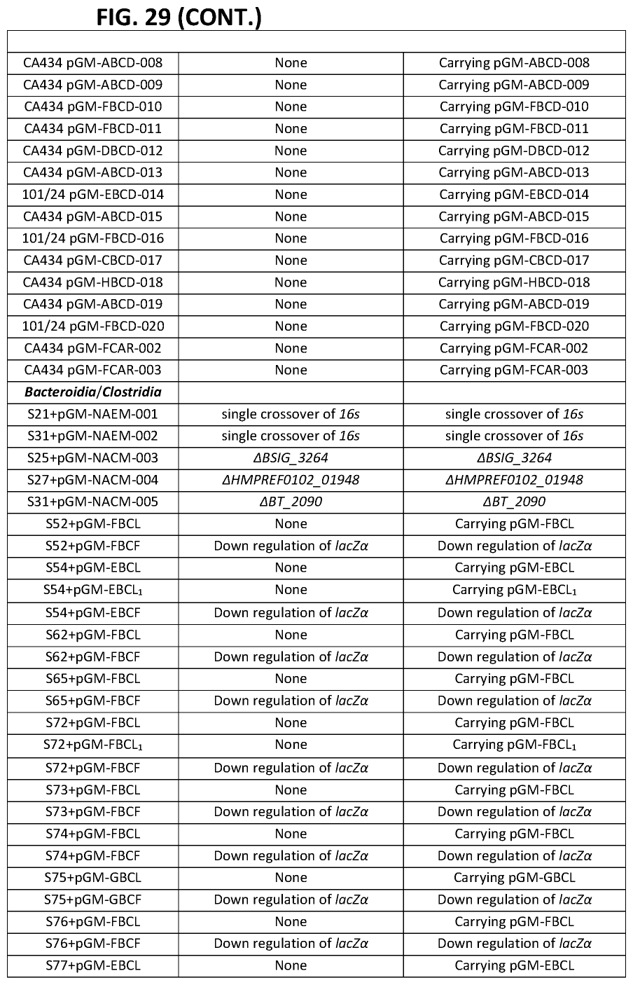

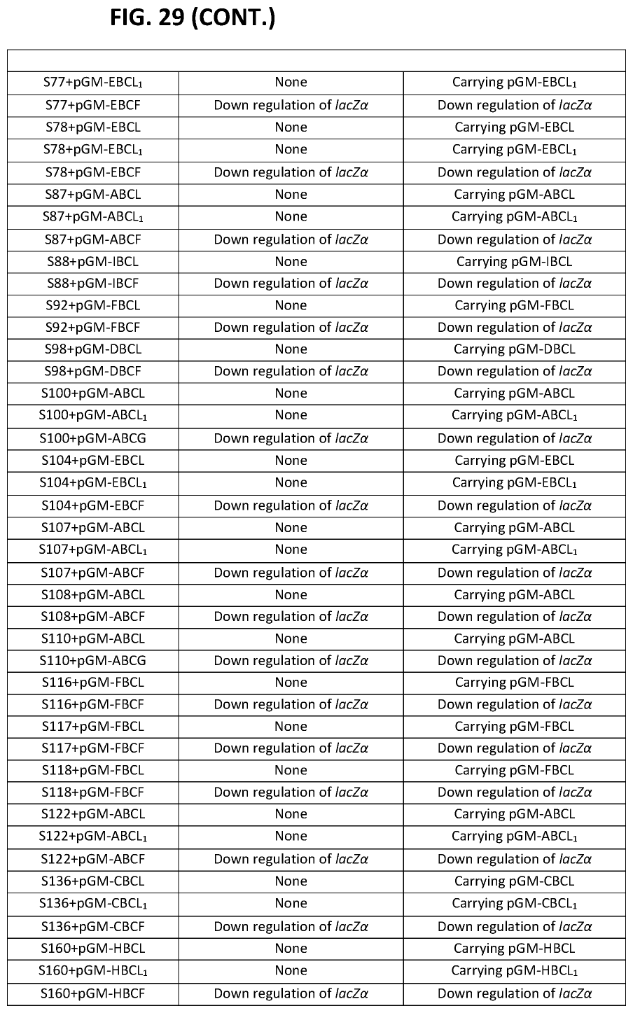

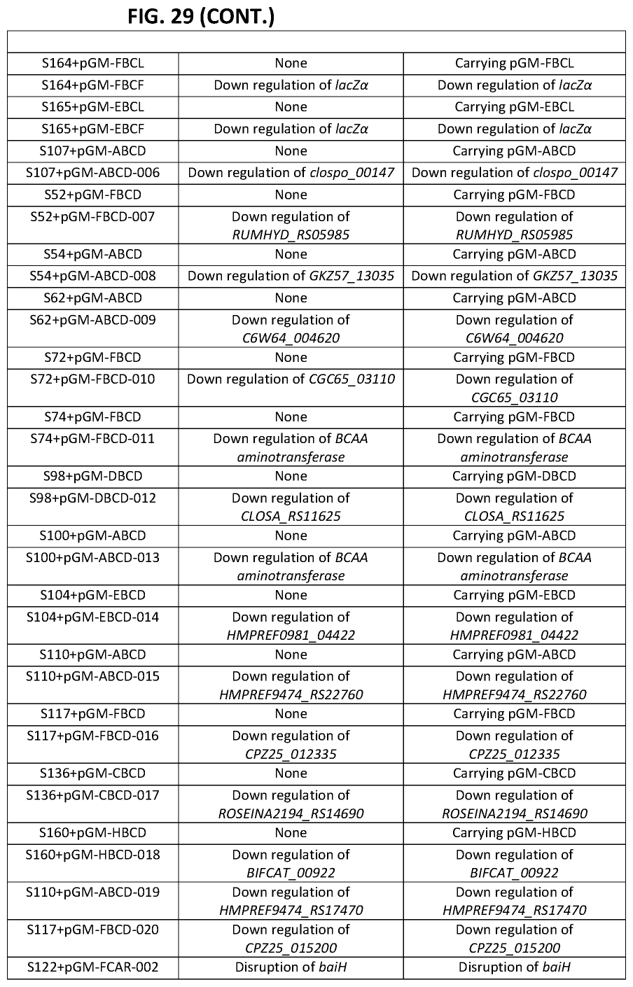

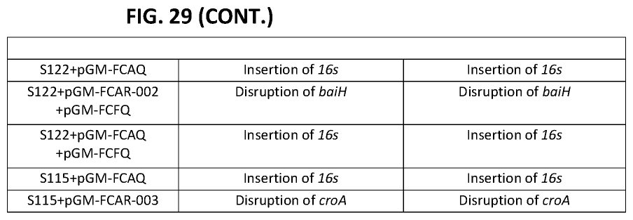

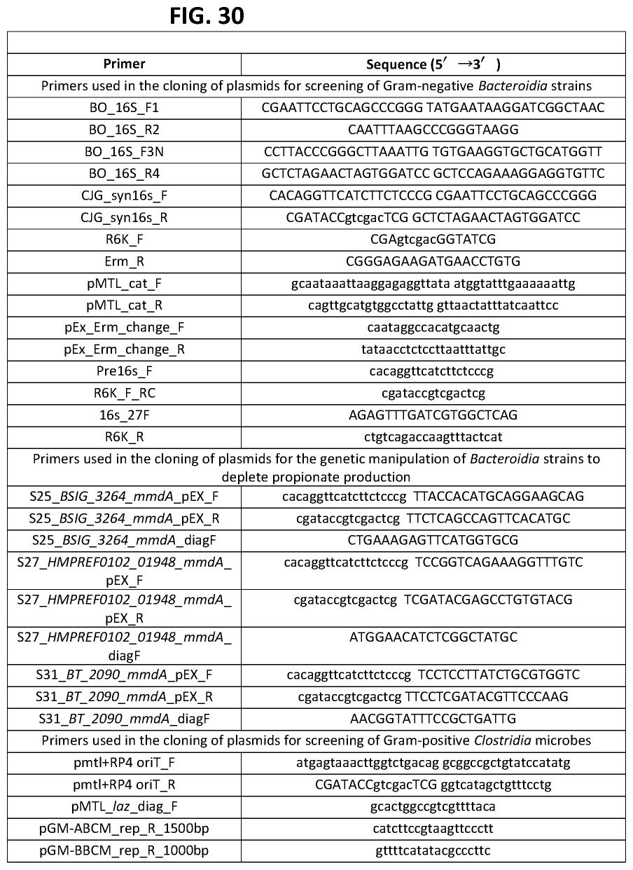

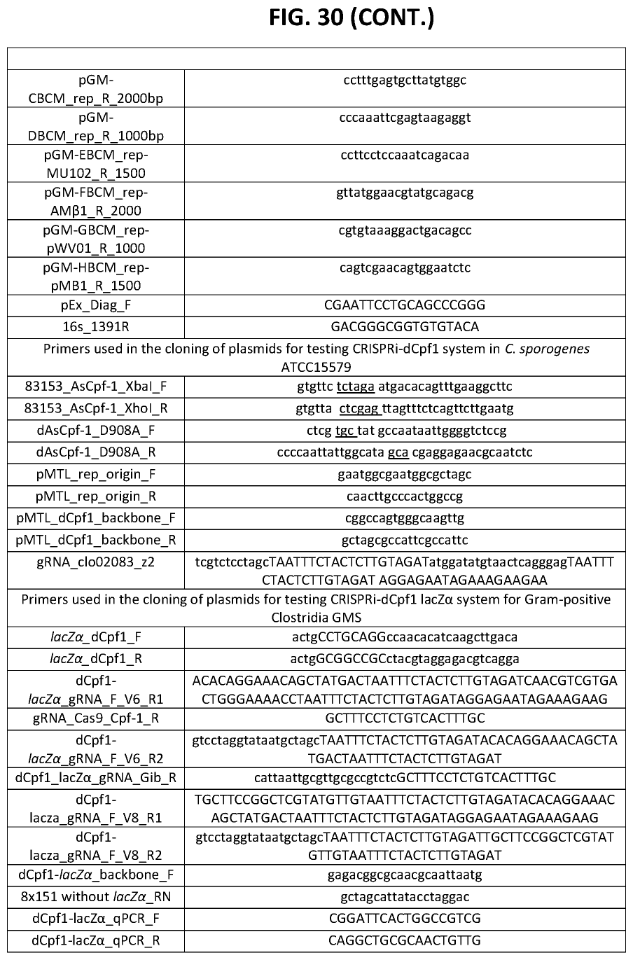

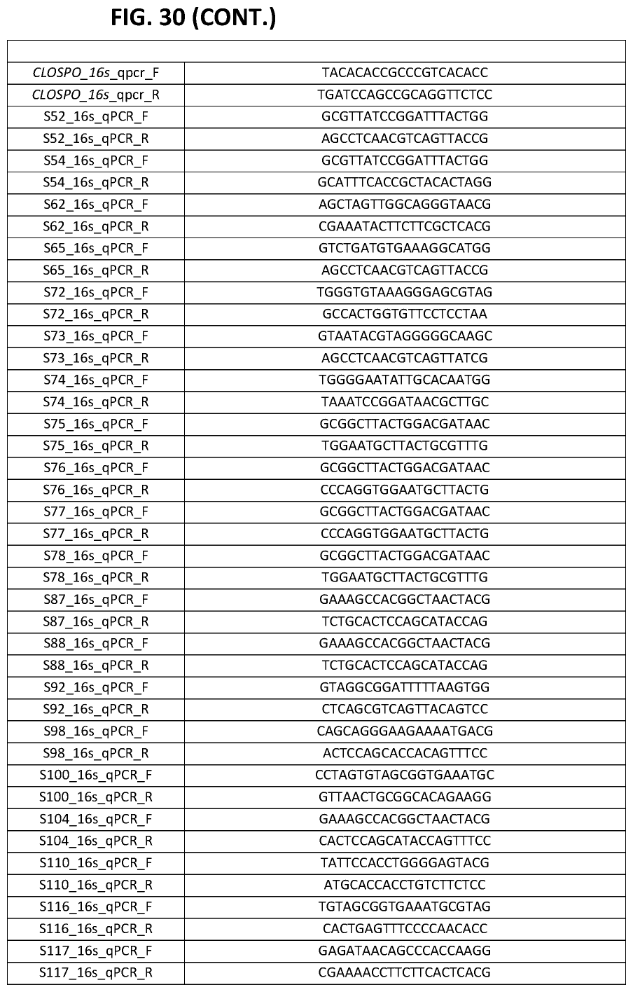

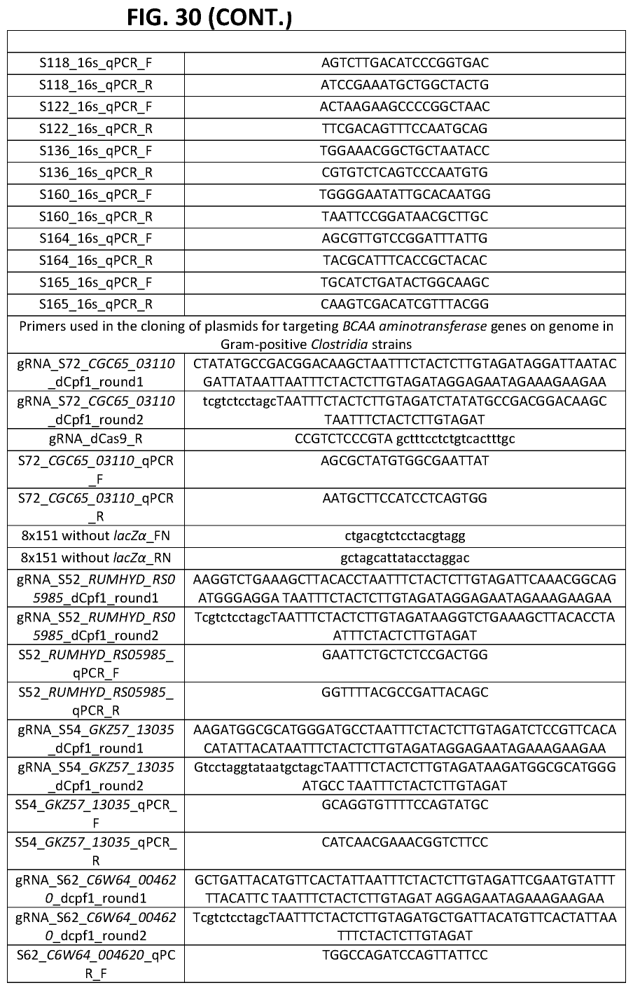

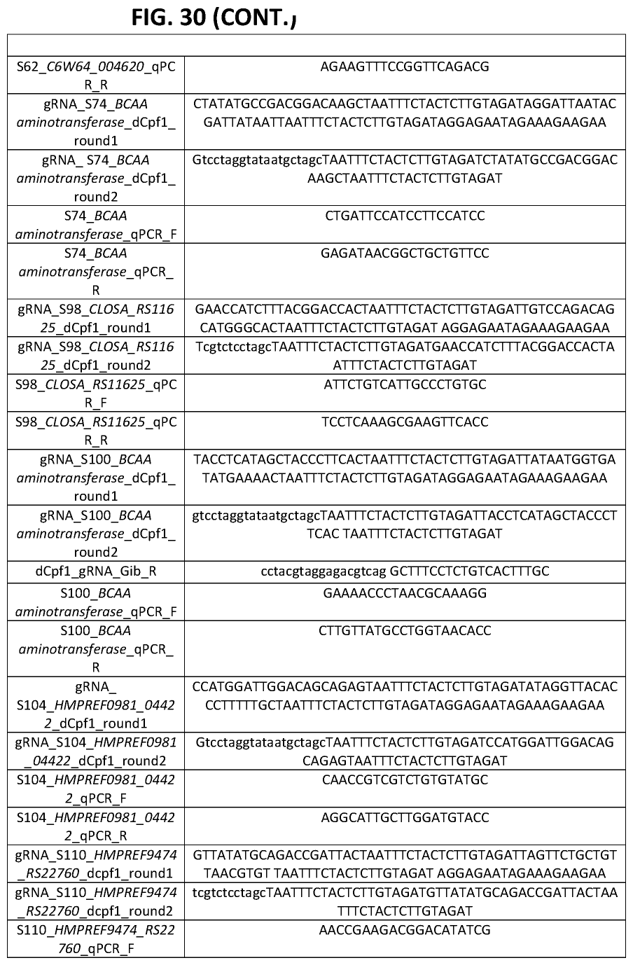

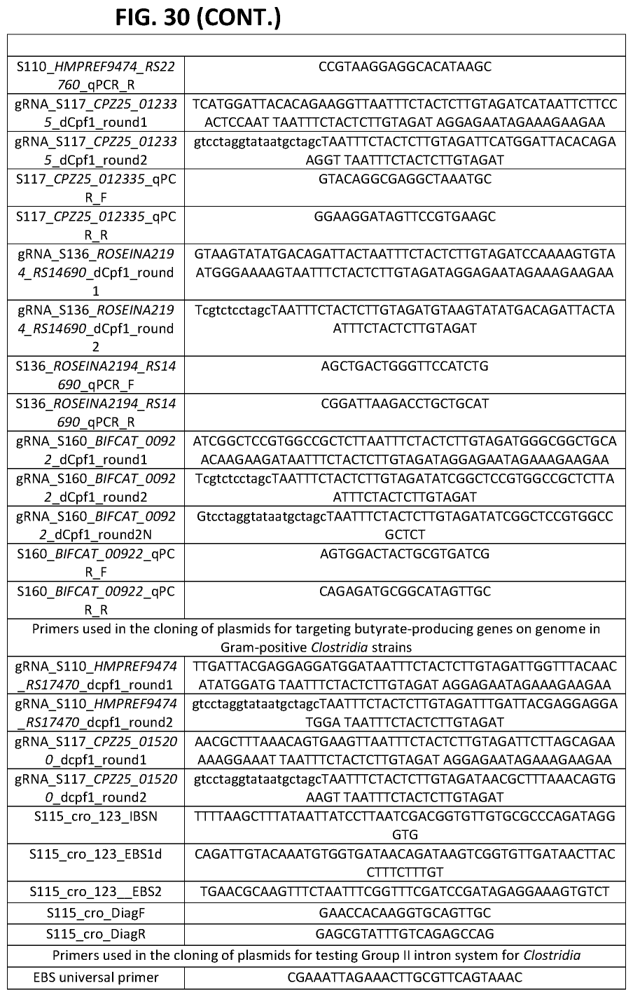

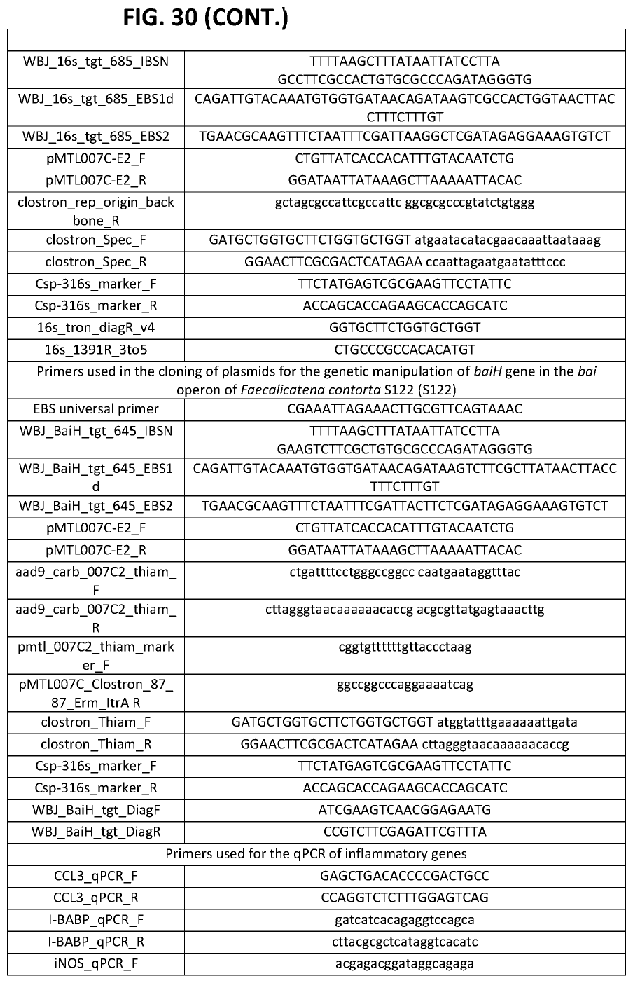

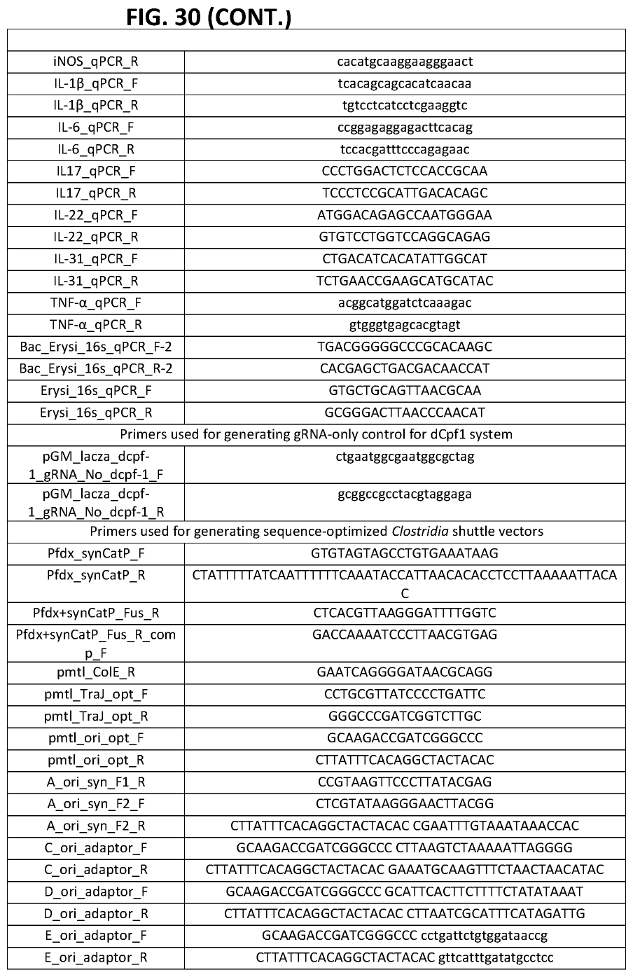

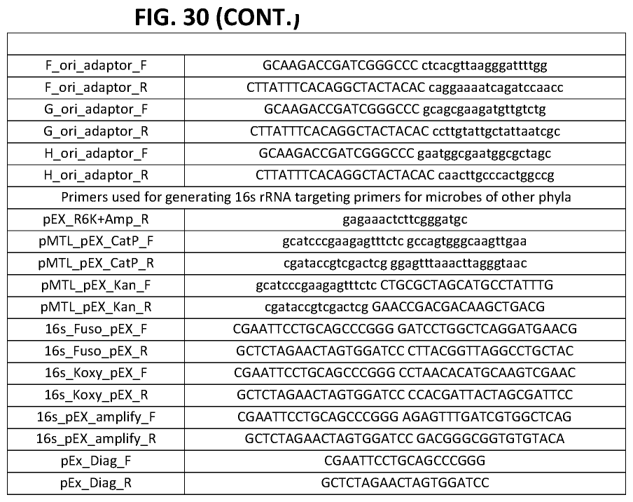

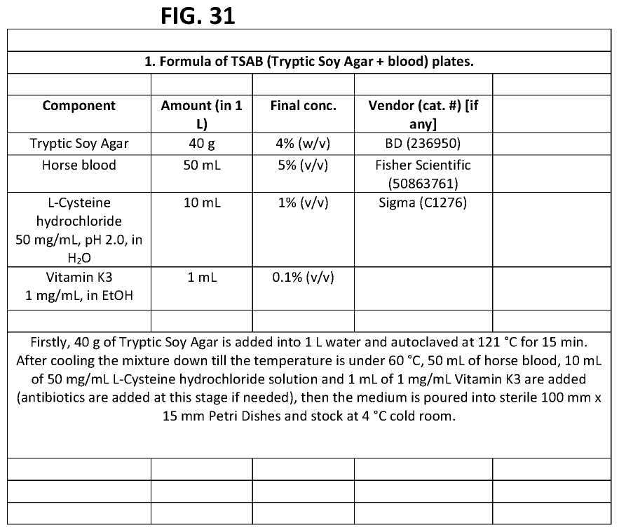

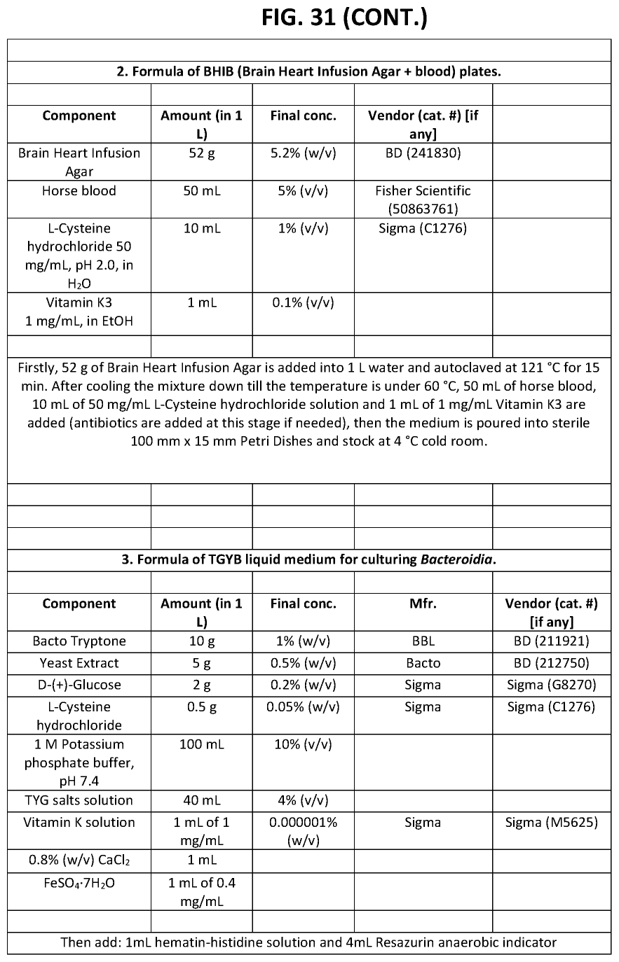

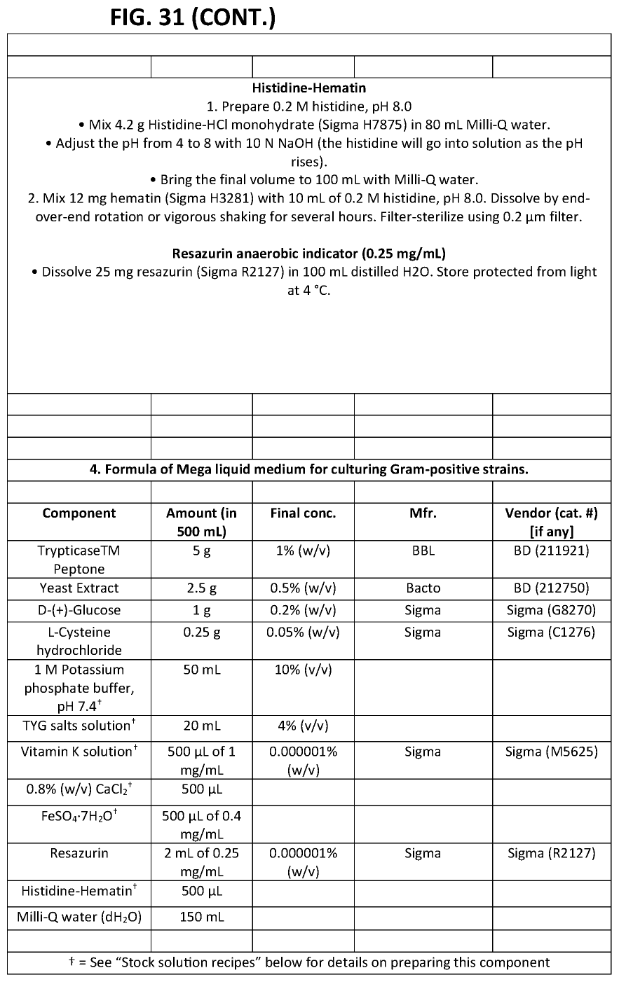

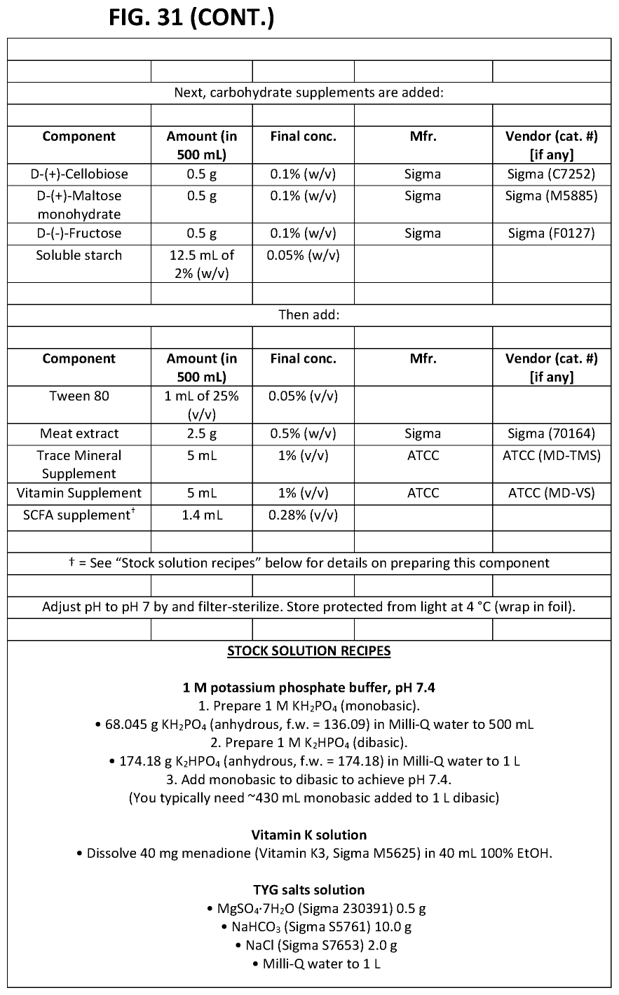

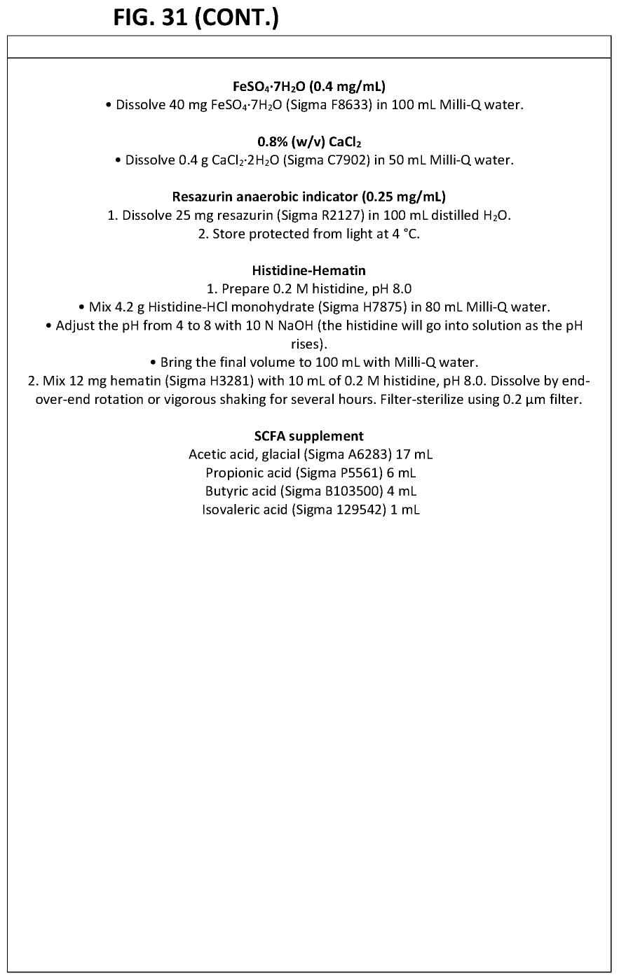

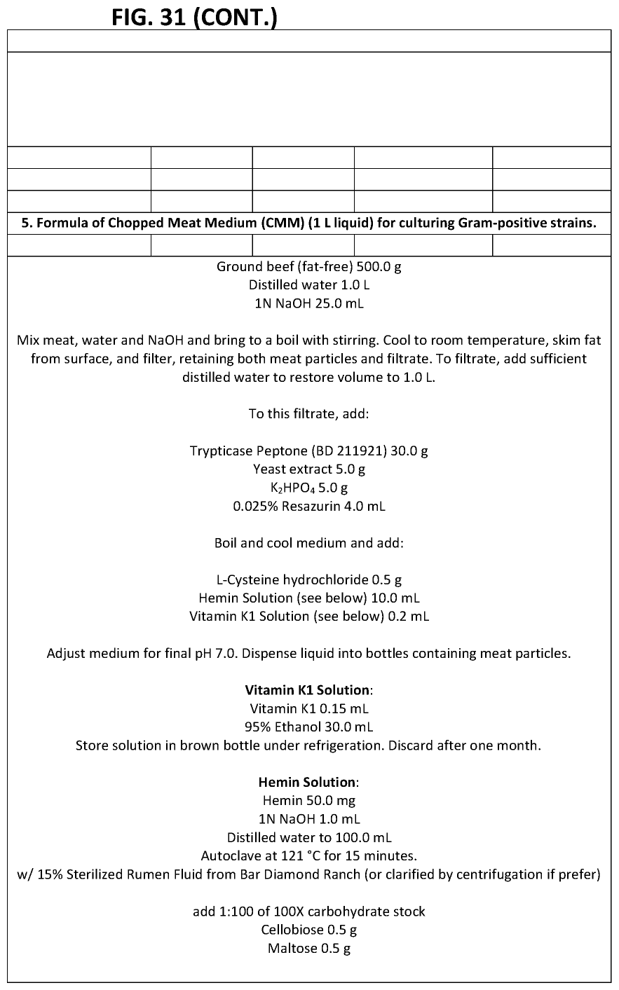

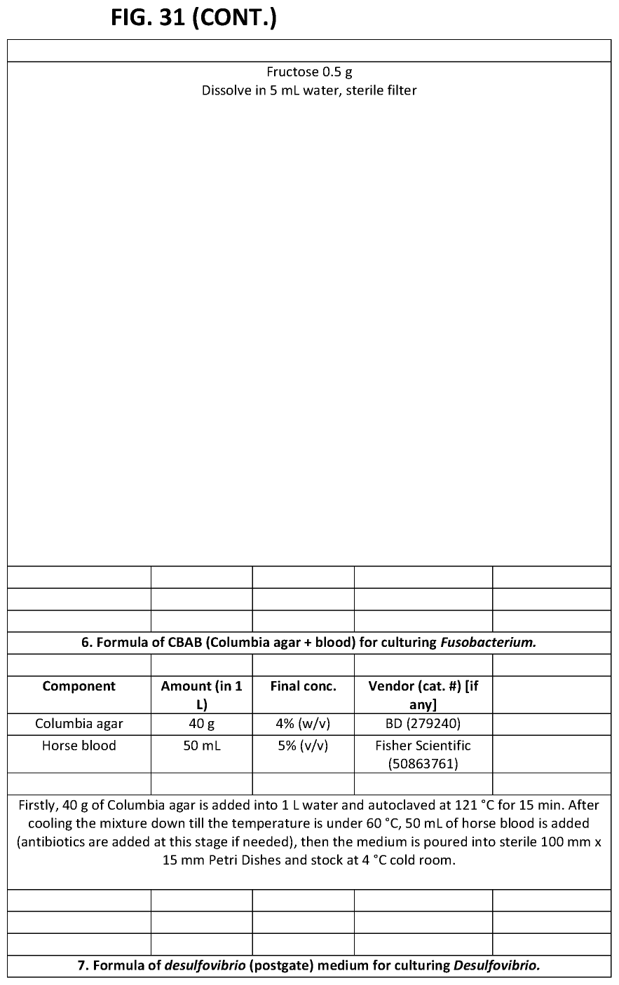

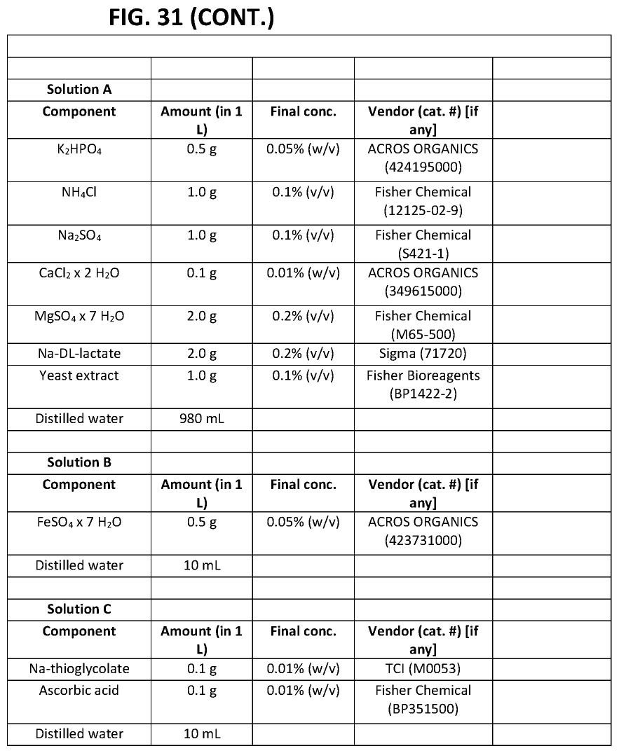

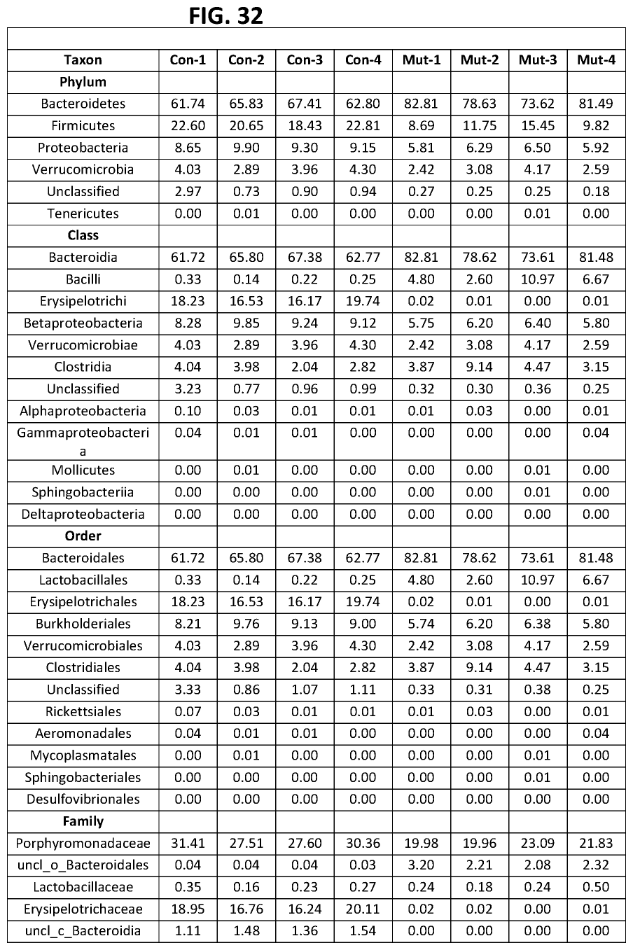

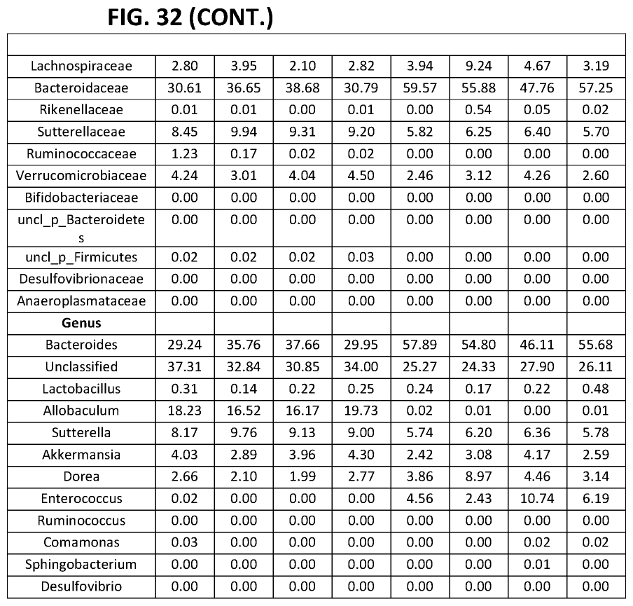

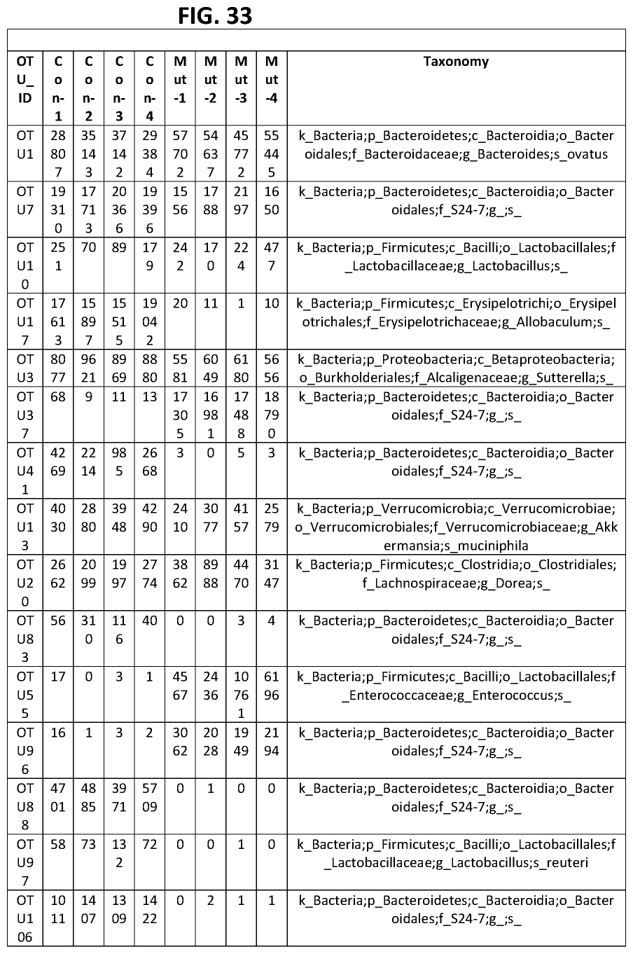

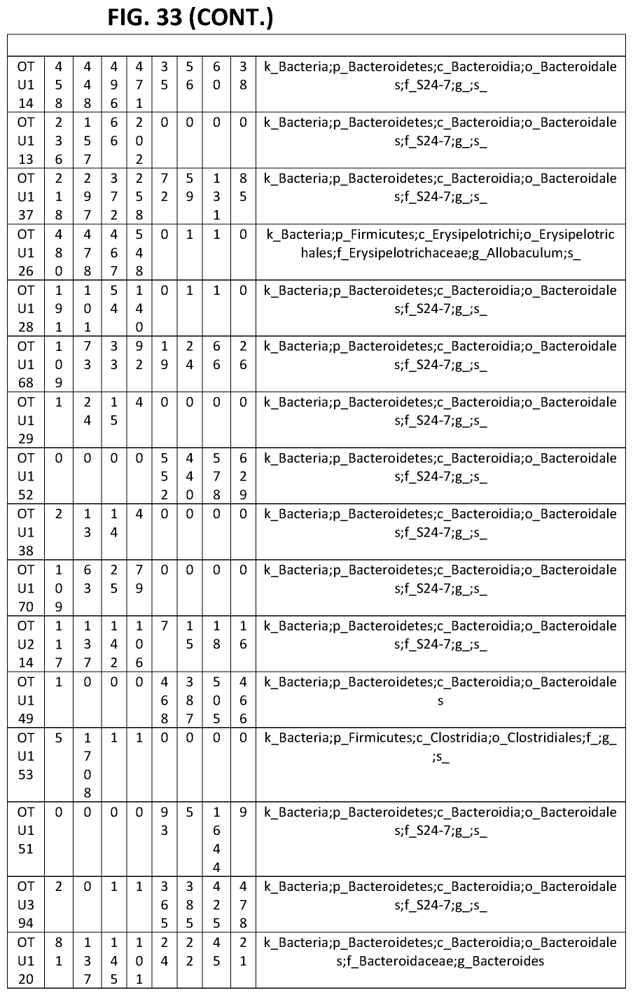

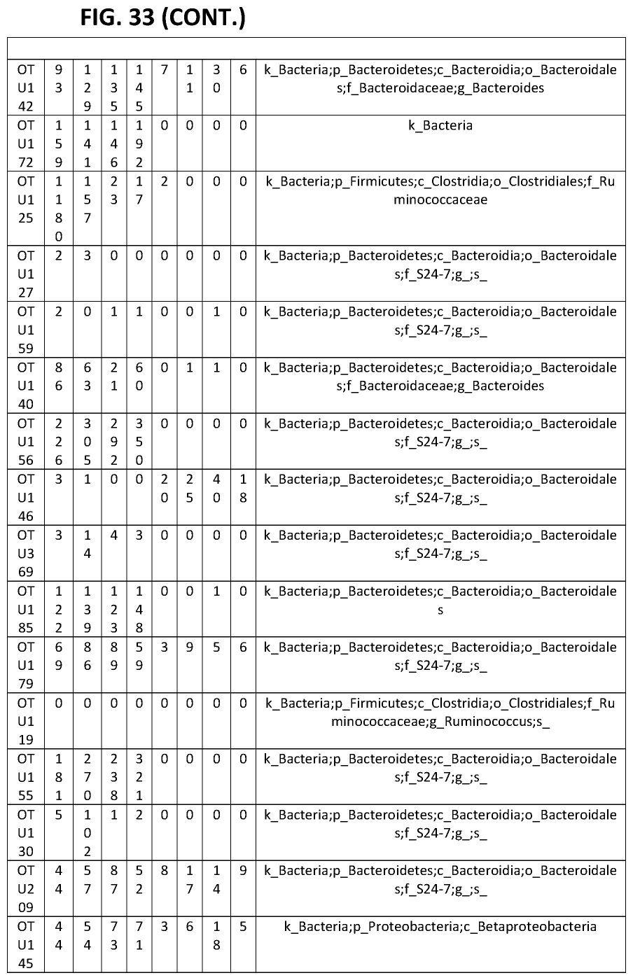

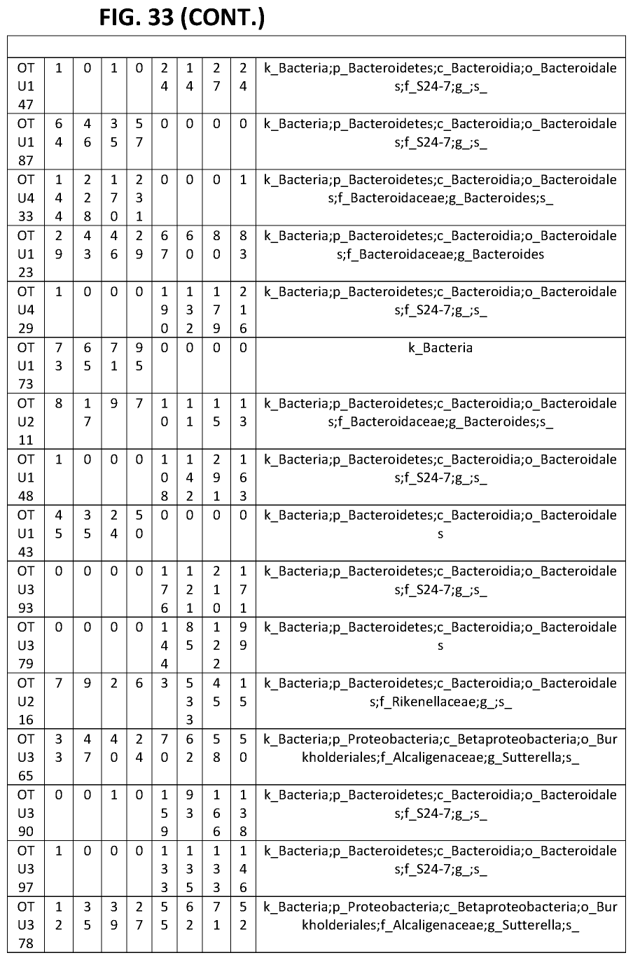

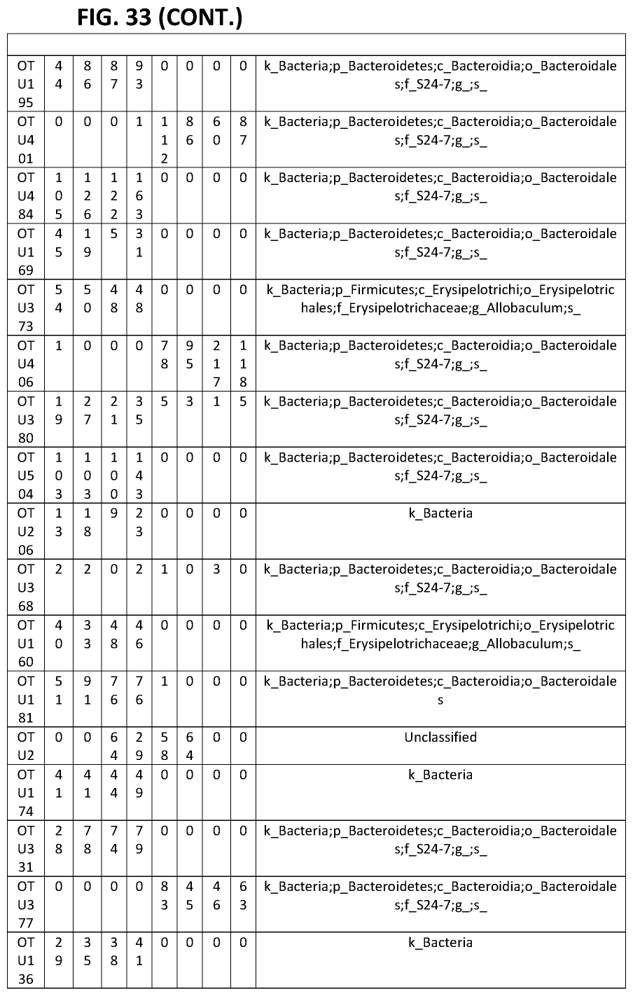

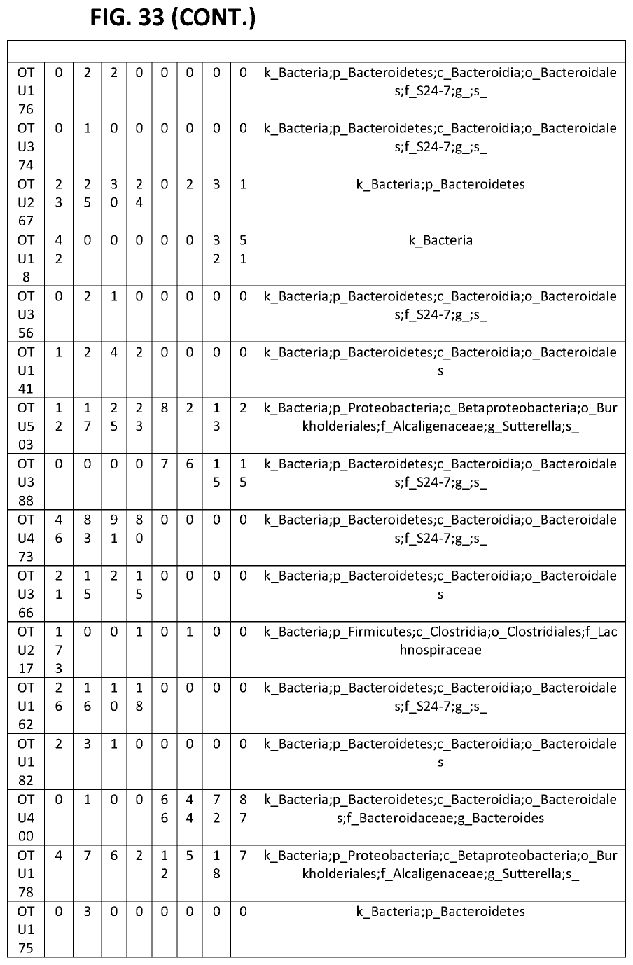

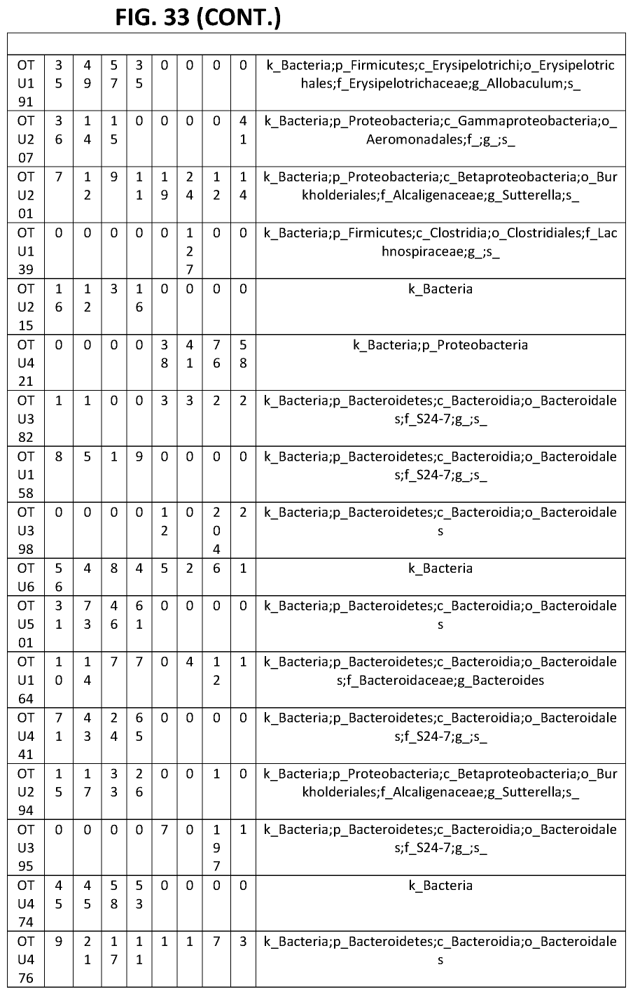

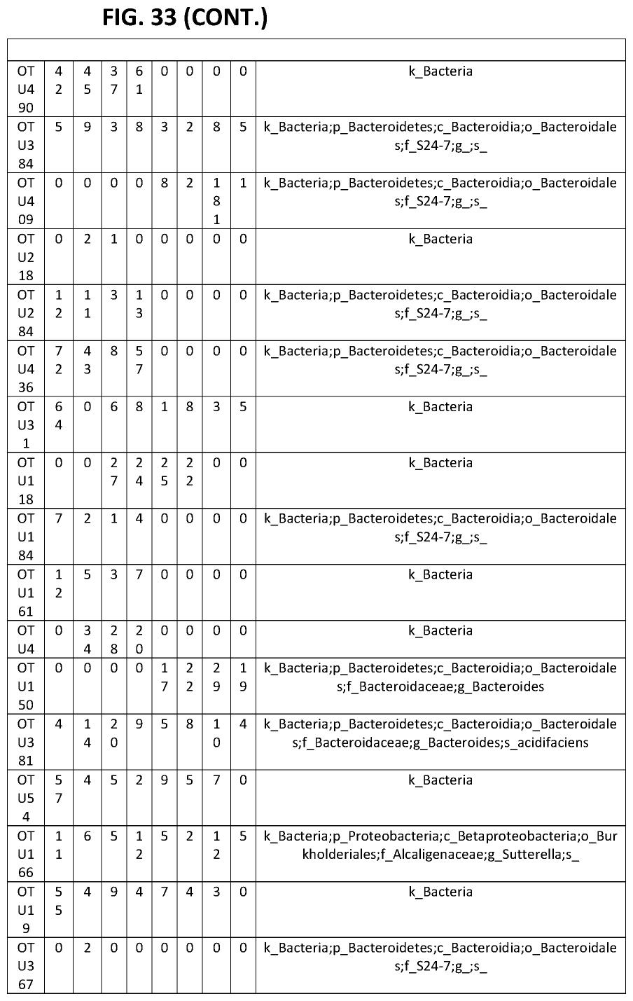

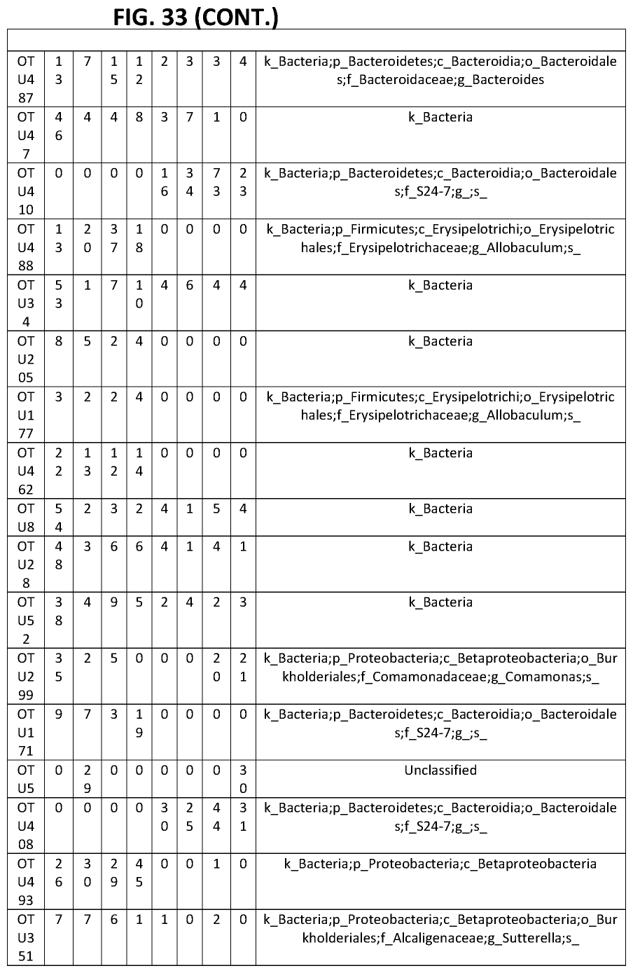

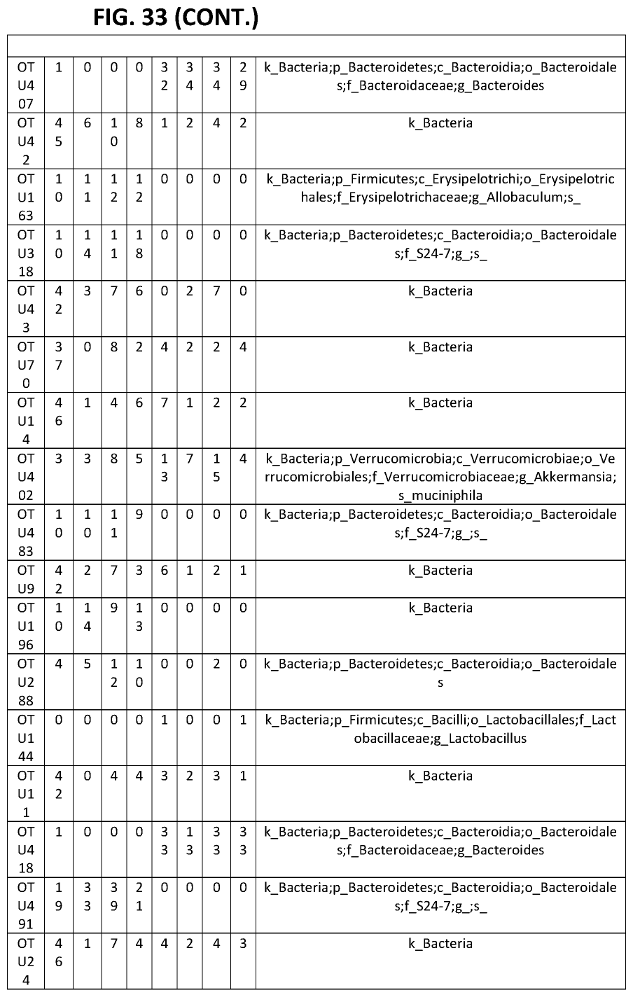

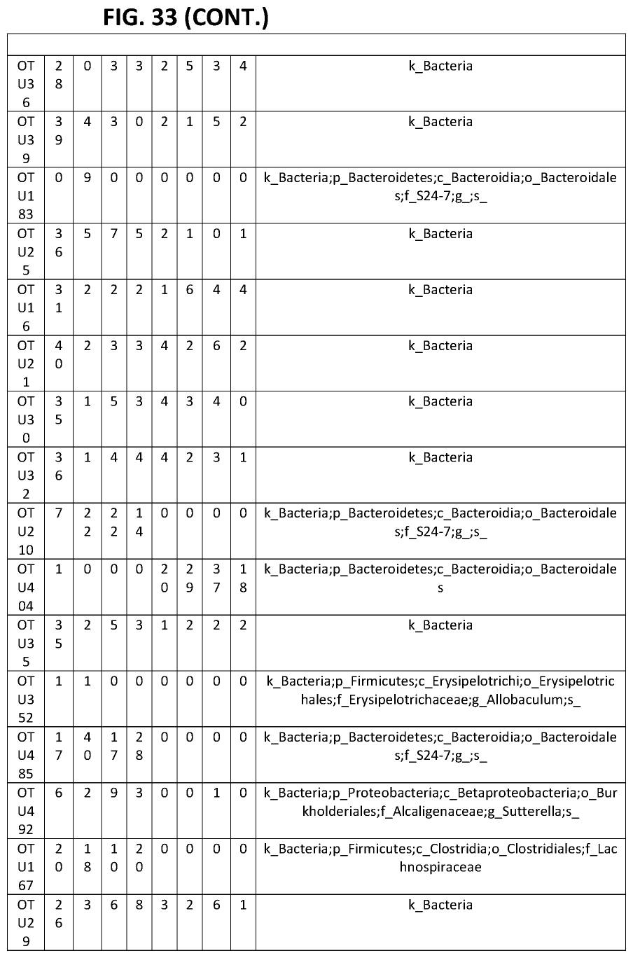

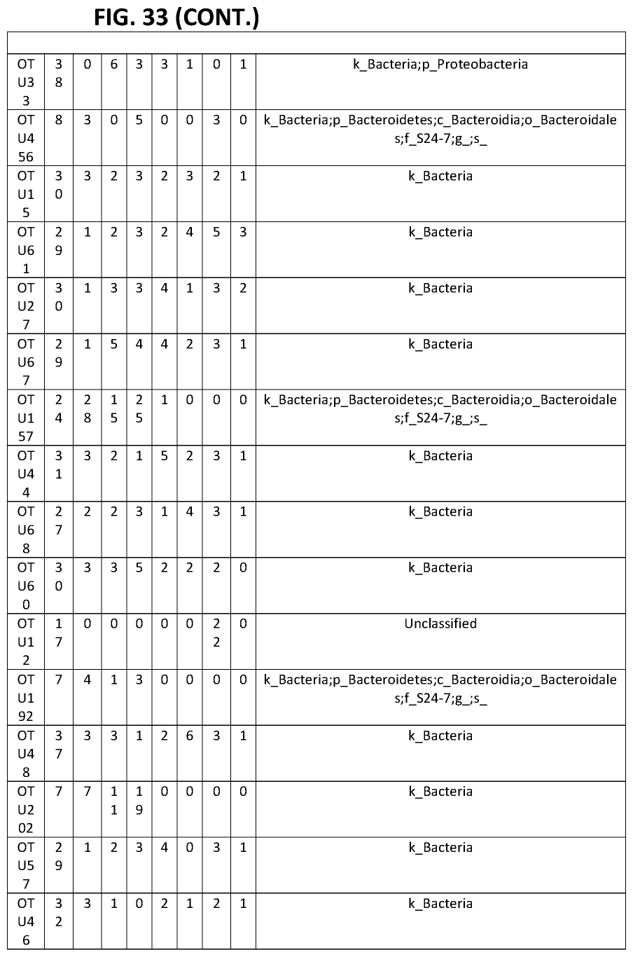

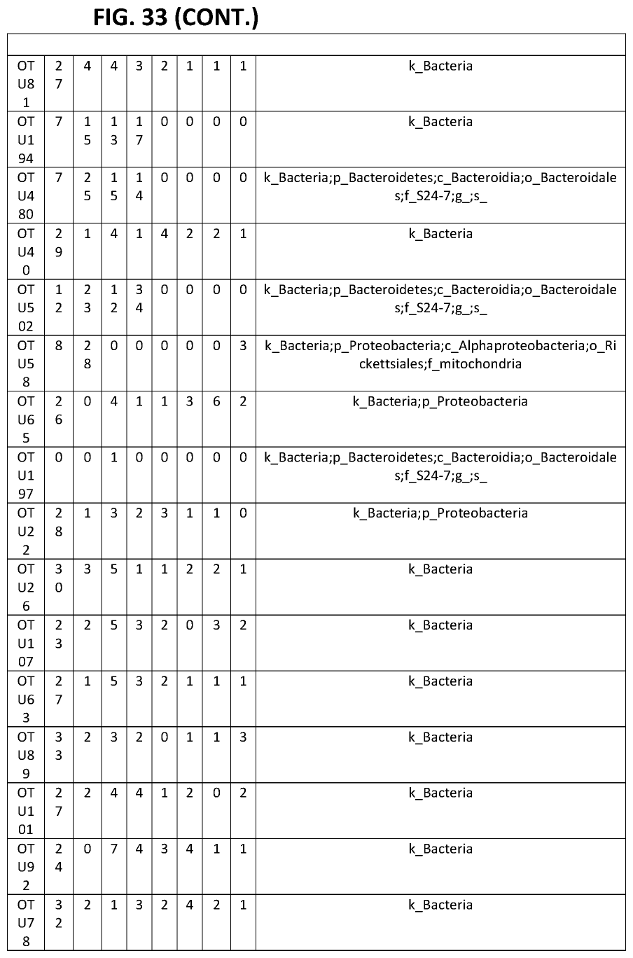

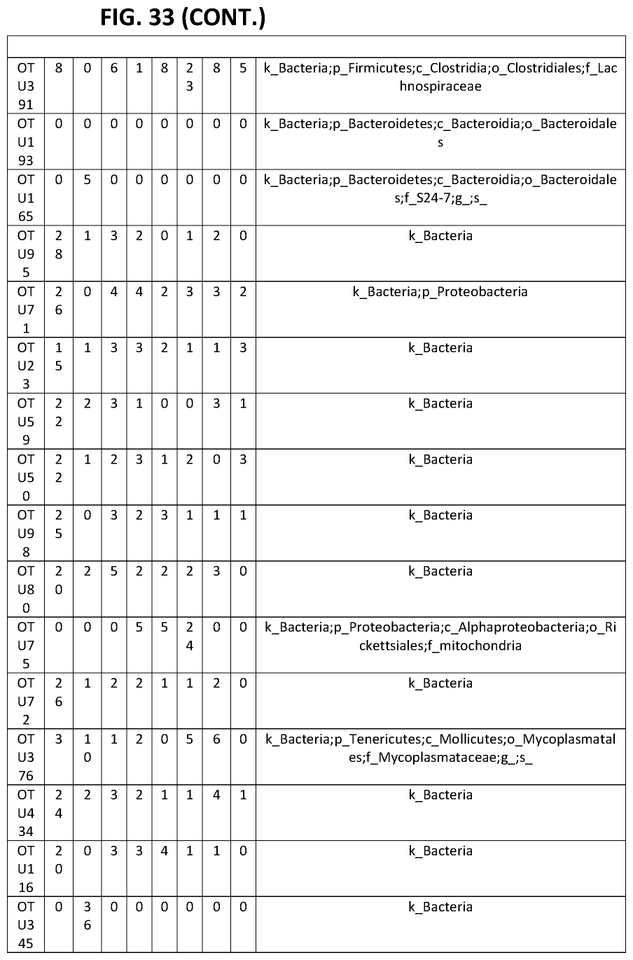

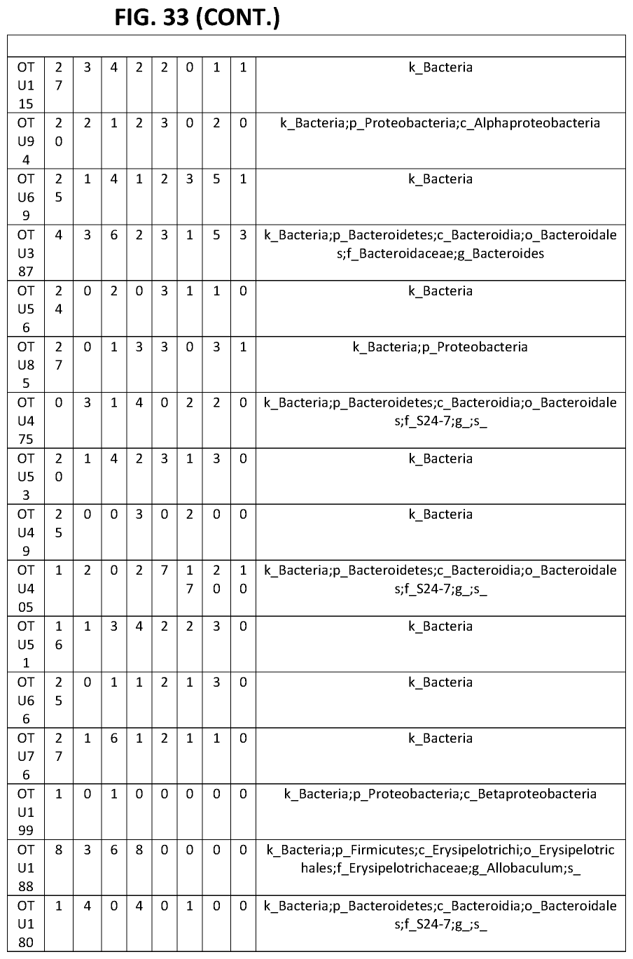

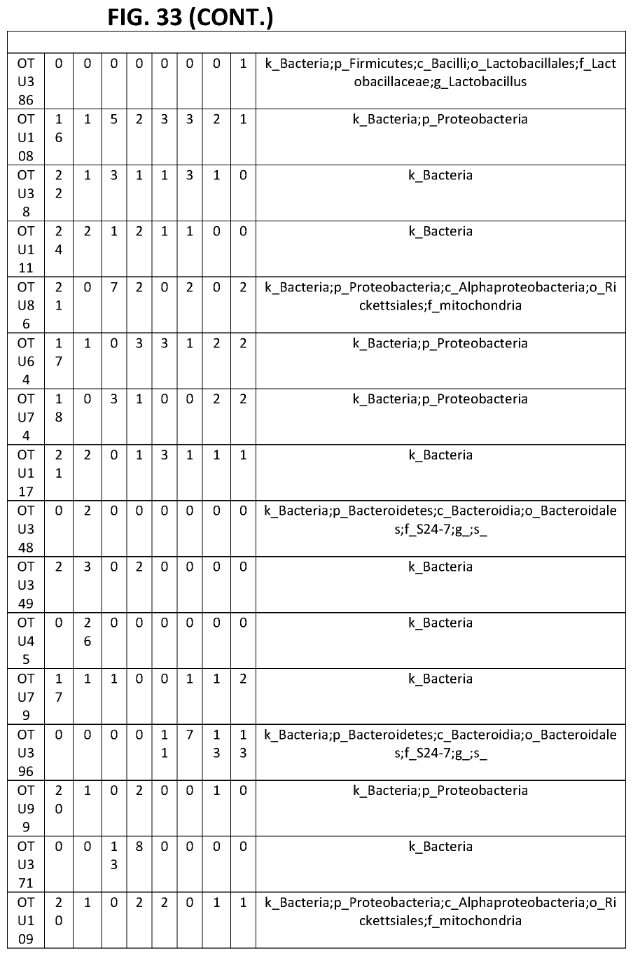

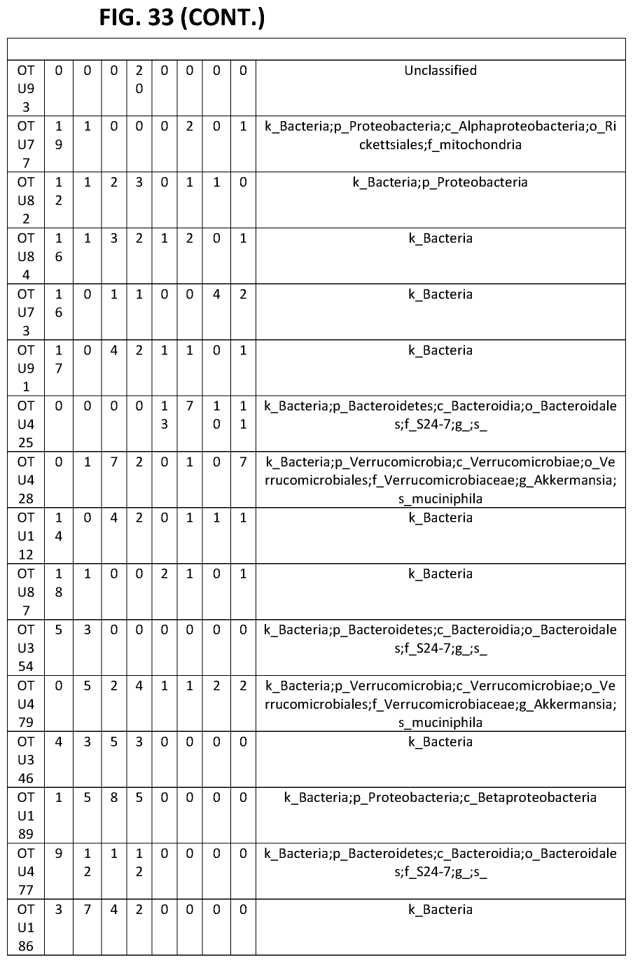

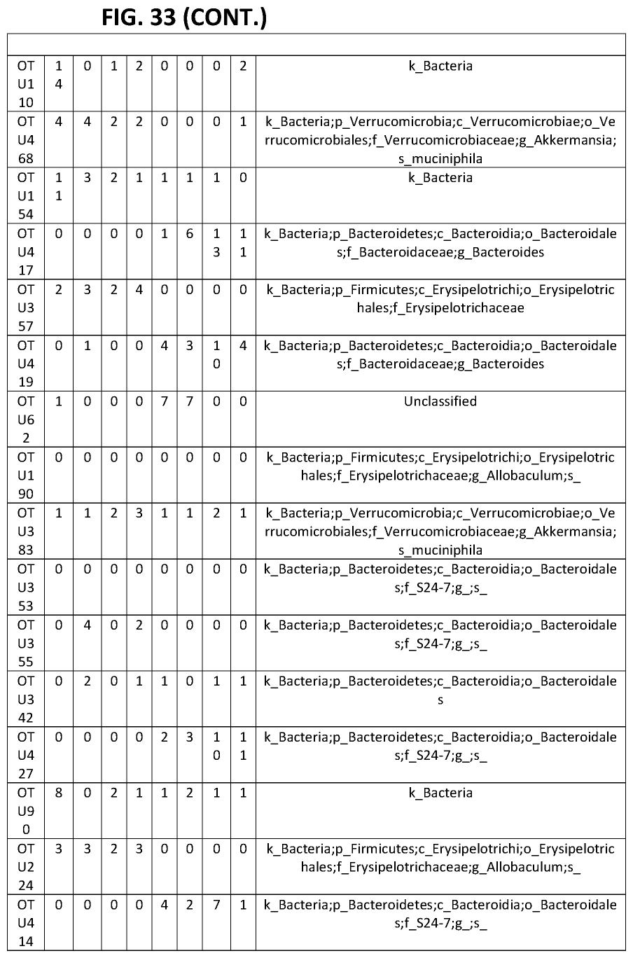

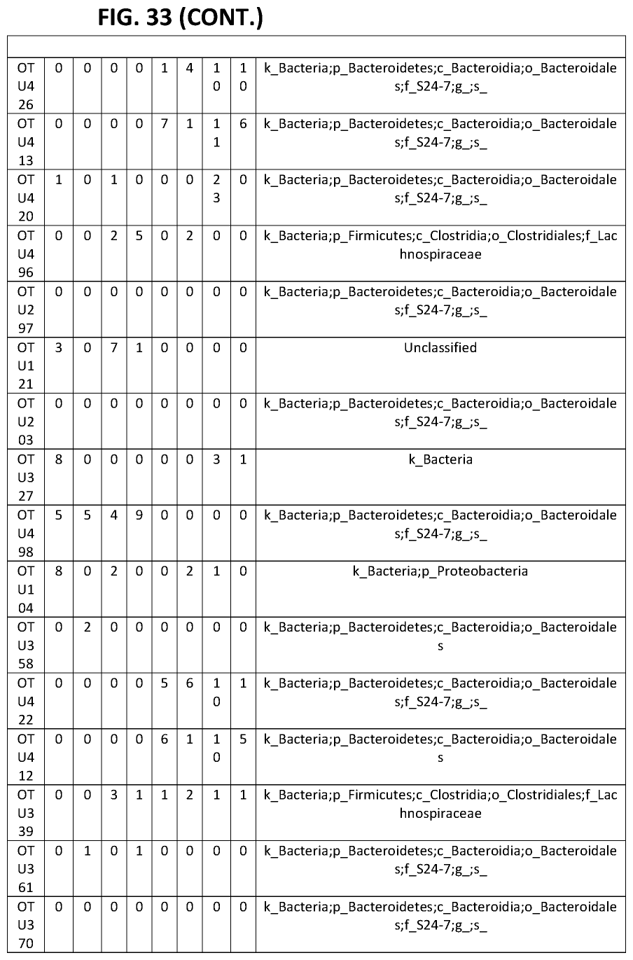

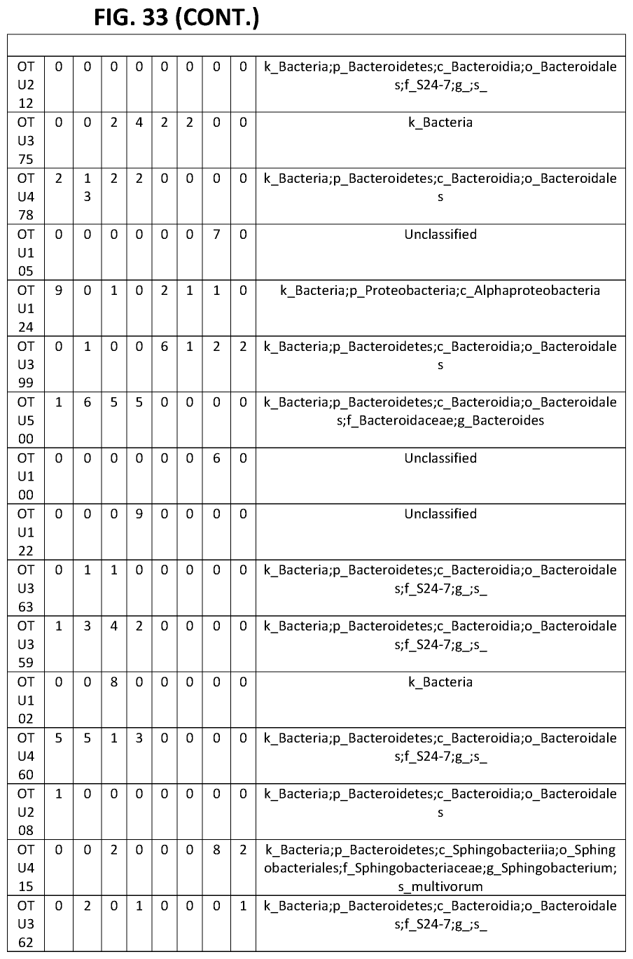

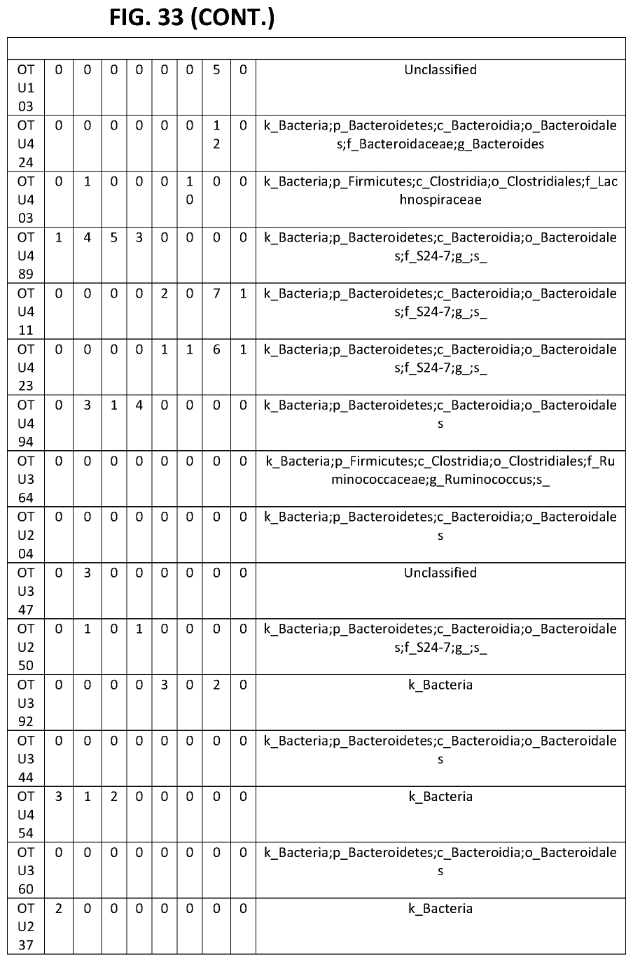

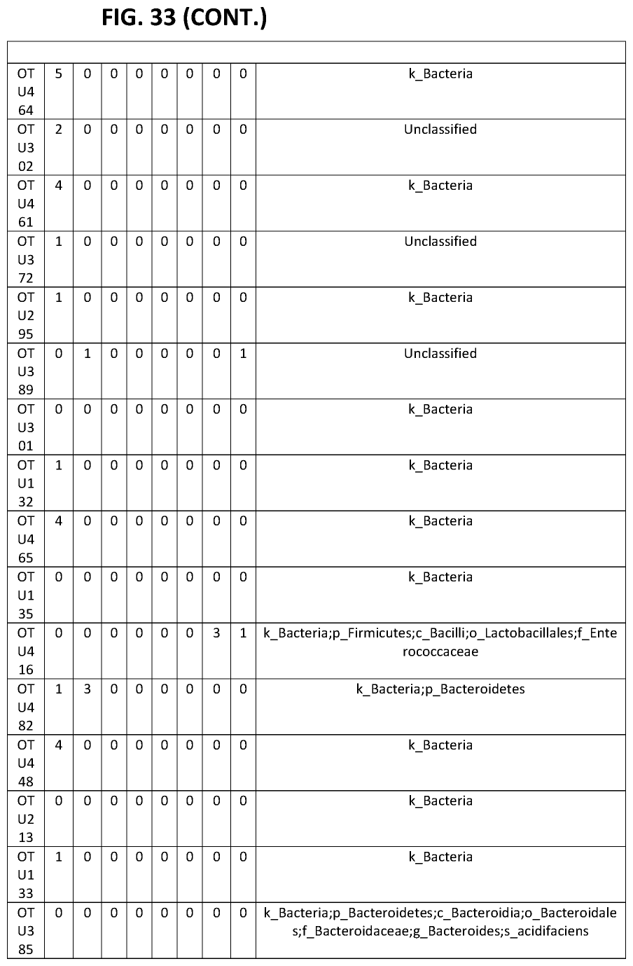

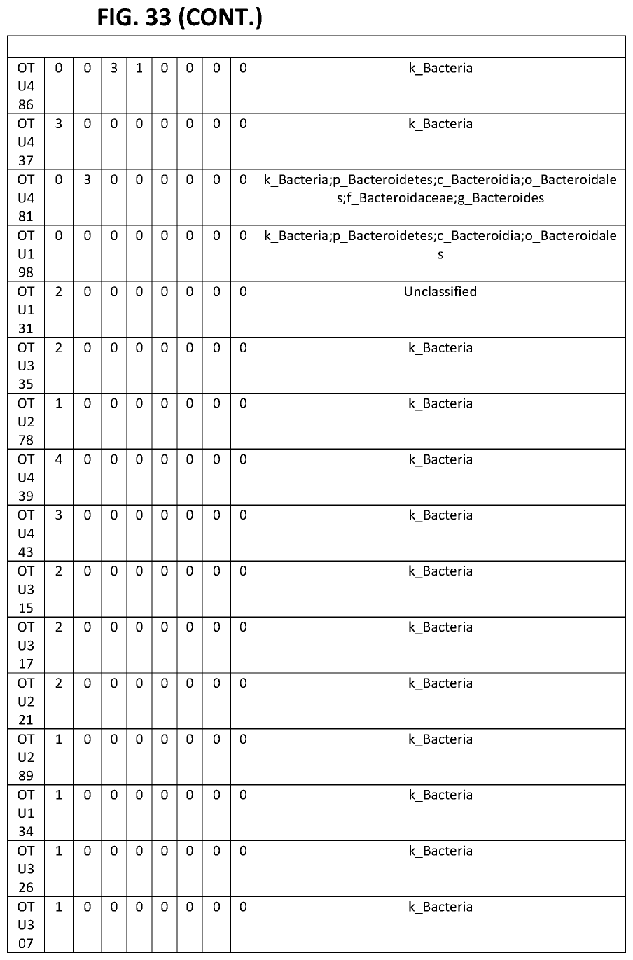

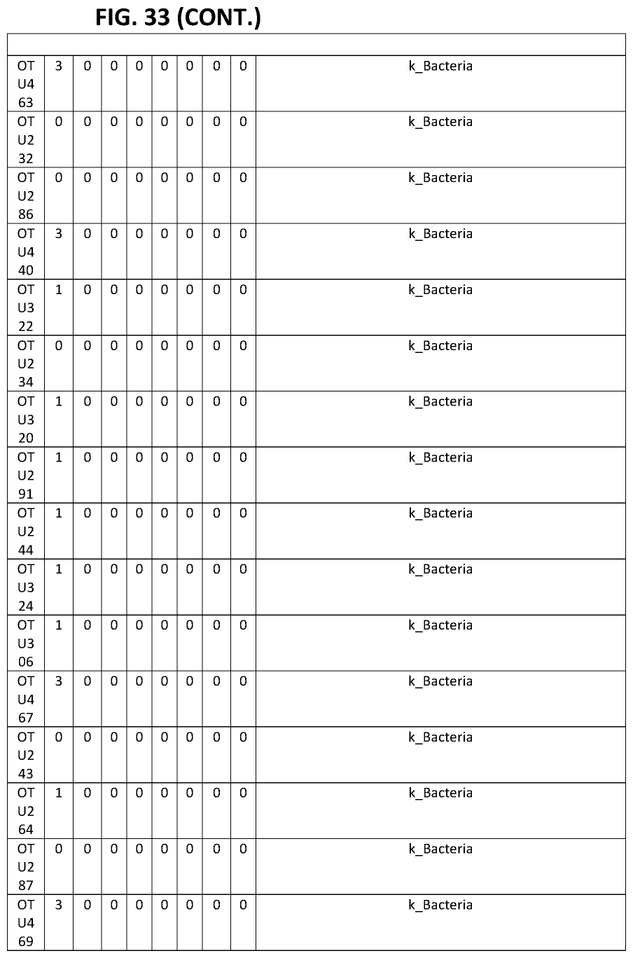

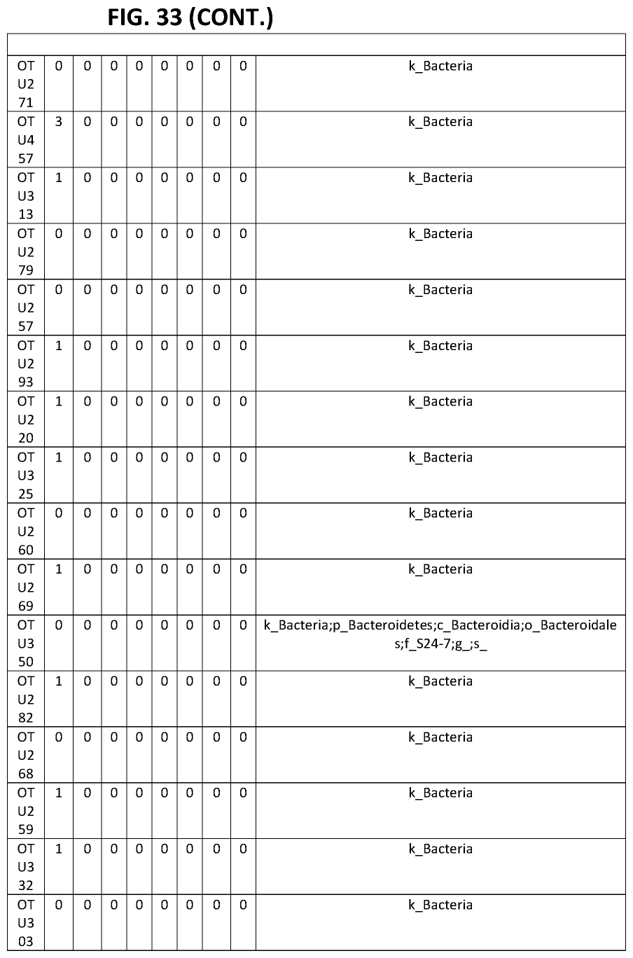

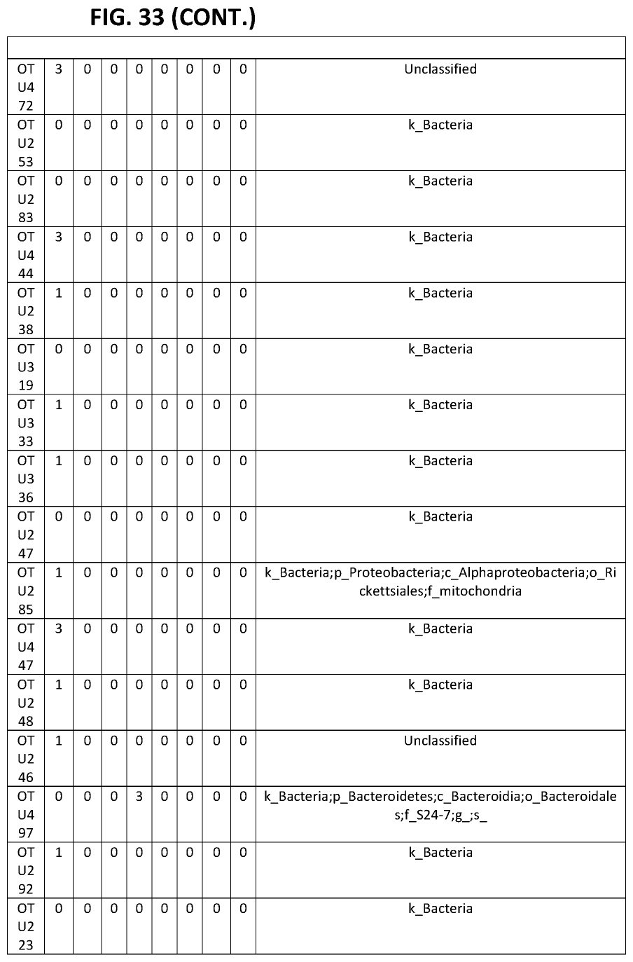

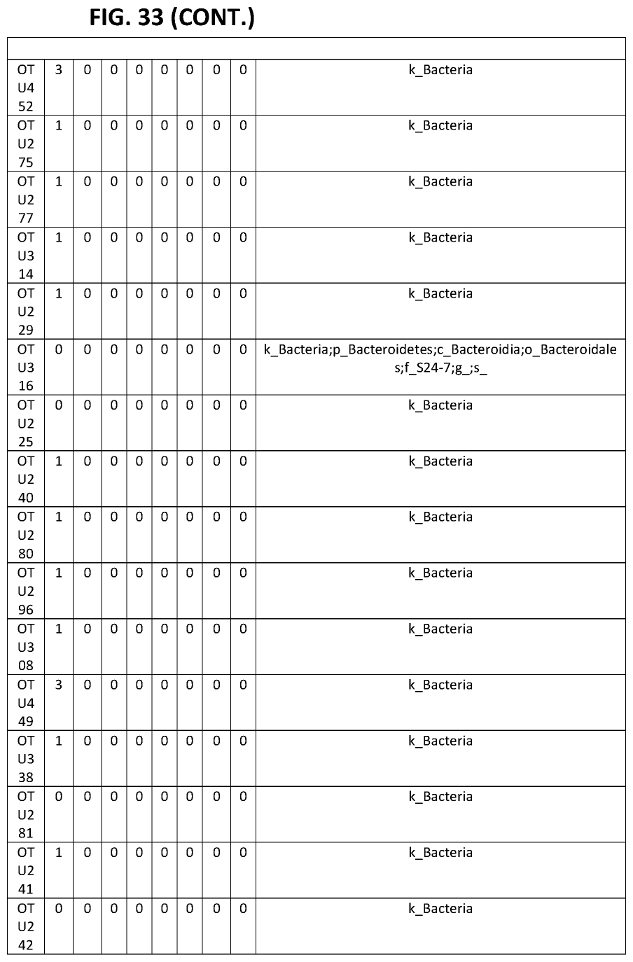

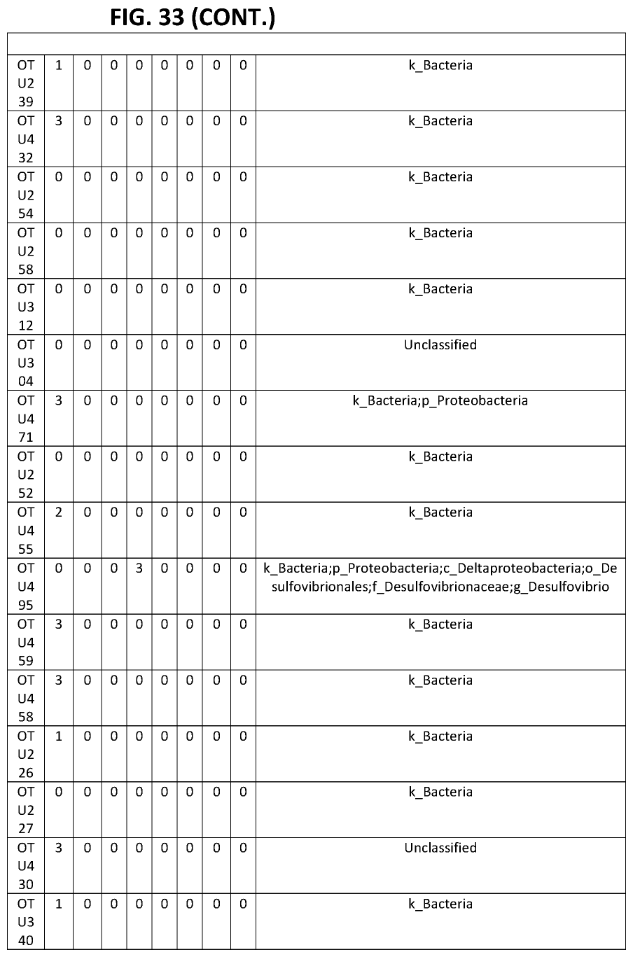

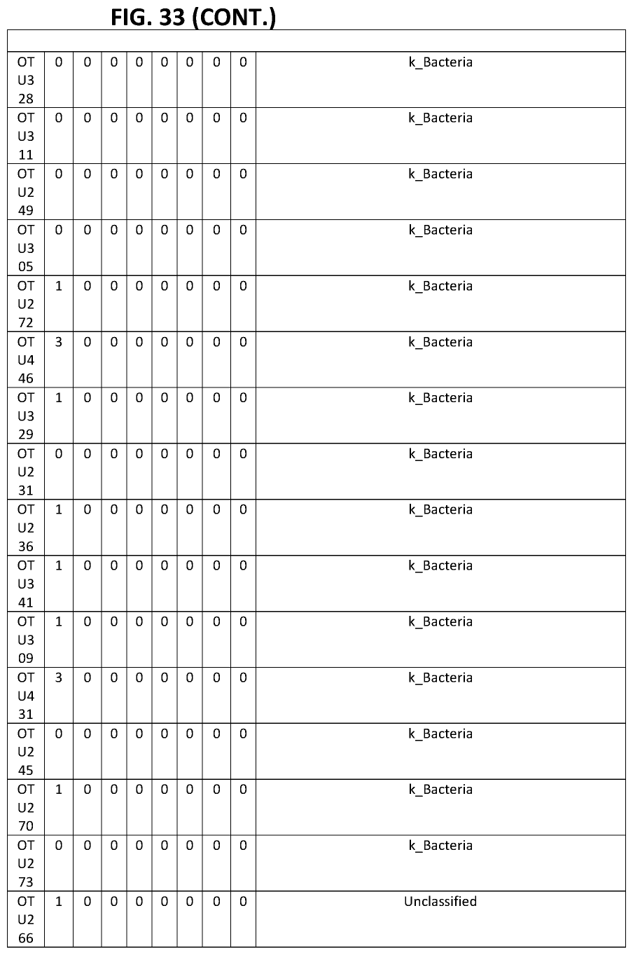

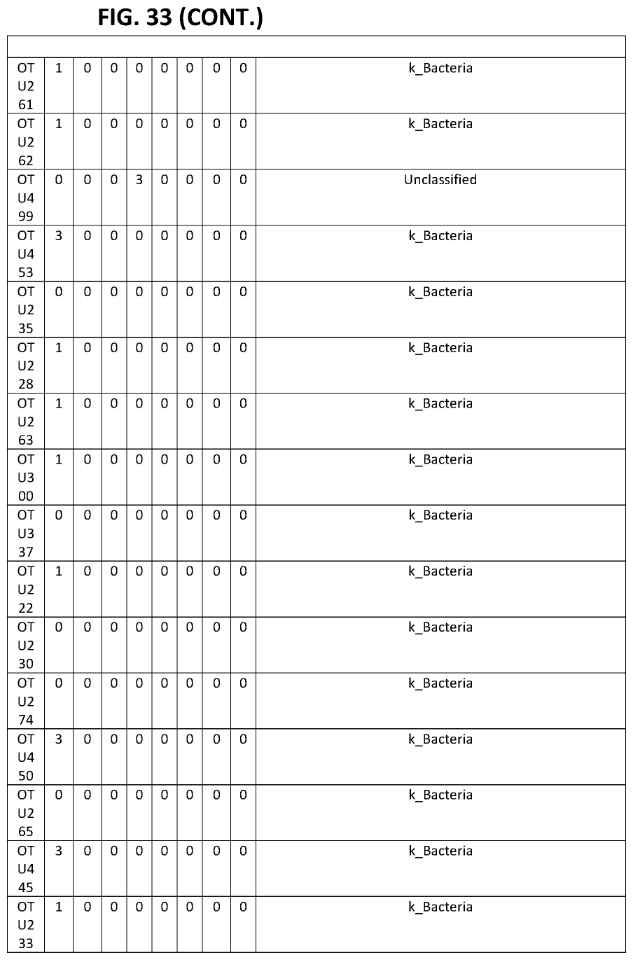

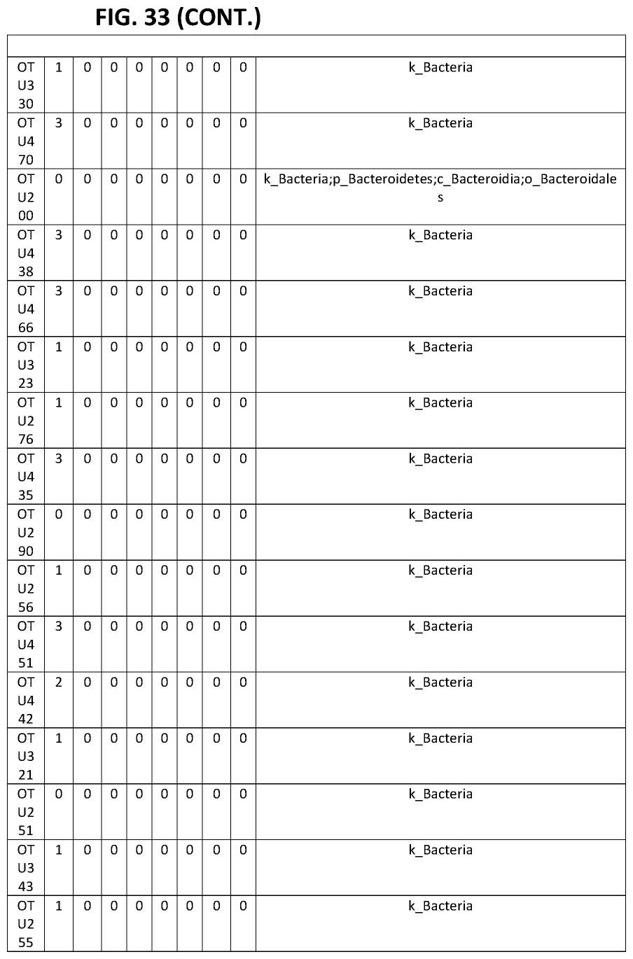

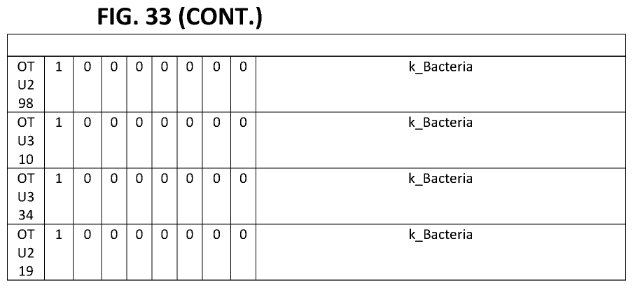

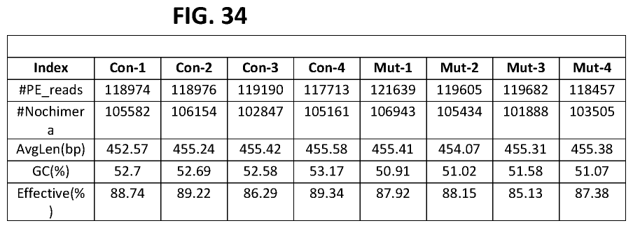

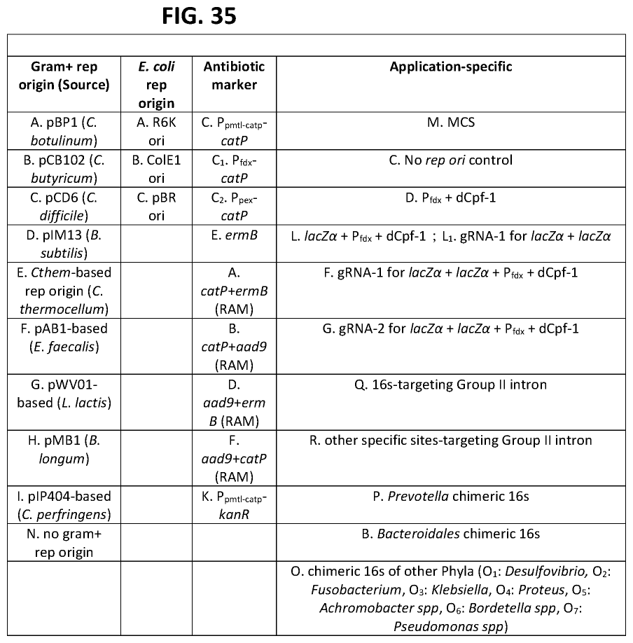

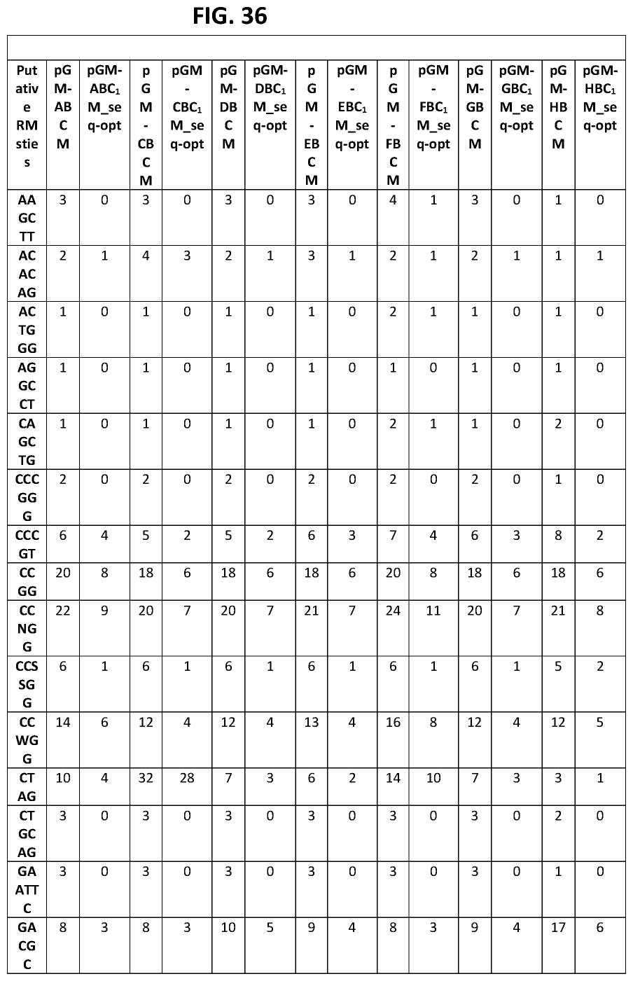

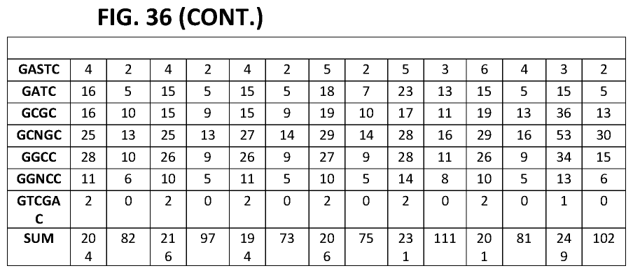

In FIGS. 6B, 6G, % of starting weight was calculated by normalizing weights at sacrifice to starting weight. In FIGS. 6D, 6I and FIGS. 6E, 6J, colon length and LCN2 data were analyzed using unpaired two-tailed Student's T-test. In FIGS. 6B, 6G and 6E, 6J, % of starting weight and hematochezia score data were analyzed using Two-way ANOVA followed by the Bonferroni post hoc test (n=4). In FIGS. 6D, 6I, histopathologic score data were analyzed using the Mann-Whitney test. Data are shown as mean±SEM. The asterisk indicates p-value <0.05 (*), <0.01 (**) or <0.001 (***). The numbering of the strains corresponds to the strain information shown in FIG. 22.

FIGS. 7A-7J. The baiH-mediated microbiota composition shift exacerbates DSS-induced colitis in gnotobiotic mice. FIG. 7A: The growth curve of two Bacteroides (bac) microbes and seven Erysipelotrichaceae (Ery) microbes in the presence of 500 μM DCA, 500 μM 3-oxo DCA, or DMSO control. The Erysipelotrichaceae microbes are more resistant to DCA and 3-oxo DCA than the Bacteroides microbes. FIG. 7B: The baiH gene drives expansion of Erysipelotrichaceae microbes in an in vitro consortium consisting of 2 Bacteroides (Bac) and 7 Erysipelotrichaceae microbes (Ery) with either the S122 control or ΩbaiH strain. 500 μM CA was supplemented as the substrate for the bai pathway. The relative fold change of Erysipelotrichaceae was assessed by qPCR. FIG. 7C: DCA drives expansion of Erysipelotrichaceae microbes in an in vitro consortium consisting of 2 Bacteroides (Bac) microbes and 7 Erysipelotrichaceae (Ery) microbes. DCA was supplemented at 0, 250, and 500 μM, respectively. The relative fold change of Erysipelotrichaceae was assessed by qPCR. FIG. 7D: DSS-induced murine colitis model was applied to the gnotobiotic mice colonized with a synthetic consortium consisting of the genetically tagged S122 control (Con) or ΩbaiH mutant (Mut) (by Group II intron) along with 2 Bacteroides (Bac) microbes and 7 Erysipelotrichaceae (Ery) microbes tested in (A), (B), and (C). Mice were colonized with the control or mutant strain for at least two weeks followed by 2.5% DSS for 8 days. FIG. 7E: The baiH gene drives expansion of Erysipelotrichaceae microbes in the context of host colonization before and during DSS treatment assessed by qPCR. The disease state was monitored by weight loss (FIG. 7F), hematoxylin and eosin (H&E) staining of the distal colon (FIG. 7G), colon shortening (FIG. 7H), fecal lipocalin-2 (FIG. 7I), and daily hematochezia score (FIG. 7J). The data in FIGS. 7A to 7C are from a representative experiment with three technical replicates (FIG. 7A), or with six or four biological replicates (FIGS. 7B, 7C). Data shown in FIGS. 7F, 7H, 7I, 7J are representations of n=4 mice per group replicated in two or more independent experiments. In FIG. 7F, % of starting weight was calculated by normalizing weights at sacrifice to starting weight. In FIG. 7H and FIG. 7I, colon length and LCN2 data were analyzed using unpaired two-tailed Student's T-test. In FIGS. 7F and 7J, % of starting weight and hematochezia score data were analyzed using Two-way ANOVA followed by the Bonferroni post hoc test (n=4). Data are shown as mean±SEM. The asterisk indicates p-value <0.05 (*), <0.01 (**) or <0.001 (***). The numbering of the strains corresponds to the strain information shown in FIG. 22.

FIG. 8. A detailed workflow and general timeline of the genetic manipulation (GM) pipeline. The dominant human gut commensals can be screened via the GM pipeline, and their targetable genetic system can be built within weeks.

FIG. 9. All the pGM vectors used in this study. Schematics of all the pGM vectors designed and used in this study are listed. “x” in pGM-xBCM (or pGM-xBCD, pGM-xBCL, pGM-xBCF/G, pGM-xBCD-xxx) represents different gram-positive replication origins, and “xxx” in pGM-xBCD-xxx corresponds to plasmid pGM-xBCD harboring different gRNA designs targeting genome of different strains (see FIGS. 28 and 35).

FIGS. 10A-10B. A mixed-conjugation strategy to identify the compatible rep oris for Clostridia microbe. FIG. 10A: A preliminary test of the mixed-conjugation strategy in a model gut commensal C. sporogenes ATCC 15579. The E. coli conjugation donors each harboring a single Clostridium rep ori and antibiotic marker gene (pMTL82254, rep ori: pBP1, antibiotic marker: ermB, erythromycin; pMTL83353, rep ori: pCB102, antibiotic marker: aad9, spectinomycin; pMTL84151, rep ori: pCD6, antibiotic marker: catP, thiamphenicol) were mixed and conjugated to a single recipient C. sporogenes ATCC 15579. After conjugation, the transconjugants were selected on agar plates supplemented with D-cycloserine and the one corresponding antibiotic (erythromycin for ermB, spectinomycin for aad9, and thiamphenicol for catP). FIG. 10B: Schematic view of a mixed-conjugation strategy to identify the Clostridia that stably maintain exogenous DNA. Ten pGM vectors, each harboring a single rep ori (9 Clostridia specific and 1 rep ori-less), were separated into three sets and mixed-conjugated to a Clostridia recipient.

FIGS. 11A-11B. Multiplex PCR strategy to identify the rep ori uptaken by the Clostridia microbes. FIG. 11A: Multiplex PCR strategy was used to identify which rep ori-contained plasmid was introduced into which Gram+ Clostridia strain in mixed-conjugation. For the mixed-conjugation with set I, primers pMTL_laz_diag_F (universal forward primer)+pGM-ABCM_rep_R_1500 bp+pGM-BBCM_rep_R_1000 bp+pGM-CBCM_rep_R_2000 bp were used for diagnostic PCR. We would see a 1.5 kb (or 1.0 kb, or 2.0 kb) PCR band if pGM-ABCM (or BBCM, or CBCM) is uptaken by the Clostridia microbe. The primers for the set II and set III mixed-conjugation are shown in FIG. 30. FIG. 11B: Distribution of Clostridial rep oris tested in this study based on phylogeny. The phylogenetic tree was constructed using the 16s rRNA sequences of the 42 gut microbes (38 Firmicutes/Clostridia, 2 Enterococcus, and 2 Actinobacteria) with a compatible rep on identified in this study. The sequences were aligned using Clustal Omega, and a neighbor-joining tree was constructed with a bootstrap test of 5000.

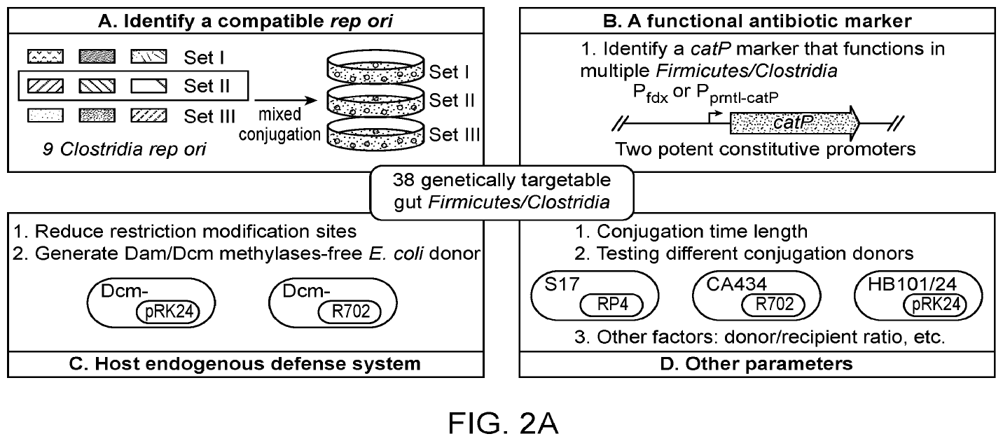

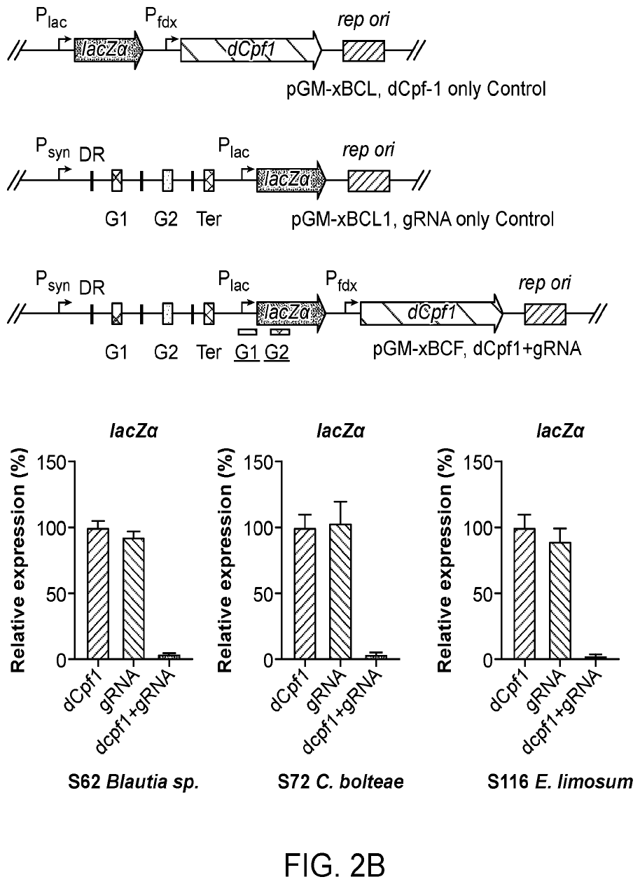

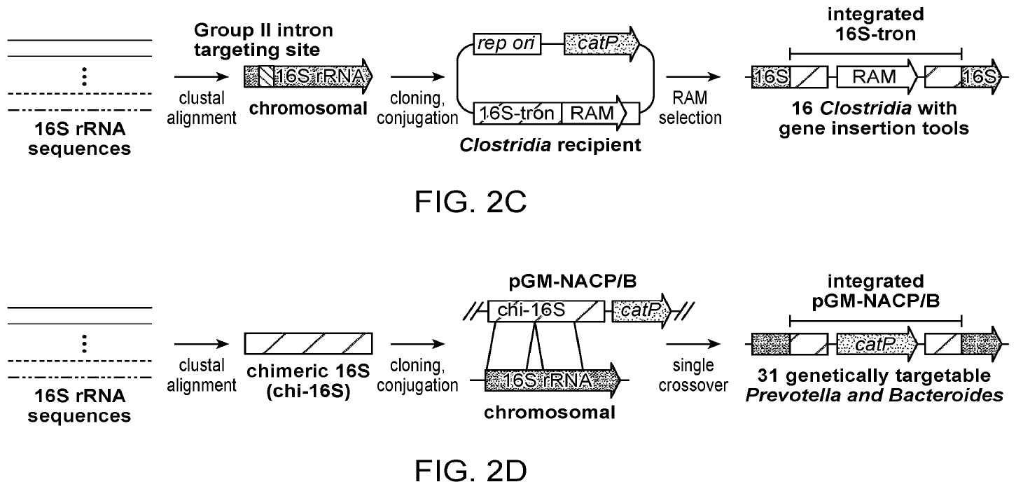

FIGS. 12A-12B. CRISPRi-dCpf1 precisely and efficiently suppressed lacZα expression in Gram-positive Clostridia and Bifidobacterium microbes. FIG. 12A: qPCR results showing that CRISPRi-dCpf1 precisely and efficiently suppressed lacZα expression in Gram-positive Clostridia and Bifidobacterium microbes, using both dCpf-1-only and gRNA-only as controls. FIG. 12B: qPCR results showing that CRISPRi-dCpf1 precisely and efficiently suppressed lacZα expression in other Gram-positive Clostridia microbes, using dCpf-1-only as control. For each strain shown in FIG. 12A, conjugation was conducted with E. coli harboring plasmids with dCpf1-lacZα (dCpf1), gRNA-lacZα (gRNA), or gRNA-dCpf1-lacZα (dCpf1+gRNA) (see FIGS. 2B and 9 for detailed information). For each strain shown in FIG. 12B, conjugation was conducted with E. coli harboring plasmids with dCpf1-lacZα (dCpf1, Con), or gRNA-dCpf1-lacZα (dCpf1+gRNA, Mut) (see FIGS. 2B and 9 for detailed information). Then transconjugants were cultured, and RNA was extracted and reverse transcribed to cDNA. Quantitative PCR (qPCR) was used to assess the expression of lacZα after normalizing to the expression of the 16s rRNA gene of each strain. Data are shown as mean±SD. The numbering of the strains corresponds to the strain information shown in FIG. 22.

FIGS. 13A-13D. Diagnostic PCR strategy of 16s-targeting Group II intron (16s-tron) integration for Clostridia and chi-16s-targeting single crossover for Bacteroidia and microbes from other phyla. FIG. 13A: Diagnostic PCR strategy to verify the 16s-targeting Group II intron (16s-tron) retrotransposition-activated marker (RAM) integration designed in targeted Clostridia commensals. The forward diagnostic primer is the sequence on the retrotransposition-activated marker, which will not bind to the genome. The reverse diagnostic primer binds to the genome and will not bind to the Group II intron plasmid. There will be a PCR product of 2.0-2.5 kb as designed for colonies that have integrated the retrotransposition-activated marker, whereas no PCR product will be found for control colonies. FIG. 13B: Three representative gel images of 16s-targeting Group II intron integration in Clostridia. There were bands of ˜2.0-2.5 kb in colonies after RAM integration using primers described in FIG. 13A, while no band was found in colonies of control. FIG. 13C: Diagnostic PCR strategy to verify the single crossover designed in targeted Bacteroidia commensals and microbes from other phyla. The diagnostic PCR strategy is the same for identifying the genetically targetable Bacteroidia and other phyla microbes using chi-16s strategy and for targeted deletion of mmdA in three Bacteroidia microbes to deplete propionate production. The forward diagnostic primer is the sequence on the target gene of the genome (16s rRNA gene or mmdA), which will not bind to the introduced suicide plasmid, the reverse diagnostic primer binds to the suicide plasmid-specific sequence and will not bind to the genome of targeted strains. There will be a PCR product of ˜2.0-2.5 kb as designed for colonies that have integrated the suicide plasmid, whereas no PCR product will be found for wild-type colonies. For screening using chi-16s, the sequencing primer is on the plasmid just downstream the chimeric 16s. As the chimeric 16s is integrated into the genome, the nucleotide sequence (from Sanger sequencing) consists of part of the original 16s rRNA sequence and part of the chimeric 16s rRNA sequence. FIG. 13D: Alignment of the nucleotide sequence of the PCR product amplified using DiagF and DiagR (as shown in FIG. 13C) with the chimeric 16s rRNA sequence and the microbial 16s rRNA sequence for the genetically targetable Bacteroidia and other phyla microbes identified using chi-16s.

FIG. 14. CRISPRi-dCpf1 precisely and efficiently suppressed bcat expression in 12 Clostridia microbes with sequenced genomes. For each strain, conjugation was conducted with E. coli harboring plasmids with dCpf1 (control, Con) or gRNA-dCpf1 (mutant, Mut) (different gRNA sequences were designed for each bcat gene in each strain, see FIG. 28 for detailed information). Colonies were cultured, and RNA was extracted and reverse transcribed to cDNA. Quantitative PCR (qPCR) was used to assess the expression of bcat after normalizing to the expression of 16s rRNA gene of each strain.

FIGS. 15A-15C. Mono-colonization of germ-free mice with the control and mutant strains of propionate, butyrate, and isovalerate. FIG. 15A: The Bacteroides sp. 1_16 (S25) ΔmmdA mutant depletes propionate in the mono-associated germ-free mice compared to that of the control mono-colonized mice. Density of intestinal colonization of gnotobiotic mice by the control (Con) and the ΔmmdA mutant (Mut) of Bacteroides sp. 1_1_6. Germ-free mice (n=3 or 4 per group) were mono-colonized with the control Bacteroides sp. 1_1_6 (the 16s rRNA has been integrated by pGM-NAC₂B) or the mutant (integrated with pGM-NAC₂B-003). The mice were fed a standard diet and supplied with water containing 15 μg/mL thiamphenicol and 2 mg/mL sugar. We calculated colony-forming units in fecal pellets to estimate the density of intestinal colonization. n.s.: not statistically significant. Propionate levels in cecal samples of germ-free mice mono-colonized with the control (Con) and the ΔmmdA mutant (Mut) of Bacteroides sp. 1_1_6. Propionate concentration was calculated using the standard curve according to AUC and normalized to each sample's weight. Data are shown as mean±SEM. Student's T-test was performed, and the asterisk indicates p-value <0.05 (*) or <0.01 (**). FIG. 15B: The Eubacterium maltosivorans DSM 105863 (S117) croA knockdown mutant depletes butyrate in the mono-associated germ-free mice compared to that of the control mono-colonized mice. Density of intestinal colonization of gnotobiotic mice by control (Con) and mutant (Mut) of Eubacterium maltosivorans DSM 105863 (S117). Germ-free mice were mono-colonized with the control Eubacterium maltosivorans DSM 105863 (containing pGM-FBCD) or the mutant (containing pGM-FBCD-020) and fed a standard diet and supplied with water containing 15 μg/mL thiamphenicol and 2 mg/mL sugar. We calculated colony-forming units in fecal pellets to estimate the density of intestinal colonization. n.s.: not statistically significant. Butyrate levels in fecal samples of mice colonized with the control (Con) and the croA knockdown mutant (Mut) of Eubacterium maltosivorans DSM 105863 (S117). Concentrations of butyrate were calculated using the standard curve according to AUC and normalized to the weight of each sample. Data are shown as mean±SEM. Student's T-test was performed, and the asterisk indicates p-value <0.05 (*) or <0.01 (**). FIG. 15C: The C. sporogenes ATCC 15579 porA knockdown mutant reduces branched-chain short-chain fatty acids production in vitro and in the mono-associated germ-free mice compared to that of the control colonized mice. C. sporogenes ATCC 15579porA knockdown mutant (Mut, carrying dCpf1 and gRNA, pGM-ABCD-006) depleted isovalerate production in vitro compared to that of the control (Con, carrying only the dCpf1, pGM-ABCD). Density of intestinal colonization of gnotobiotic mice by control (Con) and mutant (Mut) of C. sporogenes ATCC 15579. Germ-free mice were mono-colonized with control C. sporogenes ATCC 15579 (containing pGM-ABCD) or the mutant (containing pGM-ABCD-006) and fed a standard diet and supplied with water containing 15 μg/mL thiamphenicol and 2 mg/mL sugar. We calculated colony-forming units in fecal pellets to estimate the density of intestinal colonization. n.s.: not statistically significant. Isovalerate levels in cecal samples of mice colonized with the control (Con) and mutant (Mut) of C. sporogenes ATCC 15579. Concentrations of isovalerate were calculated using the standard curve according to AUC and normalized to the weight of each sample. Data are shown as mean±SEM. Student's T-test was performed, and the asterisk indicates p-value <0.05 (*) or <0.01 (**).

FIGS. 16A-16C. Gut Clostridia commensals harboring the bai operon and their prevalence and relative metagenomic abundances. FIG. 16A: Currently known gut commensals that harbor a bai operon based on their genomic sequence information. All these microbes are Clostridia commensals and have no gene transfer methodology and tractable genetic tools. Their prevalence (FIG. 16B) and percent relative abundance (FIG. 16C) were examined using a large publicly available 16s rRNA dataset (Yatsunenko et al., 2012), including stool samples from 528 individuals. The Faecalicatena contorta S122 (S122) and its closely related relatives are more prevalent than C. hylemonae, C. scindens, and D. sp. D27_3, but less than that of C. hiranonis (FIG. 16B). S122 and its closely related strains are the most abundant 7α-dehydroxylating commensal in this cohort (FIG. 16C). For FIG. 16B, the prevalence data were analyzed using Fisher's exact test, and mean±SEM was plotted. For FIG. 16C, the relative abundance data were first analyzed using the D'Agostino & Pearson test for normality. The relative abundance of S122 was compared to other commensals using the Mann-Whitney test. A Median with a 95% confidence interval (CI) was plotted. The asterisk in FIG. 16B and FIG. 16C indicates p-value <0.0001 (****).

FIGS. 17A-17E. Co-colonize gnotobiotic mice colonized with S122 and S25 or S122 and a consortium of 55 targetable gut commensals. FIG. 17A: Proposed pathway for the 7α-dehydroxylation of cholic acid (CA) to deoxycholic acid (DCA) (Funabashi et al., 2020). FIG. 17B: Successful insertion of baiH was determined by amplifying DNA using primers flanking the target gene baiH. The expected PCR product for the control is ˜2 kb, and the PCR product for the S122 ΩbaiH mutant is ˜4 kb. FIG. 17C: Metabolomics analyses of bile acids in Bacteroides sp. 116 (S25)+Faecalicatena contorta S122 (S122 ΩbaiH mutant) (Mut) and S25+S122 control (Con) co-colonized germ-free mice. Data are shown as mean±SEM. Student's T-test was performed, and the asterisk indicates p-value <0.05 (*) or <0.01 (**), n.s.: not statistically significant, n.d.: not detected. FIG. 17D: Co-colonization of Faecalicatena contorta S122 with 55 other genetically targetable microbes identified in this study in germ-free mice. Successful colonization of S122 was determined by CFU and the detection of DCA in LCMS, and colonization of other strains was confirmed by 16s rRNA sequencing. FIG. 17E: The density of total intestinal bacteria of SPF control mice compared with SPF mice colonized with Faecalicatena contorta S122 (S122) control and ΩbaiH mutant. SPF control mice (SPF) were maintained with a standard diet and water, SPF mice colonized with Faecalicatena contorta S122 (S122) control (Con) and ΩbaiH mutant (Mut) were maintained with standard diet and water with 15 μg/mL thiamphenicol and 10 μg/mL erythromycin. We calculated colony-forming units in fecal pellets on TSAB agar plates to estimate the density of intestinal bacteria. Data are shown as mean±SEM. Student's T-test was performed, and the asterisk indicates p-value <0.05 (*) or <0.01 (**), n.s.: not statistically significant.

FIG. 18. Structures of taurine-conjugated and nonconjugated bile acids in this study.

FIGS. 19A-19I. The Faecalicatena contorta S122 (S122) control and AbaiHmutant colonized SPF mice harbor a highly complex gut microbiota and analysis of the correlation between DCA and the relative abundances of gut bacterial taxonomic groups using data from healthy human stools. FIGS. 19A-19B: The Chao 1 index (FIG. 19A) and Shannon index (FIG. 19B) of the fecal microbiota of the SPF mice colonized with the control (Con) and the ΩbaiH mutant (Mut). Data are shown as mean±SEM. Student's T-test was performed, and the asterisk indicates p-value <0.05 (*) or <0.01 (**). FIGS. 19C-19D: Relative abundances of Bacteroidetes and Proteobacteria in the stool microbiome of the control and ΩbaiH mutant colonized SPF mice. ΩbaiH mutant (Mut) colonized mice harbor significantly higher abundances of Bacteroidetes and lower abundances of Proteobacteria compared to the control group (Con). Data are shown as mean±SEM. Student's T-test was performed, and the asterisk indicates p-value <0.05 (*) or <0.01 (**). FIG. 19E: Relative abundances of gut microbial taxa (at the family level) in the stool microbiome of the control and ΩbaiH mutant colonized SPF mice. Data are shown as mean±SEM. Student's T-test was performed, and the asterisk indicates p-value <0.05 (*) or <0.01 (**). FIG. 19F: Observed OTUs rarefaction curves of 16s rRNA gene sequencing of fecal samples from SPF mice colonized with Faecalicatena contorta S122 (S122) control (Con) and ΩbaiH mutant (Mut). This Fig. shows that the Con and Mut mice harbor a highly complex gut microbiota. The depth of our 16s rRNA sequencing is enough to cover the breadth of gut bacterial taxa groups in the control and mutant colonized mice. FIG. 19G: Rank-abundance curves of 16s rRNA gene sequencing of fecal samples from SPF mice colonized with Faecalicatena contorta S122 (S122) control (Con) and ΩbaiH mutant (Mut). (The X-axis is the OTU rank in descending order, and the Y-axis is the relative abundance of the OTU). FIGS. 19H-19I: In nonIBD human stools, the fecal DCA level is positively associated with the relative abundance of microbes in the Erysipelotrichaceae family and negatively associated with that of the B. eggerthii species.

FIGS. 20A-20J. baiH modulates colon inflammation in the context of complex gut microbiota. FIG. 20A: Macroscopic observations of colon length of SPF mice colonized with Faecalicatena contorta S122 (S122) control (Con) and ΩbaiH mutant (Mut) post-DSS treatment. FIG. 20B: Macroscopic observations of colon length of germ-free mice colonized with Faecalicatena contorta S122 (S122) control (Con) and ΩbaiH mutant (Mut) post-DSS treatment. FIG. 20C: Quantification of fecal lipocalin-2 (LCN-2) in SPF mice colonized with Faecalicatena contorta S122 (S122) control (Con) and ΩbaiH mutant (Mut) post-DSS treatment on the day-5, 6, 7, and day-sac, the amount of lipocalin-2 was significantly higher in the control group. Data are shown as mean±SEM. Student's T-test was performed, and the asterisk indicates p-value <0.05 (*) or <0.01 (**). FIG. 20D: Quantification of fecal lipocalin-2 (LCN-2) in germ-free mice colonized with Faecalicatena contorta S122 (S122) control (Con) and 2baiH mutant (Mut) post-DSS treatment on the day-sac, no significant difference was found. Data are shown as mean±SEM. Student's T-test was performed, n.s.: not statistically significant. FIG. 20E: Comparison of colonic expression of inflammatory genes in SPF mice colonized with Faecalicatena contorta S122 (S122) control (Con) and ΩbaiH mutant (Mut) post-DSS treatment. mRNA expression of inflammatory genes was normalized to Hprt1 and shown as the fold change relative to the mutant group. Colonic expression of inflammatory genes was significantly higher in the control group. Data are shown as mean±SEM. Student's T-test was performed, and the asterisk indicates p-value <0.05 (*) or <0.01 (**). FIG. 20F: Density of intestinal colonization of SPF mice supplemented with low dose antibiotics (15 μg/mL thiamphenicol and 10 μg/mL erythromycin in drinking water) (n=4 per group) by the S122 control (Con) and 2baiH mutant (Mut) (FIG. 6A). The colony-forming units (CFU) were calculated before DSS treatment and on day 3 and day 6 of DSS treatment. Student's T-test was performed. n.s.: not statistically significant. FIG. 20G: Relative abundances (by 16s rRNA sequencing) of Erysipelotrichaeceae in the stool microbiome of the S122 control (Con) and ΩbaiH mutant (Mut) colonized SPF mice (n=4 per group) at day 3 and day 6 of DSS treatment (FIG. 6A). Student's T-test was performed. The asterisk indicates p-value <0.01 (**) n.s.: not statistically significant. FIG. 20H: Fecal DCA levels of the SPF mice (grey bar), and the SPF mice supplemented with low dose antibiotics (n=4 per group) colonized with the S122 control (Con) strain (before and at day 3 of DSS treatment) (FIG. 6A). Student's T-test was performed. n.s.: not statistically significant.

FIG. 20I: The relative abundances of S122 in the control and mutant group (n=4 per group) of the DSS germ-free experiment (FIG. 6B) were assessed by 16s rRNA sequencing and were comparable. Student's T-test was performed. n.s.: not statistically significant. FIG. 20J: Targeted metabolomics analyses of the stool bile acid (BA) compositions of the S122 control (Con) and ΩbaiH mutant (Mut) colonized gnotobiotic mice (n=4 per group) at day 3 after the DSS treatment (FIG. 6F). Student's T-test was performed. The asterisk indicates p-value <0.01 (**) n.s.: not statistically significant, n.d.: not detected.

FIG. 21. Growth curve of Bacteroides and Erysipelotrichaceae microbes. Growth curve of Bacteroides (Bacteroides fragilis 3112 (Bac1) and Bacteroides vulgatus ATCC 8482 (Bac2)) and Erysipelotrichaceae (Clostridium ramosum ATCC 25554 (Ery1), Erysipelatoclostridium ramosum strain 113-1 (Ery2), Clostridium ramosum DSM 24812 (Ery3), Clostridium ramosum DSM 1402 (Ery4), Clostridium innocuum 6_1_30 (Ery5), Clostridium innocuum DSM 22910 (Ery6), and Holdemania filiformis DSM 12042 (Ery7)) were measured in 500 μM of DCA, 3-oxoDCA, CA and 7-oxoCA) with DMSO as control. Optical densities at 600 nm (OD₆₀₀) were recorded every 30 min until the cultures reached the stationary phase. Bacterial growth curves were performed in triplicate, with each biological replicate deriving from a single colony.

FIGS. 22A-22L. The baiH-mediated microbiota composition shift exacerbates DSS-Induced colitis in gnotobiotic mice. FIG. 22A: Density of intestinal colonization (assessed by CFU) of gnotobiotic mice colonized with Faecalicatena contorta S122 (S122) control (Con) and ΩbaiH mutant (Mut), and total intestinal bacterial load in the 10-member community as shown in FIG. 7D. We calculated colony-forming units in fecal pellets to estimate the density of intestinal colonization. n.s.: not statistically significant. FIG. 22B: Density of intestinal colonization (assessed by CFU) of gnotobiotic mice colonized with Faecalicatena contorta S122 (S122) control (Con) and ΩbaiH mutant (Mut), and total intestinal bacterial load in the 3-member community as shown in FIG. 22G. We calculated colony-forming units in fecal pellets to estimate the density of intestinal colonization. n.s.: not statistically significant. FIGS. 22C-22D: Metabolomics analyses of fecal bile acids in feces of two Bacteroides (bac) microbes and seven Erysipelotrichaceae (Ery) microbes+Faecalicatena contorta S122 control (Con)/(S122 ΩbaiH mutant) (Mut) co-colonized germ-free mice before DSS treatment (FIG. 22C) and on day 3 of DSS treatment (FIG. 22D). Data are shown as mean±SEM. Student's T-test was performed, and the asterisk indicates p-value <0.05 (*) or <0.01 (**), n.s.: not statistically significant, n.d.: not detected. FIGS. 22E-22F: Metabolomics analyses of bile acids in feces of two Bacteroides (bac) microbes+Faecalicatena contorta S122 control (Con)/(S122 ΩbaiH mutant) (Mut) co-colonized germ-free mice before DSS treatment (FIG. 22E) and on day 3 of DSS treatment (FIG. 22F). Data are shown as mean±SEM. Student's T-test was performed, and the asterisk indicates p-value <0.05 (*) or <0.01 (**), n.s.: not statistically significant, n.d.: not detected. FIGS. 22G-22L: DSS-induced murine colitis model was applied to the gnotobiotic mice colonized with a synthetic consortium consisting of the genetically tagged S122 control (Con) or ΩbaiH mutant (Mut) along with 2 Bacteroides (Bac) microbes only. Mice were colonized with the consortium for at least two weeks, followed by 2.5% DSS for 9 days. The disease state was monitored by weight loss (FIG. 22H), hematoxylin and eosin (H&E) staining of the distal colon (FIG. 22I), colon shortening (FIG. 22J), fecal lipocalin-2 (FIG. 22K), and daily hematochezia score (FIG. 22L). Data shown in FIGS. 22H-22L are representations of n=4 mice per group replicated in two independent experiments. In FIG. 22H, % of starting weight was calculated by normalizing weights at sacrifice to starting weight. In FIGS. 22J and 22K, colon length and LCN2 data were analyzed using unpaired two-tailed Student's T-test. In FIGS. 22H and 22L, % of starting weight and hematochezia score data were analyzed using Two-way ANOVA followed by the Bonferroni post hoc test (n=4). Data are shown as mean±SEM. The asterisk indicates p-value <0.05 (*), <0.01 (**) or <0.001 (***). The numbering of the strains corresponds to the strain information shown in FIG. 22.

FIG. 23. Culture conditions of all the gut commensals screened in this study.

FIG. 24. Optimized factors for introducing plasmid DNA into non-model gut microbes.

FIG. 25. 91 gut microbes and the corresponding compatible plasmids after multifactorial optimization.

FIG. 26. Genes targeted in gram-positive Firmicutes/Clostridia strains and strains from other phyla.

FIG. 27. Genes targeted in gram-negative Bacteroidia strains and strains from other phyla.

FIG. 28. Vectors for the construction of mutants in gram+ and gram− strains.

FIG. 29. Bacterial strains used in this study.

FIG. 30. Primers and gRNA sequences used in this study (SEQ ID NOs: 23-287 in order of appearance).

FIG. 31. Recipes for all the culture media used in this study and their ingredient details.

FIG. 32. Taxonomy abundances (%) of 16s rRNA gene sequencing of fecal samples from SPF mice colonized with Faecalicatena contorta S122 (S122) control (Con) and ΩbaiH mutant (Mut).

FIG. 33. Number of reads and taxonomy of each OTU of 16s rRNA gene sequencing of fecal samples from SPF mice colonized with Faecalicatena contorta S122 (S122) control (Con) and 12baiH mutant (Mut).

FIG. 34. Effective statistics of 16s rRNA gene sequencing of fecal samples from SPF mice colonized with Faecalicatena contorta S122 (S122) control (Con) and ΩbaiH mutant (Mut).

FIG. 35. Nomenclature of all the pGM vectors designed in this study.

FIG. 36. Putative RM sites (SEQ ID NOs: 288-309 in order of appearance) reduced in the sequence optimization.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

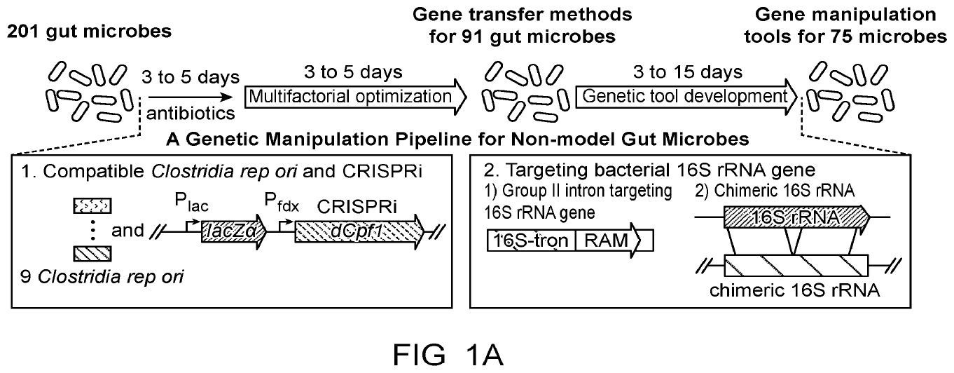

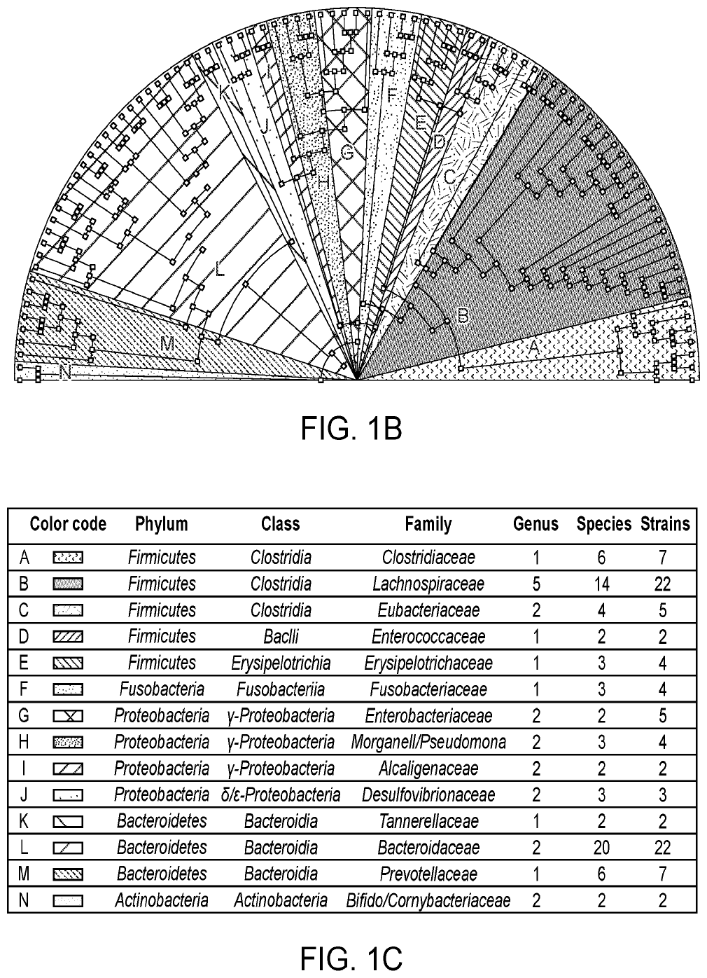

Disclosed herein is a genetic manipulation (GM) pipeline to identify gene transfer methodology and build a genetic tool for non-model human gut commensals on a large scale (201 gut isolates from >140 species in five phyla) (FIG. 1). This pipeline efficiently identified the gene transfer methods for 91 non-model gut bacterial isolates (72 species), including 81 previously untransformed microbes, and built their tools for targeted gene manipulation (FIG. 1). Of note, gut Firmicutes/Clostridia comprises one of the most abundant bacterial groups in healthy human guts, yet its genetic manipulation is largely unexplored (Waller et al., 2017). Via a multifactorial optimization of their conjugation/transformation conditions, the present disclosure identified the gene transfer methods for 38 non-model gut Clostridia, and set up CRISPRi or Group II intron-based genetic tools in 27 of them. The Examples herein demonstrated the utility of these toolsets by modulating short-chain fatty acids (SCFAs) and secondary bile acids in vitro and in the context of host colonization. As a proof of principle, one Clostridia specific pathway-bile acid 7a dihydroxylation was selected for further functional investigation. By genetically tagging the Clostridia commensal, the bai gene in a complex microbiome was manipulated. Provided herein is evidence that the bai gene significantly impacts host gut microbiome and bile acid composition and mediates colon inflammation in a complex microbiome.

The pipeline described here and the related findings represent the first large-scale identification of gene transfer methodology for non-model gut bacterial isolates. This screen greatly expands the manipulatable genes/pathways coded by the gut microbiota. For instance, microbiota pathways encoded by the gut microbes that previously had no tractable genetic tools, like that for butyrate or bile acid 7α-dehydroxylation, were identified in the library of genetically targetable commensals described herein and manipulated. This library of targetable gut isolates and their genetic tools serve as a starting point for precisely controlling microbiome molecular output and interrogating their effects on host biology. The GM pipeline efficiently identifies gene transfer methods for gut bacterial isolates and develops their gene manipulation tools without prior knowledge of their genome sequence. Both features suggest its application as a useful technology to delineate the genetics for non-model gut Firmicutes/Clostridia commensals.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. In some embodiments, a Cas9 nuclease has an inactive (e.g., an inactivated) DNA cleavage domain, that is, the Cas9 is a nickase.

A nuclease-defective Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one or two Cas9 domains: (1) the gRNA binding domain of Cas9; or (2) the DNA cleavage domain of Cas9.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, “conjugation” refers to the temporary direct contact between two bacterial cells leading to an exchange of genetic material (DNA). This exchange is unidirectional, i.e. one bacterial cell is the donor of DNA and the other is the recipient. In this way, genes are transferred laterally amongst existing bacterial as opposed to vertical gene transfer in which genes are passed on to offspring. Conjugation is a convenient means for transferring genetic material to bacteria.

“Cpf1 protein,” as used herein, refers to a Cpf1 wild-type protein derived from Class 2 Type V CRISPR-Cpf1 systems, modifications of Cpf1 proteins, variants of Cpf1 proteins, Cpf1 orthologs, and combinations thereof. Cpf1 proteins include, but not limited to, Francisella novicida (UniProtKB—A0Q7Q2 (CPF1_FRATN)), Lachnospiraceae bacterium (UniProtKB—A0A182DWE3 (A0A182DWE3_9FIRM)), and Acidaminococcus sp. (UniProtKB—U2UMQ6 (CPF1_ACISB)). Cpf1 is the signature protein characteristic for Class 2 Type V CRISPR systems. Cpf1 homologs can be identified using sequence similarity search methods known to one skilled in the art. “dCpf1,” as used herein, refers to variants of Cpf1 protein that are nuclease-deactivated Cpf1 proteins, also termed “catalytically inactive Cpf1 protein,” or “enzymatically inactive Cpf1.”

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the term “genome” refers to the whole hereditary information of an organism that is encoded in the DNA (or RNA for certain viral species) including both coding and non-coding sequences. In various embodiments, the term may include the chromosomal DNA of an organism and/or DNA that is contained in an organelle such as, for example, the mitochondria or chloroplasts and/or extrachromosomal plasmid and/or artificial chromosome.

As used herein, the term “group II intron” refers to a class of bacterial retrotransposons that insert site-specifically into DNA target sites by a mechanism termed “retrohoming” in which the excised intron RNA reverse splices into a DNA strand and is reverse transcribed by the intron-encoded protein (a reverse transcriptase). Retrohoming is mediated by a ribonucleoprotein particle that contains the intron-encoded protein and excised intron RNA, with target specificity determined largely by base pairing of the intron RNA to the DNA target sequence. This feature enabled the development of mobile group II introns into bacterial gene targeting vectors (“targetrons”) with programmable target specificity.

The term “guide sequence” refers to the portion of a crRNA or guide RNA (gRNA) that is responsible for hybridizing with the target DNA.

As used herein, a “heterologous nucleic acid sequence” is any nucleic acid sequence placed at a location where it does not normally occur. A heterologous nucleic acid sequence may comprise a sequence that does not naturally occur in a cell, or it may comprise only sequences naturally found in the cell, but placed at a non-normally occurring location in the cell. In some embodiments, the heterologous nucleic acid sequence is not an endogenous sequence. In certain embodiments, the heterologous nucleic acid sequence is an endogenous sequence that is derived from a different cell. In other embodiments, the heterologous nucleic acid sequence is a sequence that occurs naturally in a cell but is then relocated to another site where it does not naturally occur, rendering it a heterologous sequence at that new site.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule can therefore have a nucleotide sequence that facilitates the exchange of nucleotide strands between the first nucleic acid molecule and a defined position of the second nucleic acid molecule. Thus, the first nucleic acid can generally have a nucleotide sequence that is sufficiently complementary to a portion of the second nucleic acid molecule to promote nucleotide base pairing. Homologous recombination requires homologous sequences in the two recombining partner nucleic acids but does not require any specific sequences. Homologous recombination can be used to introduce a heterologous nucleic acid and/or mutations into the host genome. Such systems typically rely on sequence flanking the heterologous nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” are used interchangeably and refer to an individual organism, a vertebrate, a mammal, or a human. In a preferred embodiment, the individual, patient or subject is a human.

As used herein, “microbiome” refers to the collective genetic content of the communities of microbes that live in and on the human body, both sustainably and transiently, including eukaryotes, fungi, archaea, bacteria, and viruses (including bacterial viruses (i.e., phage)), wherein “genetic content” includes genomic DNA, RNA such as micro RNA and ribosomal RNA, the epigenome, plasmids, and all other types of genetic information. As used herein, the term “gut microbiome” refers to the collective genetic content of the communities of microbes present in the gastrointestinal tract (GIT).

As used herein, “microbiota” refers to the collective microbes that live in and on the human body, both sustainably and transiently, including eukaryotes, fungi, archaea, bacteria, and viruses (including bacterial viruses (i.e., phage)). “Gut microbiota” as used herein refers to the totality of the microbes present in the GIT, including eukaryotes, fungi, archaea, bacteria, and viruses (including bacterial viruses (i.e., phage)).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, “operably linked” means that expression control sequences are positioned relative to a nucleic acid of interest to initiate, regulate or otherwise control transcription of the nucleic acid of interest. In some embodiments, transcription of a polynucleotide operably linked to an expression control element (e.g., a promoter) is controlled, regulated, or influenced by the expression control element.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

A “protospacer sequence” refers to the target double stranded DNA and specifically to the portion of the target DNA (e.g., target region in the genome (e.g., the genome of the target bacterium)) that is fully or substantially complementary (and hybridizes) to a guide sequence of a CRISPR RNA (crRNA). In the case of Type I and II CRISPR-Cas systems, the protospacer sequence is directly flanked by a PAM.

The term “protospacer adjacent motif” (or PAM) as used herein, refers to a 2-6 base pair DNA sequence that flanks the DNA region targeted for cleavage by the CRISPR system, such as CRISPR-Cas9. The PAM is required for a Cas nuclease to cut and is generally found 3-4 nucleotides downstream from the cut site. The PAM specificity may be a function of the DNA-binding specificity of the Cas nuclease protein.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the cell of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the cell of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a cell. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the term “replication origins”, “origins of replications” or “rep origins” refers to a unique DNA sequence of a replicon at which DNA replication is initiated and proceeds bidirectionally or unidirectionally. It contains the sites where the first separation of the complementary strands occurs, a primer RNA is synthesized, and the switch from primer RNA to DNA synthesis takes place.

As used herein, a “reporter gene” refers to a polynucleotide sequence encoding a gene product (e.g., polypeptide) that can generate, under appropriate conditions, a detectable signal that allows detection of the presence and/or quantity of the gene product. Reporter genes are often used as an indication of whether a certain gene has been introduced into or expressed in the host cell or organism. Examples of commonly used reporters include: antibiotic resistance genes, fluorescent proteins, auxotropic selection modules, β-galactosidase (encoded by the bacterial gene lacZ), luciferase (from lightning bugs), chloramphenicol acetyltransferase (CAT; from bacteria), GUS (β-glucuronidase; commonly used in plants) and green fluorescent protein (GFP; from jelly fish). Reporters or selection moduless can be selectable or screenable.

The term “seed region” refers to the RNA sequence responsible for initial complexation between a target DNA sequence and CRISPR gRNA/nuclease complex. Mismatches between the seed region and a target DNA sequence have a stronger effect on target site recognition and cleavage than the remainder of the crRNA/sgRNA sequence. In some embodiments, a single mismatch in the seed region of a crRNA/gRNA can render a CRISPR complex inactive at that binding site. In some embodiments, the seed regions for Cas9 endonucleases are located along the last −12 nts of the 3′ portion of the guide sequence, which correspond (hybridize) to the portion of the protospacer target sequence that is adjacent to the PAM. In some embodiments, the seed regions for Cpf1 endonucleases are located along the first −5 nts of the 5′ portion of the guide sequence, which correspond (hybridize) to the portion of the protospacer target sequence adjacent to the PAM.

As used herein, “selection marker” refers to a gene that confers a trait suitable for artificial selection. Typically host cells expressing the selectable selection marker is protected from a selective agent that is toxic or inhibitory to cell growth. Examples of commonly used selective markers include antibiotic resistance genes. A screenable selection marker (e.g., gfp, lacZ) generally allows researchers to distinguish between wanted cells (expressing the selection module) and unwanted cells (not expressing the selection module or expressing at insufficient level).

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, “16S ribosomal RNA” or “16S rRNA”, is a component of the prokaryotic ribosome 30S subunit. The 16S rRNA gene is the DNA sequence corresponding to rRNA encoding bacteria, which exists in the genome of all bacteria. 16S rRNA is highly conserved and specific, and the gene sequence is long enough (about 1,500 base pairs) for informatics purposes. 16S rRNA sequences are used for phylogenetic reconstruction as they are generally highly conserved, but contain specific hypervariable regions that harbor sufficient nucleotide diversity to differentiate genera and species of most bacteria.

As used herein, a “vector” refers to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

### CRISPR-Cas Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et al., Nature 482: 331 (2012); Bhaya, D., et al., Annu. Rev. Genet. 45: 231 (2014); and Terms, M. P., et. al, Curr. Opin. Microbiol. 14: 321 (2011)). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et al., Science 329:1355 (2012); Gesner, E. M., et al., Nat. Struct. Mol. Biol. 18: 688 (2001); Jinek, M., et al., Science 337: 816-21 (2012)). Target recognition by crRNAs occurs through complementary base pairing with target DNA, which directs cleavage of foreign sequences by means of Cas proteins. (Jinek et al., Science 337: 816-821 (2012)).

There are at least five main CRISPR system types (Type I, II, III, IV and V) and at least 16 distinct subtypes (Makarova, K. S., et al., Nat. Rev. Microbiol. 13: 722-736 (2015)). CRISPR systems are also classified based on their effector proteins. Class 1 systems possess multi-subunit crRNA-effector complexes, whereas in class 2 systems all functions of the effector complex are carried out by a single protein (e.g., Cas9 or Cpf1). As used herein, “CRISPR enzyme”, “Cas protein” and “CRISPR-Cas protein” refer to CRISPR-associated proteins (Cas) including, but not limited to Class 1 Type I CRISPR-associated proteins, Class 1 Type III CRISPR-associated proteins, and Class 1 Type IV CRISPR-associated proteins, Class 2 Type II CRISPR-associated proteins, Class 2 Type V CRISPR-associated proteins, and Class 2 Type VI CRISPR-associated proteins. The Cas protein of the present technology can be selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the present disclosure teaches using type II and/or type V single-subunit effector systems. Thus, in some embodiments, the present disclosure teaches using class 2 CRISPR systems. Class 2 Cas proteins include Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. In some embodiments, Cas proteins are Class 2 CRISPR-associated proteins, for example one or more Class 2 Type II CRISPR-associated proteins, such as Cas9, one or more Class 2 Type V CRISPR-associated proteins, such as Cpf1, and one ore more Class 2 Type VI CRISPR-associated proteins, such as C2c2. In preferred embodiments, Cas proteins are one or more Class 2 Type II CRISPR-associated proteins, such as Cas9, and one or more Class 2 Type V CRISPR-associated proteins, such as Cpf1. Typically, for use in aspects of the present technology, a Cas protein is capable of interacting with one or more cognate polynucleotides (most typically RNA) to form a nucleoprotein complex (most typically, a ribonucleoprotein complex).

CRISPR-Cas nucleases and associated RNAs can be repurposed to edit the genomes in bacteria, yeast and human cells. These techniques all rely on the use of a Cas nucleases to introduce double strand breaks at specific loci.

In addition to gene editing, CRISPR-Cas has been further exploited for CRISPR activation (CRISPRa) and CRISPR interference (CRISPRi) using nuclease-deactivated Cas proteins. CRISPRa and CRISPRi utilize nuclease-deactivated Cas proteins (e.g., dCas9, dCpf1) that cannot generate a double strand, but instead target genomic regions resulting in RNA-directed transcriptional control. CRISPRi utilizes nuclease-deactivated Cas proteins that complexes with gRNA to target promoter regions for transcriptional repression, or knockdown, of the gene. CRISPRa employs nuclease-deactivated Cas proteins fused to different transcriptional activation domains, which can be directed to promoter regions by either standard gRNA or special gRNAs that recruit additional transcriptional activation domains to upregulate expression of the target gene.

### CRISPR Cas9

In some embodiments, the present disclosure provides gene editing methods using a Type II CRISPR system. In some embodiments, the Type II CRISPR system uses the Cas9 enzyme. Type II systems rely on a i) single endonuclease protein, ii) a tracrRNA, and iii) a crRNA where a ˜20-nucleotide (nt) portion of the 5′ end of crRNA is complementary to a target nucleic acid. The region of a crRNA strand that is complementary to its target DNA protospacer is hereby referred to as“guide sequence.” In some embodiments, the tracrRNA and crRNA components of a Type II system can be replaced by a single-guide RNA (sgRNA)

Cas9 endonucleases produce blunt end DNA breaks and are recruited to target DNA by a combination of a crRNA and a tracrRNA oligos, which tether the endonuclease via complementary hybridization of the RNA CRISPR complex. DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., et al., Science 337: 816-821 (2012)). In some embodiments, the PAM motif recognized by a Cas9 varies for different Cas9 proteins.

In some embodiments, one skilled in the art can appreciate that the Cas9 disclosed herein can be any variant derived or isolated from any source. In other embodiments, the Cas9 peptide of the present disclosure can include one or more of the mutations described in the literature, including but not limited to the functional mutations described in: Fonfara et al., Nucleic Acids Res. 42(4):2577-2590 (2014); Nishimasu H. et al., Cell 156(5): 935-949 (2014); Jinek M. et al., Science 337:816-821 (2012); and Jinek M. et al., Science 343 (6176): 1247997 (2014); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity.

The present disclosure further envisions the use of catalytically inactivated Cas9 mutants, or dCas9. A non-limiting list of mutations that reduce or eliminate nuclease in Cas9 includes: D10, G12, G17, E762, H840, N854, N863, H982, H983, A984, D986, or A987, or a mutation in a corresponding location in a Cas9 homologue or ortholog. The mutation(s) can include substitution with any natural (e.g., alanine) or non-natural amino acid, or deletion. An exemplary nuclease defective dCas9 protein is Cas9D10A&H840A (Jinek, et al., Science 337: 816-821 (2012); Qi, et al., Cell 152(5): 1173-1183 (2013)).

### CRISPR Cpf1

In other embodiments, the present disclosure teaches methods of gene editing using a Type V CRISPR system. In some embodiments, the present disclosure teaches methods of using CRISPR from Prevotella and Francisella 1 (Cpf1).

The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long. In some embodiments, the present disclosure will refer to a crRNA for Cpf1 as a “guide RNA.”

Second, Cpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B., et al., Cell 163: 759-771 (2015), which is hereby incorporated by reference in its entirety for all purposes).

Third, the cut sites for Cpf1 are staggered by about 3-5 bases, which create“sticky ends” (Kim D., et al., Nat Biotechnol. 34(8): 863-868 (2016)). These sticky ends with ˜3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the“seed” region is located within the first 5 nt of the guide sequence. Cpf1 crRNA seed regions are highly sensitive to mutations, and even single base substitutions in this region can drastically reduce cleavage activity (see Zetsche B., et al., Cell 163: 759-771 (2015)). Critically, unlike the Cas9 CRISPR target, the cleavage sites and the seed region of Cpf1 systems do not overlap. Additional guidance on designing Cpf1 crRNA targeting oligos is available on (Zetsche B., et al., Cell 163: 759-771 (2015)).

One skilled in the art will appreciate that the Cpf1 disclosed herein can be any variant derived or isolated from any source. The present disclosure further envisions the use of catalytically inactivated Cpf1 mutants. Thus in some embodiments, the present disclosure teaches dCpf1 mutants. In some embodiments, the dCpf1 of the present disclosure comprises: ddCpf1 (Zhang et al., Cell Discov. 3: 17018 (2017); Francisella novicida (UniProtKB—A0Q7Q2 (CPF 1 FRATN)), Lachnospiraceae bacterium (UniProtKB—A0A182DWE3 (A0A182DWE3 9FIRM)), and Acidaminococcus sp. (UniProtKB—U2UMQ6 (CPF1 ACISB). In preferred embodiments, the dCpf1 of the present disclosure is generated by mutating the catalytic domain AsCpfl, for example, dCpf1 having a D908A mutation, as described by Yamano, T., et al., Cell 165: 949-962 (2016), which is incorporated herein by reference in its entirety.

### Expression Vectors for Genetically Modifying Gram-Negative Commensal Human Gut Bacteria

In one aspect, the present disclosure provides a bacterial expression vector comprising (a) a nucleic acid encoding a target gene that is conserved in a plurality of human gut commensal gram-negative bacterial species and (b) a heterologous nucleic acid encoding a selectable marker, wherein the selectable marker is an antibiotic resistance gene or an auxotrophic marker. Examples of target genes that are largely conserved in human gut commensal gram-negative bacterial species include, but are not limited to 16s rRNA, 23s rRNA, mmdA, RokA (Clucokinase gene), and ABC transporter genes. Additionally or alternatively, in some embodiments, the bacterial expression vector further comprises at least one open reading frame encoding a bioluminescent protein, a chemiluminescent protein, a fluorescent protein, a CRISPR enzyme, a Group II intron-encoded protein, at least one sgRNA, at least one Group II intron, or any combination thereof.

In some embodiments, the target gene is a chimeric sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the 16S rRNA gene sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 Bacteroidia (e.g., Prevotella and Bacteroides) microbes. In other embodiments, the target gene is a chimeric sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the 23S rRNA gene sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 Bacteroidia (e.g., Prevotella and Bacteroides) microbes.

In some embodiments, the target gene is a chimeric sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the mmdA gene sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 Bacteroidia (e.g., Prevotella and Bacteroides) microbes. In other embodiments, the target gene is a chimeric sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the RokA gene sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 Bacteroidia (e.g., Prevotella and Bacteroides) microbes. In certain embodiments, the target gene is a chimeric sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to an ABC transporter gene sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 Bacteroidia (e.g., Prevotella and Bacteroides) microbes.

A non-limiting example of a chimeric 16S rRNA sequence is:

(SEQ ID NO: 11)
CGAATTCCTGCAGCCCGGGTGGGGATGCGTTCCATTAGGTAGTTGGCGG
GGTAACGGCCCACCAAGCCTACGATGGATAGGGGTTCTGAGAGGAAGGT
CCCCCACATTGGAACTGAGACACGGTCCAAACTCCTACGGGAGGCAGCA
GTGAGGAATATTGGTCAATGGGCGAGAGCCTGAACCAGCCAAGTAGCGT
GAAGGATGACTGCCCTATGGGTTGTAAACTTCTTTTATATGGGAATAAA
GTTCGGTACGTGTGGGATTTTGTATGTACCATATGAATAAGGATCGGCT
AACTCCGTGCCAGCAGCCGCGGTAATACGGAGGATCCGAGCGTTATCCG
GATTTATTGGGTTTAAAGGGAGCGTAGGTGGATCGTTAAGTCAGTTGTG
AAAGTTTGCGGCTCAACCGTAAAATTGCAGTTGATACTGGAGGTCTTGA
GTACAGTAGAGGTAGGCGGAATTCGTGGTGTAGCGGTGAAATGCTTAGA
TATCACGAAGAACTCCGATTGCGAAGGCAGCTTACTAGACTGCAACTGA
CACTGATGCTCGAAAGTGTGGGTATCAAACAGGATTAGATACCCTGGTA
GTCCACACAGTAAACGATGAATACTCGCTGTTTGCGATATACAGTAAGC
GGCCAAGCGAAAGCATTAAGTATTCCACCTGGGGAGTACGCCGGCAACG
GTGAAACTCAAAGGAATTGACGGGGGCCCGCACAAGCGGAGGAACATGT
GGTTTAATTCGATGATACGCGAGGAACCTTACCCGGGCTTAAATTGCTG
AATTGGAAACAGCTTAGCCGCAAGGCAGATGTGAAGGTGCTGCATGGTT
GTCGTCAGCTCGTGCCGTGAGGTGTCGGCTTAAGTGCCATAACGAGCGC
AACCCTTATCTTTAGTTACTAACAGGTCATGCTGAGGACTCTAGAGAGA
CTGCCGTCGTAAGATGTGAGGAAGGTGGGGATGACGTCAAATCAGCACG
GCCCTTACGTCCGGGGCTACACACGTGTTACAATGGGGGGTACAGAAGG
CAGCTACCTGGCGACAGGATGCTAATCCCAAAAACCTCTCTCAGTTCGG
ATCGGAGTCTGCAACCCGACTCCGTGAAGCTGGATTCGCTAGTAATCGC
GCATCAGCCACGGCGCGGTGAATACGTTCCCGGGCCTTGTACACACCGC
CCGTCAAGCCATGAAAGCCGGGGGTACCTGAAGTACGTGGATCCACTAG
TTCTAGAGC

Additionally or alternatively, in some embodiments, the bacterial expression vector comprises the nucleic acid sequence of SEQ ID NO: 310 (provided below):

| (SEQ ID NO: 310) | |
|---|---|
| 1 | acgcgttatg agtaaacttg gtctgacagt taccaatgct taatcagtga ggcacctatc |
| 61 | tcagcgatct gtctatttcg ttcatccata gttgcctgac tccccgtcgt gtagataact |
| 121 | acgatacggg agggcttacc atctggcccc agtgctgcaa tgataccgcg agacccacgc |
| 181 | tcaccggctc cagatttatc agcaataaac cagccagccg gaagggccga gcgcagaagt |
| 241 | ggtcctgcaa ctttatccgc ctccatccag tctattaatt gttgccggga agctagagta |
| 301 | agtagttcgc cagttaatag tttgcgcaac gttgttgcca ttgctacagg catcgtggtg |
| 361 | tcacgctcgt cgtttggtat ggcttcattc agctccggtt cccaacgatc aaggcgagtt |
| 421 | acatgatccc ccatgttgtg caaaaaagcg gttagctcct tcggtcctcc gatcgttgtc |
| 481 | agaagtaagt tggccgcagt gttatcactc atggttatgg cagcactgca taattctctt |
| 541 | actgtcatgc catccgtaag atgcttttct gtgactggtg agtactcaac caagtcattc |
| 601 | tgagaatagt gtatgcggcg accgagttgc tcttgcccgg cgtcaatacg ggataatacc |
| 661 | gcgccacata gcagaacttt aaaagtgctc atcattggaa aacgttcttc ggggcgaaaa |
| 721 | ctctcaagga tcttaccgct gttgagatcc agttcgatgt aacccactcg tgcacccaac |
| 781 | tgatcttcag catcttttac tttcaccagc gtttctgggt gagcaaaaac aggaaggcaa |
| 841 | aatgccgcaa aaaagggaat aagggcgaca cggaaatgtt gaatactcat actcttcctt |
| 901 | tttcaatcat ggccgcggga ttaaaagtcg gggattggtg aacaaaaagg tgtttctctc |
| 961 | tttaagagaa atatcgtttt gctaaacagt tgatattgag gtatcatttt atcgtaaaag |
| 1021 | acatttttgc tcaacaattg cttgacggaa atcaacaaat tttagcattt tgtaaaaaag |
| 1081 | tcgctatata atttggtgaa ttggagttat tttcatattt ttgcatcccg aagagtttct |
| 1141 | cttaaagaga gaaacatctt ttgcatacct tttccgaccg aatttttatg tcgtaaagag |
| 1201 | gggctttgca gggggtggac tcagaaagat gagaatagat gactattgta gttgaaacac |
| 1261 | atagaaagtt gctgatatac agaccgatac gcatatcggg atgaaccatg agtacgttct |
| 1321 | tttctcaaaa aacataaata ttcgaaaaga gatgcaataa attaaggaga ggttataatg |
| 1381 | gtatttgaaa aaattgataa aaatagttgg aacagaaaag agtattttga ccactacttt |
| 1441 | gcaagtgtac cttgtaccta cagcatgacc gttaaagtgg atatcacaca aataaaggaa |
| 1501 | aagggaatga aactatatcc tgcaatgctt tattatattg caatgattgt aaaccgccat |
| 1561 | tcagagttta ggacggcaat caatcaagat ggtgaattgg ggatatatga tgagatgata |
| 1621 | ccaagctata caatatttca caatgatact gaaacatttt ccagcctttg gactgagtgt |
| 1681 | aagtctgact ttaaatcatt tttagcagat tatgaaagtg atacgcaacg gtatggaaac |
| 1741 | aatcatagaa tggaaggaaa gccaaatgct ccggaaaaca tttttaatgt atctatgata |
| 1801 | ccgtggtcaa ccttcgatgg ctttaatctg aatttgcaga aaggatatga ttatttgatt |
| 1861 | cctattttta ctatggggaa atattataaa gaagataaca aaattatact tcctttggca |
| 1921 | attcaagttc atcacgcagt atgtgacgga tttcacattt gccgttttgt aaacgaattg |
| 1981 | caggaattga taaatagtta accaataggc cacatgcaac tgtaaatgtt tacgcgtcct |
| 2041 | cggtaccgct tcttccacaa cagtctgcgg ttcctgtact atcacaggtt catcttctcc |
| 2101 | cgcgaattcc tgcagcccgg gtggggatgc gttccattag gtagttggcg gggtaacggc |
| 2161 | ccaccaagcc tacgatggat aggggttctg agaggaaggt cccccacatt ggaactgaga |
| 2221 | cacggtccaa actcctacgg gaggcagcag tgaggaatat tggtcaatgg gcgagagcct |
| 2281 | gaaccagcca agtagcgtga aggatgactg ccctatgggt tgtaaacttc ttttatawgg |
| 2341 | gaataaagtt cggtacgtgt gggattttgt atgtaccata tgaataagga tcggctaact |
| 2401 | ccgtgccagc agccgcggta atacggagga tccgagcgtt atccggattt attgggttta |
| 2461 | aagggagcgt aggtggatcg ttaagtcagt tgtgaaagtt tgcggctcaa ccgtaaaatt |
| 2521 | gcagttgata ctggaggtct tgagtacagt agaggtaggc ggaattcgtg gtgtagcggt |
| 2581 | gaaatgctta gatatcacga agaactccga ttgcgaaggc agcttactag actgcaactg |
| 2641 | acactgatgc tcgaaagtgt gggtatcaaa caggattaga taccctggta gtccacacag |
| 2701 | taaacgatga atactcgctg tttgcgatat acagtaagcg gccaagcgaa agcattaagt |
| 2761 | attccacctg gggagtacgc cggcaacggt gaaactcaaa ggaattgacg ggggcccgca |
| 2821 | caagcggagg aacatgtggt ttaattcgat gatacgcgag gaaccttacc cgggcttaaa |
| 2881 | ttgctgaatt ggaaacagct tagccgcaag gcagatgtga aggtgctgca tggttgtcgt |
| 2941 | cagctcgtgc cgtgaggtgt cggcttaagt gccataacga gcgcaaccct tatctttagt |
| 3001 | tactaacagg tcatgctgag gactctagag agactgccgt cgtaagatgt gaggaaggtg |
| 3061 | gggatgacgt caaatcagca cggcccttac gtccggggct acacacgtgt tacaatgggg |
| 3121 | ggtacagaag gcagctacct ggcgacagga tgctaatccc aaaaacctct ctcagttcgg |
| 3181 | atcggagtct gcaacccgac tccgtgaagc tggattcgct agtaatcgcg catcagccac |
| 3241 | ggcgcggtga atacgttccc gggccttgta cacaccgccc gtcaagccat gaaagccggg |
| 3301 | ggtacctgaa gtacgtggat ccactagttc tagagccgag tcgacggtat cgataagctt |
| 3361 | gatatcgaat tcctgcagcc cgggggatcc actagttcta gagcggccgc caccgcggtg |
| 3421 | gaggggaatt cccatgtcag ccgttaagtg ttcctgtgtc actcaaaatt gctttgagag |
| 3481 | gctctaaggg cttctcagtg cgttacatcc ctggcttgtt gtccacaacc gttaaacctt |
| 3541 | aaaagcttta aaagccttat atattctttt ttttcttata aaacttaaaa ccttagaggc |
| 3601 | tatttaagtt gctgatttat attaatttta ttgttcaaac atgagagctt agtacgtgaa |
| 3661 | acatgagagc ttagtacgtt agccatgaga gcttagtacg ttagccatga gggtttagtt |
| 3721 | cgttaaacat gagagcttag tacgttaaac atgagagctt agtacgtgaa acatgagagc |
| 3781 | ttagtacgta ctatcaacag gttgaactgc tgatcttcag atcctctacg ccggacgcat |
| 3841 | cgtggccgga tcaattccgt tttccgctgc ataaccctgc ttcggggtca ttatagcgat |
| 3901 | tttttcggta tatccatcct ttttcgcacg atatacagga ttttgccaaa gggttcgtgt |
| 3961 | agactttcct tggtgtatcc aacggcgtca gccgggcagg ataggtgaag taggcccacc |
| 4021 | cgcgagcggg tgttccttct tcactgtccc ttattcgcac ctggcggtgc tcaacgggaa |
| 4081 | tcctgctctg cgaggctggc cggctaccgc cggcgtaaca gatgagggca agcggatggc |
| 4141 | tgatgaaacc aagccaacca ggaagggcag cccacctatc acggaattga tccccctcga |
| 4201 | attg |

In any and all embodiments of the bacterial expression vectors disclosed herein, the antibiotic resistance gene is selected from the group consisting of catP, ermB, aad9, tetA, and ampR, or the auxotrophic marker is pyrG, or pyrF.

In any of the preceding embodiments of the bacterial expression vectors disclosed herein, the CRISPR enzyme is selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

Examples of fluorescent proteins include, but are not limited to, GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOκ, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa. Examples of chemiluminescent proteins include, but are not limited to, β-galactosidase, horseradish peroxidase (RP), or alkaline phosphatase. Examples of bioluminescent protein include, but are not limited to, Aequorin, firefly luciferase, Renilla luciferase, red luciferase, luxAB, or nanoluciferase.

In any and all embodiments of the bacterial expression vectors disclosed herein, the at least one sgRNA specifically hybridizes with a heterologous or endogenous target gene expressed in a gut bacterial host cell, and/or wherein the at least one sgRNA and/or the CRISPR enzyme is operably linked to a constitutive promoter or a conditional promoter. Additionally or alternatively, in some embodiments of the bacterial expression vectors disclosed herein, the at least one Group II intron specifically targets a heterologous or endogenous gene expressed in a gut bacterial host cell, and/or wherein the at least one Group II intron and/or the Group II intron-encoded protein is operably linked to a constitutive promoter or a conditional promoter.

In another aspect, the present disclosure provides an engineered gram-negative human gut bacterial cell comprising any of the preceding embodiments of the bacterial expression vector described herein, wherein the engineered gram-negative human gut bacterial cell is derived from a family selected from the group consisting of Enterobacteriaceae, Bacteroidaceae, Tannerellaceae, and Prevotellaceae. In some embodiments, the engineered gram-negative human gut bacterial cell is derived from Bacteroides cellulosilyticus, Bacteroides cellulosilyticus, Bacteroides dorei, Bacteroides eggerthii, Bacteroides finegoldii, Bacteroides fragilis, Bacteroides intestinalis, Bacteroides nordii, Bacteroides oleiciplenus, Bacteroides ovatus, Bacteroides salyersiae, Bacteroides sp., Bacteroides thetaiotaomicron, Bacteroides unformis, Bacteroides vulgatus, Bacteroides xylanisolvens, Parabacteroides faecis, Parabacteroides merdae, or Prevotella bivia.

### Expression Vectors for Genetically Modifying Gram-Positive Commensal Human Gut Bacteria

Also disclosed herein are bacterial expression vectors comprising a gram-positive bacteria replication origin that are useful for genetically modifying a plurality of human gut commensal gram-positive bacterial species. Examples of suitable gram-positive bacteria replication origin sequences include:

pBP1
(SEQ ID NO: 1)
ggcgcgccgttctgaatccttagctaatggttcaacaggtaactatgacgaagatagcaccctggataagtctgtaatggattctaaggcattt
aatgaagacgtgtatataaaatgtgctaatgaaaaagaaaatgcgttaaaagagcctaaaatgagttcaaatggttttgaaattgattggtagtt
taatttaatatattttttctattggctatctcgatacctatagaatcttctgttcacttttgtttttgaaatataaaaaggggctttttagcccc
ttttttttaaaactccggaggagtttcttcattcttgatactatacgtaactattttcgatttgacttcattgtcaattaagctagtaaaatcaa
tggttaaaaaacaaaaaacttgcatttttctacctagtaatttataattttaagtgtcgagtttaaaagtataatttaccaggaaaggagcaagt
tttttaataaggaaaaatttttccttttaaaattctatttcgttatatgactaattataatcaaaaaaatgaaaataaacaagaggtaaaaactg
ctttagagaaatgtactgataaaaaaagaaaaaatcctagatttacgtcatacatagcacctttaactactaagaaaaatattgaaaggacttcc
acttgtggagattatttgtttatgttgagtgatgcagacttagaacattttaaattacataaaggtaatttttgcggtaatagattttgtccaat
gtgtagttggcgacttgcttgtaaggatagtttagaaatatctattcttatggagcatttaagaaaagaagaaaataaagagtttatatttttaa
ctcttacaactccaaatgtaaaaagttatgatcttaattattctattaaacaatataataaatcttttaaaaaattaatggagcgtaaggaagtt
aaggatataactaaaggttatataagaaaattagaagtaacttaccaaaaggaaaaatacataacaaaggatttatggaaaataaaaaaagatta
ttatcaaaaaaaaggacttgaaattggtgatttagaacctaattttgatacttataatcctcattttcatgtagttattgcagttaataaaagtt
attttacagataaaaattattatataaatcgagaaagatggttggaattatggaagtttgctactaaggatgattctataactcaagttgatgtt
agaaaagcaaaaattaatgattataaagaggtttacgaacttgcgaaatattcagctaaagacactgattatttaatatcgaggccagtatttga
aattttttataaagcattaaaaggcaagcaggtattagtttttagtggattttttaaagatgcacacaaattgtacaagcaaggaaaacttgatg
tttataaaaagaaagatgaaattaaatatgtctatatagtttattataattggtgcaaaaaacaatatgaaaaaactagaataagggaacttacg
gaagatgaaaaagaagaattaaatcaagatttaatagatgaaatagaaatagattaaagtgtaactatactttatatatatatgattaaaaaaat
aaaaaacaacagcctattaggttgttgttttttattttctttattaatttttttaatttttagtttttagttcttttttaaaataagtttcagcc
tctttttcaatattttttaaagaaggagtatttgcatgaattgccttttttctaacagacttaggaaatattttaacagtatcttcttgcgccgg
tgattttggaacttcataacttactaatttataattattattttcttttttaattgtaacagttgcaaaagaagctgaacctgttccttcaacta
gtttatcatcttcaatataatattcttgacctatatagtataaatatatttttattatatttttacttttttctgaatctattattttataatca
taaaaagttttaccaccaaaagaaggttgtactccttctggtccaacatatttttttactatattatctaaataatttttgggaactggtgttgt
aatttgattaatcgaacaaccagttatacttaaaggaattataactataaaaatatataggattatctttttaaatttcattattggcctccttt
ttattaaatttatgttaccataaaaaggacataacgggaatatgtagaatatttttaatgtagacaaaattttacataaatataaagaaaggaag
tgtttgtttaaattttatagcaaactatcaaaaattagggggataaaaatttatgaaaaaaaggttttcgatgttatttttatgtttaactttaa
tagtttgtggtttatttacaaattcggccggcc
pCB102
(SEQ ID NO: 2)
ggcgcgccgccattatttttttgaacaattgacaattcatttcttattttttattaagtgatagtcaaaaggcataacagtgctgaatagaaaga
aatttacagaaaagaaaattatagaatttagtatgattaattatactcatttatgaatgtttaattgaatacaaaaaaaaatacttgttatgtat
tcaattacgggttaaaatatagacaagttgaaaaatttaataaaaaaataagtcctcagctcttatatattaagctaccaacttagtatataagc
caaaacttaaatgtgctaccaacacatcaagccgttagagaactctatctatagcaatatttcaaatgtaccgacatacaagagaaacattaact
atatatattcaatttatgagattatcttaacagatataaatgtaaattgcaataagtaagatttagaagtttatagcctttgtgtattggaagca
gtacgcaaaggcttttttatttgataaaaattagaagtatatttattttttcataattaatttatgaaaatgaaagggggtgagcaaagtgacag
aggaaagcagtatcttatcaaataacaaggtattagcaatatcattattgactttagcagtaaacattatgacttttatagtgcttgtagctaag
tagtacgaaagggggagctttaaaaagctccttggaatacatagaattcataaattaatttatgaaaagaagggcgtatatgaaaacttgtaaaa
attgcaaagagtttattaaagatactgaaatatgcaaaatacattcgttgatgattcatgataaaacagtagcaacctattgcagtaaatacaat
gagtcaagatgtttacataaagggaaagtccaatgtattaattgttcaaagatgaaccgatatggatggtgtgccataaaaatgagatgttttac
agaggaagaacagaaaaaagaacgtacatgcattaaatattatgcaaggagctttaaaaaagctcatgtaaagaagagtaaaaagaaaaaataat
ttatttattaatttaatattgagagtgccgacacagtatgcactaaaaaatatatctgtggtgtagtgagccgatacaaaaggatagtcactcgc
attttcataatacatcttatgttatgattatgtgtcggtgggacttcacgacgaaaacccacaataaaaaaagagttcggggtagggttaagcat
agttgaggcaactaaacaatcaagctaggatatgcagtagcagaccgtaaggtcgttgtttaggtgtgttgtaatacatacgctattaagatgta
aaaatacggataccaatgaagggaaaagtataatttttggatgtagtttgtttgttcatctatgggcaaactacgtccaaagccgtttccaaatc
tgctaaaaagtatatcctttctaaaatcaaagtcaagtatgaaatcataaataaagtttaattttgaagttattatgatattatgtttttctatt
aaaataaattaagtatatagaatagtttaataatagtatatacttaatgtgataagtgtctgacagtgtcacagaaaggatgattgttatggatt
ataagcggccggcc
pCD6
(SEQ ID NO: 3)
ggcgcgcccgcccttaagtctaaaaattaggggagatgtaaggatttgggaaaaatagaagatgttataatcataaatatggtattcgtaggct
taaagtcaaaaaggaggtgaaatataaatagatttttagctaaattaagtaagaaataggaggagatttattgaacaaaaaattagaaaaacca
tttgtatataagagagagtacgatttgactggatatgatgttgaaattttacaaaaatatgagttagaacaagcaatatatgtttatgttgggag
tagttgtgcatataacatgagagctagaagtagtaaatggagataccatataagaacaaataataagtctatatgttgtaacattaaaaatttta
tacataacttggaattgttttataaaatggaattaaagttgtcagataatattattaatgataagctatactatagcaatatagcagagtttgaa
gaatttgaaacactagaaaaagctagagaggtagaaagtactattataagtcaatatcaatttttagattctataaatcacatgttaaaacaaaa
aataattttattgagtaataaggatagtgtgttaaacataactaaaaatggaaatacaaattatttgaaagtaaaaaataaatacatagaaaaac
ataagaacaagccaataatgagataccatatcaactgtcaattcaatacagatggaagtgtcaaaagtattacacaggagtttgaaccaatattg
gaattaaacaaaaaaaataccctaagccgaccaagcagagtatttttaaaataatattttaagataacaacaaaatgagataatactactagaca
atgacaactcaactaccaattgagtttatggagctaccaactccaatatcggtctaactgattaagtatctgtagttatataataatattgctat
caattttagcatcttaacaatattattatacatactaagctaaaattattcaatagttgtaaaagttgattagtcaataagtatatatttaatgt
agtgttatctcttaaaaaaactagataaggagataataaatatatggaacaattagattcaaaatataagttgaaaaaatttctaatggcagtat
ttagagatggtataggacaaggaaataatcttattgataatgaatatgttagagtatttcaaaataataaaagtaatagtaaacaattagaactc
ggagaagaatttaaagaatatagtaaaacaactttttttaaaaatatagatgatatagtagaatttaccttcgcaaaaaatatttattatgaaaa
tacattttttaacctatgtactactgatggaaaagcaggaaccaatgaaaacttaataaatagatatgcattaggatttgattttgacaaaaaag
aattaggacaaggttttaattataaagatataattaatttatttactaagataggattacattatcatatcctagttgatagtggaaatggattc
catgtttatgtgctaattaataaaactaataacattaagttagtatcagaagttacaaatacattaataaataaattgggtgcagataaacaagc
aaatttatctactcaagtattaagagtaccttatacatataatattaaaaatactactaaacaagtaaaaataatacaccaagacaaaaatatat
atagatatgacatagaaaagttagctaaaaaatattgcaaagatgtaaaaacagtaggtaatactaatacaaaatatatattagatagtaagcta
ccaaattgtatagtagatattttaaaaaatggtagtaaagatggacataaaaacctagatttgcaaaaaatagttgtgactttaagattgaggaa
taaaagtttaagtcaagtaatatccgttgctagagaatggaactatatatcacaaaatagtctttcaaatagtgagctagaatatcaagtcaagt
atatgtatgagaaacttaaaacggttaattttggttgtactggttgtgagtttaatagtgattgttggaataaaatagaatcagattttatatat
agtgatgaagatactttgttcaatatgccacataagcactcaaaggatttgaaatataagaataggaaaggggttaaaataatgactggtaatca
attgtttatctataatgtgttacttaacaataaagatagagaattaaacatagacgatataatggagctgataacctataaacgtaagaagaaag
ttaaaaacattgttatgagtgaaaagacattaagagaaacattaaaagaacttcaacataatgattatattacaaaaacaaaaggtgttacaaag
ctaggaataaaagatacatacaatgtaaaagaagttagatgtaatatagataaacaatatactattagttactttgttaccatggcagtaatttg
gggaataatttcaactgaagaattaagattatatactcacatgagatataagcaagatttattggtcaaagatgataaaataaaaggaaatatat
taagaattaatcaagaggaattagcaaaagatttaggagtaacacagcaaagaatttcaaatatgatagaatctttattagatactaaaatttta
gatgtatgggaaactaaaataaatgatagaggatttatgtactatacatatagattaaacaagtagatttttgataggattagaattgattttct
agtcctatttttatgcaaaaaaactaattaataaaaatttcttttggtaaaataattgtacgagaattgcaaaaaaaaaatggcatcaaagtatt
gaaattaagccgttttaaaaatttcttttggtaaaataattctacatatatatgtagtatatatatatatgttttttagagaatgtataactaga
atatagagctagaatatagagaatgtataactagaatatagagctagaatatagagaatgtataactagaatatagagctagaatatagagaatg
tataactagaatatagagctagaatatagagaatgtataactagaatatagagctagaatatagagaatgtataactagaatatagagctagaat
atagagaatgtataactagaatatagagctagaatcctaattagtaggtgcttttttaaaacaagttaaaaatcaaaaatagtattagtaagcat
tggaaatgctagattctaaaatagaaaagtaaaaaattggtgcactatctaaacttatctatatcgctttttccgtcgtttggttctctagttac
gatacaggggatatgcttatattgagttatagtactaatcagtgcttaatatagttaataaaattatagttaccatagtttagtaactatgatgt
atgttagttagaaacttgcatttcggccggcc
pIM13
(SEQ ID NO: 4)
ggcgcgccgcattcacttcttttctatataaatatgagcgaagcgaataagcgtcggaaaagcagcaaaaagtttcctttttgctgttggagcat
gggggttcagggggtgcagtatctgacgtcaatgccgagcgaaagcgagccgaagggtagcatttacgttagataaccccctgatatgctc
cgacgctttatatagaaaagaagattcaactaggtaaaatcttaatataggttgagatgataaggtttataaggaatttgtttgttctaattttt
cactcattttgttctaatttcttttaacaaatgttcttttttttttagaacagttatgatatagttagaatagtttaaaataaggagtgagaaaa
agatgaaagaaagatatggaacagtctataaaggctctcagaggctcatagacgaagaaagtggagaagtcatagaggtagacaagttataccgt
aaacaaacgtctggtaacttcgtaaaggcatatatagtgcaattaataagtatgttagatatgattggcggaaaaaaacttaaaatcgttaacta
tatcctagataatgtccacttaagtaacaatacaatgatagctacaacaagagaaatagcaaaagctacaggaacaagtctacaaacagtaataa
caacacttaaaatcttagaagaaggaaatattataaaaagaaaaactggagtattaatgttaaaccctgaactactaatgagaggcgacgaccaa
aaacaaaaatacctcttactcgaatttgggaactttgagcaagaggcaaatgaaatagattgacctcccaataacaccacgtagttattgggagg
tcaatctatgaaatgcgattaagggccggcc
pMU102/Cthem-based rep origin
(SEQ ID NO: 5)
ggcgcgcccctgattctgtggataaccgtattaccgcctttgagtgagctgataccgctcgccgcagccgaacgaccgagcgcagcgagt
cagtgagcgaggaagcggaagagcgcccaatacgcaaaccgcctctccccgcgcgttggccgattcattaatgcagtatgggaaacaaa
atattgcgtatgcgactgttcacatggacgagaaaacccctcacatgcatttaggagttgttcctatgcgcctagagggcttttcgtgcgtcag
catgagcgatcggaagaaaagaagaatactttttcgcttcaagatgttttgcagcgtgatcgagaacttcgtgagcaaagaaaagcaaagag
gaaaaaatcgcatgatttggagcgataagaaaaagcactcgaatgagtgctttttttgcgttttgagcgtagcgaaaaacgagttctttctattc
ttgatacatatagaaataacgtcatttttattttagttgctgaaaggtgcgttgaagtgttggtatgtatgtgttttaaagtattgaaaaccctt
aaaattggttgcacagaaaaaccccatctgttaaagttataagtgaccaaacaaataactaaatagatgggggtttcttttaatattatgtgtcc
taatagtagcatttattcagatgaaaaatcaagggttttagtggacaagacaaaaagtggaaaagtgagaccatggagagaaaagaaaatcgcta
atgttgattactttgaacttctgcatattcttgaatttaaaaaggctgaaagagtaaaagattgtgctgaaatattagagtataaacaaaatcgt
gaaacaggcgaaagaaagttgtatcgagtgtggttttgtaaatccaggctttgtccaatgtgcaactggaggagagcaatgaaacatggcattca
gtcacaaaaggttgttgctgaagttattaaacaaaagccaacagttcgttggttgtttctcacattaacagttaaaaatgtttatgatggcgaag
aattaaataagagtttgtcagatatggctcaaggatttcgccgaatgatgcaatataaaaaaattaataaaaatcttgttggttttatgcgtgca
acggaagtgacaataaataataaagataattcttataatcagcacatgcatgtattggtatgtgtggaaccaacttattttaagaatacagaaaa
ctacgtgaatcaaaaacaatggattcaattttggaaaaaggcaatgaaattagactatgatccaaatgtaaaagttcaaatgattcgaccgaaaa
ataaatataaatcggatatacaatcggcaattgacgaaactgcaaaatatcctgtaaaggatacggattttatgaccgatgatgaagaaaagaat
ttgaaacgtttgtctgatttggaggaaggtttacaccgtaaaaggttaatctcctatggtggtttgttaaaagaaatacataaaaaattaaacct
tgatgacacagaagaaggcgatttgattcatacagatgatgacgaaaaagccgatgaagatggattttctattattgcaatgtggaattgggaac
ggaaaaattattttattaaagagtagttcaacaaacgggattgacttttaaaaaaggattgattctaatgaagaaagcagacaagtaagcctcct
aaattcactttagataaaaatttaggaggcatatcaaatgaacggccggcc
pAMB1
(SEQ ID NO: 6)
ggcgcgccctcacgttaagggattttggtcatgagattatcaaaaaggatcttcacctagatccttttaaattaaaaatgaagttttaaatcaat
ctaaagtatatatgagtaaacttggtctgacagttaccaatgcttaatcagtgaggcacctatctcagcgatctgtctatttcgttcatccatag
ttgcctgactccccgtcgtgtagataactacgatacgggagggcttaccatctggccccagtgctgcaatgataccgcgagacccacgctcaccg
gctccagatttatcagcaataaaccagccagccggaagggccgagcgcagaagtggtcctatttaatcactttgactagcaaatactaacaa
caagacacacacaccaaaaatcaaaaattcactacttttagttaaaaaccacgtaaccacaagaactaatccaatccatgtaatcgggttcttc
aaatatttctccaagattttcctcctctaatatgctcaacttaaatgacctattcaataaatctattatgctgctaaatagtttataggacaaat
aagtatactctaatgacctataaaagatagaaaattaaaaaatcaagtgttcgcttcgctctcactgcccctcgacgttttagtagcctttccct
cacttcgttcagtccaagccaactaaaagttttcgggctactctctccttctccccctaataattaattaaaatcttactctgtatatttctgct
aatcattcgctaaacagcaaagaaaaaacaaacacgtatcatagatataaatgtaatggcatagtgcgggttttattttcagcctgtatcatagc
taaacaaatcgagttgtgtgtccgttttagggcgttctgctagcttgtttaaagtctcttgaatgaatgtatgctctaagtcaaaagaatttgtc
agcgcctttatatagctttctttttcttctttttttactttaatgatcgatagcaacaatgatttaacactagcaagttgaatgccaccatttct
tcctggtttaatcttaaagaaaatttcctgattcgccttcagtaccttcagcaatttatctaatgtccgttcaggaatgcctagcacttctctaa
tctcttttttggtcgtcactaaataaggcttgtatacatcgcttttttcgctaatataagccattaaatcttctttccattctgacaaatgaaca
cgttgacgttcgcttctttttttcttgaatttaaaccacccttgacggacaaataaatctttactggttaaatcacttgatacccaagctttgca
aagaatggtaatgtattccctattagccccttgatagttttctgaataggcacttctaacaattttgattacttctttttcttctaagggttgat
ctaatcgattattaaactcaaacatattatattcgcacgtttcgattgaatagcctgaactaaagtaggctaaagagagggtaaacatgacgtta
ttacgccctattaaacccttttctcctgaaaatttcgtttcgtgcaataagagattaaaccagggttcatctacttgttttttgccttctgtacc
gcttaaaaccgttagacttgaacgagtaaagcccttattatctgtttgtttgaaagaccaatcttgccattctttgaaagaataacggtaattag
gatcaaaaaattctacattgtccgttcttggtatgcgagcaataccaaaatgattacacgttagatcaactggcaaagactttccaaaatattct
cggatattttgcgaaattattttggctgctttgacagatttaaattctgattttgaagtcacatagactggcgtttctaaaacaaaatatgcttg
ataacctttatcagatttgataatcatagtaggcataaaacctaaatcaatagcggttgttaaaatatcgcttgctgaaatagtttcttttgccg
tgtgaatatcaaaatcaataaagaaggtattgatttgtcttaaattgttttcagaatgtcctttcgtgtatgaacggttttcgtctgcatacgtt
ccataacgataaacgttgggtgtccaatgtgtaaatgtatcttgattttcttgaatcgcttcctcggaagtcagaacaacaccacgaccgccaat
catgcttgattttgagcgatacgcaaaaatagcccctttgcttttacctggcttggtagtgattgagcgaattttactatttttaaatttgtact
ttaacaagccgtcatgaagcacagtttctacaacaaaagggatattcattcagctgttctcctttcctataaatcctataaaataggttgtttaa
ttaacttggtttgctttttcattcaactgtttcaatattgcatgttttgaaaaagatttttttcctttataagtcaatttttttccactaatcga
ataaattattttgttattttctattaacttatatatataatcttccccctccgaagaaaaatacttatctgattttgtttctaagtagatatttc
tcttttctaactctttcttaaacgtttctagtgtatagatatttgctaattttcttatctccaataaactattttttatataagttttacattca
tcatgattcatacaaactccaccttctataaatgaatacaaaaaaagcaatcaaacgatttccgattgattgcttaacaattcttaaattcagta
gcttagatacttgaaaactctctgatttccctatataatgatagtacggttatataccgtcttcaaacaaagttaattaaataacttcttacgag
ggaagagttcatctgactaactgataagcgttggtttggcaatcttatcgggctatgcatttataaaatgtcgtcaaacattttataaatgtgtc
atggctcttttttcgtttctattcagttcgttgtttcgttatatctagtataccgcttttaaaaaaaaataagcaacgatttcgtgcattattca
cacgaagtcattgcttttttcttcttccatttctaaatccaatgttacttgttctgattctgtttctggttctggttctgttggctcatttggga
ttaaatccactactagcgttgagttagttaactttgcaatttgttctagtgtttttatggttggatctgattttcctgggccggcc
pWV01
(SEQ ID NO: 7)
ggcgcgccgcagcgaagatgttgtctgttagattatgaaagccgatgactgaatgaaataataagcgcagcgcccttctatttcggttggag
gaggctcaagggagtatgagggaatgaaattccctcatgggtttgattttaaaaattgcttgcaattttgccgagcggtagcgctggaaaatttt
tgaaaaaaatttggaatttggaaaaaaatggggggaaaggaagcgaattttgcttccgtactacgaccccccattaagtgccgagtgccaatt
tttgtgccaaaaacgctctatcccaactggctcaagggtttaaggggtttttcaatcgccaacgaatcgccaacgttttcgccaacgttttttat
aaatctatatttaagtagctttattgttgtttttatgattacaaagtgatacactaactttataaaattatttgattggagttttttaaatggtg
atttcagaatcgaaaaaaagagttatgatttctctgacaaaagagcaagataaaaaattaacagatatggcgaaacaaaaaggtttttcaaaatc
tgcggttgcggcgttagctatagaagaatatgcaagaaaggaatcagaacaaaaaaaataagcgaaagctcgcgtttttagaaggatacgagttt
tcgctacttgtttttgataaggtaattatatcatggctattaaaaatactaaagctagaaattttggatttttattatatcctgactcaattcct
aatgattggaaagaaaaattagagagtttgggcgtatctatggctgtcagtcctttacacgatatggacgaaaaaaaagataaagatacatggaa
tagtagtgatgttatacgaaatggaaagcactataaaaaaccacactatcacgttatatatattgcacgaaatcctgtaacaatagaaagcgtta
ggaacaagattaagcgaaaattggggaatagttcagttgctcatgttgagatacttgattatatcaaaggttcatatgaatatttgactcatgaa
tcaaaggacgctattgctaagaataaacatatatacgacaaaaaagatattttgaacattaatgattttgatattgaccgctatataacacttga
tgaaagccaaaaaagagaattgaagaatttacttttagatatagtggatgactataatttggtaaatacaaaagatttaatggcttttattcgcc
ttaggggagcggagtttggaattttaaatacgaatgatgtaaaagatattgtttcaacaaactctagcgcctttagattatggtttgagggcaat
tatcagtgtggatatagagcaagttatgcaaaggttcttgatgctgaaacgggggaaataaaatgacaaacaaagaaaaagagttatttgctgaa
aatgaggaattaaaaaaagaaattaaggacttaaaagagcgtattgaaagatacagagaaatggaagttgaattaagtacaacaatagatttatt
gagaggagggattattgaataaataaaagccccctgacgaaagtcgaagggggtttttattttggtttgatgttgcgattaatagcaatacaagg
ccggcc
pMB1
(SEQ ID NO: 8)
ggcgcgccgccgcgggcctcagcctgcggaacgcgcagcggacgccgacggctcagacggctcagaaacgtccgtgagtggcctcc
acgcggccgaacaggtcagggaggctcgcgcatacgtgagcggcgtggagaagcggctgaaggccgtccagcggcttttcgtgcagg
atgtgctgggctgggttcagccgacgcttcgctgggctgaaatatctgacttggttcccgcgtatttgttcactgtacaaatacgatgtatgctg
tagccatgtccgatgagtattcgcagccgacgcttgagctgtcgcgcacgttcgaaggctggtggctgcccgaacgcccgctgtgctgcga
cgacgactactcccggctgcaccgcaggagccgcgccgacgcgctcaaatgcaagcacatcgaggcgaaccccgccgcgctggtgaa
cacgatcgtggtggacatcgacgacgcgaacgccaaggcgatggccctgtgggagcacgagggcatgcggccgaactggatcgcgga
gaacccggccaacgggcacgctcacgcgggctgggtgctcacctttccggtgcccagaaccgatctggcgcgtctcaagccgttgaagc
tcctgcacgccaccacggagggactgcgccgctcctgcgacggggacatgggctattcgggacttctgatgaagaaccccgagcatccg
gcgtgggcgtcggacatcatcgagtgggacacctacgacctggaacagctcgtgcagtcgctccaggaacacggggacatgccgcccg
tcagctggaagcgcaccaagcgcgcccgcacgcaggggctgggacgcaactgcacgctcttcgacaaggcccgcacgctcgcctacc
gctacgttgcggcggctgccgaccgttcggaggccagcagcgaggcattgcgcctatacgtgcgtcgcacctgccacgaactcaacgtct
cgctgttccccgatccgctgcacgcgcgtgaggtcgaggacatcgccaagagcatccacaaatggatcgtcacccgcagccgcatgtgg
cgcgacggtgccattgccaacgcagccacattcatcgccatccaatccgcacgaggacacaaacacggtgagaacaaatatcagcaggt
catgaaggaggcactggaatggtaaggacgactttgaggaagaagcgcccggtgtctgcacgtgaattagctgaagcatacggcgtctcc
acgcgcaccattcagagctgggtggcaatgaagcgcgaggattggattgatgaacaagccgctatgcgcgaagcagtccgctcatatcac
gatgacgagggccatacatggccgcagaccgccgagcatttcaacatgagccagggtgccgtgcgtcaacgctgctacagggctcgcaa
ggagcgcgaggacgaggcggcggagaaatcgaagcatctacccggcgagattccactgttcgactgacgctaaacgttgtcccaaacgc
gaacgcagcacctccctcgccttgcggctttttcctcttccatcggccttcggcactcgggttgttgctccagcgccgcagggcgcgggagg
ctgcggccggcc
pIP404
(SEQ ID NO: 9)
ggcgcgccccgaagaacgttttccaatgatgagcacttttaaattaaaaatgaagttttaaaacttcatttttaatttaaattaaaaatgaagtt
ttatcaaaaaaatttccaataatcccactctaagccacaaacacgccctataaaatcccgctttaatcccactttgagacacatgtaatattact
ttacgccctagtatagtgataattttttacattcaatgccacgcaaaaaaataaaggggcactataataaaagttccttcggaactaactaaagt
aaaaaattatctttacaacctccccaaaaaaaagaacaggtacaaagtaccctataatacaagcgtaaaaaaatgagggtaaaaataaaaaaata
aaaaaataaaaaaaaaaaaaataaaaaaaataaaaaaataaaaaaaaaaaaaataaaaaaaaaaaaaataaaaaaataaaaaaataaaaa
aatataaaaaaaaaaaatataaaaataaaaaaatataaaaataaaaaaatataaaaataaaaaaataaaaaaatataaaaataaaaaaataaa
aaaatataaaaatattttttatttaaagtttgaaaaaaatttttttatattatataatctttgaagaaaagaatataaaaaatgagcctttataa
aagcccattttttttcatatacgtaatatgacgttctaatgtttttattggtacttctaacattagagtaatttctttatttttaaagccttttt
ctttaagggcttttattttttttcttaatacatttaattcctctttttttgttgcttttcctttagcttttaattgctcttgataatttttttta
cctctaatattttctcttctcttatattcctttttagaaattattattgtcatatatttttgttcttcttctgtaatttctaataactctataag
agtttcattcttatacttatattgcttatttttatctaaataacatctttcagcacttctagttgctcttataacttctctttcacttaaatgtt
gtctaaacatactattaagttctaaaacatcatttaatgccttctcaatgtcttctgtaaagctacaaagataatatctatataaaaataatata
agctctctgtgtccttttaaatcatattctcttagttcacaaagttttattatgtcttgtattcttccataatataaacttctttctctataaat
ataatttattttgcttggtctaccctttttcctttcatatggttttaattcaggtaaaaatccattttgtatttctcttaagtcataaatatatt
cgtactcatctaatatattgactactgtttttgatttagagtttatacttcctggaactcttaatattctggttgcatctaaggcttgtctatct
gctccaaagtattttaattgattatataaatattcttgaaccgctttccataatggtaatgctttactaggtactgcatttattatccatattaa
atacattcctcttccactatctattacatagtttggtataggaatactttgattaaaataattcttttctaagtccattaatacctggtctttag
ttttgccagttttataataatccaagtctataaacagtgtatttaactcttttatattttctaatcgcctacacggcttataaaaggtatttaga
gttatatagatattttcatcactcatatctaaatcttttaattcagcgtatttatagtgccattggctatatccttttttatctataacgctcct
ggttatccaccctttacttctactatgaatattatctatatagttctttttattcagctttaatgcgtttctcacttattcacctccccttctgt
aaaactaagaaaattatatcatattttcaataattattaactattcttaaactcttaataaaaaatagagtaagtccccaattgaaacttaatct
attttttatgttttaatttattatttttattaaaatattttaaactaaattaaatgattctttttaattttttactatttcattccataatatat
tactataattatttacaaataatatttcttcatttgtaatatttagatgatttactaattttagtttttatatattaaataattaatgtataatt
tatataaaaaatcaaaggagcttataaattatgattatttccaaagatactaaagatttaattttttcaattttaacaatactttttgtaatatt
atgtttaaatttaattgtatttttttcatataataaagccgttgaagtaaaccaatccattttccttatgatgggccggcc

In some embodiments, the bacterial expression vectors of the present technology comprise a gram-positive bacteria replication origin comprising a sequence selected from among:

pBP1 (C. botulinum)
(SEQ ID NO: 311)
gaatggcgaatggcgctagcataaaaataagaagcctgcatttgcaggcttcttatttttatggcgcgccgttctgaatccttagcta
atggttcaacaggtaactatgacgaagatagcaccctggataagtctgtaatggattctaaggcatttaatgaagacgtgtatataaaatgtgct
aatgaaaaagaaaatgcgttaaaagagcctaaaatgagttcaaatggttttgaaattgattggtagtttaatttaatatattttttctattggct
atctcgatacctatagaatcttctgttcacttttgtttttgaaatataaaaaggggctttttagccccttttttttaaaactccggaggagtttc
ttcattcttgatactatacgtaactattttcgatttgacttcattgtcaattaagctagtaaaatcaatggttaaaaaacaaaaaacttgcattt
ttctacctagtaatttataattttaagtgtcgagtttaaaagtataatttaccaggaaaggagcaagttttttaataaggaaaaatttttccttt
taaaattctatttcgttatatgactaattataatcaaaaaaatgaaaataaacaagaggtaaaaactgctttagagaaatgtactgataaaaaaa
gaaaaaatcctagatttacgtcatacatagcacctttaactactaagaaaaatattgaaaggacttccacttgtggagattatttgtttatgttg
agtgatgcagacttagaacattttaaattacataaaggtaatttttgcggtaatagattttgtccaatgtgtagttggcgacttgcttgtaagga
tagtttagaaatatctattcttatggagcatttaagaaaagaagaaaataaagagtttatatttttaactcttacaactccaaatgtaaaaagtt
atgatcttaattattctattaaacaatataataaatcttttaaaaaattaatggagcgtaaggaagttaaggatataactaaaggttatataaga
aaattagaagtaacttaccaaaaggaaaaatacataacaaaggatttatggaaaataaaaaaagattattatcaaaaaaaaggacttgaaattgg
tgatttagaacctaattttgatacttataatcctcattttcatgtagttattgcagttaataaaagttattttacagataaaaattattatataa
atcgagaaagatggttggaattatggaagtttgctactaaggatgattctataactcaagttgatgttagaaaagcaaaaattaatgattataaa
gaggtttacgaacttgcgaaatattcagctaaagacactgattatttaatatcgaggccagtatttgaaattttttataaagcattaaaaggcaa
gcaggtattagtttttagtggattttttaaagatgcacacaaattgtacaagcaaggaaaacttgatgtttataaaaagaaagatgaaattaaat
atgtctatatagtttattataattggtgcaaaaaacaatatgaaaaaactagaataagggaacttacggaagatgaaaaagaagaattaaatcaa
gatttaatagatgaaatagaaatagattaaagtgtaactatactttatatatatatgattaaaaaaataaaaaacaacagcctattaggttgttg
ttttttattttctttattaatttttttaatttttagtttttagttcttttttaaaataagtttcagcctctttttcaatattttttaaagaagga
gtatttgcatgaattgccttttttctaacagacttaggaaatattttaacagtatcttcttgcgccggtgattttggaacttcataacttactaa
tttataattattattttcttttttaattgtaacagttgcaaaagaagctgaacctgttccttcaactagtttatcatcttcaatataatattctt
gacctatatagtataaatatatttttattatatttttacttttttctgaatctattattttataatcataaaaagttttaccaccaaaagaaggt
tgtactccttctggtccaacatatttttttactatattatctaaataatttttgggaactggtgttgtaatttgattaatcgaacaaccagttat
acttaaaggaattataactataaaaatatataggattatctttttaaatttcattattggcctcctttttattaaatttatgttaccataaaaag
gacataacgggaatatgtagaatatttttaatgtagacaaaattttacataaatataaagaaaggaagtgtttgtttaaattttatagcaaacta
tcaaaaattagggggataaaaatttatgaaaaaaaggttttcgatgttatttttatgtttaactttaatagtttgtggtttatttacaaattcgg
ccggccagtgggcaagttg
pCB102 (C. butyricum)
(SEQ ID NO: 312)
gaatggcgaatggcgctagcataaaaataagaagcctgcatttgcaggcttcttatttttatggcgcgccgccattatttttttgaaca
attgacaattcatttcttattttttattaagtgatagtcaaaaggcataacagtgctgaatagaaagaaatttacagaaaagaaaattatagaat
ttagtatgattaattatactcatttatgaatgtttaattgaatacaaaaaaaaatacttgttatgtattcaattacgggttaaaatatagacaag
ttgaaaaatttaataaaaaaataagtcctcagctcttatatattaagctaccaacttagtatataagccaaaacttaaatgtgctaccaacacat
caagccgttagagaactctatctatagcaatatttcaaatgtaccgacatacaagagaaacattaactatatatattcaatttatgagattatct
taacagatataaatgtaaattgcaataagtaagatttagaagtttatagcctttgtgtattggaagcagtacgcaaaggcttttttatttgataa
aaattagaagtatatttattttttcataattaatttatgaaaatgaaagggggtgagcaaagtgacagaggaaagcagtatcttatcaaataaca
aggtattagcaatatcattattgactttagcagtaaacattatgacttttatagtgcttgtagctaagtagtacgaaagggggagctttaaaaag
ctccttggaatacatagaattcataaattaatttatgaaaagaagggcgtatatgaaaacttgtaaaaattgcaaagagtttattaaagatactg
aaatatgcaaaatacattcgttgatgattcatgataaaacagtagcaacctattgcagtaaatacaatgagtcaagatgtttacataaagggaaa
gtccaatgtattaattgttcaaagatgaaccgatatggatggtgtgccataaaaatgagatgttttacagaggaagaacagaaaaaagaacgtac
atgcattaaatattatgcaaggagctttaaaaaagctcatgtaaagaagagtaaaaagaaaaaataatttatttattaatttaatattgagagtg
ccgacacagtatgcactaaaaaatatatctgtggtgtagtgagccgatacaaaaggatagtcactcgcattttcataatacatcttatgttatga
ttatgtgtcggtgggacttcacgacgaaaacccacaataaaaaaagagttcggggtagggttaagcatagttgaggcaactaaacaatcaagcta
ggatatgcagtagcagaccgtaaggtcgttgtttaggtgtgttgtaatacatacgctattaagatgtaaaaatacggataccaatgaagggaaaa
gtataatttttggatgtagtttgtttgttcatctatgggcaaactacgtccaaagccgtttccaaatctgctaaaaagtatatcctttctaaaat
caaagtcaagtatgaaatcataaataaagtttaattttgaagttattatgatattatgtttttctattaaaataaattaagtatatagaatagtt
taataatagtatatacttaatgtgataagtgtctgacagtgtcacagaaaggatgattgttatggattataagcggccggccagtgggcaagttg
pCD6 (C. difficile)
(SEQ ID NO: 313)
gaatggcgaatggcgctagcataaaaataagaagcctgcatttgcaggcttcttatttttatggcgcgcccgcccttaagtctaaaa
attaggggagatgtaaggatttgggaaaaatagaagatgttataatcataaatatggtattcgtaggcttaaagtcaaaaaggaggtgaaatat
aaatagatttttagctaaattaagtaagaaataggaggagatttattgaacaaaaaattagaaaaaccatttgtatataagagagagtacgattt
gactggatatgatgttgaaattttacaaaaatatgagttagaacaagcaatatatgtttatgttgggagtagttgtgcatataacatgagagcta
gaagtagtaaatggagataccatataagaacaaataataagtctatatgttgtaacattaaaaattttatacataacttggaattgttttataaa
atggaattaaagttgtcagataatattattaatgataagctatactatagcaatatagcagagtttgaagaatttgaaacactagaaaaagctag
agaggtagaaagtactattataagtcaatatcaatttttagattctataaatcacatgttaaaacaaaaaataattttattgagtaataaggata
gtgtgttaaacataactaaaaatggaaatacaaattatttgaaagtaaaaaataaatacatagaaaaacataagaacaagccaataatgagatac
catatcaactgtcaattcaatacagatggaagtgtcaaaagtattacacaggagtttgaaccaatattggaattaaacaaaaaaaataccctaag
ccgaccaagcagagtatttttaaaataatattttaagataacaacaaaatgagataatactactagacaatgacaactcaactaccaattgagtt
tatggagctaccaactccaatatcggtctaactgattaagtatctgtagttatataataatattgctatcaattttagcatcttaacaatattat
tatacatactaagctaaaattattcaatagttgtaaaagttgattagtcaataagtatatatttaatgtagtgttatctcttaaaaaaactagat
aaggagataataaatatatggaacaattagattcaaaatataagttgaaaaaatttctaatggcagtatttagagatggtataggacaaggaaat
aatcttattgataatgaatatgttagagtatttcaaaataataaaagtaatagtaaacaattagaactcggagaagaatttaaagaatatagtaa
aacaactttttttaaaaatatagatgatatagtagaatttaccttcgcaaaaaatatttattatgaaaatacattttttaacctatgtactactg
atggaaaagcaggaaccaatgaaaacttaataaatagatatgcattaggatttgattttgacaaaaaagaattaggacaaggttttaattataaa
gatataattaatttatttactaagataggattacattatcatatcctagttgatagtggaaatggattccatgtttatgtgctaattaataaaac
taataacattaagttagtatcagaagttacaaatacattaataaataaattgggtgcagataaacaagcaaatttatctactcaagtattaagag
taccttatacatataatattaaaaatactactaaacaagtaaaaataatacaccaagacaaaaatatatatagatatgacatagaaaagttagct
aaaaaatattgcaaagatgtaaaaacagtaggtaatactaatacaaaatatatattagatagtaagctaccaaattgtatagtagatattttaaa
aaatggtagtaaagatggacataaaaacctagatttgcaaaaaatagttgtgactttaagattgaggaataaaagtttaagtcaagtaatatccg
ttgctagagaatggaactatatatcacaaaatagtctttcaaatagtgagctagaatatcaagtcaagtatatgtatgagaaacttaaaacggtt
aattttggttgtactggttgtgagtttaatagtgattgttggaataaaatagaatcagattttatatatagtgatgaagatactttgttcaatat
gccacataagcactcaaaggatttgaaatataagaataggaaaggggttaaaataatgactggtaatcaattgtttatctataatgtgttactta
acaataaagatagagaattaaacatagacgatataatggagctgataacctataaacgtaagaagaaagttaaaaacattgttatgagtgaaaag
acattaagagaaacattaaaagaacttcaacataatgattatattacaaaaacaaaaggtgttacaaagctaggaataaaagatacatacaatgt
aaaagaagttagatgtaatatagataaacaatatactattagttactttgttaccatggcagtaatttggggaataatttcaactgaagaattaa
gattatatactcacatgagatataagcaagatttattggtcaaagatgataaaataaaaggaaatatattaagaattaatcaagaggaattagca
aaagatttaggagtaacacagcaaagaatttcaaatatgatagaatctttattagatactaaaattttagatgtatgggaaactaaaataaatga
tagaggatttatgtactatacatatagattaaacaagtagatttttgataggattagaattgattttctagtcctatttttatgcaaaaaaacta
attaataaaaatttttttggtaaaataattgtacgagaattgcaaaaaaaaaatggcatcaaagtattgaaattaagccgttttaaaaatttctt
ttggtaaaataattctacatatatatgtagtatatatatatatgttttttagagaatgtataactagaatatagagctagaatatagagaatgta
taactagaatatagagctagaatatagagaatgtataactagaatatagagctagaatatagagaatgtataactagaatatagagctagaatat
agagaatgtataactagaatatagagctagaatatagagaatgtataactagaatatagagctagaatatagagaatgtataactagaatataga
gctagaatcctaattagtaggtgcttttttaaaacaagttaaaaatcaaaaatagtattagtaagcattggaaatgctagattctaaaatagaaa
agtaaaaaattggtgcactatctaaacttatctatatcgctttttccgtcgtttggttctctagttacgatacaggggatatgcttatattgagt
tatagtactaatcagtgcttaatatagttaataaaattatagttaccatagtttagtaactatgatgtatgttagttagaaacttgcatttcggc
cggccagtgggcaagttg
pIM13 (B. subtilis)
(SEQ ID NO: 314)
gaatggcgaatggcgctagcataaaaataagaagcctgcatttgcaggcttcttatttttatggcgcgccgcattcacttcttttctat
ataaatatgagcgaagcgaataagcgtcggaaaagcagcaaaaagtttcctttttgctgttggagcatgggggttcagggggtgcagtatct
gacgtcaatgccgagcgaaagcgagccgaagggtagcatttacgttagataaccccctgatatgctccgacgctttatatagaaaagaagat
tcaactaggtaaaatcttaatataggttgagatgataaggtttataaggaatttgtttgttctaatttttcactcattttgttctaatttctttt
aacaaatgttcttttttttttagaacagttatgatatagttagaatagtttaaaataaggagtgagaaaaagatgaaagaaagatatggaacagt
ctataaaggctctcagaggctcatagacgaagaaagtggagaagtcatagaggtagacaagttataccgtaaacaaacgtctggtaacttcgtaa
aggcatatatagtgcaattaataagtatgttagatatgattggcggaaaaaaacttaaaatcgttaactatatcctagataatgtccacttaagt
aacaatacaatgatagctacaacaagagaaatagcaaaagctacaggaacaagtctacaaacagtaataacaacacttaaaatcttagaagaagg
aaatattataaaaagaaaaactggagtattaatgttaaaccctgaactactaatgagaggcgacgaccaaaaacaaaaatacctcttactcgaat
ttgggaactttgagcaagaggcaaatgaaatagattgacctcccaataacaccacgtagttattgggaggtcaatctatgaaatgcgattaaggg
ccggccagtgggcaagttg
Cthem-based rep origin (C. thermocellum)
(SEQ ID NO: 315)
gaatggcgaatggcgctagcataaaaataagaagcctgcatttgcaggcttcttatttttatggcgcgcccctgattctgtggataa
ccgtattaccgcctttgagtgagctgataccgctcgccgcagccgaacgaccgagcgcagcgagtcagtgagcgaggaagcggaagag
cgcccaatacgcaaaccgcctctccccgcgcgttggccgattcattaatgcagtatgggaaaaaaatattgcgtatgcgactgttcacatgg
acgagaaaacccctcacatgcatttaggagttgttcctatgcgcctagagggcttttcgtgcgtcagcatgagcgatcggaagaaaagaaga
atactttttcgcttcaagatgttttgcagcgtgatcgagaacttcgtgagcaaagaaaagcaaagaggaaaaaatcgcatgatttggagcgat
aagaaaaagcactcgaatgagtgctttttttgcgttttgagcgtagcgaaaaacgagttctttctattcttgatacatatagaaataacgtcatt
tttattttagttgctgaaaggtgcgttgaagtgttggtatgtatgtgttttaaagtattgaaaacccttaaaattggttgcacagaaaaacccca
tctgttaaagttataagtgaccaaacaaataactaaatagatgggggtttcttttaatattatgtgtcctaatagtagcatttattcagatgaaa
aatcaagggttttagtggacaagacaaaaagtggaaaagtgagaccatggagagaaaagaaaatcgctaatgttgattactttgaacttctgcat
attcttgaatttaaaaaggctgaaagagtaaaagattgtgctgaaatattagagtataaacaaaatcgtgaaacaggcgaaagaaagttgtatcg
agtgtggttttgtaaatccaggctttgtccaatgtgcaactggaggagagcaatgaaacatggcattcagtcacaaaaggttgttgctgaagtta
ttaaacaaaagccaacagttcgttggttgtttctcacattaacagttaaaaatgtttatgatggcgaagaattaaataagagtttgtcagatatg
gctcaaggatttcgccgaatgatgcaatataaaaaaattaataaaaatcttgttggttttatgcgtgcaacggaagtgacaataaataataaaga
taattcttataatcagcacatgcatgtattggtatgtgtggaaccaacttattttaagaatacagaaaactacgtgaatcaaaaacaatggattc
aattttggaaaaaggcaatgaaattagactatgatccaaatgtaaaagttcaaatgattcgaccgaaaaataaatataaatcggatatacaatcg
gcaattgacgaaactgcaaaatatcctgtaaaggatacggattttatgaccgatgatgaagaaaagaatttgaaacgtttgtctgatttggagga
aggtttacaccgtaaaaggttaatctcctatggtggtttgttaaaagaaatacataaaaaattaaaccttgatgacacagaagaaggcgatttga
ttcatacagatgatgacgaaaaagccgatgaagatggattttctattattgcaatgtggaattgggaacggaaaaattattttattaaagagtag
ttcaacaaacgggattgacttttaaaaaaggattgattctaatgaagaaagcagacaagtaagcctcctaaattcactttagataaaaatttagg
aggcatatcaaatgaacggccggccagtgggcaagttg
pAMB1-based (E. faecalis)
(SEQ ID NO: 316)
gaatggcgaatggcgctagcataaaaataagaagcctgcatttgcaggcttcttatttttatggcgcgccctcacgttaagggatttt
ggtcatgagattatcaaaaaggatcttcacctagatccttttaaattaaaaatgaagttttaaatcaatctaaagtatatatgagtaaacttggt
ctgacagttaccaatgcttaatcagtgaggcacctatctcagcgatctgtctatttcgttcatccatagttgcctgactccccgtcgtgtagata
actacgatacgggagggcttaccatctggccccagtgctgcaatgataccgcgagacccacgctcaccggctccagatttatcagcaataaacca
gccagccggaagggccgagcgcagaagtggtcctatttaatcactttgactagcaaatactaacaacaagacacacacaccaaaaatcaa
aaattcactacttttagttaaaaaccacgtaaccacaagaactaatccaatccatgtaatcgggttcttcaaatatttctccaagattttcctcc
tctaatatgctcaacttaaatgacctattcaataaatctattatgctgctaaatagtttataggacaaataagtatactctaatgacctataaaa
gatagaaaattaaaaaatcaagtgttcgcttcgctctcactgcccctcgacgttttagtagcctttccctcacttcgttcagtccaagccaacta
aaagttttcgggctactctctccttctccccctaataattaattaaaatcttactctgtatatttctgctaatcattcgctaaacagcaaagaaa
aaacaaacacgtatcatagatataaatgtaatggcatagtgcgggttttattttcagcctgtatcatagctaaacaaatcgagttgtgtgtccgt
tttagggcgttctgctagcttgtttaaagtctcttgaatgaatgtatgctctaagtcaaaagaatttgtcagcgcctttatatagctttcttttt
cttctttttttactttaatgatcgatagcaacaatgatttaacactagcaagttgaatgccaccatttcttcctggtttaatcttaaagaaaatt
tcctgattcgccttcagtaccttcagcaatttatctaatgtccgttcaggaatgcctagcacttctctaatctcttttttggtcgtcactaaata
aggcttgtatacatcgcttttttcgctaatataagccattaaatcttctttccattctgacaaatgaacacgttgacgttcgcttctttttttct
tgaatttaaaccacccttgacggacaaataaatctttactggttaaatcacttgatacccaagctttgcaaagaatggtaatgtattccctatta
gccccttgatagttttctgaataggcacttctaacaattttgattacttctttttcttctaagggttgatctaatcgattattaaactcaaacat
attatattcgcacgtttcgattgaatagcctgaactaaagtaggctaaagagagggtaaacatgacgttattacgccctattaaacccttttctc
ctgaaaatttcgtttcgtgcaataagagattaaaccagggttcatctacttgttttttgccttctgtaccgcttaaaaccgttagacttgaacga
gtaaagcccttattatctgtttgtttgaaagaccaatcttgccattctttgaaagaataacggtaattaggatcaaaaaattctacattgtccgt
tcttggtatgcgagcaataccaaaatgattacacgttagatcaactggcaaagactttccaaaatattctcggatattttgcgaaattattttgg
ctgctttgacagatttaaattctgattttgaagtcacatagactggcgtttctaaaacaaaatatgcttgataacctttatcagatttgataatc
atagtaggcataaaacctaaatcaatagcggttgttaaaatatcgcttgctgaaatagtttcttttgccgtgtgaatatcaaaatcaataaagaa
ggtattgatttgtcttaaattgttttcagaatgtcctttcgtgtatgaacggttttcgtctgcatacgttccataacgataaacgttgggtgtcc
aatgtgtaaatgtatcttgattttcttgaatcgcttcctcggaagtcagaacaacaccacgaccgccaatcatgcttgattttgagcgatacgca
aaaatagcccctttgcttttacctggcttggtagtgattgagcgaattttactatttttaaatttgtactttaacaagccgtcatgaagcacagt
ttctacaacaaaagggatattcattcagctgttctcctttcctataaatcctataaaataggttgtttaattaacttggtttgctttttcattca
actgtttcaatattgcatgttttgaaaaagatttttttcctttataagtcaatttttttccactaatcgaataaattattttgttattttctatt
aacttatatatataatcttccccctccgaagaaaaatacttatctgattttgtttctaagtagatatttctcttttctaactctttcttaaacgt
ttctagtgtatagatatttgctaattttcttatctccaataaactattttttatataagttttacattcatcatgattcatacaaactccacctt
ctataaatgaatacaaaaaaagcaatcaaacgatttccgattgattgcttaacaattcttaaattcagtagcttagatacttgaaaactctctga
tttccctatataatgatagtacggttatataccgtcttcaaacaaagttaattaaataacttcttacgagggaagagttcatctgactaactgat
aagcgttggtttggcaatcttatcgggctatgcatttataaaatgtcgtcaaacattttataaatgtgtcatggctcttttttcgtttctattca
gttcgttgtttcgttatatctagtataccgcttttaaaaaaaaataagcaacgatttcgtgcattattcacacgaagtcattgcttttttcttct
tccatttctaaatccaatgttacttgttctgattctgtttctggttctggttctgttggctcatttgggattaaatccactactagcgttgagtt
agttaactttgcaatttgttctagtgtttttatggttggatctgattttcctgggccggccagtgggcaagttg
pWV01-based (L. lactis)
(SEQ ID NO: 317)
gaatggcgaatggcgctagcataaaaataagaagcctgcatttgcaggcttcttatttttatggcgcgccgcagcgaagatgttgt
ctgttagattatgaaagccgatgactgaatgaaataataagcgcagcgcccttctatttcggttggaggaggctcaagggagtatgagggaa
tgaaattccctcatgggtttgattttaaaaattgcttgcaattttgccgagcggtagcgctggaaaatttttgaaaaaaatttggaatttggaaa
aaaatggggggaaaggaagcgaattttgcttccgtactacgaccccccattaagtgccgagtgccaatttttgtgccaaaaacgctctatcccaa
ctggctcaagggtttaaggggtttttcaatcgccaacgaatcgccaacgttttcgccaacgttttttataaatctatatttaagtagctttattg
ttgtttttatgattacaaagtgatacactaactttataaaattatttgattggagttttttaaatggtgatttcagaatcgaaaaaaagagttat
gatttctctgacaaaagagcaagataaaaaattaacagatatggcgaaacaaaaaggtttttcaaaatctgcggttgcggcgttagctatagaag
aatatgcaagaaaggaatcagaacaaaaaaaataagcgaaagctcgcgtttttagaaggatacgagttttcgctacttgtttttgataaggtaat
tatatcatggctattaaaaatactaaagctagaaattttggatttttattatatcctgactcaattcctaatgattggaaagaaaaattagagag
tttgggcgtatctatggctgtcagtcctttacacgatatggacgaaaaaaaagataaagatacatggaatagtagtgatgttatacgaaatggaa
agcactataaaaaaccacactatcacgttatatatattgcacgaaatcctgtaacaatagaaagcgttaggaacaagattaagcgaaaattgggg
aatagttcagttgctcatgttgagatacttgattatatcaaaggttcatatgaatatttgactcatgaatcaaaggacgctattgctaagaataa
acatatatacgacaaaaaagatattttgaacattaatgattttgatattgaccgctatataacacttgatgaaagccaaaaaagagaattgaaga
atttacttttagatatagtggatgactataatttggtaaatacaaaagatttaatggcttttattcgccttaggggagcggagtttggaatttta
aatacgaatgatgtaaaagatattgtttcaacaaactctagcgcctttagattatggtttgagggcaattatcagtgtggatatagagcaagtta
tgcaaaggttcttgatgctgaaacgggggaaataaaatgacaaacaaagaaaaagagttatttgctgaaaatgaggaattaaaaaaagaaattaa
ggacttaaaagagcgtattgaaagatacagagaaatggaagttgaattaagtacaacaatagatttattgagaggagggattattgaataaataa
aagccccctgacgaaagtcgaagggggtttttattttggtttgatgttgcgattaatagcaatacaaggccggccagtgggcaagttg
pMB1 (B. longum)
(SEQ ID NO: 318)
gaatggcgaatggcgctagcataaaaataagaagcctgcatttgcaggcttcttatttttatggcgcgccgccgcgggcctcagc
ctgcggaacgcgcagcggacgccgacggctcagacggctcagaaacgtccgtgagtggcctccacgcggccgaacaggtcagggag
gctcgcgcatacgtgagcggcgtggagaagcggctgaaggccgtccagcggcttttcgtgcaggatgtgctgggctgggttcagccgac
gcttcgctgggctgaaatatctgacttggttcccgcgtatttgttcactgtacaaatacgatgtatgctgtagccatgtccgatgagtattcgca
gccgacgcttgagctgtcgcgcacgttcgaaggctggtggctgcccgaacgcccgctgtgctgcgacgacgactactcccggctgcaccg
caggagccgcgccgacgcgctcaaatgcaagcacatcgaggcgaaccccgccgcgctggtgaacacgatcgtggtggacatcgacga
cgcgaacgccaaggcgatggccctgtgggagcacgagggcatgcggccgaactggatcgcggagaacccggccaacgggcacgctc
acgcgggctgggtgctcacctttccggtgcccagaaccgatctggcgcgtctcaagccgttgaagctcctgcacgccaccacggaggga
ctgcgccgctcctgcgacggggacatgggctattcgggacttctgatgaagaaccccgagcatccggcgtgggcgtcggacatcatcga
gtgggacacctacgacctggaacagctcgtgcagtcgctccaggaacacggggacatgccgcccgtcagctggaagcgcaccaagcgc
gcccgcacgcaggggctgggacgcaactgcacgctcttcgacaaggcccgcacgctcgcctaccgctacgttgcggggctgccgacc
gttcggaggccagcagcgaggcattgcgcctatacgtgcgtcgcacctgccacgaactcaacgtctcgctgttccccgatccgctgcacgc
gcgtgaggtcgaggacatcgccaagagcatccacaaatggatcgtcacccgcagccgcatgtggcgcgacggtgccattgccaacgca
gccacattcatcgccatccaatccgcacgaggacacaaacacggtgagaacaaatatcagcaggtcatgaaggaggcactggaatggta
aggacgactttgaggaagaagcgcccggtgtctgcacgtgaattagctgaagcatacggcgtctccacgcgcaccattcagagctgggtg
gcaatgaagcgcgaggattggattgatgaacaagccgctatgcgcgaagcagtccgctcatatcacgatgacgagggccatacatggcc
gcagaccgccgagcatttcaacatgagccagggtgccgtgcgtcaacgctgctacagggctcgcaaggagcgcgaggacgaggcggc
ggagaaatcgaagcatctacccggcgagattccactgttcgactgacgctaaacgttgtcccaaacgcgaacgcagcacctccctcgcctt
gcggctttttcctcttccatcggccttcggcactcgggttgttgctccagcgccgcagggcgcgggaggctgcggccggccagtgggcaa
gttg
pIP404-based (C. perfringens)
(SEQ ID NO: 319)
gaatggcgaatggcgctagcataaaaataagaagcctgcatttgcaggcttcttatttttatggcgcgccccgaagaacgttttcca
atgatgagcacttttaaattaaaaatgaagttttaaaacttcatttttaatttaaattaaaaatgaagttttatcaaaaaaatttccaataatcc
cactctaagccacaaacacgccctataaaatcccgctttaatcccactttgagacacatgtaatattactttacgccctagtatagtgataattt
tttacattcaatgccacgcaaaaaaataaaggggcactataataaaagttccttcggaactaactaaagtaaaaaattatctttacaacctcccc
aaaaaaaagaacaggtacaaagtaccctataatacaagcgtaaaaaaatgagggtaaaaataaaaaaataaaaaaaaaaaaaataaaaaaataaa
aaaaataaaaaaaaaaaaaataaaaaaaaaaaaaataaaaaaaaaaaaaataaaaaaaaaaaaaatataaaaaaaaaaaatataaaaata
aaaaaatataaaaataaaaaaatataaaaataaaaaaataaaaaaatataaaaataaaaaaataaaaaaatataaaaatattttttatttaaagt
ttgaaaaaaatttttttatattatataatctttgaagaaaagaatataaaaaatgagcctttataaaagcccattttttttcatatacgtaatat
gacgttctaatgtttttattggtacttctaacattagagtaatttctttatttttaaagcctttttctttaagggcttttattttttttcttaat
acatttaattcctctttttttgttgcttttcctttagcttttaattgctcttgataattttttttacctctaatattttctcttctcttatattc
ctttttagaaattattattgtcatatatttttgttcttcttctgtaatttctaataactctataagagtttcattcttatacttatattgcttat
ttttatctaaataacatctttcagcacttctagttgctcttataacttctctttcacttaaatgttgtctaaacatactattaagttctaaaaca
tcatttaatgccttctcaatgtcttctgtaaagctacaaagataatatctatataaaaataatataagctctctgtgtccttttaaatcatattc
tcttagttcacaaagttttattatgtcttgtattcttccataatataaacttctttctctataaatataatttattttgcttggtctaccctttt
tcctttcatatggttttaattcaggtaaaaatccattttgtatttctcttaagtcataaatatattcgtactcatctaatatattgactactgtt
tttgatttagagtttatacttcctggaactcttaatattctggttgcatctaaggcttgtctatctgctccaaagtattttaattgattatataa
atattcttgaaccgctttccataatggtaatgctttactaggtactgcatttattatccatattaaatacattcctcttccactatctattacat
agtttggtataggaatactttgattaaaataattcttttctaagtccattaatacctggtctttagttttgccagttttataataatccaagtct
ataaacagtgtatttaactcttttatattttctaatcgcctacacggcttataaaaggtatttagagttatatagatattttcatcactcatatc
taaatcttttaattcagcgtatttatagtgccattggctatatccttttttatctataacgctcctggttatccaccctttacttctactatgaa
tattatctatatagttctttttattcagctttaatgcgtttctcacttattcacctccccttctgtaaaactaagaaaattatatcatattttca
ataattattaactattcttaaactcttaataaaaaatagagtaagtccccaattgaaacttaatctattttttatgttttaatttattattttta
ttaaaatattttaaactaaattaaatgattctttttaattttttactatttcattccataatatattactataattatttacaaataatatttct
tcatttgtaatatttagatgatttactaattttagtttttatatattaaataattaatgtataatttatataaaaaatcaaaggagcttataaat
tatgattatttccaaagatactaaagatttaattttttcaattttaacaatactttttgtaatattatgtttaaatttaattgtatttttttcat
ataataaagccgttgaagtaaaccaatccattttccttatgatgggccggccagtgggcaagttg

In one aspect, the present disclosure provides a bacterial expression vector comprising (a) a gram-positive bacteria replication origin comprising a sequence selected from the group consisting of SEQ ID NOs. 1-9 or 311-319, (b) a heterologous nucleic acid encoding a selectable marker that is an antibiotic resistance gene or an auxotrophic marker, and (c) at least one open reading frame, wherein the at least one open reading frame encodes a bioluminescent protein, a chemiluminescent protein, a fluorescent protein, a CRISPR enzyme, a Group II intron-encoded protein, at least one sgRNA, at least one Group II intron, or any combination thereof. The bacterial expression vector of the present technology may further comprise one or more bacterial conjugation transfer genes and/or an E. coli replication origin. Examples of bacterial conjugation transfer genes include traJ and oriT, and examples of E. coli replication origin include colE1, pBR, and R6K. Additionally or alternatively, in some embodiments, the one or more bacterial conjugation transfer genes, the gram-positive bacteria replication origin, and the heterologous nucleic acid encoding the selectable marker are codon optimized. Additionally or alternatively, in some embodiments, the at least one sgRNA or the at least one Group II intron targets one or more genes selected from among 16S rRNA, porA, bcat, croA, baiA2, baiCD, baiF, baiH, baiB, baiE, baiG and bail.

In any and all embodiments of the bacterial expression vectors disclosed herein, the antibiotic resistance gene is selected from the group consisting of catP, ermB, aad9, tetA, and ampR, or the auxotrophic marker is pyrG, or pyrF.

In any of the preceding embodiments of the bacterial expression vectors disclosed herein, the CRISPR enzyme is selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

Examples of fluorescent proteins include, but are not limited to, GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOκ, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa. Examples of chemiluminescent proteins include, but are not limited to, β-galactosidase, horseradish peroxidase (RP), or alkaline phosphatase. Examples of bioluminescent protein include, but are not limited to, Aequorin, firefly luciferase, Renilla luciferase, red luciferase, luxAB, or nanoluciferase.

In any and all embodiments of the bacterial expression vectors disclosed herein, the at least one sgRNA specifically hybridizes with a heterologous or endogenous target gene expressed in a gut bacterial host cell, and/or wherein the at least one sgRNA and/or the CRISPR enzyme is operably linked to a constitutive promoter or a conditional promoter. Additionally or alternatively, in some embodiments of the bacterial expression vectors disclosed herein, the at least one Group II intron specifically targets a heterologous or endogenous gene expressed in a gut bacterial host cell, and/or wherein the at least one Group II intron and/or the Group II intron-encoded protein is operably linked to a constitutive promoter or a conditional promoter.

In another aspect, the present disclosure provides an engineered gram-positive human gut bacterial cell comprising any of the preceding embodiments of the bacterial expression vector disclosed herein, wherein the engineered gram-positive human gut bacterial cell is derived from a family selected from the group consisting of Clostridiaceae, Lachnospiraceae, Eubacteriaceae, Erysipelotrichaceae, Enterococcaceae, and Bifidobacteriaceae. In some embodiments, the engineered gram-positive human gut bacterial cell is derived from Blautia hydrogenotrophica, Blautia luti, Blautia sp., Blautia wexlerae, Clostridium bolteae, Clostridium innocuum, Clostridium paraputrificum, Clostridium saccharolyticum, Clostridium senegalense, Clostridium sp., Clostridium sporogenes, Clostridium symbiosum, Eubacterium limosum, Eubacterium maltosivorans, Eubacterium ramulus, Eubacterium sp., Roseburia inulinivorans, Bifidobacterium catenulatum, Enterococcus faecium, Escherichia fergusonii, Roseburia inulinivorans, or Bifidobacterium catenulatum.

### Methods for Genetically Modifying Commensal Human Gut Bacteria

In one aspect, the present disclosure provides a method for modifying a gram-negative human gut bacteria cell genome comprising transferring at least one gram-negative specific bacterial expression vector described herein into a gram-negative human gut bacteria cell via conjugation. In some embodiments, the at least one bacterial expression vector is integrated into the genome of the gram-negative human gut bacteria cell.

In another aspect, the present disclosure provides a method for genetically modifying a gram-positive human gut bacteria cell comprising transferring two or more distinct bacterial expression vectors into a gram-positive human gut bacteria cell simultaneously via conjugation, wherein each of the two or more distinct bacterial expression vectors comprise: (a) a gram-positive bacteria replication origin comprising a sequence selected from the group consisting of SEQ ID NOs: 1-9 or 311-319, (b) a heterologous nucleic acid encoding a selectable marker that is an antibiotic resistance gene or an auxotrophic marker, and (c) at least one open reading frame, wherein the at least one open reading frame encodes a bioluminescent protein, a chemiluminescent protein, a fluorescent protein, a CRISPR enzyme, a Group II intron-encoded protein, at least one sgRNA, at least one Group II intron, or any combination thereof. The antibiotic resistance gene or the auxotrophic marker of each of the two or more distinct bacterial expression vectors may be independently selected from the group consisting of catP, ermB, aad9, tetA, ampR, pyrG, and pyrF.

In some embodiments, each of the two or more distinct bacterial expression vectors further comprise one or more bacterial conjugation transfer genes and/or an E. coli replication origin, optionally wherein the one or more bacterial conjugation transfer genes are selected from the group consisting of traJ, and oriT and/or the E. coli replication origin is selected from the group consisting of colE1, pBR, and R6K.

Additionally or alternatively, in some embodiments, the CRISPR enzyme of each of the two or more distinct bacterial expression vectors is independently selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

Additionally or alternatively, in some embodiments of the methods disclosed herein, the fluorescent protein of each of the two or more distinct bacterial expression vectors is independently selected from the group consisting of GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOκ, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, and Dronpa. Additionally or alternatively, in certain embodiments of the methods disclosed herein, the chemiluminescent protein of each of the two or more distinct bacterial expression vectors is independently β-galactosidase, horseradish peroxidase (HRP), or alkaline phosphatase. Additionally or alternatively, in some embodiments of the methods of the present technology, the bioluminescent protein of each of the two or more distinct bacterial expression vectors is independently Aequorin, firefly luciferase, Renilla luciferase, red luciferase, luxAB, or nanoluciferase.

In any and all embodiments of the methods disclosed herein, the at least one sgRNA sequence of the two or more distinct bacterial expression vectors specifically hybridizes with a heterologous or endogenous target gene expressed in a gut bacterial host cell, and/or wherein the at least one sgRNA and/or the CRISPR enzyme is operably linked to a constitutive promoter or a conditional promoter. Additionally or alternatively, in some embodiments of the methods disclosed herein, the at least one Group II intron of the two or more distinct bacterial expression vectors specifically targets a heterologous or endogenous gene expressed in a gut bacterial host cell, and/or wherein the at least one Group II intron and/or the Group II intron-encoded protein is operably linked to a constitutive promoter or a conditional promoter. In some embodiments, three or four distinct bacterial expression vectors are simultaneously transferred into a gram-positive human gut bacteria cell simultaneously via conjugation.

In any and all embodiments of the methods disclosed herein, the gram-negative or gram-positive human gut bacteria cell is isolated from a colonic mucosa-enriched lavage sample, a fecal sample, a rectal swab, or an intestinal sample obtained from a human subject.

Also disclosed herein are engineered human gut bacterial cells generated by any and all embodiments of the methods of the present technology. Additionally or alternatively, in some embodiments of the methods disclosed herein, the engineered human gut bacterial cells are generated using at least two, at least three, at least four, at least five, at least six, at least eight, at least ten, or at least twelve or more primers and/or gRNAs of any one of SEQ ID NOs: 23-287.

### Kits of the Present Technology

Also provided herein are kits comprising any and all embodiments of the bacterial expression vectors of the present technology and instructions for using the bacterial expression vectors to genetically modify human gut bacteria. The kits may further comprise one or more primers and/or gRNAs comprising the sequence of any one of SEQ ID NOs: 23-287.

In some embodiments, the kits further comprise buffers, enzymes having polymerase activity, enzymes having polymerase activity and lacking 5′→3′ exonuclease activity or both 5′→3′ and 3′→5′ exonuclease activity, CRISPR enzymes, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or engineering alterations (e.g., knock-in or knock-out alterations) in target nucleic acid sequences corresponding to specific human gut bacterial genes disclosed herein.

In one embodiment, the kits of the present technology further comprise a positive control nucleic acid sequence and a negative control nucleic acid sequence to ensure the integrity of the assay during experimental runs. A kit may further contain a means for comparing the levels and/or activity of one or more of the preselected set of human gut bacterial genes described herein in a sample obtained from a subject with a reference nucleic acid sample (e.g., from a control sample or isolated culture). The kit may also comprise instructions for use, software for automated analysis, containers, packages such as packaging intended for commercial sale and the like.

The kits of the present technology can also include other necessary reagents to perform any of the NGS techniques disclosed herein. For example, the kit may further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kits of the present technology may include components that are used to prepare nucleic acids from a colonic mucosa-enriched lavage sample, a fecal sample, a rectal swab, or an intestinal sample obtained from a human subject for the subsequent amplification and/or detection of engineered alterations (e.g., knock-in or knock-out alterations) in target nucleic acid sequences corresponding to specific human gut bacterial genes disclosed herein. Such sample preparation components can be used to produce nucleic acid extracts from tissue samples. The test samples used in the above-described methods will vary based on factors such as the assay format, nature of the detection method, and the specific tissues, cells or extracts used as the test sample to be assayed. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

### EXAMPLES

The present technology is further illustrated by the following Examples, which should not be construed as limiting in any way.

Hundreds of microbiota genes are associated with host biology/disease. Unraveling the causal contribution of a microbiota gene to host biology remains difficult because many are encoded by non-model gut commensals and not genetically targetable. A general approach to identify their gene transfer methodology and build their gene manipulation tools would enable mechanistic dissections of their impact on host physiology.

We developed a pipeline that identifies the gene transfer methods for 91 non-model microbes spanning >70 species and 5 phyla, and we demonstrated the utility of their genetic tools by modulating microbiome-derived short-chain fatty acids and bile acids in vitro and in the host. In a proof-of-principle study, by deleting a commensal gene for bile acid synthesis in a complex microbiome, we discover an unprecedented role of this gene in regulating colon inflammation. This technology will enable genetically engineering the non-model gut microbiome and facilitate mechanistic dissection of microbiota-host interactions.

The pipeline disclosed herein would not only facilitate dissection of the effect of microbiota on the associated treatments but would also enable genetic engineering of the gut microbiome, as a whole, for improved therapeutics.

### Example 1: Materials and Methods

### Screen the Culture Conditions of Gram-Positive Clostridia Strains

The culture was incubated in an anaerobic chamber at 37° C. under an atmosphere of 5% CO₂, 7.5% H₂, 87.5% N₂. To pre-reduce, the plates were left in the chamber overnight before being used, and the liquid medium was left in the chamber with a loosened cap for at least 48 hrs before inoculation. Firstly, we screened the culture conditions of the agar plate for the Gram-positive Clostridia strains. Strains were restreaked (from original glycerol stock or medium suspension of freeze-dried powder) onto pre-reduced TSAB (Tryptic Soy Agar+blood) plates (FIG. 31) or BHIB (Brain Heart Infusion Agar+blood) plates (FIG. 31). Then, those that can grow on either TSAB or BHIB plates were sub-cultured into 1 mL pre-reduced liquid medium: TYGB (FIG. 31), Mega (FIG. 31), Chopped Meat Medium (CMM) (FIG. 31), and Reinforced Clostridial Medium (RCM) (BD 218081), strains that can grow in any one of the four liquid cultures were subjected to the antibiotics test.

### Antibiotic Test of Gram-Positive Clostridia Strains

We tested the antibiotic resistance of 109 Clostridia microbes. To find the antibiotic and its optimal concentration that suppresses the growth of conjugation donor E. coli, the Clostridia strains were restreaked on TSAB or BHIB plates supplemented with 250 μg/mL D-cycloserine or 200 μg/mL gentamicin (to suppress the growth of conjugation donor E. coli CA434 during conjugation), or with 200 μg/mL kanamycin (to suppress the growth of conjugation donor E. coli HB101/pRK24). Both E. coli have been shown to successfully transform exogenous genomic DNA into Clostridium bacteria like C. sporogenes or C. acetobutylicum in previous studies (Canadas et al., 2019; Guo et al., 2019; Heap et al., 2007). We found that 92 out of 109 strains are resistant to either D-cycloserine, gentamicin, or kanamycin. We next screened these 92 microbes against TSAB or BHIB plates with 15 μg/mL thiamphenicol. We found that they are all sensitive to thiamphenicol, so the thiamphenicol resistant gene can be exploited as a universal marker to select transconjugants that can uptake and stably maintain extracellular plasmid DNA. Further, the minimum inhibitory concentrations (MICs) of thiamphenicol of the 92 Clostridia candidates were tested with TSAB or BHIB plates containing thiamphenicol at different concentrations (FIG. 24).

### Vector Assembly for Clostridia GM Screening

### (i) Expand the Replication Origins (Rep Oris)

We first amplified the RP4 oriT component from the pExchange vector using primers R6K_F+R6K_R, and the amplified PCR product was Gibson assembled with the backbone amplified from pMTL82151 using primers pmtl+RP4 oriT_F and pmtl+RP4 oriT_R. The assembled vector was then double-digested with AscI and FseI and used as a backbone to fuse with nine replication origins (FIG. 35) that are PCR amplified from a synthetic DNA fragment (Twist Bioscience) or Addgene vectors. This gave a series of vectors pGM-ABCM, BBCM, CBCM, DBCM, EBCM, FBCM, GBCM, HBCM, and IBCM that would be used in the following mixed-conjugation experiment to identify Clostridia gut microbes that uptake and stably maintain exogenous DNA (FIG. 9). All this set of shuttle plasmids and the plasmids described in the latter sections are verified by restriction digestion and Sanger sequencing of their core functional components.

### (ii) Sequence Optimization

We further sequence-optimized the set of Clostridia conjugation plasmids by 1) codon-optimizing the coding sequences (CDSs) of catP, traJ, and Clostridial rep oris to reduce their putative Clostridial Type II-RM sites (FIGS. 35 and 36), and 2) replacing the catP promoter Ppmtl-catP with Pfdx (the most potent promoter identified via a promoter screen). In brief, we searched the REBASE database and found 23 cutting sites that are most often recognized by the Type II-RM of Clostridia bacteria (including the solventogenic Clostridium genus) (FIG. 36). We then codon-optimized the CDSs of catP, traJ, and rep oris to reduce the number of these restriction sites by at least half (FIG. 35). Of note, the promoter and terminator of the CDS like catP or traJ and some highly repetitive motifs in the rep oris were left untouched. These sequences play a key role in regulating the functions of catP and rep ori, and any mutation (or nucleotide switch) could potentially cause dysfunction of catP or rep ori and lead to unsuccessful transformation. These set of plasmids are labeled with ‘seq-opt’. Please refer to FIG. 25 for the Clostridia that uptakes this set of vectors.

Testing if Clostridium sporogenes ATCC 15579 can Uptake Multiple Replication Origins (Plasmids) in One Conjugation Using Mixed-Conjugation Strategies

We did a preliminary test to assess if a model gut commensal C. sporogenes ATCC 15579 can uptake plasmids with a compatible replication from three E. coli conjugation donors in one conjugation. (FIG. 10A). We inoculated three E. coli HB 101/pRK24 donors harboring three different vectors pMTL82254 (rep ori: pBP1; antibiotic: erythromycin), pMTL83353 (rep ori: pCB102; antibiotic: spectinomycin), and pMTL84151 (rep ori: pCD6; antibiotic: thiamphenicol) (Heap et al., 2009), respectively. C. sporogenes ATCC 15579 was inoculated in 1 mL TYGC liquid broth and grown anaerobically at 37° C. for 12˜18 hrs. The three E. coli donors were inoculated into LB liquid broth supplemented with the corresponding antibiotics (erythromycin: 250 μg/mL; spectinomycin: 100 μg/mL; chloramphenicol: 25 μg/mL) and shaken at 220 rpm for overnight. The next day, 700 μL of each E. coli culture were mixed and centrifuged at 1500×g for 2 min. The cell pellet was washed with 1.5 mL PBS (pH 7.4) and centrifuged again at 1500×g for 2 min. The PBS supernatant was removed, and the cell pellet was transferred on ice into the anaerobic chamber. The cell pellet was mixed with 300 μL of the overnight C. sporogenes culture, and a 35 μL cell mixture was dotted on pre-reduced TYG agar plates. After 48 hrs, the cell dots were scraped using a sterile inoculation loop and resuspended in 300 μL pre-reduced PBS (pH 7.4) buffer. 50 μL of the cell suspension was plated onto three TYG agar plates that were supplemented with D-cycloserine (250 μg/mL)+erythromycin (10 μg/mL, to select for pMTL82254), or spectinomycin (500 μg/mL, to select for pMTL83353), or thiamphenicol (15 μg/mL, to select for pMTL84151).

Identify Gene Transfer Methods for Non-Model Clostridia Gut Commensals that Uptake and Maintain Exogenous Plasmid DNA

### (i) Mixed-Conjugation Strategies for Clostridia Gut Commensals

A series of plasmids (FIG. 9) harboring different rep oris but the same antibiotic marker catP (against thiamphenicol) were transformed into chemical competent E. coli CA434, E. coli HB101/pRK24 or E. coli T7 Express harboring R702 or pRK24 (plasmid R702 or pRK24 was transferred from E. coli CA434 or E. coli HB 101/pRK24 into T7 Express via conjugation) (Woods et al., 2019). We used the aforementioned mixed-conjugation strategies to identify the compatible rep ori for each Clostridia microbe of interest. For the Clostridia microbes resistant to D-cycloserine (250 μg/mL) or gentamicin (200 μg/mL), we would use E. coli CA434 as the conjugation donor. For the microbes that are not resistant to D-cycloserine (250 μg/mL) but resistant to kanamycin (200 μg/mL), we used E. coli HB101/pRK24 as their conjugation donors.

We began the conjugation by restreaking the target Clostridia microbe on a pre-reduced TSAB or BHIB agar plate. After 24-48 hrs, a single colony was inoculated in 1 mL of liquid broth (Mega/RCM/CMM) that supports its growth (mostly Mega, see FIGS. 23 and 31) in an anaerobic chamber at 37° C. under an atmosphere consisting of 5% CO2, 7.5% H2, 87.5% N2. On the same day, E. coli strains, each has a different rep on (as described in the main text and FIG. 9), were inoculated into 6 mL of LB supplemented with tetracycline (15 μg/mL) and chloramphenicol (25 μg/mL) and shaken aerobically at 37° C. for 12-18 hrs (overnight). The next day, these E. coli donors were separated into three groups, including group I: pGM-ABCM, BBCM, and CBCM; group II: pGM-DBCM, EBCM, and FBCM; and group III: pGM-GBCM, HBCM, IBCM, and a negative rep on-less control. For conjugating one Clostridia microbe, a 1.0 mL culture of each E. coli within the same group was mixed and centrifuged at 1500×g for 2 min. The culture supernatant was discarded, and the cell pellet was gently washed with 500 μL PBS buffer (pH=7.4). The PBS supernatant was then removed after centrifugation at 1500×g for 2 min, and the cell pellet was transferred on ice into the anaerobic chamber. Next, the cell pellet (a total of three cell pellets) was mixed gently with 300 μL overnight culture of the targeting Clostridia microbe, and a 35 μL cell mixture was dotted on pre-reduced TSAB or BHIB agar plates. After 48 hrs, the cell dots were scraped using a sterile inoculation loop and resuspended in 300 μL pre-reduced PBS (pH 7.4) buffer. 100 μL of the cell suspension was plated on TSAB or BHIB plate supplemented with 15 μg/mL thiamphenicol (or MIC, see FIG. 24) and 250 μg/mL D-cycloserine (if E. coli CA434 is the conjugation donor), or 200 μg/mL kanamycin (if E. coli HB101/pRK24 is the conjugation donor). Colonies typically appeared after 36-48 hrs. Four colonies were picked and restreaked onto TSAB or BHIB plates with the same antibiotics to isolate single colonies.

Attempts to expand the number of either the conjugation donor E. coli or the recipient Clostridia were made, for instance, conjugating 5 or more E. coli to 2 Clostridia in one conjugation. We obtained some transconjugants, but this set-up decreases the conjugation efficiency and makes the followed-up diagnostic PCR (to identify which rep on gets uptaken) more complicated and less efficient. All the working or non-working conjugations have been repeated at least three times in our experiment.

### (ii) Electroporation of Clostridia Microbes

To make electroporation competent cells, Clostridia microbe was first streaked on a pre-reduced TSAB or BHIB agar plate. After 24-48 hrs, a single colony was inoculated in 1 mL of pre-reduced liquid broth (Mega/RCM/CMM) that supports its growth (mostly Mega, see FIGS. 23 and 31) and incubated in an anaerobic chamber at 37° C. overnight, then 1 mL of the seed culture was inoculated in 45 mL of liquid broth supplied with 0.4 M sucrose and 0.625% or 1.25% glycine (see FIG. 24). When the culture attained an OD600 of 0.6-0.8, culture was chilled on ice for at least 10 min (from this time point, all manipulations were performed at 4° C. using an ice-bath and pre-chilled buffer). Cells were harvested by centrifugation at 8000×g and 4° C. for 10 min. The resulting cell pellet was washed twice with 10 mL of pre-reduced, filter-sterilized SMP buffer (270 mM sucrose, 1 mM MgCl2, and 5 mM sodium phosphate, pH 6.9). Following centrifugation, the final cell pellet was resuspended in 1.8 mL SMP buffer.

Plasmids harboring different replication origins were extracted and purified from E. coli CA434 using Plasmid Midiprep Kit (Zymo Research). Plasmid was pre-methylated using CpG (M. SssI) and GpC (M.CviPI) methyltransferases following the manufacturer's protocol (by NEB). After DNA purification, plasmids were separated into three groups, including group I: pGM-ABC1M_seq-opt, BBCM, and CBC1M_seq-opt; group II: pGM-DBC1M_seq-opt, EBC1M_seq-opt, and FBC1M_seq-opt; and group III: pGM-GBC1M_seq-opt, HBC1M_seq-opt, IBCM.

All the experimental procedures described below are carried out in an anaerobic chamber under an atmosphere consisting of 5% CO2, 7.5% H2, 87.5% N2. Each group of plasmid mixtures (containing 2 μg of each plasmid) were added into 600 μL electroporation competent cell and mixed gently by flicking, and the DNA-cell mixture was transferred to a pre-chilled electroporation cuvette (4 mm, Fisher Scientific). After incubated on ice for at least 10 min, a single exponential decay pulse was applied under anaerobic condition using an ECM 630 Electroporation System (BTX) set at 2.0 kV, 25 μF, and 400Ω. Immediately following pulse delivery, 900 μL of liquid broth containing 0.2 M sucrose was added into the electroporation cuvette, and the entire suspension was transferred to 400 μL of the same medium. The cell suspension was recovered at 37° C. overnight, then 200 μL of the recovery culture was plated onto TSAB or BHIB agar plates with 15 μg/mL thiamphenicol (or MIC, see FIG. 24). Colonies typically appeared after 36-48 hrs.

Eight colonies were picked and restreaked onto TSAB or BHIB plates with the same antibiotics to isolate single colonies. The isolated single colony was cultivated in 3 mL liquid broth supplemented with the same antibiotics. The genomic DNA was isolated from the resulting cell material using the Quick DNA fungal/bacterial kit (Zymo Research). Then multiplex diagnostic PCR was conducted to assess which plasmid was incorporated by the recipient Clostridia microbe. PCR products of rep oris were purified and verified by sanger sequencing. Additionally, we confirmed that the colonies we picked and restreaked are the target Clostridia strain by amplifying the 16s rRNA region of the colony using primers 16s_27F+16s_1391R, and the PCR product was purified and sent for Sanger sequencing using primer 16s_1391R.

### Diagnostic PCR and Sanger Sequencing to Verify the Plasmids Uptaken by the Clostridia Strains

The isolated single colony was cultivated in 3 mL Mega/RCM/CMM broth supplemented with the corresponding antibiotics 250 μg/mL D-cycloserine (or 200 μg/mL kanamycin)+15 g/mL thiamphenicol (or MIC, see FIG. 24; For electroporation, colonies were plated on the plates with only thiamphenicol). The genomic DNA was isolated from the resulting cell material using the Quick DNA fungal/bacterial kit (Zymo Research). Then we performed multiplex diagnostic PCR to assess which plasmid was uptaken by the conjugation recipient Clostridia microbe. For the mixed-conjugation with group I, primers pMTL_laz_diag_F (universal forward primer)+pGM-ABCM_rep_R_1500 bp+pGM-BBCM_rep_R_1000 bp+pGM-CBCM_rep_R_2000 bp (for 15 μL PCR reaction, the amount of the four primers is: 0.75 μL, 0.3 μL, 0.3 μL and 0.3 μL (10 μM)) were used for diagnostic PCR. We would see a 1.5 kb (or 1.0 kb, or 2.0 kb) PCR band if pGM-ABCM (or BBCM, or CBCM) is uptaken by the Clostridia microbe (FIGS. 11A-11B). In the meantime, we confirmed that the colonies we picked and restreaked are the target Clostridia strain but not the E. coli that escapes the antibiotics. We amplified the 16s rRNA region of the colony using primers 16s_27F+16s_1391R, and the PCR product was purified and sent for Sanger sequencing using primer 16s_1391R.

Validating the Mixed-Conjugation Result by Conjugating the E. coli Donor that Harbors the Identified Plasmid(s) to the Targeted Clostridia Microbe

We next did the single strain conjugation (one E. coli donor to one Clostridia recipient) to validate that the PCR-identified plasmid(s) can indeed be transformed into the targeted Clostridia microbe. A single colony of targeted Clostridia strain was inoculated in a 1 mL Mega (or RCM or CMM) broth in an anaerobic chamber at 37° C. under an atmosphere consisting of 5% CO2, 7.5% H2, 87.5% N2. The conjugation donor E. coli (CA434 or HB101/pRK24) harboring the PCR-identified plasmid was inoculated into 6 mL of LB supplemented with tetracycline (15 μg/mL) and chloramphenicol (25 μg/mL) and shaken aerobically at 37° C. for 12-18 hrs (overnight). After 12-18 hrs, 1.5 mL of the E. coli culture was centrifuged at 1500×g for 2 min. The supernatant was discarded, and the cell pellet was washed with 500 μL PBS buffer (pH=7.4). The PBS supernatant was then removed after centrifugation at 1500×g for 2 min, and the cell pellet was transferred on ice into the anaerobic chamber. Next, the cell pellet was mixed gently with a 300 μL overnight culture of the targeting Clostridia microbe, and a 35 L cell mixture was dotted on pre-reduced TSAB or BHIB agar plates. After 48 hrs, the cell dots were scraped using a sterile inoculation loop and resuspended in 300 μL pre-reduced PBS (pH 7.4) buffer. 100 μL of the cell suspension was plated on TSAB or BHIB plate supplemented with 15 μg/mL thiamphenicol (or MICs, see FIG. 24) and 250 μg/mL D-cycloserine (if E. coli CA434 is the conjugation donor), or 200 μg/mL kanamycin (if E. coli HB101/pRK24 is the conjugation donor). Colonies typically appeared after 36-48 hrs. Four colonies were picked and restreaked onto TSAB or BHIB plates with the same antibiotics to isolate single colonies. The isolated single colonies will be cultured in 1 mL of pre-reduced Mega (or RCM or CMM) with the same antibiotics, and the glycerol stock will be prepared using the culture.

Developing and Testing a CRISPRi-dCpf1 lacZα System for Clostridia GM

Vector assembly for utilizing dCpf1 to suppress the lacZα transcription in Clostridia Strains

We followed a previously reported literature to mutate the aspartic acid (D) catalytic site at 908 position of the Cpf1 amino acid sequence to alanine (A) to get the deactivated Cpf1 (dCpf1) (Tang et al., 2017). We amplified the Cpf1 coding sequence (CDS) from the vector pDEST-hisMBP-AsCpf1-EC (Hur et al., 2016) using primers 83153_AsCpf-1_XbaI_F+dAsCpf-1_D908A_R and dAsCpf-1_D908A_F+83153_AsCpf-1_XhoI_R. The two fragments were assembled via fusion PCR using primers 83153_AsCpf-1_XbaI_F+83153_AsCpf-1_XhoI_R. The purified PCR product and plasmid pMTL83153 were double-digested with XbaI/XhoI and ligated together using Instant Sticky-end Ligase (NEB), yielding plasmid pGM-BBCD. Then, we amplified the rep on fragments from plasmid pGM-ABCM using primers pMTL_rep_origin_F and pMTL_rep_origin_R. The purified PCR products were then Gibson assembled with the pGM-BBCD backbone amplified using primers pMTL_dCpf1_backbone_F and pMTL_dCpf1_backbone_R to give plasmid pGM-ABCD (FIG. 9). The rep on fragments from plasmids pGM-CBCM, DBCM, EBCM, FBCM, GBCM, HBCM, and IBCM were amplified using primers pMTL_rep_origin_F and pMTL_rep_origin_R. The purified PCR products were then Gibson assembled with the pGM-BBCD backbone amplified using primers pMTL_dCpf1_backbone_F and pMTL_dCpf1_backbone_R, yielding a new set of vectors pGM-CBCD, DBCD, EBCD, FBCD, GBCD, HBCD, and IBCD (including the aforementioned pGM-ABCD) (FIG. 9). Each of these vectors carries a Clostridia-specific rep on and the coding sequence of dCpf1 driven by a strong and constitutive promoter Pfdx (Heap et al., 2009).

We next assembled this set of plasmids with the lacZα. One fragment (see details below) includes the gRNA promoter, the lacZα promoter, the lacZα coding sequence and was amplified from the plasmid pMTL82254_lacZα (obtained from from pMTL82254) using primers lacZα_dCpf1_F and lacZα_dCpf1_R. The sequence is shown below:

(SEQ ID NO: 13)
cctgcaggccaacacatcaagcTTGACAGCTAGCTCAGTCCTAGGTATAATGCTAGCCGA
GACGGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAAT
TTCACACAGGAAACAGCTATGACCATGATTACGGATTCACTGGCCGTCGTTTTACAACG
TCGTGACTGGGAAAACCCTGGCGTTACCCAACTTAATCGCCTTGCAGCACATCCCCCTTTC
GCCAGCTGGCGTAATAGCGAAGAGGCCCGCACCGATCGCCCTTCCCAACAGTTGCGCAG
CCTGAATGGCGAATGGTAATAGTCCAGGCATCAAATAAAACGAAAGGCTCAGTCGAA
AGACTGGGCCTTTCGTTTTATCTGTTGTTTGTCGGTGAACGCTCTCctgacgtctcctacgtaggc
ggccgc

The lowercase italicized sequences are restriction sites of SbfI and NotI, respectively. The underlined sequence is the gRNA promoter PJ23119. The bold sequence is the lacZα promoter. The italicized uppercase sequence is the coding sequence of lacZα. The double underlined sequence is the lacZα terminator. This fragment and the plasmid pGM-ABCD were digested with SbfI/NotI and ligated together using Instant Sticky-end Ligase (NEB), yielding plasmid pGM-ABCL. Then the rep on fragments from plasmids pGM-BBCM, CBCM, DBCM, EBCM, FBCM, GBCM, HBCM and IBCM were amplified using primers pMTL_rep_origin_F and pMTL_rep_origin_R. The purified PCR products were then Gibson assembled with the backbone amplified from vector pGM-ABCL using primers pMTL_dCpf1_backbone_F and pMTL_dCpf1_backbone_R, yielding a whole set of plasmids that carry the CRISPRi-dCpf1 machinery and the lacZα reporter gene (FIG. 9).

The gRNA fragment targeting the promoter region and CDS of lacZα was introduced into the set of plasmids harboring dCpf-1 and lacZα. First, we used primers dCpf1-lacZα_gRNA_F_V6_R1 and gRNA_Cas9_Cpf1_R and a synthetic fragment (gBlocks, IDT) containing the terminator region as a template to amplify a PCR product that has one direct repeat sequence and gRNA fused with the terminator. Next, this PCR product was purified and used as the template for the second PCR, using primers dCpf1-lacZα_gRNA_F_V6_R2 and dCpf1_lacZα_gRNA_Gib_R, to get this gRNA fragment. The sequence of this fragment is shown below:

(SEQ ID NO: 14)
gtcctaggtataatgctagcTAATTTCTACTCTTGTAGATACACAGGAAACAGCTATGACTAAT
TTCTACTCTTGTAGATCAACGTCGTGACTGGGAAAACCTAATTTCTACTCTTGTA
GATAGGAGAATAGAAAGAAGAAAATTCTTTCTAAAGGCTGAATTCTCTGTTTAATTTTGAGA
GACCATTCTCTCAAAATTGAAACTTCTCAATAAAAATTGAGAAGTAGCTGACCATCACAAAA
TCGTAGATTTTGGATGTCTAGCTATGTTCTTTGAAAATTGCACAGTGAATAAGTAAAGCTAA
AGGTATATAAAAATCCTTTGTAAGAATACAATTTGCAAAGTGACAGAGGAAAGCgagacggc
gcaacgcaattaatg

The lowercase sequences are homologous to the sequence in pGM-ABCL. The bold sequences are the dCpf1 direct repeat sequence. The double underlined sequences are two gRNA targeting both the promoter region and the template strand of lacZα. The italicized sequence is a terminator region obtained from the Cs 16s rRNA gene (CLOSPO_00916).

This gRNA fragment was then Gibson assembled with the backbone amplified from pGM-ABCL using primers dCpf1-lacZα_backbone_F and 8×151 without lacZα_RN to get pGM-ABCF. The previously PCR amplified replication origin fragments were then Gibson assembled with the backbone amplified from pGM-ABCF using primers pMTL_dCpf1_backbone_F and pMTL_dCpf1_backbone_R. This generated a whole set of plasmids carrying nine different rep oris, dCpf1, lacZα, and the lacZα targeting gRNA (FIG. 9). We found that this set of plasmids were not uptaken by 2 microbes during our first round of conjugation. Because the same set of vectors without the lacZα targeting gRNA can be successfully transformed into these two microbes, we thus changed the gRNA sequence (double underlined) in the lacZα targeting gRNA fragment, and the new gRNA sequence is TGCTTCCGGCTCGTATGTTG (SEQ ID NO: 17) and ACACAGGAAACAGCTATGAC (SEQ ID NO: 18). This set of plasmids were then used to generate the gRNA-only control plasmids (FIG. 2B) by excising the dCpf-1 CDSs using PCR amplification (primers 8×153_lacza_dcpf-1_gRNA_No_dcpf-1_F and 8×153_lacza_dcpf-1_gRNA_No_dcpf-1_R) followed by T4 ligation.

Perform GM Screen in Clostridia Microbes Using the CRISPRi-dCpf1 lacZα System

Using the Gram-positive strain Clostridium bolteae DSM 29485 (S74) as an example, pGM-ABCL and pGM-ABCF were transformed into chemically competent E. coli CA434, respectively. E. coli CA434 harboring pGM-ABCL and pGM-ABCF were conjugated to Clostridium bolteae DSM 29485. The transconjugants were picked and restreaked onto TSAB supplemented with D-cycloserine (250 μg/mL)+thiamphenicol (15 μg/mL) (or MICs, see FIG. 24). Then, we cultivated three isolated single colonies in 5 mL Mega liquid broth supplemented with 15 μg/mL thiamphenicol (or MICs, see FIG. 24) for 36 hrs, extracted the RNA using Quick RNA fungal/bacterial kit (Zymo Research), and performed qPCR to quantify the relative expression of lacZα after normalizing to 16s rRNA gene, using primers dCpf1-lacZα_qPCR_F and dCpf1-lacZα_qPCR_R for lacZα gene and S74_16s_qPCR_F and S74 16s_qPCR_R for the control 16s rRNA (FIG. 30). All the non-working conjugations have been repeated at least twice in our experiment.

Developing and Testing a 16s-Tron Strategy for Clostridia GM
Assemble Vectors to Test a Group II Intron Targeting a Conserved Target Site in the Clostridia 16s rRNA Genes

We first performed multiple sequence alignment using 16s rRNAs of Clostridia that can uptake plasmids and identified a highly conserved target site of Group II intron. Then we used the Intron targeting and design tool on the ClosTron website (http://www.clostron.com/clostron2.php) to design the Group II introns targeting the conserved 16s sequence. The 16s-targeting intron was amplified using primers EBS universal primer+WBJ_16s_tgt_685_IBSN+WBJ_16s_tgt_685_EBS1d+WBJ_16s_tgt_685_EBS2, and the purified PCR product was then Gibson assembled with backbone that amplified from the plasmid pGM-BCAR-001 using primers pMTL007C-E2_F and pMTL007C-E2_R to get the plasmid pGM-BCAQ. The rep on fragments from plasmids pGM-ABCM, CBCM, DBCM, EBCM, FBCM, GBCM, HBCM, and IBCM were amplified using primers pMTL_rep_origin_F and pMTL_rep_origin_R. The purified PCR products were then Gibson assembled with the pGM-BCAQ backbone amplified using primers pMTL_dCpf1_backbone_F and clostron_rep_origin_backbone_R, yielding a new set of vectors pGM-ACAQ, CCAQ, DCAQ, ECAQ, FCAQ, GCAQ, HCAQ, ICAQ (whose conjugation-selection marker is catp, and retrotransposition-activated marker (RAM) is ermB) (FIG. 9). Then, we changed the RAM in plasmid pGM-FCAQ from ermB (antibiotic: erythromycin) into aad9 (antibiotic: spectinomycin) by assembling antibiotic marker aad9 amplified using primers clostron_Spec_F+clostron_Spec_R with the backbone of pGM-FCAQ amplified using primers Csp-316s_marker_F+Csp-316s_marker_R to get plasmid pGM-FCBQ (whose conjugation-selection marker is catP, and RAM is aad9) (FIG. 9), and then the replication origin of plasmid pGM-FCBQ was replaced to get a new set of vectors pGM-ACBQ, BCBQ, CCBQ, DCBQ, ECBQ, GCBQ, HCBQ, ICBQ.

Introduce the Assembled 16s-Tron Vectors into Clostridia and Select the RAM Integrated Mutants

Using the strain Blautia luti DSM 14534 (S54) as an example. The assembled vectors pGM-FCAQ was transformed into chemically competent E. coli CA434. Then E. coli CA434 harboring plasmid pGM-FCAQ was conjugated to S54. The transconjugants were picked and restreaked onto TSAB supplemented with D-cycloserine (250 μg/mL)+thiamphenicol (15 g/mL). Then, we cultivated three single colonies into 1 mL Mega supplied with 15 μg/mL thiamphenicol and 250 μg/mL D-cycloserine. After 24-36 hrs, 50 μL of cultures were spread onto TSAB plates supplemented with 250 μg/mL D-cycloserine and 10 μg/mL erythromycin. The transconjugants typically appeared after 36-48 hrs. Eight colonies were picked to inoculate 3 mL Mega supplemented with 250 μg/mL D-cycloserine and 10 μg/mL erythromycin. After 24-36 hrs, we extracted their genomic DNA using Quick DNA fungal/bacterial kit (Zymo Research) and performed diagnostic PCR using primers 16s_tron_diagR_v4+16s_1391R+16s_1391R_3to5 (with 16s tron_diagR_v4 binding the integrated intron part and 16s_1391R+16s_1391R_3to5 binding the target 16s site, only colonies that undergo RAM integration will have the band of ˜2.5 kb) (FIGS. 13A and 13B).

Identifying the Gene Transfer Methods for Gram-Negative Bacteroidia and Building their Gene Insertion Tools

### Screen the Culture Conditions of Gram-Negative Bacteroidia Strains

Strains were restreaked (from original glycerol stock or medium suspension of freeze-dried powder) onto pre-reduced TSAB (Tryptic Soy Agar+blood) plates (FIG. 31) or BHIB (Brain Heart Infusion Agar+blood) plates (FIGS. 24 and 31) to screen the culture conditions of the agar plate for these Gram-negative Bacteroidia strains. Then, those that can grow on either TSAB or BHIB plates were sub-cultured into pre-reduced liquid medium (see FIGS. 24 and 31): TYGB (FIG. 31), Mega (FIG. 31), Chopped Meat Medium (CMM) (FIG. 31), and Reinforced Clostridial Medium (RCM) (BD 218081). Strains that can of Bacteroidia strains in our library can grow on a TSAB plate and in the TYGB or Mega liquid medium (see FIGS. 24 and 31).

### Antibiotics Test of Target Gram-Negative Bacteroidia Strains

We tested the antibiotic resistance of 66 Bacteroidia (Prevotella and Bacteroides) microbes. To find the antibiotic and its optimal concentration that suppresses the growth of conjugation donor E. coli, the Bacteroidia strains were restreaked on TSAB plates supplemented with 200 μg/mL gentamycin or 250 μg/mL D-cycloserine. We found that all of them are resistant to either gentamycin or D-cycloserine. We next screened these microbes against TSAB plates with 15 μg/mL thiamphenicol (or MICs, see FIG. 24). We expect that they are sensitive to thiamphenicol, so the thiamphenicol resistant gene can be a universal marker to select transconjugants whose genome has been integrated by the suicide vector after the conjugation. All Bacteroidia strains tested are not resistant against thiamphenicol, and they were selected as candidates for the GM screening.

### Vector Assembly for Bacteroidia GM Screening

We first amplified ˜1 kb fragment of the 16s rRNA gene of Bacteroides theta VPI-5482 (Bt) and Bacteroides ovatus ATCC8483 (Bo) using primers BO_16S_F1+BO_16S_R2, BO_16S_F3N+BO_16S_R4 (two fragments, fused using fusion PCR, FIG. 30). The fragment was assembled with the pExchange vector to get the pGM vectors pGM-NAEM-001 and pGM-NAEM-002 for testing whether the assembled vector will integrate into the 16s rRNA loci of Bt and Bo (FIG. 28).

To generate the chimeric 16s rRNA sequence (chi-16s) for the Bacteroidia GM screening, we first performed multiple sequence alignment using 16s rRNAs of Prevotella (and Bacteroides) and synthesized ˜1 kb fragments containing the nucleotides that are conserved in at least 50% of the aligned 16s sequences for both Prevotella and Bacteroides. Then, the synthetic Bacteroides chi-16s was amplified using primers CJG_syn16s_F and CJG_syn16s_R. The purified PCR product was then Gibson assembled with the backbone amplified from the vector pExchange using primers R6K_F and Erm_R to get the plasmid pGM-NAEB (FIG. 9). Because multiple target strains are resistant against erythromycin but not against thiamphenicol, we replaced the antibiotic marker ermB with catP to use thiamphenicol as a universal selective antibiotic for the Bacteroidia GM screen. The catP coding sequence was amplified from the vector pGM-ABCM using the primers pMTL_cat_F and pMTL_cat_R. The purified PCR product was then Gibson assembled with the backbone amplified from pGM-NAEB using the primers pEx_Erm_change_F and pEx_Erm_change_R to give pGM-NAC2B (FIG. 9). The suicide plasmid pGM-NAC2B was used for the Bacteroides GMS screen. Likewise, synthesized ˜1 kb chi-16s for Prevotella amplified using primers Pre16s_F and R6K_F_RC. The purified PCR product was then Gibson assembled with the backbone amplified from the plasmid pGM-NAC2B using primers R6K_F and Erm_R to get the plasmid pGM-NAC2P (FIG. 9).

Introducing Suicide Vectors into the Bacteroidia Commensals by E. coli Conjugation

We introduced the suicide vectors pGM-NAC2P/B into the target Prevotella/Bacteroides using E. coli conjugation following the previously published protocol (Martens et al., 2008). A single colony of the target commensal was inoculated in 3 mL TYGB broth and cultured in an anaerobic chamber at 37° C. The E. coli S17 harboring the pGM-NAC2P/B vector was inoculated in the LB broth supplemented with carbenicillin (100 μg/mL) grown at 37° C. with aerobic shaking at 220 rpm. After ˜12-16 hrs, when the OD600 of E. coli S17 reached 0.8-1.0, 6 mL of E. coli S17 culture was centrifuged at 1500×g for 2 min. The supernatant was discarded, and the cell pellet was washed twice with 3 mL PBS buffer (pH=7.4). The washed E. coli S17 cell pellet was resuspended in 3 mL overnight culture of the target Bacteroidia strain and gently mixed by pipetting. The mixture was filtered through a 0.2 m filter. The filtered liquor was discarded, and the filter with the mixture of donor and recipient cells was placed onto the surface of a pre-reduced TSAB plate. The plate was incubated in a 37° C. incubator aerobically.

After incubation aerobically at 37° C. for 24 hrs, the filter was soaked in 2 mL of pre-reduced TYGB medium. The cell on the filter was resuspended into the medium by gentle vortexing. The mixture was then transferred into the anaerobic chamber, and 100 μL was plate onto a pre-reduced TSAB plate+200 μg/mL gentamycin+15 μg/mL thiamphenicol (or MICs, see FIG. 24). Colonies of the target strain typically appeared after 36-48 hrs. Four colonies were picked and restreaked on a pre-reduced TSAB plate+200 μg/mL gentamycin+15 μg/mL thiamphenicol (or MICs, see FIG. 24) to isolate single colonies. All the working or non-working conjugations have been repeated at least twice in our experiment.

Diagnostic PCR and Sequencing to Verify the Single Crossover Integration of pGM-NAC2PB

The isolated single colony was inoculated in 3 mL TYBG broth supplemented with 200 g/mL gentamycin+15 μg/mL thiamphenicol (or MICs, see FIG. 24). After 12 hrs, we extracted their genomic DNA using Quick DNA fungal/bacterial kit (Zymo Research) and performed diagnostic PCR using primers 16s_27F and R6K_R to verify the single crossover integration of pGM-NAC2P/B at their 16s rRNA loci. (FIG. 13C) We would see a ˜2.5 kb PCR band in the transconjugants, one of whose chromosomal 16s rRNA loci was integrated by pGM-NAC2P/B. The 2.5 kb PCR product was purified using DNA Clean & Concentrator kit (Zymo Research) and sent for sequencing using primer R6K_F_RC. The sequencing results showed that the partial sequence of the 2.5 kb fragment came from the synthetic chi-16s in pGM-NAC2P/B, and the partial sequence of the 16s rRNA gene of the target strain, suggesting a single crossover of pGM-NAC2P/B into one of its 16s rRNA loci. (FIG. 13D) If the single crossover takes place at the 5′ of the synthetic chi-16s, we would see that most of the resulting sequence will be the synthetic chi-16s. If the single crossover takes place at the 3′ end of the synthetic 16s or if the chi-16s is highly similar to the 16s rRNA of the targeting microbes, most of the resulting sequence will be the original 16s (FIG. 13D).

Identifying the Gene Transfer Methods for Microbes of Other Phyla and Building their Gene Insertion Tools

### Culture Conditions of Candidate Strains of Other Phyla

In addition to strains mentioned above in phyla of Firmicutes and Bacteroidetes, we also applied our pipeline to screen a batch of microbes of other phyla, including Fusobacteria (8 Fusobacterium), Proteobacteria (8 Desulfovibrio, 6 Klebsiella, 10 Proteus, and 3 clinical isolates) and one Actinobacteria 5201 (FIG. 22). Strains were restreaked from the medium suspension of freeze-dried powder onto agar plates and then sub-cultured into liquid broth recommended by DSMZ, all Fusobacterium can grow on Columbia agar with 5% blood (CBAB) and in Columbia Broth (CB) or CMM anaerobically, all Desulfovibrio can grow on Desulfovibrio (postgate) medium+1.5% agar and in Desulfovibrio (postgate) medium anaerobically, and all Klebsiella, Proteus and, 3 clinical isolates can grow on LB agar and in LB broth aerobically (for Proteus, 3% agar was added in LB to avoid swarm) (FIG. 22).

### Antibiotics Test of Target Strains of Other Phyla

To find the antibiotic and its optimal concentration that suppresses the growth of donor E. coli in conjugation (conjugation was applied for Desulfovibrio, Proteus, and 3 clinical isolates), the strains were restreaked on corresponding agar plates supplemented with 250 μg/mL D-cycloserine or 30 μg/mL kanamycin. We found that all of them are resistant to either D-cycloserine or kanamycin (see FIG. 24). To find the antibiotic and its optimal concentration that selects the growth of recipient strains in conjugation and electroporation (electroporation was applied for Fusobacterium, Klebsiella, and one Proteus), we screened all these microbes against agar plates with different concentrations of thiamphenicol (for Fusobacterium), chloramphenicol (for Desulfovibrio, Proteus and clinical isolates), carbenicillin (for clinical isolates), and kanamycin (for Klebsiella) (see FIG. 24). We expect that they are sensitive to the antibiotics tested, so that the corresponding resistant gene can be used as a marker to select transconjugants whose genome has been integrated by the suicide vector after conjugation or electroporation. Antibiotics and concentrations for each candidate strain are listed in FIG. 24.

Introducing Suicide Vectors into the Candidate Microbes of Other Phyla by Conjugation and Electroporation

For Fusobacterium, the suicide vector pGM-NACO2 was introduced into target microbes via electroporation. A single colony of the target Fusobacterium was inoculated in 1 mL liquid broth and cultured in an anaerobic chamber at 37° C. overnight. Then the 1 mL seed culture was inoculated into 45 mL of the same liquid broth and incubated at 37° C. till the OD600 reached ˜1.2, culture was chilled on ice for at least 10 min (from this time point, all manipulations were performed at 4° C. using an ice-bath and pre-chilled buffer.), then the cell was harvested by centrifugation at 8000×g and 4° C. for 10 min, the resulting cell pellet was washed twice with 25 mL of pre-reduced, filter-sterilized water. Following centrifugation, the final cell pellet was resuspended in 1 mL of 10% (v/v) cold glycerol. Then 2 μg of plasmid pGM-NACO2 was added into 100 μL electroporation competent cell and mixed gently by flicking, and the DNA-cell mixture was transferred to a pre-chilled electroporation cuvette (1 mm, Fisher Scientific). After incubated on ice for at least 10 min, a single exponential decay pulse was applied under anaerobic condition using an ECM 630 Electroporation System (BTX) set at 2.5 kV, 25 μF, and 200Ω. Immediately following pulse delivery, 1 mL of liquid broth supporting growth was added into the electroporation cuvette, and the entire suspension was recovered at 37° C. for 3 hrs, then 200 μL of the recovery culture was plated onto CBAB agar plates containing thiamphenicol (with different recipient supplied with their corresponding MICs). Colonies typically appeared after 48-72 hrs.

Similarly, for Klebsiella and one Proteus, the suicide vector pGM-NACO3 and pGM-NACO4 were also introduced into target strains via electroporation. A single colony of the target Klebsiella and Proteus was inoculated in 1 mL LB and cultured aerobically at 37° C. overnight. Then the 1 mL seed culture was inoculated into 45 mL LB and incubated at 37° C. till the OD600 reached ˜0.6, culture was chilled on ice for at least 10 min (from this time point, all manipulations were performed at 4° C. using an ice-bath and pre-chilled buffer.), then the cell was harvested by centrifugation at 5500 rpm and 4° C. for 10 min, the resulting cell pellet was washed with 25 mL of pre-reduced, filter-sterilized water and 2 mL of 10% (v/v) cold glycerol for twice. Following centrifugation, the final cell pellet was resuspended in 1 mL of 10% (v/v) cold glycerol. Then 2 μg of plasmid pGM-NACO3 and pGM-NACO4 was added into 70 μL electroporation competent cell and mixed gently by flicking, and the DNA-cell mixture was transferred to a pre-chilled electroporation cuvette (1 mm, Fisher Scientific). After incubated on ice for at least 10 min, a single exponential decay pulse was applied using an ECM 630 Electroporation System (BTX) set at 2.5 kV, 25 μF, and 200Ω. Immediately following pulse delivery, 500 μL of LB was added into the electroporation cuvette, and the entire suspension was recovered at 37° C. for 1 hr, then 100 μL of the recovery culture was plated onto LB agar plates containing selective antibiotics 30 μg/mL kanamycin or thiamphenicol. Colonies typically appeared after 36-48 hrs.

For Desulfovibrio and clinical isolates, the suicide vector pGM-NACO1 and pGM-NACO5,6,7 were introduced into target strains via conjugation, and we also applied conjugation to transfer plasmid pGM-NACO4 for two Proteus. A single colony of target strain was inoculated in 1 mL of liquid broth that supports its growth (FIGS. 23 and 31). On the same day, E. coli S17 containing the corresponding suicide vector was inoculated into 6 mL of LB supplemented with 25 μg/mL chloramphenicol and shaken aerobically at 37° C. for 12-18 hrs (overnight). The next day, 1.5 mL of S17 donor was centrifuged at 1500×g for 2 min. The culture supernatant was discarded, and the cell pellet was gently washed with 500 μL PBS buffer (pH=7.4). The PBS supernatant was then removed after centrifugation at 1500×g for 2 min, and the cell pellet was mixed gently with 300 μL overnight culture of the targeting microbe. A L cell mixture was dotted on agar plates. After 48 hrs, the cell dots were scraped using a sterile inoculation loop and resuspended in 300 μL pre-reduced PBS (pH 7.4) buffer. 100 μL of the cell suspension was plated on agar plate supplemented with 250 μg/mL D-cycloserine (for Proteus and clinical isolates) or 30 μg/mL kanamycin (for Desulfovibrio) plus chloramphenicol (with their corresponding MICs). Colonies typically appeared after 48-72 hrs.

### Diagnostic PCR and Sequencing to Verify the Single Crossover Integration of pGM Plasmids

After electroporation or conjugation plating, at least eight colonies were picked and restreaked onto agar plates with the same antibiotics used for plating. The isolated single colony was cultivated in 3 mL liquid broth supplemented with the same antibiotics. The genomic DNA was isolated from the resulting cell material using the Quick DNA fungal/bacterial kit (Zymo Research). Diagnostic PCR was performed using primers 16s_27F and R6K_R to verify the single crossover integration of suicide plasmids at their 16s rRNA loci. (FIG. 13C) We would see a ˜2.5 kb PCR band in the transconjugants, one of whose chromosomal 16s rRNA loci was integrated. The 2.5 kb PCR product was purified using DNA Clean & Concentrator kit (Zymo Research) and sent for sequencing using primer pEx_diag_R.

### Modulating Clostridia Bcat Expression and Microbiome-Derived Metabolites Using Gene Manipulation Tools Developed Via the GM Pipeline.

### Targeted Suppression of BCAA Aminotransferase (Bcat) Gene and Modulate Butyrate Production in Non-Model Gut Clostridia

(i) Vector Assembly for Utilizing dCpf1 to Suppress the Bcat and croA Transcription or Utilizing Group II Intron to Deplete croA in Clostridia Strains

The design of targeting gRNA for targeting bcat and croA in genome-sequenced Clostridia strains is about the same as that of lacZα. We used Golden Gate Ligation or Gibson assembly to introduce the targeting gRNA into the dCpf1 harboring plasmids.

The sequence of targeting gRNA that is introduced by Golden Gate ligation is shown as below:

(SEQ ID NO: 15)
tcgtctcctagcTAATTTCTACTCTTGTAGATCTATATGCCGACGGACAAGCTAATTTCTA
CTCTTGTAGATAGGATTAATACGATTATAATTAATTTCTACTCTTGTAGATAGGAG
AATAGAAAGAAGAAAATTCTTTCTAAAGGCTGAATTCTCTGTTTAATTTTGAGAGACCATTCT
CTCAAAATTGAAACTTCTCAATAAAAATTGAGAAGTAGCTGACCATCACAAAATCGTAGATT
TTGGATGTCTAGCTATGTTCTTTGAAAATTGCACAGTGAATAAGTAAAGCTAAAGGTATATA
AAAATCCTTTGTAAGAATACAATTTGCAAAGTGACAGAGGAAAGCtacgggagacgg

The lowercase sequences are Esp3I restriction sites. The bold sequences are dCpf1 direct repeat sequences. The underlined sequences are duplex gRNA targeting the bcat or croA gene. The italicized sequence is a terminator region obtained from the Cs 16s rRNA gene (CLOSPO_00916). Take Clostridium bolteae ATCC BAA-613 (S72) as an example. First, we used primers gRNA_S72_CGC65_03110_dCpf1_round1 and gRNA_Cas9_Cpf1_R and a synthetic fragment (gBlocks, IDT) containing the terminator region as a template to amplify a PCR product that has one direct repeat sequence and gRNA fused with the terminator. Next, this PCR product was purified and used as the template for the second PCR, using primers gRNA_S72_CGC65_03110_dCpf1_round2 and gRNA_dCas9_R, to get this gRNA fragment. The purified PCR product was ligated using Esp3I and T4 ligase with pGM-FBCL to give pGM-FBCD-010 (FIGS. 28 and S8).

The sequence of targeting gRNA that is introduced by Gibson assembly is shown as below:

(SEQ ID NO: 16)
gtcctaggtataatgctagcTAATTTCTACTCTTGTAGATTACCTCATAGCTACCCTTCACTAAT
TTCTACTCTTGTAGATTATAATGGTGATATGAAAACTAATTTCTACTCTTGTAGAT
AGGAGAATAGAAAGAAGAAAATTCTTTCTAAAGGCTGAATTCTCTGTTTAATTTTGAGAGAC
CATTCTCTCAAAATTGAAACTTCTCAATAAAAATTGAGAAGTAGCTGACCATCACAAAATCG
TAGATTTTGGATGTCTAGCTATGTTCTTTGAAAATTGCACAGTGAATAAGTAAAGCTAAAGG
TATATAAAAATCCTTTGTAAGAATACAATTTGCAAAGTGACAGAGGAAAGCctgacgtctcctacg
tagg

The lowercase sequences are homologous to regions in pGM-xBCL. The boldface sequences are dCpf1 direct repeat sequences. The underlined sequences are duplex gRNAs targeting bcat or croA gene. The italicized sequence is a terminator region obtained from the Cs 16s rRNA gene (CLOSPO 00916). To assemble the dCpf1 targeting vector for bcat in C. senegalense DSM 25507 (S100), we first used primers gRNA_S100_BCAA aminotransferase_dCpf1_round1 and gRNA_Cas9_Cpf1_R and a synthetic fragment (gBlocks, IDT) containing the terminator region as a template to amplify a PCR product that has one direct repeat sequence and one gRNA fused with the terminator. Next, this PCR product was purified and used as the template for the second PCR, using primers gRNA_S100_BCAA aminotransferase_dCpf1_round2 and dCpf1_gRNA_Gib_R, to get the above gRNA fragment. The purified PCR product was then Gibson-assembled with the backbone amplified from vector pGM-ABCL using primers 8×151 without lacZα_FN and 8×151 without lacZα RN, yielding plasmid pGM-ABCD-013 (FIGS. 28 and S8).

To deplete croA in Clostridia strains utilizing Group II intron, we used the Intron targeting and design tool on the ClosTron website (http://www.clostron.com/clostron2.php) to design the Group II introns targeting the croA gene. The croA-targeting intron was amplified using primers EBS universal primer+5115_cro_123_IBSN+5115_cro_123_EBS1d+5115_cro_123_EBS2, and the purified PCR product was then Gibson assembled with the backbone that amplified from the plasmid pGM-FCAQ using primers pMTL007C-E2_F and pMTL007C-E2_R to get the croA-targeting ClosTron plasmid pGM-FCAR-003. Then plasmid pGM-FCAR-003 was introduced into 5115 following the aforementioned conjugation procedure (see Example 1 and FIG. 24). After conjugation, the 5115 colonies harboring pGM-FCAR-003 appeared on the TSAB plate supplemented with 9 μg/mL thiamphenicol and 200 μg/mL gentamycin. Next, four colonies were restreaked onto a TSAB plate with the same antibiotics to isolate a single colony. The single colonies were inoculated into 1 mL Mega supplied with 9 g/mL thiamphenicol and 200 μg/mL gentamycin. After 24-36 hrs, 50 μL of cultures were spread onto TSAB plates supplemented with 200 μg/mL gentamycin and 10 μg/mL erythromycin. The integrated colonies typically appeared after 48-72 hrs. Eight colonies were picked to inoculate 3 mL Mega supplemented with 200 μg/mL gentamycin and 10 μg/mL erythromycin. After 24-36 hrs, we extracted their genomic DNA using Quick DNA fungal/bacterial kit (Zymo Research) and performed diagnostic PCR using primers S115_cro_DiagF and S115_cro_DiagR.

The butyrate production was evaluated by glucose assay with PBS washed cell of control and croA mutant, 3 mL of culture was first centrifuged at 1500×g for 3 min. The cell pellet was washed twice with 1 mL PBS (pH 7.4) and centrifuged again at 1500×g for 3 min. The PBS supernatant was removed, and the cell pellet was resuspended with 500 μL PBS and then glucose was added to the concentration of 5 mM. The mixture was incubated anaerobically at 37° C. for 1 h. The PBS suspension was subjected to SCFAs derivatization and LCMS measurement.

(ii). Transform the Assembled Vectors into the Clostridia Microbes Via E. coli Conjugation

We use the strain Clostridium bolteae ATCC BAA-613 (S72) as an example. The assembled vectors pGM-FBCD and pGM-FBCD-010 were transformed into chemically competent E. coli CA434, respectively. E. coli CA434 harboring pGM-FBCD and pGM-FBCD-010 were conjugated to S72. The transconjugants were picked and restreaked onto TSAB supplemented with D-cycloserine (250 μg/mL)+thiamphenicol (15 μg/mL). Then, we cultivated three isolated single colonies in 5 mL Mega liquid broth supplemented with 15 μg/mL thiamphenicol for 36 hrs, extracted the RNA using Quick RNA fungal/bacterial kit (Zymo Research), and performed qPCR to quantify the relative expression of lacZα after normalizing to 16s rRNA gene, using primers S72_CGC65_03110_qPCR_F and S72_CGC65_03110_qPCR_R for bcat and S72_16s_qPCR_F and S72_16s_qPCR_R for the control 16s rRNA (FIG. 30).

### Genetic Manipulation of Bacteroidia Strains to Deplete Propionate Production

(i). Assemble pGM Vectors to Generate Bacteroidia Mutants that Abolish Propionate

Take Bacteroides sp. 1_1_6 (strain 25, abbreviated as S25) as an example, a ˜-kb fragment of gene BSIG_3264 that encodes a methylmalonate mutase (mmdA), was amplified from S25 genomic DNA using primers S25_BSIG_3264_mmdA_pEX_F and S25_BSIG_3264_mmdA_pEX_R. The purified PCR product was Gibson assembled with the backbone amplified from the vector pGM-NAC2B using primers R6K_F and Erm_R to give pGM-NACM-003 (FIG. 28).

(ii). Introducing Propionate Deletion Vector pGM-NACM-003 into S25 Via E. coli Conjugation

We used the same protocol above to introduce pGM-NACM-003 into S25 via E. coli conjugation. About 48 hrs after plating the conjugation cell mixture, we picked four colonies and restreaked them on a pre-reduced TSAB plate+200 μg/mL gentamycin+15 μg/mL thiamphenicol to isolate single colonies. A single colony was inoculated in 3 mL TYBG broth supplemented with 200 μg/mL gentamycin+15 μg/mL thiamphenicol, and we extracted the bacterial genomic DNA using Quick DNA fungal/bacterial kit (Zymo Research). We performed the diagnostic PCR using primers S25_BSIG 3264 mmdA_diagF (for Bacteroides sp. 1_1_6 (S25)) and R6K_R to verify the single crossover integration of pGM-NACM-003 into mmdA. There was a ˜2.0 kb PCR band in the colonies whose mmdA gene was inserted and mutated by pGM-NACM-003.

Targeted Suppression of porA Gene in C. sporogenes ATCC 15579 (S107)

### (i) Vector Assembly

To introduce the gRNA targeting the metabolic gene porA responsible for the branched short-chain fatty acid synthesis (Guo et al., 2019), we used primers gRNA_clo02083_z2 and gRNA_dCas9_R and a synthetic fragment (gBlocks, IDT) containing the terminator region as a template to amplify a PCR product that has two direct repeat sequences and gRNA fused with the terminator. Next, this PCR product was purified and ligated using Esp3I and T4 ligase with pGM-ABCL to give pGM-ABCD-006 (FIGS. 28 and S8).

(SEQ ID NO: 12)
tcgtctcctagcTAATTTCTACTCTTGTAGATATAAGAATGCCTTACAAGTCTTAATTTCT
ACTCTTGTAGATAGGAGAATAGAAAGAAGAAAATTCTTTCTAAAGGCTGAATTCTCTGTT
TAATTTTGAGAGACCATTCTCTCAAAATTGAAACTTCTCAATAAAAATTGAGAAGTAGCTGA
CCATCACAAAATCGTAGATTTTGGATGTCTAGCTATGTTCTTTGAAAATTGCACAGTGAATA
AGTAAAGCTAAAGGTATATAAAAATCCTTTGTAAGAATACAATTTGCAAAGTGACAGAGGA
AAGCtacgggagacgg

The lowercase sequences are Esp3I restriction sites. The boldface sequence is the dCpf1 direct repeat sequence. The underlined sequences are the gRNA targeting the promoter region of the porA metabolic gene cluster. The italicized sequence is a 16s rRNA terminator region obtained from the Cs 16s rRNA gene (CLOSPO_00916).

(ii). Introduce the Vectors pGM-ABCD and pGM-ABCD-006 into C. sporogenes ATCC 15579 and Quantify their Production of Branched Short-Chain Fatty Acid

We used the same protocol as described herein to introduce the vectors pGM-ABCD (control) and pGM-ABCD-006 (porA transcription repression mutant) into C. sporogenes ATCC 15579. For each conjugation, we cultivated three isolated single colonies in 5 mL TYGC liquid broth supplemented with 15 μg/mL thiamphenicol for 36 hrs. We extracted RNA from 5 mL of liquid culture using Quick RNA fungal/bacterial kit (Zymo Research). We quantified the relative expression of porA in the control strain and its transcription repression mutant. To quantify the production of branched short-chain fatty acid, 10 μL supernatant of both the control strain and the porA transcription repression mutant was derivatized and subject to LC-qTOF analysis.

Genetic Manipulation of baiH Gene in the Bai Operon of Faecalicatena Contorta S122 (S122)
Screening 27 Genetically Targetable Clostridia Strains that 7α-Dehydroxylate Primary Bile Acid Cholic Acid (CA) and Chenodeoxycholic Acid (CDCA)

To identify if there are any 7α-dehydroxylating bacteria in the group of 27 genetically targetable Clostridia commensals characterized via the GM pipeline, we restreaked the bacteria on the TSAB or BHIB agar, and a single colony of each strain was cultivated in 1 mL liquid medium supplemented with 100 μM CA and 100 μM CDCA. After 48 hrs, 1 ml of the culture was centrifuged at 15000 g for 20 min, and the supernatant was subjected to LC-MS analysis to examine if CA and CDCA were 7α-dehydroxylated to DCA and LCA (see Example 1 for detailed information).

Whole-Genome Sequencing of Faecalicatena contorta S122 (S122)

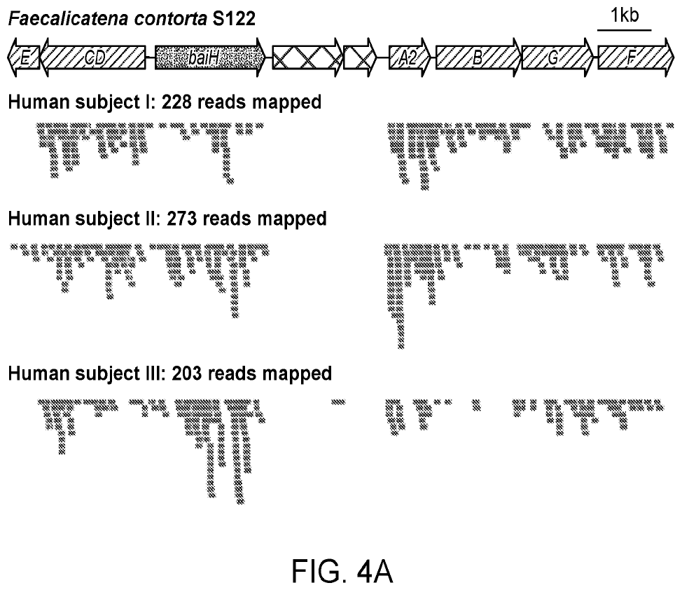

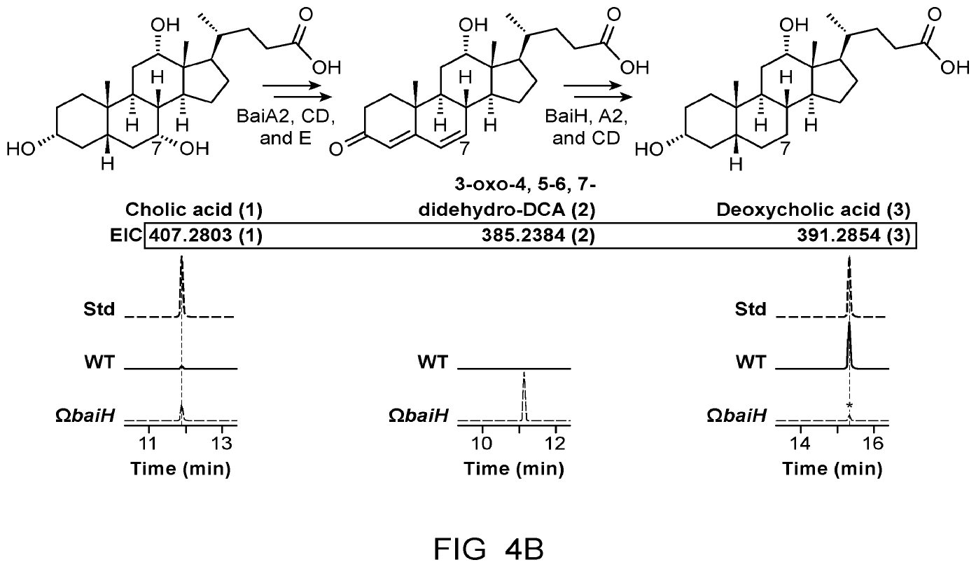

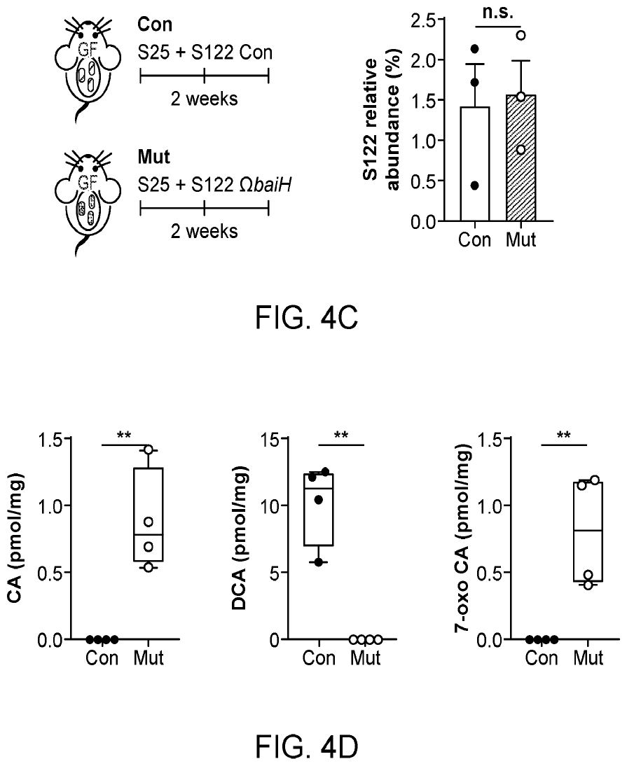

The biosafety level 1 Faecalicatena contorta S122 was isolated from healthy human stool. We cultivated a single colony of Faecalicatena contorta S122 (S122) in 3 mL Mega liquid broth for 24 hrs and extracted the genomic DNA using Quick DNA fungal/bacterial kit (Zymo Research). The S122 genomic DNA was sent for whole genome sequencing (BGI). The raw sequencing reads were filtered (for quality control), and de novo assembled (Geneious). The assembled contig in fasta format was further annotated using Prokka (v1.12) (Seemann, 2014). To locate the bai operon in the S122 genome, we performed a tblastn search of each bai gene annotated in the genome of C. scindens ATCC 35704 and identified a cluster of nine genes as a candidate bai operon in the S122 genome (FIG. 4A).

Vector assembly for utilizing Group II intron to disrupt baiH in Faecalicatena contorta S122 (S122)

We used the Intron targeting and design tool on the ClosTron website (http://www.clostron.com/clostron2.php) to design the Group II introns targeting the S122 baiH gene. The baiH-targeting intron was amplified using primers EBS universal primer+WBJ_BaiH_tgt_645_IBSN+WBJ_BaiH_tgt_645_EBS1d+WBJ_BaiH_tgt_645_EBS2, and the purified PCR product was then Gibson assembled with the backbone that amplified from the plasmid pGM-FCAQ using primers pMTL007C-E2_F and pMTL007C-E2_R to get the plasmid pGM-FCAR-002.

To introduce thiamphenicol resistance to S122, we generated a plasmid pGM-FCFQ by replacing the original conjugation-selection marker catP marker of pGM-FCAQ with aad9-ampR, and retrotransposition-activated marker (RAM) is changed from ermB to catP. (FIG. 9) The antibiotic marker aad9-ampR was amplified using primers aad9_carb_007C2_thiam_F and aad9_carb_007C2_thiam_R, and the purified PCR product was then Gibson assembled with the backbone that amplified from the plasmid pGM-FCAQ using primers pmtl_007C2_thiam_marker_F and pMTL007C_Clostron_87_87_Erm_ItrA R to get the plasmid pGM-FCDQ. And the antibiotic marker catP was then amplified using primers clostron_Thiam_F and clostron_Thiam_R, and the purified PCR product was then Gibson assembled with the backbone that amplified from the plasmid pGM-FCDQ using primers Csp-316s_marker_F and Csp-316s_marker_R to get the plasmid pGM-FCFQ.

Genetic Disruption of baiH in Faecalicatena contorta S122 (S122)

To disrupt the baiH gene, baiH-targeting plasmid pGM-FCAR-002 was first introduced into S122 following the aforementioned conjugation procedure (see Example 1 and FIG. 24). After conjugation, the S122 colonies harboring pGM-FCAR-002 appeared on the TSAB plate supplemented with 15 μg/mL thiamphenicol and 250 μg/mL D-cycloserine. Next, four colonies were restreaked onto a TSAB plate with the same antibiotics to isolate a single colony. The single colonies were inoculated into 1 mL Mega supplied with 15 μg/mL thiamphenicol and 250 g/mL D-cycloserine. After 24-36 hrs, 50 μL of cultures were spread onto TSAB plates supplemented with 250 μg/mL D-cycloserine and 10 μg/mL erythromycin. The integrated colonies typically appeared after 36-48 hrs. Eight colonies were picked to inoculate 3 mL Mega supplemented with 250 μg/mL D-cycloserine and 10 μg/mL erythromycin. After 24-36 hrs, we extracted their genomic DNA using Quick DNA fungal/bacterial kit (Zymo Research) and performed diagnostic PCR using primers WBJ_BaiH_tgt_DiagF and WBJ_BaiH_tgt_DiagR (FIG. 17B).

To confer the baiH mutant strain with thiamphenicol resistance, the plasmid pGM-FCFQ was first introduced into S122+pGM-FCAR-002 following the aforementioned conjugation procedure (FIG. 24). After conjugation, the S122+pGM-FCAR-002 colonies harboring pGM-FCFQ appeared on the TSAB plate supplemented with 10 μg/mL erythromycin, 300 μg/mL spectinomycin, and 250 μg/mL D-cycloserine. Next, four colonies were restreaked onto a TSAB plate with the same antibiotics to isolate single colony. The single colonies were inoculated into 1 mL Mega supplied with 10 μg/mL erythromycin, 10 μg/mL spectinomycin, and 250 μg/mL D-cycloserine. After 24-36 hrs, 50 μL of cultures were spread onto TSAB plates supplemented with 250 μg/mL D-cycloserine, 15 μg/mL thiamphenicol, and 10 μg/mL erythromycin. The integrated colonies typically appeared after 36-48 hrs. Colonies were picked to inoculate Mega supplemented with 250 μg/mL D-cycloserine, 15 μg/mL thiamphenicol, and 10 μg/mL erythromycin.

Likewise, we constructed the S122 control strain with both erythromycin and thiamphenicol resistance using 16s-targeting plasmid pGM-FCAQ and pGM-FCFQ. Both Group II introns in these two plasmids are targeting the 16s rRNA genes, we have validated that the engineered strains with thiamphenicol and erythromycin resistance still carry at least one copy of intact 16s rRNA gene in their genomes by diagnostic PCR.

### Quantification of Microbiome-Derived SCFAs, BSCFAs, and Bile Acids Using LC-MS

### Quantification of Isovalerate, Propionate, Butyrate, and Bile Acids Production in the Culture Supernatant of the Control and Mutant Strains

A single colony of control or mutant strain was used to inoculate 1 mL pre-reduced liquid medium (TYGB or Mega), if needed, supplemented with 200 μg/mL gentamycin+15 μg/mL thiamphenicol for Bacteroidia strains, or 250 μg/mL D-cycloserine+15 μg/mL thiamphenicol for Clostridia strains, or for bile acids measurement in Faecalicatena contorta S122 (S122), Mega with 250 μg/mL D-cycloserine+10 μg/mL erythromycin was used. To pre-reduce, the liquid medium was left in the chamber with a loosened cap for at least 48 hrs before inoculation. The culture was incubated in an anaerobic chamber at 37° C. under an atmosphere consisting of 5% CO2, 7.5% H2, 87.5% N2.

The cultures were incubated for 48 hrs in the anaerobic chamber. For the quantification of isovalerate, propionate, and butyrate, a 10 μL aliquot of the culture was mixed with 190 μL of short-chain fatty acids (SCFAs) derivatization solution (1 mM 2,2′-dipyridyl disulfide, 1 mM triphenylphosphine, and 1 mM 2-hydrazinoquinoline dissolved in acetonitrile) (Lu et al., 2013). The resulting mixture was vortexed and incubated at 60° C. for 1 hr. The mixture was centrifuged at 21000×g for 20 min, and the supernatant was analyzed using an Agilent 1290 LC system coupled to an Agilent 6530 quadrupole time-of-flight (QTOF) mass spectrometer with a 130Å, 1.7 μm, 2.1 mm×100 mm ACQUITY UPLC BEH C18 column (Waters). We used the following solvent system: A: H2O with 0.1% formic acid; B: Methanol with 0.1% formic acid. 1 μL of each sample was injected, and the flow rate was 0.35 mL/min with a column temperature of 40° C. The gradient for HPLC-MS analysis was: 0-6.0 min, 99.5%-70.0% A; 6.0-9.0 min, 70.0%-2.0% A; 9.0-9.4 min, 2.0% A; 9.4-9.6 min, 2.0%-99.5% A. Peaks were assigned by comparison with authentic standards and relative analyte concentrations were quantified by comparing their peak areas with those of internal standards.

For bile acids detection and quantification, 100 μL of culture was centrifuged at 21000×g for 20 min, and the supernatant was analyzed using an Agilent 1290 LC system coupled to an Agilent 6530 quadrupole time-of-flight (QTOF) mass spectrometer with a 1.7 μm, 2.1 mm×100 mm Kinetex C18 column (Phenomenex). We used the following solvent system: A: H2O with 0.05% formic acid; B: Acetone with 0.05% formic acid. 1 μL of each sample was injected, and the flow rate was 0.35 mL/min with a column temperature of 40° C. 0-1 min, 75% A; 1-25 min, 75%-25% A; 25-26 min, 25%-0% A; 26-30 min, 0% A; 30-32 min 0%-75% A. Peaks were assigned by comparison with authentic standards. Their concentrations were calculated using the standard curve and normalized to the fecal/cecal weight.

### Quantification of Isovalerate, Propionate, Butyrate, and Bile Acids in Mouse Biological Samples

For the quantification of isovalerate, propionate, and butyrate, we made standard curves of isovalerate, propionate, and butyrate based on the Area Under Curve (AUC) of true chemical standards at different concentrations. A ˜ 10 mg fecal samples (or cecal samples) were resuspended in 50 μL of 50% MeOH (in H2O) and vortexed for 10 min (some beads were added to disperse the fecal/cecal material). Then the mixture was spun down, and 10 μL of supernatant was mixed with 190 μL short-chain fatty acids (SCFAs) derivatization solution. The resulting mixture was vortexed and incubated at 60° C. for 1 hr, then the mixture was centrifuged at 21000×g for 20 min, and the supernatant was analyzed by LC-MS. The method and column for LC-MS are the same as described above. The concentrations of isovalerate, propionate, and butyrate were calculated using the standard curve and normalized to the fecal/cecal weight.

For the detection of bile acids, A ˜10 mg fecal samples were resuspended in 100 μL of 50% MeOH (in H2O) and vortexed for 10 min (some beads were added to disperse the fecal material). Then the mixture was centrifuged at 21000×g for 20 min, and the supernatant was analyzed by LC-MS. The method and column for LC-MS are the same as described above. Their concentrations were calculated using the standard curve and normalized to the fecal/cecal weight.

### Growth Curve of Bacteria

Bacteroides (Bacteroides fragilis 3112 (Bac1) and Bacteroides vulgatus ATCC 8482 (Bac2)) and Erysipelotrichaceae (Clostridium ramosum ATCC 25554 (Ery1), Erysipelatoclostridium ramosum strain 113-1 (Ery2), Clostridium ramosum DSM 24812 (Ery3), Clostridium ramosum DSM 1402 (Ery4), HM-173 Clostridium innocuum 6_1_30 (Ery5), Clostridium innocuum DSM 22910 (Ery6) and Holdemania filiformis DSM 12042 (Ery7)) were streaked from a glycerol stock onto TSAB agar plates and incubated anaerobically for ˜ 24 h at 37° C. Three colonies were inoculated into 1 ml of Mega broth and were anaerobically cultured for overnight at 37° C. Cells were diluted 1,000-fold into Mega broth to reach late-log phase. Then 5 μL of the culture was resuspended in 145 μL broth, loaded into a 96-well plated, and incubated anaerobically at 37° C. in Multiskan Sky Microplate Spectrophotometer (Thermo Fisher Scientific). Four bile acids (CA, 7-oxoCA, DCA, and 3-oxoDCA, 500 μM each) were tested with their solvent DMSO as control. Optical densities at 600 nm (OD600) were recorded every 60 min until the cultures reached the stationary phase. Bacterial growth curves were performed in triplicate with each biological replicate deriving from a single colony.

Quantitative PCR (qPCR)
qPCR of dCpf1 Targeting Genes

Three isolated single colonies of control or mutant strain were used to inoculate 5 mL pre-reduced Mega medium supplemented with 15 μg/mL thiamphenicol. The cultures were incubated for 36 hrs in the anaerobic chamber. Following incubation, the cultures were centrifuged at 1500×g for 5 min, and the supernatant was discarded. RNA was extracted from the resulting bacterial pellet using Quick RNA fungal/bacterial kit (Zymo Research) following the manufacturer's protocol. Reverse transcription of extracted RNA into cDNA was performed using PrimeScript™ RT Reagent Kit (TaKaRa) following the manufacturer's protocol. Real-time quantitative PCR (qPCR) was performed on cDNA using SYBR green chemistry (Applied Biosystems). Reactions were run on a real-time quantitative PCR system (ABI 7500; Applied Biosystems). Samples were normalized to 16s rRNA of each strain.

qPCR for the Comparison of Erysipelotrichaceae Relative Fold Change Between Groups

For the qPCR of Erysipelotrichaceae abundance in liquid culture, overnight culture of two Bacteroides (Bacteroides fragilis 3112 (Bac1) and Bacteroides vulgatus ATCC 8482 (Bac2)) and seven Erysipelotrichaceae (Clostridium ramosum ATCC 25554 (Ery1), Erysipelatoclostridium ramosum strain 113-1 (Ery2), Clostridium ramosum DSM 24812 (Ery3), Clostridium ramosum DSM 1402 (Ery4), HM-173 Clostridium innocuum 6_1_30 (Ery5), Clostridium innocuum DSM 22910 (Ery6) and Holdemania filiformis DSM 12042 (Ery7)) in Mega (˜1×10⁷ CFU) were inoculated into Mega with different concentrations of DCA (0 μM, 250 μM, 500 μM), or co-inoculated together with S122 control or S122 ΩbaiH mutant into Mega with 500 μM CA. After incubation for 24 h, gDNA was extracted using Quick RNA fungal/bacterial kit (Zymo Research) and qPCR was performed using primers Bac_Erysi_16s_qPCR_F-2+Bac_Erysi_16s_qPCR_R-2 to amplify total 16s of both Bacteroides and Erysipelotrichaceae as reference, and primers Erysi_16s_qPCR_F+Erysi_16s_qPCR_R to amplify Erysipelotrichaceae-specific 16s for the comparison of Erysipelotrichaceae abundance between groups.

For the qPCR of Erysipelotrichaceae relative fold change in fecal samples, gDNA in fecal samples was extracted using QIAamp Fast DNA Stool Mini Kit (Cat. #51604), and qPCR was performed using primers Bac_Erysi_16s_qPCR_F-2+Bac_Erysi_16s_qPCR_R-2 to amplify total 16s of both Bacteroides and Erysipelotrichaceae as a reference, and primers Erysi_16s_qPCR_F+Erysi_16s_qPCR_R to amplify Erysipelotrichaceae-specific 16s for the comparison of Erysipelotrichaceae relative fold change between groups.

Colonize Germ-Free and SPF Mice with the Control and Mutant Bacteria

Germ-free mouse experiments were performed on gnotobiotic Swiss Webster or C57BL/6 mice, which were bred within sterile vinyl isolators and maintained at the gnotobiotic facility at Weill Cornell Medicine. SPF mice on a C57BL/6 background were purchased from the Jackson Laboratory and were bred and maintained in specific-pathogen-free facilities at Weill Cornell Medicine. Sex- and age-matched mice between 8 and 14 weeks of age were used for experiments if not otherwise indicated (n=4 or 5 per group).

For mono-colonization in germ-free mice, taking Eubacterium maltosivorans DSM 105863 control (S117+pGM-FBCD) and its SCFAs mutant (S117+pGM-FBCD-020) as an example, a 200 μL portion of their overnight RCM culture (˜1×10⁷ CFU) were mono-colonized with germ-free mice (n=4 per group) via oral gavage. The germ-free mice were maintained on standard chow and water containing minimal thiamphenicol (15 μg/mL). Successful colonization was determined by colony-forming unit (CFU) counting. After two weeks of colonization, mice were euthanized humanely by CO2 asphyxiation. Blood was collected by cardiac puncture, and serum was prepared using microtainer serum separator tubes obtained from Becton Dickinson (Cat. #365967). The urine, cecal contents, and feces were collected and snap-frozen in liquid nitrogen and stored at −80° C. until use.

For co-colonization of Faecalicatena contorta S122 (S122) control or its baiH mutant together with Bacteroides sp. 116 (S25) in germ-free mice, three verified transconjugants were restreaked and subcultured in Mega broth, and 1 mL of their overnight Mega culture (˜1×10⁷ CFU) were mixed and co-colonized with germ-free mice (n=5 per group) via oral gavage (300 L per mouse). The germ-free mice were maintained on standard chow and water supplemented with 15 g/ml thiamphenicol. Successful colonization was determined by the quantitative PCR (qPCR) of 16s gene of S122 and S25 (data not shown) and 16s rRNA sequencing.

For co-colonization of Faecalicatena contorta S122 (S122) control or its baiH mutant together with two Bacteroides (3-member community) mentioned in FIG. 22G, and co-colonization of Faecalicatena contorta S122 (S122) control or its baiH mutant together with two Bacteroides (Bac1-2) and seven Erysipelotrichaceae (Ery1-7) (10-member community) in FIG. 7D, 1 mL of their overnight Mega culture (˜1×10⁷ CFU) were mixed and co-colonized with germ-free mice (n=4 per group) via oral gavage (300 μL per mouse). The germ-free mice were maintained on standard chow, and cholic acid sodium salt (5 mM for the 10-member community and 0.5 mM for the 3-member community) was supplied in water to facilitate S122 colonization and ensure both gnotobiotic mice settings have comparable gut bile acid profiles. Successful colonization of S122 was determined by the Colony-forming unit (CFU) and LCMS. After 14 days, mice were administered with DSS for 8 or 9 days. After DSS was removed and mice were recovered with regular drinking water for 1 or 2 days, mice were euthanized humanely by CO2 asphyxiation. Blood was collected by cardiac puncture, and serum was prepared using microtainer serum separator tubes obtained from Becton Dickinson (Cat. #365967). Colon length was measured, proximal colon/distal colon/ileum tissue samples were collected for histology, and colon/ileum tissue samples were collected for qPCR. The urine, cecal contents, ileal content, and feces were collected and snap-frozen in liquid nitrogen and stored at −80° C. until use.

For co-colonization of Faecalicatena contorta S122 (S122) together with 55 other genetically targetable strains identified in this study, 1 mL of their overnight Mega/RCM/CMM culture (˜1×10⁷ CFU) were mixed and co-colonized with germ-free mice (n=5 per group) via oral gavage (300 μL per mouse). The germ-free mice were maintained on standard chow. Successful colonization of S122 was determined by LCMS, and colonization of other strains was confirmed by 16s rRNA sequencing. After two weeks of colonization, mice were euthanized humanely by CO2 asphyxiation. Blood was collected by cardiac puncture, and serum was prepared using microtainer serum separator tubes obtained from Becton Dickinson (Cat. #365967). The urine, cecal contents, and feces were collected and snap-frozen in liquid nitrogen and stored at −80° C. until use.

For the colonization of Faecalicatena contorta S122 (S122) control or its baiH mutant in SPF mice, three verified transconjugants were restreaked and subcultured in Mega broth, and a 300 μL portion of their overnight Mega culture (˜1×10⁷ CFU) were colonized with SPF mice (n=4 per group) via oral gavage, twice per day for 3 days in a row. The SPF mice were maintained on standard chow (Lab Diet 5053) and water containing thiamphenicol (15 μg/mL) and erythromycin (10 μg/mL). Successful colonization was determined by colony-forming unit (CFU) counting. After 14 days, mice were administered with DSS for 8 days. After DSS was removed and mice were recovered with regular drinking water (water containing 15 μg/mL thiamphenicol and 10 μg/mL erythromycin) for 3 days, mice were euthanized humanely by CO2 asphyxiation. Blood was collected by cardiac puncture, and serum was prepared using microtainer serum separator tubes obtained from Becton Dickinson (Cat. #365967). Colon length was measured, proximal colon/distal colon/ileum tissue samples were collected for histology, and colon/ileum tissue samples were collected for qPCR. The urine, cecal contents, ileal content, and feces were collected and snap-frozen in liquid nitrogen and stored at −80° C. until use.

### Colony-Forming Unit (CFU) Quantification of Mouse Fecal Samples

For CFU of mono-colonized germ-free mice, a ˜5 mg fecal material was resuspended in 200 μL pre-reduced Gibco™ phosphate-buffered saline buffer, pH 7.4. A 10-fold serial dilution (to 10-4) was made in the same buffer on a 96-well plate, and 50 μL from each well was plated on pre-reduced TSAB agar and was incubated anaerobically at 37° C. After 24 hrs, colonies will appear, and the CFU of fecal samples from control and mutant strains colonized germ-free mice is calculated after normalizing to fecal weight.

For CFU of SPF mice colonized with Faecalicatena contorta S122 (S122) control (S122+pGM-FCAQ+pGM-FCFQ) or its baiH mutant (S122+pGM-FCAR-002+pGM-FCFQ, S122 ΩbaiH mutant), a ˜5 mg fecal material was resuspended in 200 μL pre-reduced Gibco™ phosphate-buffered saline buffer, pH 7.4. A 10-fold serial dilution (to 10-4) was made in the same buffer on a 96-well plate and 50 μL from each well was plated on pre-reduced TSAB agar supplemented with 250 μg/mL D-cycloserine+15 μg/mL thiamphenicol+10 μg/mL erythromycin and was incubated anaerobically at 37° C. After 24 hrs, colonies will appear and colonies were inoculated in 3 mL Mega broth supplemented with 250 μg/mL D-cycloserine+15 g/mL thiamphenicol+10 μg/mL erythromycin. After 12 hrs, we extracted their genomic DNA using Quick DNA fungal/bacterial kit (Zymo Research). We performed diagnostic PCR using primers WBJ_BaiH_tgt_DiagF and WBJ_BaiH_tgt_DiagR to verify that colonies on plates were the control and the baiH mutant strain of S122. The CFU of fecal samples from control and mutant strains colonized SPF mice is calculated after normalizing to fecal weight.

Isolation of Gut Bacterial Strains from Collected Fecal Samples

Fecal samples (from human or mouse) were suspended in PBS (1:10 w/v), the suspension was then restreaked on TSAB/BHIB plates and incubated in an anaerobic chamber at 37° C. under an atmosphere consisting of 5% CO2, 7.5% H2, 87.5% N2, in the meantime, the suspension was also restreaked on LB plates and incubated aerobically at 37° C. Colonies typically appeared after 24-36 hrs, the isolated single colonies were inoculated in 3 mL Mega/RCM/CMM/TYBG or LB broth. After ˜12 hrs, we extracted their genomic DNA using Quick DNA fungal/bacterial kit (Zymo Research), and amplified the 16s rRNA region of the colony using primers 16s_27F+16s_1391R. The PCR product was purified and sent for Sanger sequencing using primer 16s_1391R to identify the species of the isolated strains.

### DSS Treatment of GF and SPF Mice

### DSS Administration

Dextran sulfate sodium salt (DSS) of colitis-grade with an average MW of 36,000-50,000 Da (MP Biomedicals) was added to drinking water at day 0. DSS was administered until substantial inflammation was induced as evidenced by significant weight loss. For the GF mice experiment in FIG. 6F, DSS was used at a concentration of 2% (in water supplemented with 15 g/ml thiamphenicol) for 7 days, and for SPF mice experiment in FIG. 6A, DSS was used at a concentration of 2.5% (in water supplemented with 15 μg/ml thiamphenicol and 10 μg/ml erythromycin) for 8 days. After DSS treatment, DSS was removed from the drinking water, GF mice in FIG. 6F were recovered with regular water (with 15 μg/ml thiamphenicol) for 3 days and SPF mice in FIG. 6A were recovered with water containing 15 μg/mL thiamphenicol and 10 g/mL erythromycin for 3 days. For the experiment in FIG. 7D and FIG. 22G, DSS was used at a concentration of 2.5% for 9 days. After DSS treatment, DSS was removed from the drinking water, mice in FIG. 7D and FIG. 22G were recovered with regular water for 1 or 2 days. Throughout DSS treatment and recovery, mice were weighed daily at the same time of day at indicated time points, and feces were collected daily at the same time points. Mice were then euthanized by CO2 asphyxiation. Blood was collected by cardiac puncture, and serum was prepared using microtainer serum separator tubes obtained from Becton Dickinson (Cat. #365967). Colon length was measured, proximal colon/distal colon/ileum tissue samples were collected for histology, and colon/ileum tissue samples were collected for qPCR. The urine, cecal contents, ileal content, and feces were collected and snap-frozen in liquid nitrogen and stored at −80° C. until use.

Quantitative PCR (qPCR) of Colonic Inflammatory Genes Expression Post DSS-Treatment

For the qPCR of inflammatory genes in the colon, colonic samples were homogenized, and then RNA was extracted from the resulting homogenate using Quick RNA fungal/bacterial kit (Zymo Research) following the manufacturer's protocol. Reverse transcription of extracted RNA into cDNA was performed using PrimeScript™ RT Reagent Kit (TaKaRa) following the manufacturer's protocol. Real-time quantitative PCR (qPCR) was performed on cDNA using SYBR green chemistry (Applied Biosystems). Reactions were run on a real-time quantitative PCR system (ABI 7500; Applied Biosystems). Samples were normalized to Hprt1.

### Quantification of Fecal Lipocalin-2 (LCN-2) by ELISA

For fecal lipocalin-2 quantification, fecal samples were collected and suspended in PBS containing 1% Bovine Serum Albumin (1 g/100 mL) to a final concentration of 100 mg/mL and vortexed for 20 min to get a homogenous fecal suspension. These samples were then centrifuged for 10 min at 14 000 g and 4° C. to remove aggregates, and the resulting supernatant was collected. Afterward, according to the manufacturer's instructions, a sandwich ELISA was performed following appropriate dilution using mouse lipocalin-2/NGAL DuoSet ELISA (R & R&D Systems).

### Assessment of Fecal Hematochezia Score

Fecal samples were collected daily at the same time of day at indicated time points and subjected to Hemoccult II SENSA Dispensapak Plus kit (Backman Coulter) to assess hematochezia scores following the manufacturer's instructions.

### Histology

Distal colon sections were obtained and fixed in 10% neutral buffered formalin overnight at room temperature and then were transferred to 70% ethanol. Then sections were paraffin-embedded, sectioned, and stained with hematoxylin and eosin by IDEXX BioAnalytics company. Blinded histological evaluation was conducted on a scale of 1-3 or 4 for the following histologic parameters: area involved (0-4), erosion/ulceration (0-4), follicles (0-3), edema (0-3), fibrosis (0-3), crypt loss (0-4), granulocytes (0-3), mononuclear cells (0-3), and crypt damage/apoptosis (0-4). Scores were accumulated to give a total score of inflammation.

16s rRNA Gene Sequencing of Fecal Samples

For the 16s rRNA gene sequencing of fecal samples from SPF mice colonized with Faecalicatena contorta S122 (S122) control (Con) and ΩbaiH mutant (Mut), gDNA in fecal samples was extracted using QIAamp Fast DNA Stool Mini Kit (Cat. #51604), and the concentration of double-stranded gDNA in the extracted gDNA was measured using Quant-iT™ dsDNA Assay Kit, high sensitivity (Cat. #Q33120). Then gDNA was normalized to 20 ng/μL and sent for 16s rRNA gene sequencing.

Next generation sequencing library preparations and Illumina MiSeq sequencing were conducted at GENEWIZ, Inc. (Suzhou, China). DNA samples were quantified using a Qubit 2.0 Fluorometer (Invitrogen, Carlsbad, CA, USA). 30-50 ng DNA was used to generate amplicons using a MetaVx™ Library Preparation kit (GENEWIZ, Inc., South Plainfield, NJ, USA).

V3, V4, and V5 hypervariable regions of prokaryotic 16s rDNA were selected for generating amplicons and following taxonomy analysis. GENEWIZ designed a panel of proprietary primers aimed at relatively conserved regions bordering the V3, V4, and V5 hypervariable regions of bacteria 16s rDNA. The v3 and v4 regions were amplified using forward primers containing the sequence “CCTACGGRRBGCASCAGKVRVGAAT” (SEQ ID NO: 19) and reverse primers containing the sequence “GGACTACNVGGGTWTCTAATCC” (SEQ ID NO: 20). The v4 and v5 regions were amplified using forward primers containing the sequence “GTGYCAGCMGCCGCGGTAA” (SEQ ID NO: 21) and reverse primers containing the sequence “CTTGTGCGGKCCCCCGYCAATTC” (SEQ ID NO: 22). 1st round PCR products were used as templates for 2nd round amplicon enrichment PCR. At the same time, indexed adapters were added to the ends of the 16s rDNA amplicons to generate indexed libraries ready for downstream NGS sequencing on Illumina Miseq.

DNA libraries were validated by Agilent 2100 Bioanalyzer (Agilent Technologies, Palo Alto, CA, USA), and quantified by Qubit 2.0 Fluorometer. DNA libraries were multiplexed and loaded on an Illumina MiSeq instrument according to manufacturer's instructions (Illumina, San Diego, CA, USA). Sequencing was performed using a 2×300/250 paired-end (PE) configuration.

The QIIME data analysis package was used for 16s rRNA data analysis. The forward and reverse reads were joined and assigned to samples based on barcode and truncated by cutting off the barcode and primer sequence. Quality filtering on joined sequences was performed and sequence which did not fulfill the following criteria were discarded: sequence length <200 bp, no ambiguous bases, mean quality score >=20. Then the sequences were compared with the reference database (RDP Gold database) using UCHIlVE algorithm to detect chimeric sequence, then the chimeric sequences were removed (FIG. 34).

The effective sequences were used in the final analysis. Sequences were grouped into operational taxonomic units (OTUs) using the clustering program VSEARCH (1.9.6) against the Silva 119 database pre-clustered at 97% sequence identity. The Ribosomal Database Program (RDP) classifier was used to assign taxonomic category to all OTUs at a confidence threshold of 0.8. The RDP classifier uses the Silva 119 database which has taxonomic categories predicted to the species level (FIGS. 32 and 33).

Sequences were rarefied prior to the calculation of alpha and beta diversity statistics. Alpha diversity indexes were calculated in QIIME from rarefied samples using for diversity the Shannon index, for richness the Chao1 index. Beta diversity was calculated using principal coordinate analysis (PCoA) performed.

Bioinformatics Analyses Performed in this Study
Phylogenetic Analyses of the 91 Non-Model Gut Commensals that are Genetically Targetable

To construct the phylogenetic tree in FIG. 1, we extracted their 16s rRNA sequences from their sequenced genomes. If the bacteria have no sequenced genome information, their 16s rRNA sequences were downloaded from the Silva database (Joseph et al., 2018) or from the Sanger sequencing result of the PCR product amplified from their extracted genomic DNA. These 16s rRNA sequences were aligned using Clustal Omega, and the aligned sequences were used to construct a phylogenetic tree (neighbor-joining) with a bootstrap test of 5000 using Mega11 (Kumar et al., 2016).

### Analyses of the Prevalence and Relative Abundances of Clostridia Commensals Harboring the Bai Operon in Human Stool Datasets

The publicly available 16s rRNA sequencing reads were downloaded and mapped to the 16s rRNA sequences of five Clostridia commensals, including Faecalicatena contorta S122 (S122), Clostridium hylemonae DSM15053, Clostridium hiranonis DSM 13275, Clostridium scindens ATCC35704, and Dorea sp. D27 using Geneious. We used a very stringent setting, and only reads with >95% quality and minimal 100% overlap identity will be mapped to their 16s rRNA sequences. The prevalence of the strain and their closely related microbes is calculated by dividing the number of stool samples with at least one mapped read by the total number of stool samples. Their relative abundances were calculated by dividing the total mapped reads by the stool sample's total reads. Similarly, the relative abundances of S122 control or mutant strain shown in FIG. 4E were analyzed and calculated by dividing the total mapped high-quality reads by the total nonchimeric reads of the mouse stool samples.

### Metatranscriptomic Analyses of the S122 Bai Operon

To determine if the S122 bai operon is actively transcribed under the condition of host colonization, we built a local DNA sequence database consisting of all the bai operons identified so far (FIGS. 16A-16C) metatranscriptomic analyzes. We mapped metatranscriptomic reads from the stool of healthy human subjects (David et al., 2014) to this database using Bowtie 2 (local, high sensitivity); representative mapping results are shown in FIG. 4A.

### Analyses of the Correlation Between Human Fecal DCA Level and Specific Taxonomic Groups of Gut Microbes and Fecal Calprotectin

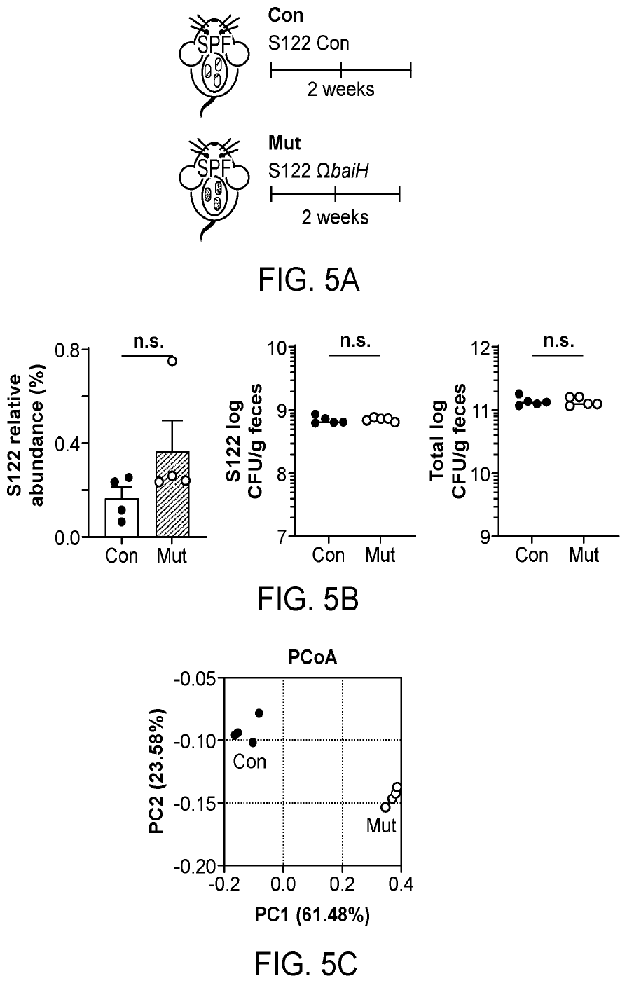

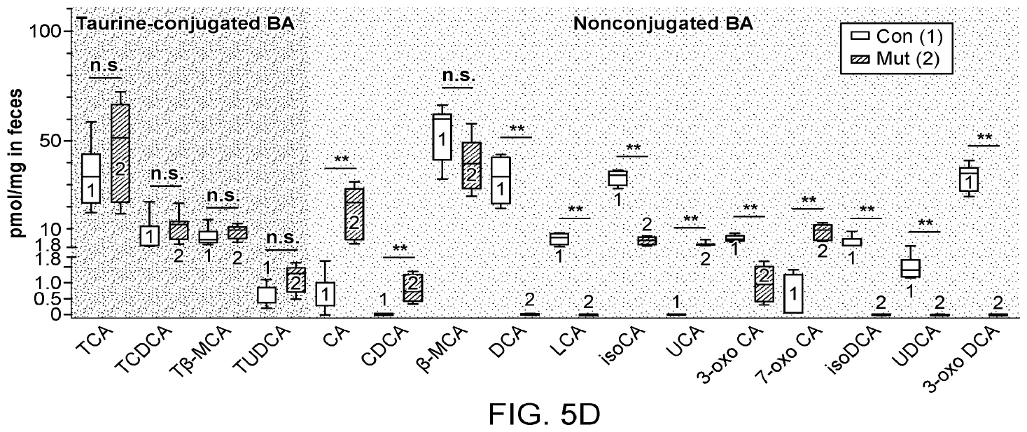

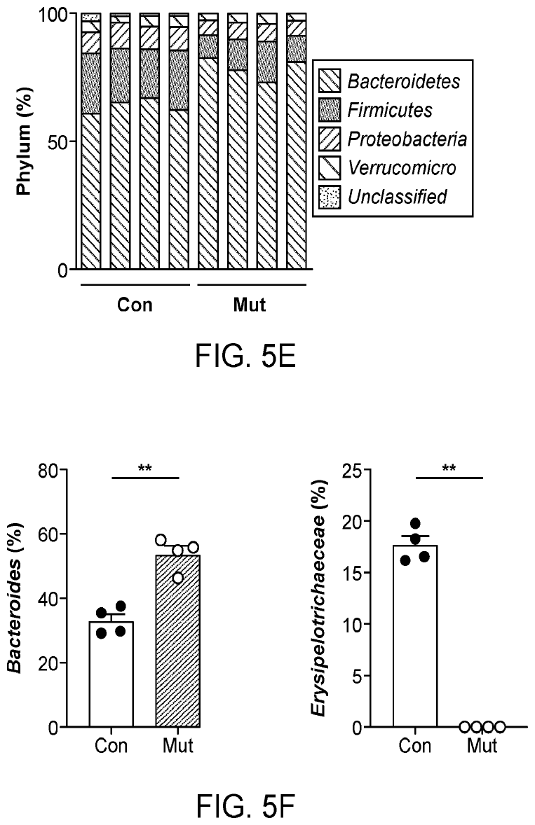

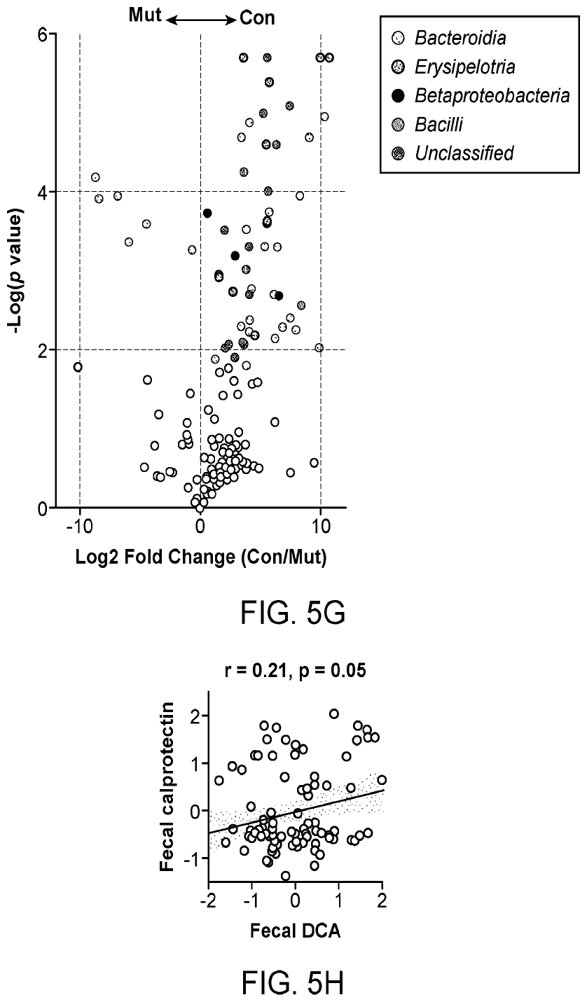

The metabolomics data, fecal calprotectin data, and relative abundances of specific taxonomic groups of gut microbes whose relative abundances are affected by baiH depletion in our experiment were downloaded and extracted from the iHMP-IBD website (https://ibdmdb.org/). Longitudinal data of the same participant were Z-transformed (with a mean of 0 and an SD of 1). The correlation between 1) fecal DCA and fecal calprotectin level, and 2) fecal DCA and specific taxonomic groups of gut microbes whose abundances have been affected by baiH depletion, were assessed using Pearson correlation, with a pre-specific alpha level of 0.05 to assess statistical significance. A correlation of 0.2 or higher or of ˜0.2 or lower was considered moderate. In addition, a linear mixed model with random intercept was used to assess the association between fecal DCA and fecal calprotectin/gut microbe relative abundances, accounting for the longitudinal measurements obtained from the same individual. Z-transformed values (dots) and the fitted values based on the linear mixed model (line) were presented in FIGS. 5H and 19A-19I.

### Example 2: An Overview of the GM Pipeline

The overall GM workflow is summarized in FIG. 1A. There are three challenges toward building such a pipeline. First, there is no previously reported antibiotic marker that universally functions in most of the non-model microbes. By assessing the antibiotic resistance and testing different conjugation donors to introduce antibiotic markers into 201 gut isolates, we found that one chloramphenicol resistant marker operates in majority of the 91 transformed microbes (FIG. 1B). Second, Firmicutes/Clostridia microbes are highly abundant in healthy human guts, yet the genetic manipulation of this physiologically important, host-associated bacterial group remains largely unexplored (Waller et al., 2017). Lacking their genetic tools greatly limits mechanistic dissection of the effects of Firmicutes/Clostridia genes on host biology. By optimizing multiple factors, we managed to identify gene transfer methods for 38 non-model gut Clostridia microbes (FIG. 1). Third, when we started building the pipeline, the genomes of many isolates had not been sequenced, posing a considerable roadblock in establishing their targeted gene manipulation tools on a large scale. To overcome this hurdle, we incorporated CRISPRi and a lacZα transcription reporter or developed strategies to genetically target the bacterial 16S rRNA gene (FIG. 1A). For consistency, we consider a gut microbe as genetically targetable if exogenous DNA (shuttle or suicide plasmids) can be repeatedly introduced into the microbe in vitro. A genetic manipulation tool is established if targeted manipulation of its gene/gene expression is achieved in the microbes of interest.

### Example 3: Selection of Gut Microbes and Screening their Culture Conditions and Antibiotic Resistance

We prioritized Firmicutes and Bacteroidetes microbes that dominate healthy human guts (Cho and Blaser, 2012), but many (like Clostridia and Prevotella) do not have gene transfer methods and tractable genetic tools. We diversified the screened pool by selecting commensals from a variety of genera/species (FIG. 22). We first identified the culture conditions supporting the growth of these gut microbes (FIGS. 1A and 8, FIGS. 23 and 31). Next, we screened these microbes against a collection of antibiotics to identify the following (FIGS. 1A and 8, FIG. 24): 1) the MIC of an antibiotic to which they are susceptible, allowing its resistance gene to be used as a universal selective marker, and 2) an antibiotic which they are resistant to but is active against E. coli, enabling suppression of E. coli growth after conjugation. For 1), we determined the MIC of thiamphenicol that inhibits the growth of almost all the tested microbes (FIG. 24), and for 2), almost all the Clostridia are resistant to D-cycloserine or kanamycin, and all the Prevotella to gentamycin or D-cycloserine (FIG. 24).

### Example 4: A Multifactorial Optimization to Identify Gene Transfer Methodology for Non-Model Clostridia

Multiple reasons, including incompatible origins of replication (rep oris) and antibiotic markers, host endogenous defense systems, and very inefficient homologous recombination (HR), cause the genetics of gut Firmicutes/Clostridia commensals to be poorly investigated compared to its counterpart Bacteroides. (Waller et al., 2017b). Therefore, herein we have performed a multifactorial optimization of the transformation/conjugation parameters to identify gene transfer conditions for previously untransformed gut Firmicutes/Clostridia commensals (FIG. 2A):

First, because our initial attempt to introduce the four most-used Clostridium rep oris (Heap et al., 2009) into several gut Clostridia like C. bolteae were unsuccessful, we expanded the repertoire of Clostridia rep oris and developed a mixed-conjugation strategy to introduce compatible rep on into non-model gut Clostridia (FIGS. 2A, 10A-10B, and 11A-11B, see Example 1 for more details). Second, we utilized a universal catP marker regulated by a potent constitutive promoter, P_{pmtl-catp} or P_{fdx-cs} (identified via a promoter library screen in multiple non-model Clostridia, data not shown), to confer antibiotic resistance during conjugation/transformation (see Example 1 for more details). This effort significantly reduced the workload of marker-switching when applying the pipeline to a large number of previously non-targetable Clostridial microbes (FIG. 2A). Third, we attempted different approaches, including utilization of an E. coli methylase-free ‘sExpress’ conjugation donor (Woods et al., 2019), decreasing restriction-modification (RM) recognition sites (Mermelstein et al., 1992; Purdy et al., 2002; Yang et al., 2016), and/or pre-methylate transforming DNA (Jennert et al., 2000; Pyne et al., 2014), to reduce the effect of Clostridia host defense systems during conjugation/transformation (FIG. 2A, see Example 1 for more details). Last, several other parameters are optimized in this study, including conjugation time length, conjugation donor/recipient ratio, different conjugative plasmids, etc. (FIG. 2A, see Example 1 and FIG. 24 for more details). These optimized parameters are summarized in the FIG. 24, and detailed protocols for conjugation/electroporation are reported in Example 1.

These concerted efforts allowed us to identify gene transfer conditions for 38 Clostridia commensals (of 27 species) out of 92 Clostridia (of 66 species) tested (an overall 41.3% success rate) (FIGS. 2A and 11A-11B), suggesting the possibility of developing associated gene manipulation systems. As may be anticipated, multiple factors need to be optimized simultaneously to successfully introduce plasmids into previously untransformed Clostridia (FIG. 24). For instance, introducing plasmids into S71 C. barlette (that harbors a putative Type-IV RM system) requires a compatible Clostridia rep ori, a functional catP marker driven by a strong promoter (and plating on plates supplemented with thiamphenicol at MIC), an E. coli ‘sExpress’ donor that does not methylate plasmid DNA and harbors R702 conjugative plasmid, and combination with other optimized parameters such as conjugation time and conjugation antibiotics detailed in FIG. 24. Interestingly, some Clostridia accept different rep oris even if they are closely related (e.g., C. bolteae isolates, see FIG. 25), demonstrating the necessity of expanding the collection of Clostridia rep origins.

### Example 5: Testing CRISPRi-dCpf1 System in Multiple Clostridia Commensals

The following critical step toward developing a Clostridia GM pipeline is identifying a genetic manipulation tool that enables targeted gene manipulation in most Clostridia. As with Cas9-initiated cutting and dCas9-mediated interference, CRISPR-based systems have been recently applied to C. sporogenes (Canadas et al., 2019; Guo et al., 2019) and C. difficile (McAllister et al., 2017). We prioritized the CRISPRi-dCpf1 (deactivated Cpf1) system (Hong et al., 2018; Hur et al., 2016; Kim et al., 2017; Tang et al., 2017; Zetsche et al., 2015; Zhang et al., 2017) mainly because the dCpf1 does not initiate a DNA double-strand break, and the dCpf1 plasmids showed less toxicity and higher conjugation efficiency than Cas9 or Cpf1. In comparison, our preliminary test found that the double-stranded cut by Cas9/Cpf1 is lethal to many Clostridia because of their very inefficient HR. The CRISPRi-dCpf1 system incorporates a catalytically dead dCpf1 and a guide RNA (gRNA) repurposed for gene regulation in bacteria. During regulation, the dCpf1/crRNA complex binds to the template strand of a target gene and blocks the transcription elongation, thus suppressing gene expression (Kim et al., 2017; Zhang et al., 2017).

To test CRISPRi-dCpf1 in the genetically targetable Clostridia, we assembled the dCpf1 and lacZα (as a transcription reporter) with the pGM plasmids harboring the nine rep origins (FIGS. 2B (left) and 9, see Example 1 for more details). LacZα was selected because of its small size (˜300 bp) and robust expression in multiple Clostridia. We designed a duplex gRNA targeting both the promoter and the template strand of lacZα (FIGS. 2B (right), and 12A-12B). We found that dCpf1 leads to efficient knockdowns (˜3 to over 100 fold) of lacZα transcription in 25 Clostridia (FIGS. 2B and 12A-12B, FIG. 26). Several tested Clostridia could not take in this set of vectors, probably because the conjugation efficiency is greatly diminished due to this vector's large size (>10 kb) (Guo et al., 2019; Zhang et al., 2018). Altogether our data suggest that the CRISPRi-dCpf1 system regulates gene transcription in almost all the Clostridia that uptake extracellular plasmid DNA.

### Example 6: A Strategy Targeting Bacterial 16s rRNA Genes to Generate Targeted Gene Insertion Tools in Non-Model Gut Commensals

Besides CRISPRi, a targeted gene insertion tool will also facilitate studying the molecular functions of Clostridia genes. Over half of the 38 targetable Clostridia are not genome sequenced. We considered whether targeting their universally conserved DNA sequences (as ‘an archery target’) could enable selective genetic insertion of a Clostridia gene without prior knowledge of its genome sequence. However, highly conserved genes are generally functionally essential (Isenbarger et al., 2008), and a genetic mutation to these genes could be lethal. To find such a target, we interrogated the 16s rRNA gene that has been used to assess microbiome diversity and construct bacterial phylogeny. We believe that the 16s rRNA gene is an optimal target for two reasons: 1) a microbe usually has multiple copies, such that the disruption of one will not be lethal; 2) it is highly conserved among bacteria (Isenbarger et al., 2008). The same set of 16s-targeting vectors can be applied to different bacteria, thus significantly saving time and effort in sequencing and cloning. One example of a Clostridia 16s rRNA is provided below:

(SEQ ID NO: 10)
caggaaacagctatgacctgagtggcggacgggtgagtaacgcgtgggtaacctgcctcatacagggggataacagttggaa
acggatgctaataccgcataagaccacagcaccgcatggtgcgggggtaaaaactccggcggtatgagatggacccgcgtctgattagct
agttggtgaggtaacggcccaccaaggcgacgatcagtagccgacctgagagggtgaccggccacattgggactgagacacggcccaa
actcctacgggaggcagcagtggggaatattgcacaatgggcgaaagcctgatgcagcgacgccgcgtgagtgaagaaggatttcggttt
gtaaagctcttttatcagggaagaaaatgacggtacctgactaagaagccccggctaactacgtgccagcagccgcggtaatacgtaggg
ggcaagcgttatccggatttactgggcgtaaagggagcgtaggcggcaagtctgatgtgaaagcccggggctcaaccgcgggactgcttt
ggaaactgtgagtgcaggagaggtaagtggaattcctagtgtagcggtgaaatgcgtagatattaggaggaacaccagtggcgaaggcg
gcttactggactgtaactgacgctgaggctcgaaagcgtggggagcaaacaggattagataccctggtagtccacgccgtaaacgatgaat
actaggtgtygggagcccttcggtgccgcagctaacgcagtaagtattccgcctggggagtacgttcgcaagaatgaaactcaaaggaatt
gacgggggcccgcacaagcggtggagcatgtggtttaattcgaagcaacgcgaagaaccttaccaggtcttgacatccatctgaccgaga
gatggggccttcccttcgggcaggggagacaggtggtgcatggttgtcgtcagctcgtgtcgtgagatgttgggttaagtcccgcaacgag
cgcaacccttatcyttagttgccagcattaagctgggcactctagggagactgccggggataacccggaggaaggtggggatgacgtcaa
atcatcatgccccttatgacctgggctacacacgtgctacaatggcgtaaacaaagggaagcgagaccgcgaggccgagcaaatcccaa
aaagtctcagttcggattgtagtctgcaactcgactacatgaagctggaatcgctagtaatcgcggatcagaatgccgcggtgaatacgttcc
cgggccttgtacacaccgcccgtcacaccatgggagtcagtaacgcccgaagtcggtgacctaaccaaggagggagctgccgaaggtg
ggachgatgactggggtgaagtcgtaacaaggtagccgtatcggaaggtgcggctggatcacctcctttctaaggaatacaaattcggccg
gccag

Group II intron-directed mutagenesis systems, such as Targetron (Zhong et al., 2003) or Clostron (Heap et al., 2007), utilize base-pairing (between the excised intron lariat RNA and the target site DNA) for DNA target recognition to direct the site-specific insertion of a retrotransposition-activated selectable marker (RAM) into the targeted DNA loci. The RAM itself is interrupted by a self-splicing group I intron and only confers the corresponding antibiotic resistance after splicing out the group I intron and successful insertion into the Clostridial chromosome (Heap et al., 2007; Zhong et al., 2003). We proposed that a Group II intron targeting the 16s gene will likely integrate into the 16s loci of multiple Clostridia. To test this assumption, we aligned their 16s rRNA genes (from the HMP reference genomes (Turnbaugh et al., 2007)) and identified one potential, highly conserved target site of Group II intron (FIG. 2C). We then introduced the 16s-targeting Group II intron (16s-tron), along with their compatible rep origins and antibiotic RAM, into 19 targetable Clostridia (FIGS. 2C, 9, and 13A). The RAM provides antibiotic resistance only upon integration into the Clostridia chromosome. We found 16 Clostridia whose chromosomes were targetedly inserted by the 16s-tron (FIGS. 2C and 13, FIG. 26, and Example 1 for detailed information), suggesting that this strategy is efficient in developing gene insertion tools for many Clostridia.

We tested whether a similar strategy can be applied to non-model Gram-negative gut commensals. We prioritized Prevotella microbes because there are limited genetic tools available for this genus (Li et al., 2021). Gram-negative bacteria, in general, have more efficient HR. We synthesized a chimeric 16s (Chi-16s) sequence with high homology to the Prevotella 16s rRNA genes (FIG. 2D). We introduced the Chi-16s with a suicide vector into 21 human-associated Prevotella isolates (Kraal et al., 2014) (FIG. 22). We found 7 Prevotella whose 16s loci were inserted by pGM-NAC₂P (FIG. 2D, FIGS. 24 and 27, and Example 1 for detailed information). The Chi-16s strategy was also applied to 45 Bacteroides Parabacteroides microbes (some with genetic tools (Bencivenga-Barry et al., 2020; Garcia-Bayona and Comstock, 2019; Salyers et al., 1999; Taketani et al., 2020)), and 35 gut-associated Gram-negative microbes from other phyla (Fung et al., 2016), leading us to identify the gene transfer methods for 41 of them (FIGS. 1C, FIG. 22, S2, S3, and S5 and Example 1 for detailed information). These data demonstrate that the HR-based Chi-16s strategy efficiently identifies their gene transfer methods and generates gene insertion tools in non-model Prevotella and Bacteroides, and gut-associated microbes from other phyla.

### Example 7: Constructing Mutants to Modulate Clostridia Gene Transcription and Microbiome Metabolites Production

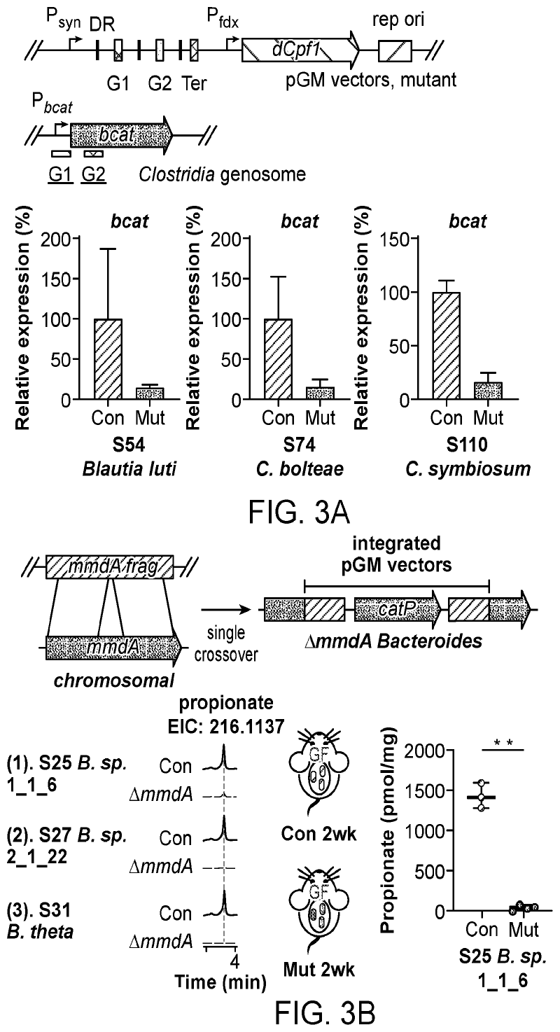

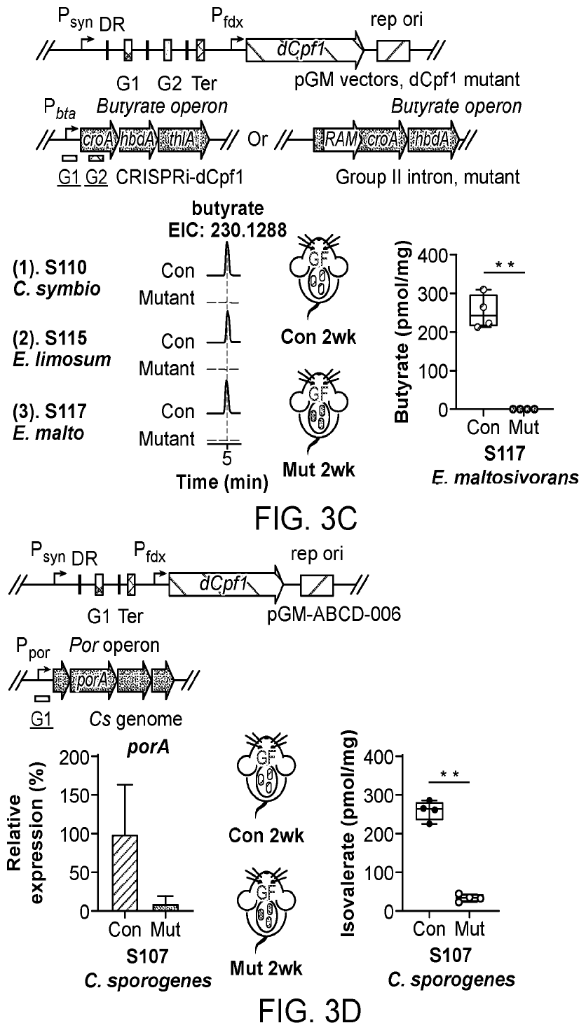

To demonstrate the utility of genetic tools developed in this study, we selected a widely distributed gene bcat and modulated its expression in 12 Clostridia. (FIG. 3A (top)). The BCAT protein deaminates branched-chain amino acids into their keto acid form (Hur et al., 2016). (FIGS. 3A (bottom), 14, and FIGS. 26 and 28). A duplex bcat-targeting gRNA along with dCpf1 was introduced into 12 Clostridia, and bcat transcription was repressed in all the mutants with the dCpf1+gRNAs, compared to control with only dCpf1 (FIG. 3A (bottom)).

We next sought to utilize these gene insertion tools to modulate the production of microbiome-derived metabolites in vitro and in the context of host colonization. We selected short-chain fatty acids (SCFAs) propionate and butyrate, as well as branched-SCFAs (BSCFAs), because of their vital role in maintaining host immune homeostasis and metabolic health (Blander et al., 2017; Cani et al., 2019; Rooks and Garrett, 2016). We first identified several gut commensals as abundant producers of the corresponding metabolites by analyzing the SCFA profiles of our targetable commensals. Next, we generated a series of mutants that reduce their production in vitro by targeting the corresponding metabolic genes. For propionate, we deleted the methylmalonate mutase (mmdA) genes of three Bacteroides microbes that convert methylmalonate to propionate (FIGS. 3B and 15A-15C, and FIGS. 27 and 28) (Fischbach and Sonnenburg, 2011) (Reichardt et al., 2014). For butyrate, we targeted the crotonase gene (croA) (Vital et al., 2014) and used either dCpf1 to downregulate its expression or group II intron to knock out the gene (FIGS. 3C and 15A-15C, and FIGS. 26 and 28). For BSCFAs, we applied the dCpf1 tool to suppress porA expression in C. sporogenes (Guo et al., 2019). For all the mutants we generated, we found that the in vitro production of the corresponding metabolites is significantly reduced compared to the control, and their levels in the mono-colonized mice are also much less than that of the control (FIGS. 3D and 15A-15C, and FIGS. 26, 27, and 28). Taken together, these data show that we can utilize the genetic tools developed via the GM pipeline to modulate microbiome-derived metabolites in vitro and in the context of host colonization, suggesting their potential in systematically linking microbiota genes with their responsible metabolites and associated host biology.

### Example 8: A Case Study of Clostridia-Specific Bile Acid 7α-Dehydroxylation

We sought to use these genetic tools to study the effect of one microbiota gene on host biology. We selected the bai operon for 7α-dehydroxylating of CA (cholate)/CDCA (chenodeoxycholate) to DCA (deoxycholate)/LCA (lithocholate) for follow-up studies. Three reasons motivated us to choose this pathway (FIGS. 4, 16A, and 17A): 1) Interestingly, we found one commensal S122 that efficiently converts CA(1)/CDCA to DCA(3)/LCA (FIGS. 4A and 4B). Previous works have stepwise elucidated the chemistry and enzymology of 7α-dehydroxylation (Funabashi et al., 2020; Ridlon et al., 2006, 2016). However, a key impediment to investigating bai operon biology is that all the identified bai-coding Clostridia (FIG. 16A) have no published gene transfer methods and tractable genetic tools (Ridlon et al., 2016). 2) DCA/LCA and their derivatives dominate the host secondary bile acid pool (Arab et al., 2017). 3) Amphipathic bile acids have intriguing biological activities: they inhibit the growth of enteric pathogens (Buffie et al., 2015), regulate mucosal immunity (Chen et al., 2019; Fiorucci et al., 2018), and promote liver cancer (Yoshimoto et al., 2013).

We sequenced S122 and identified its bai operon (FIG. 4A, see Example 1 for detailed information). Our bioinformatic analyses revealed three unique features of S122: 1) The strain is widely distributed among the healthy human population in two independent cohorts (41.30% (Lloyd-Price et al., 2019), 85.98% (Yatsunenko et al., 2012). 2) Like other 7α-dehydroxylating Clostridia, S122 has a low intestinal abundance (˜0.016%), but its bai operon is actively transcribed under conditions of host colonization (FIG. 4A). 3) S122 and its close relatives are more prevalent and abundant than C. hiranonis or C. scindens (FIGS. 16B-16C), indicating they play a significant role in regulating gut 7α-dehydroxylating activity.

To manipulate the bai pathway in vivo, we generated a baiH insertion mutant (S122 ΩbaiH) (FIGS. 4B and 17B, see Example 1 for detailed information). The baiH gene encodes an oxidoreductase that reduces 3-oxo-4,5-6,7-didehydro-DCA (2) to 3-oxo-4,5-didehydro-DCA (FIGS. 4B, 17A, and 18) (Funabashi et al., 2020; Kang et al., 2008). The ΩbaiH mutant depleted DCA and accumulated the intermediate (2) and 7-oxo CA in vitro (FIGS. 4B and 17A). Unexpectedly, our attempt to efficiently mono-associate GF mice with S122 to induce in vivo DCA production proved unsuccessful. Instead, we found S122 can stably co-colonize the GF mice with S25, and knocking out baiH eliminates gut 7α-dehydroxylating activity: The control accumulated ˜12 pmol/mg DCA (FIGS. 4C and 4D), while the mutant abolished DCA but accumulated 7-oxo-CA (FIGS. 4D and 17C). Moreover, S122 can stably co-colonize GF mice with 55 other genetically targetable microbes identified in this study (FIG. 17D). The relative abundance of S122 is low in both cases (FIGS. 4C and 17D), but robust CA to DCA conversion can be detected, suggesting that S122 is a highly active 7α-dehydroxylating bacterium in the host.

### Example 9: The baiH Gene has Significant Effects on the Host Bile Acid Pool and Microbiota Composition

This finding motivates us to knock out baiH in complex microbiota, like that of Specific Pathogen Free (SPF) mice. Manipulating microbiota genes in a complex microbiome provides a direct readout of their effects on microbial composition, which can be critical to explaining its impact on host biology. Unlike GF mice, the GI tract of SPF mice already harbors a complex microbiome with robust 7α-dehydroxylating activity, leaving a limited niche for S122 to occupy. To overcome this challenge, we genetically tagged the control and ΩbaiH mutant with a thiamphenicol-resistant marker. We supplemented their drinking water with thiamphenicol (15 g/ml) and erythromycin (10 μg/ml) at very low concentrations for two reasons: 1) to facilitate the colonization of the tagged strains that are resistant to these two antibiotics, and 2) to eliminate the background 7α-dehydroxylating activity conferred by the existing bai-coding Clostridia. This strategy led us to stably colonize the SPF mice with S122 control and the ΩbaiH mutant at about the same level with comparable total bacterial load (FIGS. 5A, and 5B) for at least 4 weeks. Because supplemented antibiotics minimally accumulate in the feces (˜5 pmol/mg for thiamphenicol and not detectable for erythromycin), they do not reduce the total bacterial load compared to the SPF mice (FIG. 17E). Additionally, their effect on the gut microbiome is also controlled under this experimental setting.

To examine whether baiH deletion affects gut bile acid composition and the microbiome, we performed metabolomics and 16s rRNA sequencing analyses on stool samples (FIGS. 5 and 19A-19I). Principal coordinate analysis (PCoA) demonstrated that stool samples are clustered by genotype (FIG. 5C). We drew two observations from these data: First, the control and ΩbaiH colonized mice have different intestinal bile acid pools. Both groups have similar levels of conjugated bile acids like TCA and TCDCA (FIG. 5D), indicating baiH depletion does not significantly modify microbiome bile salt hydrolyzing activity. DCA and its derivatives (such as isoDCA and 3-oxo DCA) are accumulated in the control group at levels comparable to host physiological levels. In contrast, the mutant group has higher levels of CA and its derivatives, including 7-oxo CA and UCA. (FIG. 5D)

Second, knocking out baiH modifies host gut microbiome composition. Both the control and mutant groups harbor a highly complex stool microbiota, and their overall phylum composition was maintained (FIG. 5E). The control group has a lower abundance of Bacteroidetes, higher Proteobacteria, and a significantly elevated Erysipelotrichaceae (FIGS. 5F, 19C, and 19D). This compositional shift has been associated with worsened intestinal inflammation (Kaakoush, 2015; Kaser et al., 2010; Palm et al., 2014). A total of 56 operational taxonomic units (OTUs) were differentially abundant between groups, and they belong predominantly to the Bacteroidia, Betaproteobacteria, and Erysipelotria (FIG. 5G). Of note, the control has significantly more Erysipelotrichaceae that have high IgA coating and are associated with exacerbated colon inflammation (FIGS. 5F and 5G) (Kaakoush, 2015; Palm et al., 2014). Aligned with our findings in the SPF mice, a higher stool DCA is positively associated with Erysipelotrichaceae abundance (FIG. 19H) and fecal calprotectin (a marker for the level of intestinal inflammation) (FIG. 5H) in nonIBD human stools. However, this correlation is not observed in patients with ulcerative colitis or Crohn's disease whose gut microbiota are usually structurally altered and whose gut 7α-dehydroxylation activity is disrupted because of an exaggerated immune response (Lloyd-Price et al., 2019). These data indicate a potential modulatory role of bai operon in human gut microbiota and the onset of intestinal inflammation.

### Example 10: Assessing the Effect of baiH on Intestinal Inflammation

Because knocking out baiH shifts the gut microbiome to a less proinflammatory state (Kaser et al., 2010), we assessed whether baiH regulates intestinal inflammatory responses in a dextran sodium sulfate (DSS)-induced murine colitis model. The control and ΩbaiH mutant colonized SPF mice were given drinking water with DSS (FIGS. 6A and 6F). We found that both the S122 control and ΩbaiH mutant strains stably colonized the mice (FIGS. 13F and 13I), and the control group still has significantly higher Erysipelotrichaceae during DSS treatment (FIG. 13G). As colonic inflammation progressed, baiH indeed played a modulatory role in intestinal inflammation: The control lost more weight and experienced more severe inflammation as shown by enhanced colonic pathology, shorter colon lengths, increased fecal lipocalin-2 levels, higher hematochezia score, and upregulation of inflammatory genes (FIGS. 6B-6E, 20A, 20C, and 20E). Interestingly, the same DSS treatment successfully triggered an inflammation response in the GF C57BL/6 mice co-colonized with the control or ΩbaiH mutant and S25, but knocking out baiH has no notable effect on intestinal inflammation (FIGS. 6G-6J, 20B, and 20D). Taken together, these data indicate that baiH-mediated inflammatory responses are microbiota dependent, and baiH depletion in a complex microbiota reshapes host bile acid profiles and presets gut microbiome composition to a more protective state against DSS-induced colitis. More importantly, using a combination of microbiome genetics, metabolomics, and colitis mouse models, we demonstrate how a single commensal gene of a low intestinal abundance may significantly impact host biology by reshaping bile acid metabolism and the gut microbiota ecosystem.

### Example 11: The baiH-Mediated Microbiota Composition Shift Exacerbates DSS-Induced Colitis in Gnotobiotic Mice

Motivated by our findings that baiH mediates colon inflammation in a complex microbiome, we proceeded to examine if microbiota composition shift induced by baiH deletion (FIGS. 5F and 20G) could be related to the different intestinal inflammatory responses in the SPF mice under DSS treatment. First, we determined whether Erysipelotrichaceae expansion is baiH-dependent. Indeed, Erysipelotrichaceae isolates are more resistant to high concentrations of DCA and 3-oxo DCA compared to Bacteroides (FIGS. 7A and 21). In a 10-member synthetic consortium we prepared in vitro, Erysipelotrichaceae also expands in the presence of baiH and its product DCA (FIGS. 7B and 7C).

Next, we asked whether baiH drives Erysipelotrichaceae expansion in vivo, and whether this microbiota composition shift affects colon inflammatory responses in the DSS colitis model. We colonized two groups of germ-free C57BL6/N mice with the same 10-member synthetic consortium (S122 control or ΩbaiH mutant with 7 Erysipelotrichaceae and 2 Bacteroides, FIGS. 7B and 7D) and applied the DSS treatment two weeks post colonization (FIG. 7D). As expected, baiH also drives the expansion of Erysipelotrichaeceae in the context of host colonization (FIG. 7E). The control group has exacerbated colon inflammation in this gnotobiotic setting as evaluated by severe weight loss (FIG. 7F), enhanced colonic pathology (FIG. 7G), shorter colon lengths (FIG. 711), increased fecal lipocalin-2 levels (FIG. 7I), and higher hematochezia score (FIG. 7J). The same DSS treatment also induced a robust inflammation response in the GF C57BL/6 mice co-colonized with the S122 control or ΩbaiH mutant with only the two Bacteroides (three-member, FIG. 22G), however depleting baiH in this gnotobiotic setting has no notable effect on intestinal inflammation (FIGS. 22H to 22L). The S122 control and ΩbaiH mutant colonized the mice at comparable levels under both gnotobiotic settings (10-member vs. 3 member) (FIGS. 22A and 22B), and their fecal bile acid profiles are comparable (FIGS. 22C to 22F), suggesting that the different intestinal inflammatory response observed in the 10-member consortium colonized mice is more likely due to the expansion of Erysipelotrichaeceae driven by baiH. Altogether, these data indicate that a baiH-mediated microbiota composition shift could exacerbate DSS-Induced colitis in the gnotobiotic mice, and the similar shift observed in the SPF mice (FIGS. 5F and 20G) could be potentially related to the different intestinal inflammatory responses induced by baiH depletion in a complex microbiota. Of note, members of the synthetic consortium were selected based on the information we obtained by depleting baiH in a highly diverse microbiome, demonstrating the usefulness and necessity of studying the function of a microbiota gene in the background of a complex microbiota.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Arab, J. P., Karpen, S. J., Dawson, P. A., Arrese, M., and Trauner, M. (2017). Bile acids and nonalcoholic fatty liver disease: Molecular insights and therapeutic perspectives. Hepatology 65, 350-362.
2. Bencivenga-Barry, N. A., Lim, B., Herrera, C. M., Stephen Trent, M., and Goodman, A. L. (2020). Genetic manipulation of wild human gut bacteroides. Journal of Bacteriology 202.
3. Blander, J. M., Longman, R. S., Iliev, I. D., Sonnenberg, G. F., and Artis, D. (2017). Regulation of inflammation by microbiota interactions with the host. Nature Immunology 18, 851-860.
4. Buffie, C. G., Bucci, V., Stein, R. R., McKenney, P. T., Ling, L., Gobourne, A., No, D., Liu, H., Kinnebrew, M., Viale, A., et al. (2015). Precision microbiome reconstitution restores bile acid mediated resistance to Clostridium difficile. Nature 517, 205-208.
5. Campbell, C., McKenney, P. T., Konstantinovsky, D., Isaeva, O. I., Schizas, M., Verter, J., Mai, C., Jin, W. B., Guo, C. J., Violante, S., et al. (2020). Bacterial metabolism of bile acids promotes generation of peripheral regulatory T cells. Nature 581, 475-479.
6. Canadas, I. C., Groothuis, D., Zygouropoulou, M., Rodrigues, R., and Minton, N. P. (2019). RiboCas: A Universal CRISPR-Based Editing Tool for Clostridium. ACS Synthetic Biology 8, 1379-1390.
7. Cani, P. D., Van Hul, M., Lefort, C., Depommier, C., Rastelli, M., and Everard, A. (2019). Microbial regulation of organismal energy homeostasis. Nature Metabolism 1, 34-46.
8. Chen, M. L., Takeda, K., and Sundrud, M. S. (2019). Emerging roles of bile acids in mucosal immunity and inflammation. Mucosal Immunology 12, 851-861.
9. Cho, I., and Blaser, M. J. (2012). The human microbiome: At the interface of health and disease. Nature Reviews Genetics 13, 260-270.
10. Fiorucci, S., Biagioli, M., Zampella, A., and Distrutti, E. (2018). Bile acids activated receptors regulate innate immunity. Frontiers in Immunology 9, 1.
11. Fischbach, M. A., and Sonnenburg, J. L. (2011). Eating for two: How metabolism establishes interspecies interactions in the gut. Cell Host and Microbe 10, 336-347.
12. Funabashi, M., Grove, T. L., Wang, M., Varma, Y., McFadden, M. E., Brown, L. C., Guo, C., Higginbottom, S., Almo, S. C., and Fischbach, M. A. (2020). A metabolic pathway for bile acid dehydroxylation by the gut microbiome. Nature 582, 566-570.
13. Fung, T. C., Bessman, N.J., Hepworth, M. R., Kumar, N., Shibata, N., Kobuley, D., Wang, K., Ziegler, C. G. K., Goc, J., Shima, T., et al. (2016). Lymphoid-Tissue-Resident Commensal Bacteria Promote Members of the IL-10 Cytokine Family to Establish Mutualism.

### Immunity 44, 634-646.

14. Garcia-Bayona, L., and Comstock, L. E. (2019). Streamlined genetic manipulation of diverse bacteroides and parabacteroides isolates from the human gut microbiota. MBio 10.
15. Guo, C.-J., Allen, B. M., Hiam, K. J., Dodd, D., Van Treuren, W., Higginbottom, S., Nagashima, K., Fischer, C. R., Sonnenburg, J. L., Spitzer, M. H., et al. (2019). Depletion of microbiome-derived molecules in the host using Clostridium genetics. Science 366, eaav1282.
16. Hang, S., Paik, D., Yao, L., Kim, E., Jamma, T., Lu, J., Ha, S., Nelson, B. N., Kelly, S. P., Wu, L., et al. (2019). Bile acid metabolites control TH17 and Treg cell differentiation. Nature 576, 143-148.
17. Heap, J. T., Pennington, O. J., Cartman, ST., Carter, G. P., and Minton, N. P. (2007).

The ClosTron: A universal gene knockout system for the genus Clostridium. Journal of Microbiological Methods 70, 452-464.
18. Helmink, B. A., Khan, M. A. W., Hermann, A., Gopalakrishnan, V., and Wargo, J. A. (2019). The microbiome, cancer, and cancer therapy. Nature Medicine 25, 377-388.
19. Hong, W., Zhang, J., Cui, G., Wang, L., and Wang, Y. (2018). Multiplexed CRISPR-Cpf1-Mediated Genome Editing in Clostridium difficile toward the Understanding of Pathogenesis of C. difficile Infection. ACS Synthetic Biology 7, 1588-1600.
20. Hur, J. K., Kim, K., Been, K. W., Baek, G., Ye, S., Hur, J. W., Ryu, S. M., Lee, Y. S., and Kim, J. S. (2016). Targeted mutagenesis in mice by electroporation of Cpf1 ribonucleoproteins. Nature Biotechnology 34, 807-808.
21. Isenbarger, T. A., Carr, C. E., Johnson, S. S., Finney, M., Church, G. M., Gilbert, W., Zuber, M. T., and Ruvkun, G. (2008). The most conserved genome segments for life detection on earth and other planets. Origins of Life and Evolution of Biospheres 38, 517-533.
22. Jennert, K. C. B., Tardif, C., Young, D. I., and Young, M. (2000). Gene transfer to Clostridium cellulolyticum ATCC 35319. Microbiology 146, 3071-3080.
23. Johnston, C. D., Cotton, S. L., Rittling, S. R., Starr, J. R., Borisy, G. G., Dewhirst, F. E., and Lemon, K. P. (2019). Systematic evasion of the restriction-modification barrier in bacteria. Proceedings of the National Academy of Sciences 116, 11454-11459.
24. Kaakoush, N. O. (2015). Insights into the role of Erysipelotrichaceae in the human host. Frontiers in Cellular and Infection Microbiology 5, 1-4.
25. Kang, D.-J., Ridlon, J. M., Moore, D. R. 2nd, Barnes, S., and Hylemon, P. B. (2008). Clostridium scindens baiCD and baiH genes encode stereo-specific 7alpha/7beta-hydroxy-3-oxo-delta4-cholenoic acid oxidoreductases. Biochimica et Biophysica Acta 1781, 16-25.
26. Kaser, A., Zeissig, S., and Blumberg, R. S. (2010). Inflammatory bowel disease. Annual Review of Immunology 28, 573-621.
27. Kim, S. K., Kim, H., Ahn, W. C., Park, K. H., Woo, E. J., Lee, D. H., and Lee, S. G. (2017). Efficient Transcriptional Gene Repression by Type V-A CRISPR-Cpf1 from Eubacterium eligens. ACS Synthetic Biology 6, 1273-1282.
28. Klompe, S. E., Vo, P. L. H., Halpin-Healy, T. S., and Sternberg, S. H. (2019). Transposon-encoded CRISPR-Cas systems direct RNA-guided DNA integration. Nature 571, 219-225.
29. Kraal, L., Abubucker, S., Kota, K., Fischbach, M. A., and Mitreva, M. (2014). The prevalence of species and strains in the human microbiome: A resource for experimental efforts. PLoS ONE 9.
30. Li, J., Gilvez, E. J. C., Amend, L., Almasi, E., Iljazovic, A., Lesker, T. R., Bielecka, A. A., and Strowig, T. (2021). A versatile genetic toolbox for &lt;em&gt;Prevotella copri&lt;/em&gt; enables studying polysaccharide utilization systems. BioRxiv 2021.03.19.436125.
31. Lim, B., Zimmermann, M., Barry, N. A., and Goodman, A. L. (2017). Engineered Regulatory Systems Modulate Gene Expression of Human Commensals in the Gut. Cell 169, 547-558.e15.
32. Lloyd-Price, J., Arze, C., Ananthakrishnan, A. N., Schirmer, M., Avila-Pacheco, J., Poon, T. W., Andrews, E., Ajami, N.J., Bonham, K. S., Brislawn, C. J., et al. (2019). Multi-omics of the gut microbial ecosystem in inflammatory bowel diseases. Nature 569, 655-662.
33. McAllister, K. N., Bouillaut, L., Kahn, J. N., Self, W. T., and Sorg, J. A. (2017). Using CRISPR-Cas9-mediated genome editing to generate C. difficile mutants defective in selenoproteins synthesis. Scientific Reports 7, 1-12.
34. Mermelstein, L. D., Welker, N. E., Bennett, G. N., and Papoutsakis, E. T. (1992). Expression of cloned homologous fermentative genes in Clostridium acetobutylicum ATCC 824. Bio/Technology 10, 190.
35. Mimee, M., Tucker, A. C., Voigt, C. A., and Lu, T. K. (2015). Programming a Human Commensal Bacterium, Bacteroides thetaiotaomicron, to Sense and Respond to Stimuli in the Murine Gut Microbiota. Cell Systems 1, 62-71.
36. Palm, N. W., De Zoete, M. R., Cullen, T. W., Barry, N. A., Stefanowski, J., Hao, L., Degnan, P. H., Hu, J., Peter, I., Zhang, W., et al. (2014). Immunoglobulin A coating identifies colitogenic bacteria in inflammatory bowel disease. Cell 158, 1000-1010.
37. Purdy, D., O'Keeffe, T. A. T., Elmore, M., Herbert, M., McLeod, A., Bokori-Brown, M., Ostrowski, A., and Minton, N. P. (2002). Conjugative transfer of clostridial shuttle vectors from Escherichia coli to Clostridium difficile through circumvention of the restriction barrier. Molecular Microbiology 46, 439-452.
38. Pyne, M. E., Bruder, M., Moo-Young, M., Chung, D. A., and Chou, C. P. (2014). Technical guide for genetic advancement of underdeveloped and intractable Clostridium. Biotechnology Advances 32, 623-641.
39. Reichardt, N., Duncan, S. H., Young, P., Belenguer, A., McWilliam Leitch, C., Scott, K. P., Flint, H. J., and Louis, P. (2014). Phylogenetic distribution of three pathways for propionate production within the human gut microbiota. ISME Journal 8, 1323-1335.
40. Ridlon, J. M., Kang, D. J., and Hylemon, P. B. (2006). Bile salt biotransformations by human intestinal bacteria. Journal of Lipid Research 47, 241-259.
41. Ridlon, J. M., Harris, S. C., Bhowmik, S., Kang, D. J., and Hylemon, P. B. (2016). Consequences of bile salt biotransformations by intestinal bacteria. Gut Microbes 7, 22-39.
42. Rooks, M. G., and Garrett, W. S. (2016). Gut microbiota, metabolites and host immunity. Nature Reviews Immunology 16, 341-352.
43. Roy, S., and Trinchieri, G. (2017). Microbiota: A key orchestrator of cancer therapy. Nature Reviews Cancer 17, 271-285.
44. Salyers, A. A., Shoemaker, N., Cooper, A., D'Elia, J., and Shipman, J. A. (1999). 8 Genetic Methods for Bacteroides Species. Methods in Microbiology 29, 229-249.
45. Sinha, S. R., Haileselassie, Y., Nguyen, L. P., Tropini, C., Wang, M., Becker, L. S., Sim, D., Jarr, K., Spear, E. T., Singh, G., et al. (2020). Dysbiosis-Induced Secondary Bile Acid Deficiency Promotes Intestinal Inflammation. Cell Host and Microbe 27, 659-670.e5.
46. Song, X., Sun, X., Oh, S. F., Wu, M., Zhang, Y., Zheng, W., Geva-Zatorsky, N., Jupp, R., Mathis, D., Benoist, C., et al. (2020). Microbial bile acid metabolites modulate gut RORγ+ regulatory T cell homeostasis. Nature 577, 410-415.
47. Strecker, J., Ladha, A., Gardner, Z., Schmid-Burgk, J. L., Makarova, K. S., Koonin, E. v., and Zhang, F. (2019). RNA-guided DNA insertion with CRISPR-associated transposases. Science 364, 48-53.
48. Taketani, M., Zhang, J., Zhang, S., Triassi, A. J., Huang, Y. J., Griffith, L. G., and Voigt, C. A. (2020). Genetic circuit design automation for the gut resident species Bacteroides thetaiotaomicron. Nature Biotechnology 1-8.
49. Tang, X., Lowder, L. G., Zhang, T., Malzahn, A. A., Zheng, X., Voytas, D. F., Zhong, Z., Chen, Y., Ren, Q., Li, Q., et al. (2017). A CRISPR-Cpf1 system for efficient genome editing and transcriptional repression in plants. Nature Plants 2017 3:3 3, 1-5.
50. Thomas, A. M., Manghi, P., Asnicar, F., Pasolli, E., Armanini, F., Zolfo, M., Beghini, F., Manara, S., Karcher, N., Pozzi, C., et al. (2019). Metagenomic analysis of colorectal cancer datasets identifies cross-cohort microbial diagnostic signatures and a link with choline degradation. Nature Medicine 25, 667-678.
51. Turnbaugh, P. J., Ley, R. E., Hamady, M., Fraser-Liggett, C. M., Knight, R., and Gordon, J. I. (2007). The Human Microbiome Project. Nature 449, 804-810.
52. Vital, M., Howe, A., and Tiedje, J. (2014). Revealing the Bacterial Synthesis Pathways by Analyzing (Meta) Genomic Data. MBio 5, 1-11.
53. Vo, P. L. H., Ronda, C., Klompe, S. E., Chen, E. E., Acree, C., Wang, H. H., and Sternberg, S. H. (2021). CRISPR RNA-guided integrases for high-efficiency, multiplexed bacterial genome engineering. Nature Biotechnology 39, 480-489.
54. Waller, M. C., Bober, J. R., Nair, N. U., and Beisel, C. L. (2017a). Toward a genetic tool development pipeline for host-associated bacteria. Current Opinion in Microbiology 38, 156-164.
55. Waller, M. C., Bober, J. R., Nair, N. U., and Beisel, C. L. (2017b). Toward a genetic tool development pipeline for host-associated bacteria. Current Opinion in Microbiology 38, 156-164.
56. Wang, J., Qin, J., Li, Y., Cai, Z., Li, S., Zhu, J., Zhang, F., Liang, S., Zhang, W., Guan, Y., et al. (2012). A metagenome-wide association study of gut microbiota in type 2 diabetes. Nature 490, 55-60.
57. Whitaker, W. R., Shepherd, E. S., and Sonnenburg, J. L. (2017). Tunable Expression Tools Enable Single-Cell Strain Distinction in the Gut Microbiome. Cell 169, 538-546.e12.
58. Wirbel, J., Pyl, P. T., Kartal, E., Zych, K., Kashani, A., Milanese, A., Fleck, J. S., Voigt, A. Y., Palleja, A., Ponnudurai, R., et al. (2019). Meta-analysis of fecal metagenomes reveals global microbial signatures that are specific for colorectal cancer. Nature Medicine 25, 679-689.
59. Woods, C., Humphreys, C. M., Rodrigues, R. M., Ingle, P., Rowe, P., Henstra, A. M., Kopke, M., Simpson, S. D., Winzer, K., and Minton, N. P. (2019). A novel conjugal donor strain for improved DNA transfer into Clostridium spp. Anaerobe 59, 184-191.
60. Yachida, S., Mizutani, S., Shiroma, H., Shiba, S., Nakajima, T., Sakamoto, T., Watanabe, H., Masuda, K., Nishimoto, Y., Kubo, M., et al. (2019). Metagenomic and metabolomic analyses reveal distinct stage-specific phenotypes of the gut microbiota in colorectal cancer. Nature Medicine 25, 968-976.
61. Yang, X., Xu, M., and Yang, S. T. (2016). Restriction modification system analysis and development of in vivo methylation for the transformation of Clostridium cellulovorans. Applied Microbiology and Biotechnology 100, 2289-2299.
62. Yatsunenko, T., Rey, F. E., Manary, M. J., Trehan, I., Dominguez-Bello, M. G., Contreras, M., Magris, M., Hidalgo, G., Baldassano, R. N., Anokhin, A. P., et al. (2012). Human gut microbiome viewed across age and geography. Nature 486, 222-227.
63. Yoshimoto, S., Loo, T. M., Atarashi, K., Kanda, H., Sato, S., Oyadomari, S., Iwakura, Y., Oshima, K., Morita, H., Hattori, M., et al. (2013). Obesity-induced gut microbial metabolite promotes liver cancer through senescence secretome. Nature 499, 97-101.
64. Zetsche, B., Gootenberg, J. S., Abudayyeh, O. O., Slaymaker, I. M., Makarova, K. S., Essletzbichler, P., Volz, S. E., Joung, J., Van Der Oost, J., Regev, A., et al. (2015). Cpf1 Is a Single RNA-Guided Endonuclease of a Class 2 CRISPR-Cas System. Cell 163, 759-771.
65. Zhang, J., Zong, W., Hong, W., Zhang, Z. T., and Wang, Y. (2018). Exploiting endogenous CRISPR-Cas system for multiplex genome editing in Clostridium tyrobutyricum and engineer the strain for high-level butanol production. Metabolic Engineering 47, 49-59.
66. Zhang, X., Wang, J., Cheng, Q., Zheng, X., Zhao, G., and Wang, J. (2017). Multiplex gene regulation by CRISPR-ddCpf1. Cell Discovery 2017 3:1 3, 1-9.
67. Zhao, S., Gong, Z., Du, X., Tian, C., Wang, L., Zhou, J., Xu, C., Chen, Y., Cai, W., and Wu, J. (2018). Deoxycholic acid-mediated sphingosine-1-phosphate receptor 2 signaling exacerbates DSS-induced colitis through promoting cathepsin b release. Journal of Immunology Research 2018.
68. Zhong, J., Karberg, M., and Lambowitz, A. M. (2003). Targeted and random bacterial gene disruption using a group II intron (targetron) vector containing a retrotransposition-activated selectable marker. Nucleic Acids Research 31, 1656-1664.
69. Zhou, W., Sailani, M. R., Contrepois, K., Zhou, Y., Ahadi, S., Leopold, S. R., Zhang, M. J., Rao, V., Avina, M., Mishra, T., et al. (2019). Longitudinal multi-omics of host-microbe dynamics in prediabetes. Nature 569, 663-671.
70. Canadas, I. C., Groothuis, D., Zygouropoulou, M., Rodrigues, R., and Minton, N. P. (2019). RiboCas: A Universal CRISPR-Based Editing Tool for Clostridium. ACS Synthetic Biology 8, 1379-1390.
71. David, L. A., Maurice, C. F., Carmody, R. N., Gootenberg, D. B., Button, J. E., Wolfe, B. E., Ling, A. V, Devlin, A. S., Varma, Y., Fischbach, M. A., et al. (2014). Diet rapidly and reproducibly alters the human gut microbiome. Nature 505, 559-563.
72. Funabashi, M., Grove, T. L., Wang, M., Varma, Y., McFadden, M. E., Brown, L. C., Guo, C., Higginbottom, S., Almo, S. C., and Fischbach, M. A. (2020). A metabolic pathway for bile acid dehydroxylation by the gut microbiome. Nature 582, 566-570.
73. Guo, C.-J., Allen, B. M., Hiam, K. J., Dodd, D., Van Treuren, W., Higginbottom, S., Nagashima, K., Fischer, C. R., Sonnenburg, J. L., Spitzer, M. H., et al. (2019). Depletion of microbiome-derived molecules in the host using Clostridium genetics. Science 366, eaav1282.
74. Heap, J. T., Pennington, O. J., Cartman, S. T., Carter, G. P., and Minton, N. P. (2007).

The ClosTron: A universal gene knock-out system for the genus Clostridium. Journal of Microbiological Methods 70, 452-464.
75. Heap, J. T., Pennington, O. J., Cartman, S T., and Minton, N. P. (2009). A modular system for Clostridium shuttle plasmids. Journal of Microbiological Methods 78, 79-85.
76. Hur, J. K., Kim, K., Been, K. W., Baek, G., Ye, S., Hur, J. W., Ryu, S. M., Lee, Y. S., and Kim, J. S. (2016). Targeted mutagenesis in mice by electroporation of Cpf1 ribonucleoproteins. Nature Biotechnology 34, 807-808.
77. Lu, Y., Yao, D., and Chen, C. (2013). 2-Hydrazinoquinoline as a Derivatization Agent for LC-MS-Based Metabolomic Investigation of Diabetic Ketoacidosis. Metabolites 3, 993-1010.
78. Martens, E. C., Chiang, H. C., and Gordon, J. I. (2008). Mucosal Glycan Foraging Enhances Fitness and Transmission of a Saccharolytic Human Gut Bacterial Symbiont. Cell Host and Microbe 4, 447-457.
79. Seemann, T. (2014). Prokka: Rapid prokaryotic genome annotation. Bioinformatics 30, 2068-2069.
80. Tang, X., Lowder, L. G., Zhang, T., Malzahn, A. A., Zheng, X., Voytas, D. F., Zhong, Z., Chen, Y., Ren, Q., Li, Q., et al. (2017). A CRISPR-Cpf1 system for efficient genome editing and transcriptional repression in plants. Nature Plants 3, 17018.
81. Woods, C., Humphreys, C. M., Rodrigues, R. M., Ingle, P., Rowe, P., Henstra, A. M., Kopke, M., Simpson, S. D., Winzer, K., and Minton, N. P. (2019). A novel conjugal donor strain for improved DNA transfer into Clostridium spp. Anaerobe 59, 184-191.
82. Yatsunenko, T., Rey, F. E., Manary, M. J., Trehan, I., Dominguez-Bello, M. G., Contreras, M., Magris, M., Hidalgo, G., Baldassano, R. N., Anokhin, A. P., et al. (2012). Human gut microbiome viewed across age and geography. Nature 486, 222-227.

1. A bacterial expression vector comprising (a) a nucleic acid encoding a target gene that is conserved in a plurality of human gut commensal gram-negative bacterial species and (b) a heterologous nucleic acid encoding a selectable marker, wherein the selectable marker is an antibiotic resistance gene or an auxotrophic marker, and optionally wherein the target gene is selected from the group consisting of 16s rRNA, 23s rRNA, mmdA, RokA (Clucokinase gene), and an ABC transporter gene.

2. The bacterial expression vector of claim 1, wherein the 16s rRNA comprises the nucleic acid sequence of SEQ ID NO: 11.

3. The bacterial expression vector of claim 1, wherein the bacterial expression vector comprises the nucleic acid sequence of SEQ ID NO: 310.

4. The bacterial expression vector of claim 1, further comprising at least one open reading frame encoding a bioluminescent protein, a chemiluminescent protein, a fluorescent protein, a CRISPR enzyme, a Group II intron-encoded protein, at least one sgRNA, at least one Group II intron, or any combination thereof.

5. A bacterial expression vector comprising (a) a gram-positive bacteria replication origin comprising a sequence selected from the group consisting of SEQ ID NOs: 1-9 or 311-319, (b) a heterologous nucleic acid encoding a selectable marker that is an antibiotic resistance gene or an auxotrophic marker, and (c) at least one open reading frame, wherein the at least one open reading frame encodes a bioluminescent protein, a chemiluminescent protein, a fluorescent protein, a CRISPR enzyme, a Group II intron-encoded protein, at least one sgRNA, at least one Group II intron, or any combination thereof.

6. The bacterial expression vector of claim 5, wherein the at least one sgRNA or the at least one Group II intron targets one or more genes selected from among 16S rRNA, porA, bcat, croA, baiA2, baiCD, baiF, baiH, baiB, baiE, baiG and bail.

7. The bacterial expression vector of claim 5, further comprising one or more bacterial conjugation transfer genes and/or an E. coli replication origin, optionally wherein the one or more bacterial conjugation transfer genes are selected from the group consisting of traJ, and oriT and/or the E. coli replication origin is selected from the group consisting of colE1, pBR, and R6K.

8. The bacterial expression vector of claim 5, wherein the one or more bacterial conjugation transfer genes, the gram-positive bacteria replication origin, and the heterologous nucleic acid encoding the selectable marker are codon optimized.

9. The bacterial expression vector of claim 1, wherein the antibiotic resistance gene is selected from the group consisting of catP, ermB, aad9, tetA, and ampR, or wherein the auxotrophic marker is pyrG, or pyrF.

10. The bacterial expression vector of claim 4, wherein the CRISPR enzyme is selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

11. The bacterial expression vector of claim 4, wherein the fluorescent protein is GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOκ, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa.

12. The bacterial expression vector of claim 4, wherein the chemiluminescent protein is β-galactosidase, horseradish peroxidase (HRP), or alkaline phosphatase; or
wherein the bioluminescent protein is Aequorin, firefly luciferase, Renilla luciferase, red luciferase, luxAB, or nanoluciferase; or
wherein the at least one sgRNA specifically hybridizes with a heterologous or endogenous target gene expressed in a gut bacterial host cell, and/or wherein the at least one sgRNA and/or the CRISPR enzyme is operably linked to a constitutive promoter or a conditional promoter; or
wherein the at least one Group II intron specifically targets a heterologous or endogenous gene expressed in a gut bacterial host cell, and/or wherein the at least one Group II intron and/or the Group II intron-encoded protein is operably linked to a constitutive promoter or a conditional promoter.

13. (canceled)

14. (canceled)

15. (canceled)

16. An engineered gram-negative human gut bacterial cell comprising the bacterial expression vector of claim 1, wherein the engineered gram-negative human gut bacterial cell is derived from a family selected from the group consisting of Enterobacteriaceae, Bacteroidaceae, Tannerellaceae, and Prevotellaceae, optionally wherein the engineered gram-negative human gut bacterial cell is derived from Bacteroides cellulosilyticus, Bacteroides cellulosilyticus, Bacteroides dorei, Bacteroides eggerthii, Bacteroides finegoldii, Bacteroides frygilis, Bacteroides intestinalis, Bacteroides nordii, Bacteroides oleiciplenus, Bacteroides ovatus, Bacteroides salyersiae, Bacteroides sp. Bacteroides thetaiotaomicron, Bacteroides uniformis, Bacteroides vulgatus, Bacteroides xylanisolvens, Parabacteroides faecis, Parabacteroides merdae, or Prevotella bivia.

17. (canceled)

18. An engineered gram-positive human gut bacterial cell comprising the bacterial expression vector of claim 5, wherein the engineered gram-positive human gut bacterial cell is derived from a family selected from the group consisting of Clostridiaceae, Lachnospiraceae, Eubacteriaceae, Erysipelotrichaceae, Enterococcaceae, and Bifidobacteriaceae, optionally wherein the engineered gram-positive human gut bacterial cell is derived from Blautia hydrogenotrophica, Blautia luti, Blautia sp., Blautia wexlerae, Clostridium bolteae, Clostridium innocuum, Clostridium paraputrificum, Clostridium saccharolyticum, Clostridium senegalense, Clostridium sp., Clostridium sporogenes, Clostridium symbiosum, Eubacterium limosum, Eubacterium maltosivorans, Eubacterium ramulus, Eubacterium sp. Roseburia inulinivorans, Bifidobacterium catenulatum, Enterococcus faecium, Escherichia fergusonii, Roseburia inulinivorans, or Bifidobacterium catenulatum.

19. (canceled)

20. A kit comprising the bacterial expression vector of claim 1 and instructions for using the bacterial expression vector to genetically modify human gut bacteria, optionally wherein the kit further comprises one or more primers and/or gRNAs comprising the sequence of any one of SEQ ID NOs: 23-287.

21. (canceled)

22. A method for modifying a gram-negative human gut bacteria cell genome comprising transferring at least one bacterial expression vector of claim 1 into a gram negative human gut bacteria cell via conjugation, optionally wherein the at least one bacterial expression vector is integrated into the genome of the gram-negative human gut bacteria cell.

23. (canceled)

24. A method for genetically modifying a gram-positive human gut bacteria cell comprising transferring two or more distinct bacterial expression vectors into a gram-positive human gut bacteria cell simultaneously via conjugation, wherein each of the two or more distinct bacterial expression vectors comprise:
(a) a gram-positive bacteria replication origin comprising a sequence selected from the group consisting of SEQ ID NOs: 1-9 or 311-319,
(b) a heterologous nucleic acid encoding a selectable marker that is an antibiotic resistance gene or an auxotrophic marker, and
(c) at least one open reading frame, wherein the at least one open reading frame encodes a bioluminescent protein, a chemiluminescent protein, a fluorescent protein, a CRISPR enzyme, a Group II intron-encoded protein, at least one sgRNA, at least one Group II intron, or any combination thereof.

25. The method of claim 24, wherein each of the two or more distinct bacterial expression vectors further comprise one or more bacterial conjugation transfer genes and/or an E. coli replication origin,
optionally wherein
the one or more bacterial conjugation transfer genes are selected from the group consisting of traJ, and oriT and/or the E. coli replication origin is selected from the group consisting of colE1, pBR, and R6K; or
the antibiotic resistance gene or the auxotrophic marker of each of the two or more distinct bacterial expression vectors is independently selected from the group consisting of catP, ermB, aad9, tetA, ampR, pyrG, and pyrF; or
the CRISPR enzyme of each of the two or more distinct bacterial expression vectors is independently selected from the group consisting of Cas9, dCas9, Cpf1, dCpf1, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4: or
the fluorescent protein of each of the two or more distinct bacterial expression vectors is independently selected from the group consisting of GFP, YFP, CFP, RFP, TagBFP, Azurite, EBFP2, mKalama1, Sirius, Sapphire, TSapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKO1, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PSCFP2, PS-CFP2, mEos2 (green), mEos2 (red), PSmOrange, and Dronpa; or
the chemiluminescent protein of each of the two or more distinct bacterial expression vectors is independently β-galactosidase, horseradish peroxidase (HRP), or alkaline phosphatase; or
the bioluminescent protein of each of the two or more distinct bacterial expression vectors is independently Aequorin, firefly luciferase, Renilla luciferase, red luciferase, luxAB, or nanoluciferase; or
the at least one sgRNA sequence of the two or more distinct bacterial expression vectors specifically hybridizes with a heterologous or endogenous target gene expressed in a gut bacterial host cell, and/or wherein the at least one sgRNA and/or the CRISPR enzyme is operably linked to a constitutive promoter or a conditional promoter; or
the at least one Group II intron of the two or more distinct bacterial expression vectors specifically targets a heterologous or endogenous gene expressed in a gut bacterial host cell, and/or wherein the at least one Group II intron and/or the Group II intron-encoded protein is operably linked to a constitutive promoter or a conditional promoter; or
three or four distinct bacterial expression vectors are simultaneously transferred into a gram-positive human gut bacteria cell simultaneously via conjugation.

26. (canceled)

27. (canceled)

28. (canceled)

29. (canceled)

30. (canceled)

31. (canceled)

32. (canceled)

33. (canceled)

34. The method of claim 24, wherein the gram-positive human gut bacteria cell is isolated from a colonic mucosa-enriched lavage sample, a fecal sample, a rectal swab, or an intestinal sample obtained from a human subject.

35. An engineered human gut bacterial cell generated by the method of claim 24.


SEQUENCE LISTING (319 sequences)

SEQ ID NO: 1 - DNA, 2407 bp, Clostridium botulinum
  source 1..2407: mol_type = genomic DNA
ggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggcc

SEQ ID NO: 2 - DNA, 1629 bp, Clostridium butyricum
  source 1..1629: mol_type = genomic DNA
ggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggcc

SEQ ID NO: 3 - DNA, 3450 bp, Clostridium difficile
  source 1..3450: mol_type = genomic DNA
ggcgcgcccgcccttaagtctaaaaattaggggagatgtaaggatttgggaaaaatagaagatgttataatcataaatatggtattcgtaggcttaaagtcaaaaaggaggtgaaatataaatagatttttagctaaattaagtaagaaataggaggagatttattgaacaaaaaattagaaaaaccatttgtatataagagagagtacgatttgactggatatgatgttgaaattttacaaaaatatgagttagaacaagcaatatatgtttatgttgggagtagttgtgcatataacatgagagctagaagtagtaaatggagataccatataagaacaaataataagtctatatgttgtaacattaaaaattttatacataacttggaattgttttataaaatggaattaaagttgtcagataatattattaatgataagctatactatagcaatatagcagagtttgaagaatttgaaacactagaaaaagctagagaggtagaaagtactattataagtcaatatcaatttttagattctataaatcacatgttaaaacaaaaaataattttattgagtaataaggatagtgtgttaaacataactaaaaatggaaatacaaattatttgaaagtaaaaaataaatacatagaaaaacataagaacaagccaataatgagataccatatcaactgtcaattcaatacagatggaagtgtcaaaagtattacacaggagtttgaaccaatattggaattaaacaaaaaaaataccctaagccgaccaagcagagtatttttaaaataatattttaagataacaacaaaatgagataatactactagacaatgacaactcaactaccaattgagtttatggagctaccaactccaatatcggtctaactgattaagtatctgtagttatataataatattgctatcaattttagcatcttaacaatattattatacatactaagctaaaattattcaatagttgtaaaagttgattagtcaataagtatatatttaatgtagtgttatctcttaaaaaaactagataaggagataataaatatatggaacaattagattcaaaatataagttgaaaaaatttctaatggcagtatttagagatggtataggacaaggaaataatcttattgataatgaatatgttagagtatttcaaaataataaaagtaatagtaaacaattagaactcggagaagaatttaaagaatatagtaaaacaactttttttaaaaatatagatgatatagtagaatttaccttcgcaaaaaatatttattatgaaaatacattttttaacctatgtactactgatggaaaagcaggaaccaatgaaaacttaataaatagatatgcattaggatttgattttgacaaaaaagaattaggacaaggttttaattataaagatataattaatttatttactaagataggattacattatcatatcctagttgatagtggaaatggattccatgtttatgtgctaattaataaaactaataacattaagttagtatcagaagttacaaatacattaataaataaattgggtgcagataaacaagcaaatttatctactcaagtattaagagtaccttatacatataatattaaaaatactactaaacaagtaaaaataatacaccaagacaaaaatatatatagatatgacatagaaaagttagctaaaaaatattgcaaagatgtaaaaacagtaggtaatactaatacaaaatatatattagatagtaagctaccaaattgtatagtagatattttaaaaaatggtagtaaagatggacataaaaacctagatttgcaaaaaatagttgtgactttaagattgaggaataaaagtttaagtcaagtaatatccgttgctagagaatggaactatatatcacaaaatagtctttcaaatagtgagctagaatatcaagtcaagtatatgtatgagaaacttaaaacggttaattttggttgtactggttgtgagtttaatagtgattgttggaataaaatagaatcagattttatatatagtgatgaagatactttgttcaatatgccacataagcactcaaaggatttgaaatataagaataggaaaggggttaaaataatgactggtaatcaattgtttatctataatgtgttacttaacaataaagatagagaattaaacatagacgatataatggagctgataacctataaacgtaagaagaaagttaaaaacattgttatgagtgaaaagacattaagagaaacattaaaagaacttcaacataatgattatattacaaaaacaaaaggtgttacaaagctaggaataaaagatacatacaatgtaaaagaagttagatgtaatatagataaacaatatactattagttactttgttaccatggcagtaatttggggaataatttcaactgaagaattaagattatatactcacatgagatataagcaagatttattggtcaaagatgataaaataaaaggaaatatattaagaattaatcaagaggaattagcaaaagatttaggagtaacacagcaaagaatttcaaatatgatagaatctttattagatactaaaattttagatgtatgggaaactaaaataaatgatagaggatttatgtactatacatatagattaaacaagtagatttttgataggattagaattgattttctagtcctatttttatgcaaaaaaactaattaataaaaatttcttttggtaaaataattgtacgagaattgcaaaaaaaaaatggcatcaaagtattgaaattaagccgttttaaaaatttcttttggtaaaataattctacatatatatgtagtatatatatatatgttttttagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatcctaattagtaggtgcttttttaaaacaagttaaaaatcaaaaatagtattagtaagcattggaaatgctagattctaaaatagaaaagtaaaaaattggtgcactatctaaacttatctatatcgctttttccgtcgtttggttctctagttacgatacaggggatatgcttatattgagttatagtactaatcagtgcttaatatagttaataaaattatagttaccatagtttagtaactatgatgtatgttagttagaaacttgcatttcggccggcc

SEQ ID NO: 4 - DNA, 882 bp, Bacillus subtilis
  source 1..882: mol_type = genomic DNA
ggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggcc

SEQ ID NO: 5 - DNA, 1746 bp, synthetic construct
  source 1..1746: mol_type = other DNA
ggcgcgcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctcctaaattcactttagataaaaatttaggaggcatatcaaatgaacggccggcc

SEQ ID NO: 6 - DNA, 3217 bp, Enterococcus faecalis
  source 1..3217: mol_type = genomic DNA
ggcgcgccctcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaaataagcaacgatttcgtgcattattcacacgaagtcattgcttttttcttcttccatttctaaatccaatgttacttgttctgattctgtttctggttctggttctgttggctcatttgggattaaatccactactagcgttgagttagttaactttgcaatttgttctagtgtttttatggttggatctgattttcctgggccggcc

SEQ ID NO: 7 - DNA, 1616 bp, Lactococcus lactis
  source 1..1616: mol_type = genomic DNA
ggcgcgccgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagccccctgacgaaagtcgaagggggtttttattttggtttgatgttgcgattaatagcaatacaaggccggcc

SEQ ID NO: 8 - DNA, 1616 bp, Bifidobacterium longum
  source 1..1616: mol_type = genomic DNA
ggcgcgccgccgcgggcctcagcctgcggaacgcgcagcggacgccgacggctcagacggctcagaaacgtccgtgagtggcctccacgcggccgaacaggtcagggaggctcgcgcatacgtgagcggcgtggagaagcggctgaaggccgtccagcggcttttcgtgcaggatgtgctgggctgggttcagccgacgcttcgctgggctgaaatatctgacttggttcccgcgtatttgttcactgtacaaatacgatgtatgctgtagccatgtccgatgagtattcgcagccgacgcttgagctgtcgcgcacgttcgaaggctggtggctgcccgaacgcccgctgtgctgcgacgacgactactcccggctgcaccgcaggagccgcgccgacgcgctcaaatgcaagcacatcgaggcgaaccccgccgcgctggtgaacacgatcgtggtggacatcgacgacgcgaacgccaaggcgatggccctgtgggagcacgagggcatgcggccgaactggatcgcggagaacccggccaacgggcacgctcacgcgggctgggtgctcacctttccggtgcccagaaccgatctggcgcgtctcaagccgttgaagctcctgcacgccaccacggagggactgcgccgctcctgcgacggggacatgggctattcgggacttctgatgaagaaccccgagcatccggcgtgggcgtcggacatcatcgagtgggacacctacgacctggaacagctcgtgcagtcgctccaggaacacggggacatgccgcccgtcagctggaagcgcaccaagcgcgcccgcacgcaggggctgggacgcaactgcacgctcttcgacaaggcccgcacgctcgcctaccgctacgttgcggcggctgccgaccgttcggaggccagcagcgaggcattgcgcctatacgtgcgtcgcacctgccacgaactcaacgtctcgctgttccccgatccgctgcacgcgcgtgaggtcgaggacatcgccaagagcatccacaaatggatcgtcacccgcagccgcatgtggcgcgacggtgccattgccaacgcagccacattcatcgccatccaatccgcacgaggacacaaacacggtgagaacaaatatcagcaggtcatgaaggaggcactggaatggtaaggacgactttgaggaagaagcgcccggtgtctgcacgtgaattagctgaagcatacggcgtctccacgcgcaccattcagagctgggtggcaatgaagcgcgaggattggattgatgaacaagccgctatgcgcgaagcagtccgctcatatcacgatgacgagggccatacatggccgcagaccgccgagcatttcaacatgagccagggtgccgtgcgtcaacgctgctacagggctcgcaaggagcgcgaggacgaggcggcggagaaatcgaagcatctacccggcgagattccactgttcgactgacgctaaacgttgtcccaaacgcgaacgcagcacctccctcgccttgcggctttttcctcttccatcggccttcggcactcgggttgttgctccagcgccgcagggcgcgggaggctgcggccggcc

SEQ ID NO: 9 - DNA, 2358 bp, Clostridium perfringens
  source 1..2358: mol_type = genomic DNA
ggcgcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgggccggcc

SEQ ID NO: 10 - DNA, 1438 bp, synthetic construct
  source 1..1438: mol_type = other DNA
caggaaacagctatgacctgagtggcggacgggtgagtaacgcgtgggtaacctgcctcatacagggggataacagttggaaacggatgctaataccgcataagaccacagcaccgcatggtgcgggggtaaaaactccggcggtatgagatggacccgcgtctgattagctagttggtgaggtaacggcccaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaaggatttcggtttgtaaagctcttttatcagggaagaaaatgacggtacctgactaagaagccccggctaactacgtgccagcagccgcggtaatacgtagggggcaagcgttatccggatttactgggcgtaaagggagcgtaggcggcaagtctgatgtgaaagcccggggctcaaccgcgggactgctttggaaactgtgagtgcaggagaggtaagtggaattcctagtgtagcggtgaaatgcgtagatattaggaggaacaccagtggcgaaggcggcttactggactgtaactgacgctgaggctcgaaagcgtggggagcaaacaggattagataccctggtagtccacgccgtaaacgatgaatactaggtgtygggagcccttcggtgccgcagctaacgcagtaagtattccgcctggggagtacgttcgcaagaatgaaactcaaaggaattgacgggggcccgcacaagcggtggagcatgtggtttaattcgaagcaacgcgaagaaccttaccaggtcttgacatccatctgaccgagagatggggccttcccttcgggcaggggagacaggtggtgcatggttgtcgtcagctcgtgtcgtgagatgttgggttaagtcccgcaacgagcgcaacccttatcyttagttgccagcattaagctgggcactctagggagactgccggggataacccggaggaaggtggggatgacgtcaaatcatcatgccccttatgacctgggctacacacgtgctacaatggcgtaaacaaagggaagcgagaccgcgaggccgagcaaatcccaaaaagtctcagttcggattgtagtctgcaactcgactacatgaagctggaatcgctagtaatcgcggatcagaatgccgcggtgaatacgttcccgggccttgtacacaccgcccgtcacaccatgggagtcagtaacgcccgaagtcggtgacctaaccaaggagggagctgccgaaggtgggachgatgactggggtgaagtcgtaacaaggtagccgtatcggaaggtgcggctggatcacctcctttctaaggaatacaaattcggccggccag

SEQ ID NO: 11 - DNA, 1234 bp, synthetic construct
  source 1..1234: mol_type = other DNA
cgaattcctgcagcccgggtggggatgcgttccattaggtagttggcggggtaacggcccaccaagcctacgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttatatgggaataaagttcggtacgtgtgggattttgtatgtaccatatgaataaggatcggctaactccgtgccagcagccgcggtaatacggaggatccgagcgttatccggatttattgggtttaaagggagcgtaggtggatcgttaagtcagttgtgaaagtttgcggctcaaccgtaaaattgcagttgatactggaggtcttgagtacagtagaggtaggcggaattcgtggtgtagcggtgaaatgcttagatatcacgaagaactccgattgcgaaggcagcttactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgctgaattggaaacagcttagccgcaaggcagatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcggagtctgcaacccgactccgtgaagctggattcgctagtaatcgcgcatcagccacggcgcggtgaatacgttcccgggccttgtacacaccgcccgtcaagccatgaaagccgggggtacctgaagtacgtggatccactagttctagagc

SEQ ID NO: 12 - DNA, 322 bp, synthetic construct
  source 1..322: mol_type = other DNA
tcgtctcctagctaatttctactcttgtagatataagaatgccttacaagtcttaatttctactcttgtagataggagaatagaaagaagaaaattctttctaaaggctgaattctctgtttaattttgagagaccattctctcaaaattgaaacttctcaataaaaattgagaagtagctgaccatcacaaaatcgtagattttggatgtctagctatgttctttgaaaattgcacagtgaataagtaaagctaaaggtatataaaaatcctttgtaagaatacaatttgcaaagtgacagaggaaagctacgggagacgg

SEQ ID NO: 13 - DNA, 476 bp, synthetic construct
  source 1..476: mol_type = other DNA
cctgcaggccaacacatcaagcttgacagctagctcagtcctaggtataatgctagccgagacggcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggtaatagtccaggcatcaaataaaacgaaaggctcagtcgaaagactgggcctttcgttttatctgttgtttgtcggtgaacgctctcctgacgtctcctacgtaggcggccgc

SEQ ID NO: 14 - DNA, 382 bp, synthetic construct
  source 1..382: mol_type = other DNA
gtcctaggtataatgctagctaatttctactcttgtagatacacaggaaacagctatgactaatttctactcttgtagatcaacgtcgtgactgggaaaacctaatttctactcttgtagataggagaatagaaagaagaaaattctttctaaaggctgaattctctgtttaattttgagagaccattctctcaaaattgaaacttctcaataaaaattgagaagtagctgaccatcacaaaatcgtagattttggatgtctagctatgttctttgaaaattgcacagtgaataagtaaagctaaaggtatataaaaatcctttgtaagaatacaatttgcaaagtgacagaggaaagcgagacggcgcaacgcaattaatg

SEQ ID NO: 15 - DNA, 361 bp, synthetic construct
  source 1..361: mol_type = other DNA
tcgtctcctagctaatttctactcttgtagatctatatgccgacggacaagctaatttctactcttgtagataggattaatacgattataattaatttctactcttgtagataggagaatagaaagaagaaaattctttctaaaggctgaattctctgtttaattttgagagaccattctctcaaaattgaaacttctcaataaaaattgagaagtagctgaccatcacaaaatcgtagattttggatgtctagctatgttctttgaaaattgcacagtgaataagtaaagctaaaggtatataaaaatcctttgtaagaatacaatttgcaaagtgacagaggaaagctacgggagacgg

SEQ ID NO: 16 - DNA, 377 bp, synthetic construct
  source 1..377: mol_type = other DNA
gtcctaggtataatgctagctaatttctactcttgtagattacctcatagctacccttcactaatttctactcttgtagattataatggtgatatgaaaactaatttctactcttgtagataggagaatagaaagaagaaaattctttctaaaggctgaattctctgtttaattttgagagaccattctctcaaaattgaaacttctcaataaaaattgagaagtagctgaccatcacaaaatcgtagattttggatgtctagctatgttctttgaaaattgcacagtgaataagtaaagctaaaggtatataaaaatcctttgtaagaatacaatttgcaaagtgacagaggaaagcctgacgtctcctacgtagg

SEQ ID NO: 17 - DNA, 20 bp, synthetic construct
  source 1..20: mol_type = other DNA
tgcttccggctcgtatgttg

SEQ ID NO: 18 - DNA, 20 bp, synthetic construct
  source 1..20: mol_type = other DNA
acacaggaaacagctatgac

SEQ ID NO: 19 - DNA, 25 bp, synthetic construct
  source 1..25: mol_type = other DNA
cctacggrrbgcascagkvrvgaat

SEQ ID NO: 20 - DNA, 22 bp, synthetic construct
  source 1..22: mol_type = other DNA
  misc_difference 8: note = a, c, t, g, unknown, or other
ggactacnvgggtwtctaatcc

SEQ ID NO: 21 - DNA, 19 bp, synthetic construct
  source 1..19: mol_type = other DNA
gtgycagcmgccgcggtaa

SEQ ID NO: 22 - DNA, 23 bp, synthetic construct
  source 1..23: mol_type = other DNA
cttgtgcggkcccccgycaattc

SEQ ID NO: 23 - DNA, 40 bp, synthetic construct
  source 1..40: mol_type = other DNA
cgaattcctgcagcccgggtatgaataaggatcggctaac

SEQ ID NO: 24 - DNA, 20 bp, synthetic construct
  source 1..20: mol_type = other DNA
caatttaagcccgggtaagg

SEQ ID NO: 25 - DNA, 40 bp, synthetic construct
  source 1..40: mol_type = other DNA
ccttacccgggcttaaattgtgtgaaggtgctgcatggtt

SEQ ID NO: 26 - DNA, 40 bp, synthetic construct
  source 1..40: mol_type = other DNA
gctctagaactagtggatccgctccagaaaggaggtgttc

SEQ ID NO: 27 - DNA, 39 bp, synthetic construct
  source 1..39: mol_type = other DNA
cacaggttcatcttctcccgcgaattcctgcagcccggg

SEQ ID NO: 28 - DNA, 36 bp, synthetic construct
  source 1..36: mol_type = other DNA
cgataccgtcgactcggctctagaactagtggatcc

SEQ ID NO: 29 - DNA, 16 bp, synthetic construct
  source 1..16: mol_type = other DNA
cgagtcgacggtatcg

SEQ ID NO: 30 - DNA, 20 bp, synthetic construct
  source 1..20: mol_type = other DNA
cgggagaagatgaacctgtg

SEQ ID NO: 31 - DNA, 43 bp, synthetic construct
  source 1..43: mol_type = other DNA
gcaataaattaaggagaggttataatggtatttgaaaaaattg

SEQ ID NO: 32 - DNA, 40 bp, synthetic construct
  source 1..40: mol_type = other DNA
cagttgcatgtggcctattggttaactatttatcaattcc

SEQ ID NO: 33 - DNA, 20 bp, synthetic construct
  source 1..20: mol_type = other DNA
caataggccacatgcaactg

SEQ ID NO: 34 - DNA, 24 bp, synthetic construct
  source 1..24: mol_type = other DNA
tataacctctccttaatttattgc

SEQ ID NO: 35 - DNA, 20 bp, synthetic construct
  source 1..20: mol_type = other DNA
cacaggttcatcttctcccg

SEQ ID NO: 36 - DNA, 16 bp, synthetic construct
  source 1..16: mol_type = other DNA
cgataccgtcgactcg

SEQ ID NO: 37 - DNA, 20 bp, synthetic construct
  source 1..20: mol_type = other DNA
agagtttgatcgtggctcag

SEQ ID NO: 38 - DNA, 22 bp, synthetic construct
  source 1..22: mol_type = other DNA
ctgtcagaccaagtttactcat

SEQ ID NO: 39 - DNA, 40 bp, synthetic construct
  source 1..40: mol_type = other DNA
cacaggttcatcttctcccgttaccacatgcaggaagcag

SEQ ID NO: 40 - DNA, 36 bp, synthetic construct
  source 1..36: mol_type = other DNA
cgataccgtcgactcgttctcagccagttcacatgc

SEQ ID NO: 41 - DNA, 20 bp, synthetic construct
  source 1..20: mol_type = other DNA
ctgaaagagttcatggtgcg

SEQ ID NO: 42 - DNA, 40 bp, synthetic construct
  source 1..40: mol_type = other DNA
cacaggttcatcttctcccgtccggtcagaaaggtttgtc

SEQ ID NO: 43 - DNA, 36 bp, synthetic construct
  source 1..36: mol_type = other DNA
cgataccgtcgactcgtcgatacgagcctgtgtacg

SEQ ID NO: 44 - DNA, 20 bp, synthetic construct
  source 1..20: mol_type = other DNA
atggaacatctcggctatgc

SEQ ID NO: 45 - DNA, 40 bp, synthetic construct
  source 1..40: mol_type = other DNA
cacaggttcatcttctcccgtcctccttatctgcgtggtc

SEQ ID NO: 46 - DNA, 36 bp, synthetic construct
  source 1..36: mol_type = other DNA
cgataccgtcgactcgttcctcgatacgttcccaag

SEQ ID NO: 47 - DNA, 20 bp, synthetic construct
  source 1..20: mol_type = other DNA
aacggtatttccgctgattg

SEQ ID NO: 48 - DNA, 42 bp, synthetic construct
  source 1..42: mol_type = other DNA
atgagtaaacttggtctgacaggcggccgctgtatccatatg

SEQ ID NO: 49 - DNA, 34 bp, synthetic construct
  source 1..34: mol_type = other DNA
cgataccgtcgactcgggtcatagctgtttcctg

SEQ ID NO: 50 - DNA, 20 bp, synthetic construct
  source 1..20: mol_type = other DNA
gcactggccgtcgttttaca

SEQ ID NO: 51 - DNA, 20 bp, synthetic construct
  source 1..20: mol_type = other DNA
catcttccgtaagttccctt

SEQ ID NO: 52 - DNA, 19 bp, synthetic construct
  source 1..19: mol_type = other DNA
gttttcatatacgcccttc

SEQ ID NO: 53 - DNA, 20 bp, synthetic construct
  source 1..20: mol_type = other DNA
cctttgagtgcttatgtggc

SEQ ID NO: 54 - DNA, 20 bp, synthetic construct
  source 1..20: mol_type = other DNA
cccaaattcgagtaagaggt

SEQ ID NO: 55 - DNA, 20 bp, synthetic construct
  source 1..20: mol_type = other DNA
ccttcctccaaatcagacaa

SEQ ID NO: 56 - DNA, 21 bp, synthetic construct
  source 1..21: mol_type = other DNA
gttatggaacgtatgcagacg

SEQ ID NO: 57 - DNA, 20 bp, synthetic construct
  source 1..20: mol_type = other DNA
cgtgtaaaggactgacagcc

SEQ ID NO: 58 - DNA, 20 bp, synthetic construct
  source 1..20: mol_type = other DNA
cagtcgaacagtggaatctc

SEQ ID NO: 59 - DNA, 19 bp, synthetic construct
  source 1..19: mol_type = other DNA
cgaattcctgcagcccggg

SEQ ID NO: 60 - DNA, 17 bp, synthetic construct
  source 1..17: mol_type = other DNA
gacgggcggtgtgtaca

SEQ ID NO: 61 - DNA, 33 bp, synthetic construct
  source 1..33: mol_type = other DNA
gtgttctctagaatgacacagtttgaaggcttc

SEQ ID NO: 62 - DNA, 34 bp, synthetic construct
  source 1..34: mol_type = other DNA
gtgttactcgagttagtttctcagttcttgaatg

SEQ ID NO: 63 - DNA, 30 bp, synthetic construct
  source 1..30: mol_type = other DNA
ctcgtgctatgccaataattggggtctccg

SEQ ID NO: 64 - DNA, 38 bp, synthetic construct
  source 1..38: mol_type = other DNA
ccccaattattggcatagcacgaggagaacgcaatctc

SEQ ID NO: 65 - DNA, 20 bp, synthetic construct
  source 1..20: mol_type = other DNA
gaatggcgaatggcgctagc

SEQ ID NO: 66 - DNA, 18 bp, synthetic construct
  source 1..18: mol_type = other DNA
caacttgcccactggccg

SEQ ID NO: 67 - DNA, 18 bp, synthetic construct
  source 1..18: mol_type = other DNA
cggccagtgggcaagttg

SEQ ID NO: 68 - DNA, 20 bp, synthetic construct
  source 1..20: mol_type = other DNA
gctagcgccattcgccattc

SEQ ID NO: 69 - DNA, 92 bp, synthetic construct
  source 1..92: mol_type = other DNA
tcgtctcctagctaatttctactcttgtagatatggatatgtaactcagggagtaatttctactcttgtagataggagaatagaaagaagaa

SEQ ID NO: 70 - DNA, 32 bp, synthetic construct
  source 1..32: mol_type = other DNA
actgcctgcaggccaacacatcaagcttgaca

SEQ ID NO: 71 - DNA, 32 bp, synthetic construct
  source 1..32: mol_type = other DNA
actggcggccgcctacgtaggagacgtcagga

SEQ ID NO: 72 - DNA, 99 bp, synthetic construct
  source 1..99: mol_type = other DNA
acacaggaaacagctatgactaatttctactcttgtagatcaacgtcgtgactgggaaaacctaatttctactcttgtagataggagaatagaaagaag

SEQ ID NO: 73 - DNA, 20 bp, synthetic construct
  source 1..20: mol_type = other DNA
gctttcctctgtcactttgc

SEQ ID NO: 74 - DNA, 80 bp, synthetic construct
  source 1..80: mol_type = other DNA
gtcctaggtataatgctagctaatttctactcttgtagatacacaggaaacagctatgactaatttctactcttgtagat

SEQ ID NO: 75 - DNA, 43 bp, synthetic construct
  source 1..43: mol_type = other DNA
cattaattgcgttgcgccgtctcgctttcctctgtcactttgc

SEQ ID NO: 76 - DNA, 97 bp, synthetic construct
  source 1..97: mol_type = other DNA
tgcttccggctcgtatgttgtaatttctactcttgtagatacacaggaaacagctatgactaatttctactcttgtagataggagaatagaaagaag

SEQ ID NO: 77 - DNA, 80 bp, synthetic construct
  source 1..80: mol_type = other DNA
gtcctaggtataatgctagctaatttctactcttgtagattgcttccggctcgtatgttgtaatttctactcttgtagat

SEQ ID NO: 78 - DNA, 23 bp, synthetic construct
  source 1..23: mol_type = other DNA
gagacggcgcaacgcaattaatg

SEQ ID NO: 79 - DNA, 20 bp, synthetic construct
  source 1..20: mol_type = other DNA
gctagcattatacctaggac

SEQ ID NO: 80 - DNA, 18 bp, synthetic construct
  source 1..18: mol_type = other DNA
cggattcactggccgtcg

SEQ ID NO: 81 - DNA, 18 bp, synthetic construct
  source 1..18: mol_type = other DNA
caggctgcgcaactgttg

SEQ ID NO: 82 - DNA, 20 bp, synthetic construct
  source 1..20: mol_type = other DNA
tacacaccgcccgtcacacc

SEQ ID NO: 83 - DNA, 21 bp, synthetic construct
  source 1..21: mol_type = other DNA
tgatccagccgcaggttctcc

SEQ ID NO: 84 - DNA, 20 bp, synthetic construct
  source 1..20: mol_type = other DNA
gcgttatccggatttactgg

SEQ ID NO: 85 - DNA, 20 bp, synthetic construct
  source 1..20: mol_type = other DNA
agcctcaacgtcagttaccg

SEQ ID NO: 86 - DNA, 20 bp, synthetic construct
  source 1..20: mol_type = other DNA
gcgttatccggatttactgg

SEQ ID NO: 87 - DNA, 21 bp, synthetic construct
  source 1..21: mol_type = other DNA
gcatttcaccgctacactagg

SEQ ID NO: 88 - DNA, 20 bp, synthetic construct
  source 1..20: mol_type = other DNA
agctagttggcagggtaacg

SEQ ID NO: 89 - DNA, 21 bp, synthetic construct
  source 1..21: mol_type = other DNA
cgaaatacttcttcgctcacg

SEQ ID NO: 90 - DNA, 20 bp, synthetic construct
  source 1..20: mol_type = other DNA
gtctgatgtgaaaggcatgg

SEQ ID NO: 91 - DNA, 20 bp, synthetic construct
  source 1..20: mol_type = other DNA
agcctcaacgtcagttaccg

SEQ ID NO: 92 - DNA, 20 bp, synthetic construct
  source 1..20: mol_type = other DNA
tgggtgtaaagggagcgtag

SEQ ID NO: 93 - DNA, 20 bp, synthetic construct
  source 1..20: mol_type = other DNA
gccactggtgttcctcctaa

SEQ ID NO: 94 - DNA, 20 bp, synthetic construct
  source 1..20: mol_type = other DNA
gtaatacgtagggggcaagc

SEQ ID NO: 95 - DNA, 20 bp, synthetic construct
  source 1..20: mol_type = other DNA
agcctcaacgtcagttatcg

SEQ ID NO: 96 - DNA, 20 bp, synthetic construct
  source 1..20: mol_type = other DNA
tggggaatattgcacaatgg

SEQ ID NO: 97 - DNA, 20 bp, synthetic construct
  source 1..20: mol_type = other DNA
taaatccggataacgcttgc

SEQ ID NO: 98 - DNA, 20 bp, synthetic construct
  source 1..20: mol_type = other DNA
gcggcttactggacgataac

SEQ ID NO: 99 - DNA, 20 bp, synthetic construct
  source 1..20: mol_type = other DNA
tggaatgcttactgcgtttg

SEQ ID NO: 100 - DNA, 20 bp, synthetic construct
  source 1..20: mol_type = other DNA
gcggcttactggacgataac

SEQ ID NO: 101 - DNA, 20 bp, synthetic construct
  source 1..20: mol_type = other DNA
cccaggtggaatgcttactg

SEQ ID NO: 102 - DNA, 20 bp, synthetic construct
  source 1..20: mol_type = other DNA
gcggcttactggacgataac

SEQ ID NO: 103 - DNA, 20 bp, synthetic construct
  source 1..20: mol_type = other DNA
cccaggtggaatgcttactg

SEQ ID NO: 104 - DNA, 20 bp, synthetic construct
  source 1..20: mol_type = other DNA
gcggcttactggacgataac

SEQ ID NO: 105 - DNA, 20 bp, synthetic construct
  source 1..20: mol_type = other DNA
tggaatgcttactgcgtttg

SEQ ID NO: 106 - DNA, 20 bp, synthetic construct
  source 1..20: mol_type = other DNA
gaaagccacggctaactacg

SEQ ID NO: 107 - DNA, 20 bp, synthetic construct
  source 1..20: mol_type = other DNA
tctgcactccagcataccag

SEQ ID NO: 108 - DNA, 20 bp, synthetic construct
  source 1..20: mol_type = other DNA
gaaagccacggctaactacg

SEQ ID NO: 109 - DNA, 20 bp, synthetic construct
  source 1..20: mol_type = other DNA
tctgcactccagcataccag

SEQ ID NO: 110 - DNA, 20 bp, synthetic construct
  source 1..20: mol_type = other DNA
gtaggcggatttttaagtgg

SEQ ID NO: 111 - DNA, 20 bp, synthetic construct
  source 1..20: mol_type = other DNA
ctcagcgtcagttacagtcc

SEQ ID NO: 112 - DNA, 20 bp, synthetic construct
  source 1..20: mol_type = other DNA
cagcagggaagaaaatgacg

SEQ ID NO: 113 - DNA, 20 bp, synthetic construct
  source 1..20: mol_type = other DNA
actccagcaccacagtttcc

SEQ ID NO: 114 - DNA, 21 bp, synthetic construct
  source 1..21: mol_type = other DNA
cctagtgtagcggtgaaatgc

SEQ ID NO: 115 - DNA, 20 bp, synthetic construct
  source 1..20: mol_type = other DNA
gttaactgcggcacagaagg

SEQ ID NO: 116 - DNA, 20 bp, synthetic construct
  source 1..20: mol_type = other DNA
gaaagccacggctaactacg

SEQ ID NO: 117 - DNA, 21 bp, synthetic construct
  source 1..21: mol_type = other DNA
cactccagcataccagtttcc

SEQ ID NO: 118 - DNA, 20 bp, synthetic construct
  source 1..20: mol_type = other DNA
tattccacctggggagtacg

SEQ ID NO: 119 - DNA, 20 bp, synthetic construct
  source 1..20: mol_type = other DNA
atgcaccacctgtcttctcc

SEQ ID NO: 120 - DNA, 20 bp, synthetic construct
  source 1..20: mol_type = other DNA
tgtagcggtgaaatgcgtag

SEQ ID NO: 121 - DNA, 20 bp, synthetic construct
  source 1..20: mol_type = other DNA
cactgagtttccccaacacc

SEQ ID NO: 122 - DNA, 20 bp, synthetic construct
  source 1..20: mol_type = other DNA
gagataacagcccaccaagg

SEQ ID NO: 123 - DNA, 21 bp, synthetic construct
  source 1..21: mol_type = other DNA
cgaaaaccttcttcactcacg

SEQ ID NO: 124 - DNA, 20 bp, synthetic construct
  source 1..20: mol_type = other DNA
agtcttgacatcccggtgac

SEQ ID NO: 125 - DNA, 20 bp, synthetic construct
  source 1..20: mol_type = other DNA
atccgaaatgctggctactg

SEQ ID NO: 126 - DNA, 20 bp, synthetic construct
  source 1..20: mol_type = other DNA
actaagaagccccggctaac

SEQ ID NO: 127 - DNA, 20 bp, synthetic construct
  source 1..20: mol_type = other DNA
ttcgacagtttccaatgcag

SEQ ID NO: 128 - DNA, 20 bp, synthetic construct
  source 1..20: mol_type = other DNA
tggaaacggctgctaatacc

SEQ ID NO: 129 - DNA, 20 bp, synthetic construct
  source 1..20: mol_type = other DNA
cgtgtctcagtcccaatgtg

SEQ ID NO: 130 - DNA, 20 bp, synthetic construct
  source 1..20: mol_type = other DNA
tggggaatattgcacaatgg

SEQ ID NO: 131 - DNA, 20 bp, synthetic construct
  source 1..20: mol_type = other DNA
taattccggataacgcttgc

SEQ ID NO: 132 - DNA, 20 bp, synthetic construct
  source 1..20: mol_type = other DNA
agcgttgtccggatttattg

SEQ ID NO: 133 - DNA, 20 bp, synthetic construct
  source 1..20: mol_type = other DNA
tacgcatttcaccgctacac

SEQ ID NO: 134 - DNA, 20 bp, synthetic construct
  source 1..20: mol_type = other DNA
tgcatctgatactggcaagc

SEQ ID NO: 135 - DNA, 20 bp, synthetic construct
  source 1..20: mol_type = other DNA
caagtcgacatcgtttacgg

SEQ ID NO: 136 - DNA, 99 bp, synthetic construct
  source 1..99: mol_type = other DNA
ctatatgccgacggacaagctaatttctactcttgtagataggattaatacgattataattaatttctactcttgtagataggagaatagaaagaagaa

SEQ ID NO: 137 - DNA, 72 bp, synthetic construct
  source 1..72: mol_type = other DNA
tcgtctcctagctaatttctactcttgtagatctatatgccgacggacaagctaatttctactcttgtagat

SEQ ID NO: 138 - DNA, 32 bp, synthetic construct
  source 1..32: mol_type = other DNA
ccgtctcccgtagctttcctctgtcactttgc

SEQ ID NO: 139 - DNA, 20 bp, synthetic construct
  source 1..20: mol_type = other DNA
agcgctatgtggcgaattat

SEQ ID NO: 140 - DNA, 20 bp, synthetic construct
  source 1..20: mol_type = other DNA
aatgcttccatcctcagtgg

SEQ ID NO: 141 - DNA, 19 bp, synthetic construct
  source 1..19: mol_type = other DNA
ctgacgtctcctacgtagg

SEQ ID NO: 142 - DNA, 20 bp, synthetic construct
  source 1..20: mol_type = other DNA
gctagcattatacctaggac

SEQ ID NO: 143 - DNA, 99 bp, synthetic construct
  source 1..99: mol_type = other DNA
aaggtctgaaagcttacacctaatttctactcttgtagattcaaacggcagatgggaggataatttctactcttgtagataggagaatagaaagaagaa

SEQ ID NO: 144 - DNA, 72 bp, synthetic construct
  source 1..72: mol_type = other DNA
tcgtctcctagctaatttctactcttgtagataaggtctgaaagcttacacctaatttctactcttgtagat

SEQ ID NO: 145 - DNA, 20 bp, synthetic construct
  source 1..20: mol_type = other DNA
gaattctgctctccgactgg

SEQ ID NO: 146 - DNA, 20 bp, synthetic construct
  source 1..20: mol_type = other DNA
ggttttacgccgattacagc

SEQ ID NO: 147 - DNA, 99 bp, synthetic construct
  source 1..99: mol_type = other DNA
aagatggcgcatgggatgcctaatttctactcttgtagatctccgttcacacatattacataatttctactcttgtagataggagaatagaaagaagaa

SEQ ID NO: 148 - DNA, 80 bp, synthetic construct
  source 1..80: mol_type = other DNA
gtcctaggtataatgctagctaatttctactcttgtagataagatggcgcatgggatgcctaatttctactcttgtagat

SEQ ID NO: 149 - DNA, 20 bp, synthetic construct
  source 1..20: mol_type = other DNA
gcaggtgttttccagtatgc

SEQ ID NO: 150 - DNA, 20 bp, synthetic construct
  source 1..20: mol_type = other DNA
catcaacgaaacggtcttcc

SEQ ID NO: 151 - DNA, 99 bp, synthetic construct
  source 1..99: mol_type = other DNA
gctgattacatgttcactattaatttctactcttgtagattcgaatgtatttttacattctaatttctactcttgtagataggagaatagaaagaagaa

SEQ ID NO: 152 - DNA, 72 bp, synthetic construct
  source 1..72: mol_type = other DNA
tcgtctcctagctaatttctactcttgtagatgctgattacatgttcactattaatttctactcttgtagat

SEQ ID NO: 153 - DNA, 20 bp, synthetic construct
  source 1..20: mol_type = other DNA
tggccagatccagttattcc

SEQ ID NO: 154 - DNA, 20 bp, synthetic construct
  source 1..20: mol_type = other DNA
agaagtttccggttcagacg

SEQ ID NO: 155 - DNA, 99 bp, synthetic construct; identical to SEQ ID NO: 136
  source 1..99: mol_type = other DNA

SEQ ID NO: 156 - DNA, 80 bp, synthetic construct
  source 1..80: mol_type = other DNA
gtcctaggtataatgctagctaatttctactcttgtagatctatatgccgacggacaagctaatttctactcttgtagat

SEQ ID NO: 157 - DNA, 20 bp, synthetic construct
  source 1..20: mol_type = other DNA
ctgattccatccttccatcc

SEQ ID NO: 158 - DNA, 20 bp, synthetic construct
  source 1..20: mol_type = other DNA
gagataacggctgctgttcc

SEQ ID NO: 159 - DNA, 99 bp, synthetic construct
  source 1..99: mol_type = other DNA
gaaccatctttacggaccactaatttctactcttgtagattgtccagacagcatgggcactaatttctactcttgtagataggagaatagaaagaagaa

SEQ ID NO: 160 - DNA, 72 bp, synthetic construct
  source 1..72: mol_type = other DNA
tcgtctcctagctaatttctactcttgtagatgaaccatctttacggaccactaatttctactcttgtagat

SEQ ID NO: 161 - DNA, 20 bp, synthetic construct
  source 1..20: mol_type = other DNA
attctgtcattgccctgtgc

SEQ ID NO: 162 - DNA, 20 bp, synthetic construct
  source 1..20: mol_type = other DNA
tcctcaaagcgaagttcacc

SEQ ID NO: 163 - DNA, 100 bp, synthetic construct
  source 1..100: mol_type = other DNA
tacctcatagctacccttcactaatttctactcttgtagattataatggtgatatgaaaactaatttctactcttgtagataggagaatagaaagaagaa

SEQ ID NO: 164 - DNA, 81 bp, synthetic construct
  source 1..81: mol_type = other DNA
gtcctaggtataatgctagctaatttctactcttgtagattacctcatagctacccttcactaatttctactcttgtagat

SEQ ID NO: 165 - DNA, 39 bp, synthetic construct
  source 1..39: mol_type = other DNA
cctacgtaggagacgtcaggctttcctctgtcactttgc

SEQ ID NO: 166 - DNA, 19 bp, synthetic construct
  source 1..19: mol_type = other DNA
gaaaaccctaacgcaaagg

SEQ ID NO: 167 - DNA, 21 bp, synthetic construct
  source 1..21: mol_type = other DNA
cttgttatgcctggtaacacc

SEQ ID NO: 168 - DNA, 99 bp, synthetic construct
  source 1..99: mol_type = other DNA
ccatggattggacagcagagtaatttctactcttgtagatataggttacaccctttttgctaatttctactcttgtagataggagaatagaaagaagaa

SEQ ID NO: 169 - DNA, 80 bp, synthetic construct
  source 1..80: mol_type = other DNA
gtcctaggtataatgctagctaatttctactcttgtagatccatggattggacagcagagtaatttctactcttgtagat

SEQ ID NO: 170 - DNA, 20 bp, synthetic construct
  source 1..20: mol_type = other DNA
caaccgtcgtctgtgtatgc

SEQ ID NO: 171 - DNA, 20 bp, synthetic construct
  source 1..20: mol_type = other DNA
aggcattgcttggatgtacc

SEQ ID NO: 172 - DNA, 99 bp, synthetic construct
  source 1..99: mol_type = other DNA
gttatatgcagaccgattactaatttctactcttgtagattagttctgctgttaacgtgttaatttctactcttgtagataggagaatagaaagaagaa

SEQ ID NO: 173 - DNA, 72 bp, synthetic construct
  source 1..72: mol_type = other DNA
tcgtctcctagctaatttctactcttgtagatgttatatgcagaccgattactaatttctactcttgtagat

SEQ ID NO: 174 - DNA, 20 bp, synthetic construct
  source 1..20: mol_type = other DNA
aaccgaagacggacatatcg

SEQ ID NO: 175 - DNA, 20 bp, synthetic construct
  source 1..20: mol_type = other DNA
ccgtaaggaggcacataagc

SEQ ID NO: 176 - DNA, 99 bp, synthetic construct
  source 1..99: mol_type = other DNA
tcatggattacacagaaggttaatttctactcttgtagatcataattcttccactccaattaatttctactcttgtagataggagaatagaaagaagaa

SEQ ID NO: 177 - DNA, 80 bp, synthetic construct
  source 1..80: mol_type = other DNA
gtcctaggtataatgctagctaatttctactcttgtagattcatggattacacagaaggttaatttctactcttgtagat

SEQ ID NO: 178 - DNA, 20 bp, synthetic construct
  source 1..20: mol_type = other DNA
gtacaggcgaggctaaatgc

SEQ ID NO: 179 - DNA, 21 bp, synthetic construct
  source 1..21: mol_type = other DNA
ggaaggatagttccgtgaagc

SEQ ID NO: 180 - DNA, 100 bp, synthetic construct
  source 1..100: mol_type = other DNA
gtaagtatatgacagattactaatttctactcttgtagatccaaaagtgtaatgggaaaagtaatttctactcttgtagataggagaatagaaagaagaa

SEQ ID NO: 181 - DNA, 72 bp, synthetic construct
  source 1..72: mol_type = other DNA
tcgtctcctagctaatttctactcttgtagatgtaagtatatgacagattactaatttctactcttgtagat

SEQ ID NO: 182 - DNA, 20 bp, synthetic construct
  source 1..20: mol_type = other DNA
agctgactgggttccatctg

SEQ ID NO: 183 - DNA, 20 bp, synthetic construct
  source 1..20: mol_type = other DNA
cggattaagacctgctgcat

SEQ ID NO: 184 - DNA, 99 bp, synthetic construct
  source 1..99: mol_type = other DNA
atcggctccgtggccgctcttaatttctactcttgtagatgggcggctgcaacaagaagataatttctactcttgtagataggagaatagaaagaagaa

SEQ ID NO: 185 - DNA, 72 bp, synthetic construct
  source 1..72: mol_type = other DNA
tcgtctcctagctaatttctactcttgtagatatcggctccgtggccgctcttaatttctactcttgtagat

SEQ ID NO: 186 - DNA, 60 bp, synthetic construct
  source 1..60: mol_type = other DNA
gtcctaggtataatgctagctaatttctactcttgtagatatcggctccgtggccgctct

SEQ ID NO: 187 - DNA, 20 bp, synthetic construct
  source 1..20: mol_type = other DNA
agtggactactgcgtgatcg

SEQ ID NO: 188 - DNA, 20 bp, synthetic construct
  source 1..20: mol_type = other DNA
cagagatgcggcatagttgc

SEQ ID NO: 189 - DNA, 99 bp, synthetic construct
  source 1..99: mol_type = other DNA
ttgattacgaggaggatggataatttctactcttgtagattggtttacaacatatggatgtaatttctactcttgtagataggagaatagaaagaagaa

SEQ ID NO: 190 - DNA, 80 bp, synthetic construct
  source 1..80: mol_type = other DNA
gtcctaggtataatgctagctaatttctactcttgtagatttgattacgaggaggatggataatttctactcttgtagat

SEQ ID NO: 191 - DNA, 99 bp, synthetic construct
  source 1..99: mol_type = other DNA
aacgctttaaacagtgaagttaatttctactcttgtagattcttagcagaaaaaggaaattaatttctactcttgtagataggagaatagaaagaagaa

SEQ ID NO: 192 - DNA, 80 bp, synthetic construct
  source 1..80: mol_type = other DNA
gtcctaggtataatgctagctaatttctactcttgtagataacgctttaaacagtgaagttaatttctactcttgtagat

SEQ ID NO: 193 - DNA, 54 bp, synthetic construct
  source 1..54: mol_type = other DNA
ttttaagctttataattatccttaatcgacggtgttgtgcgcccagatagggtg

SEQ ID NO: 194 - DNA, 60 bp, synthetic construct
  source 1..60: mol_type = other DNA
cagattgtacaaatgtggtgataacagataagtcggtgttgataacttacctttctttgt

SEQ ID NO: 195 - DNA, 49 bp, synthetic construct
  source 1..49: mol_type = other DNA
tgaacgcaagtttctaatttcggtttcgatccgatagaggaaagtgtct

SEQ ID NO: 196 - DNA, 20 bp, synthetic construct
  source 1..20: mol_type = other DNA
gaaccacaaggtgcagttgc

SEQ ID NO: 197 - DNA, 21 bp, synthetic construct
  source 1..21: mol_type = other DNA
gagcgtatttgtcagagccag

SEQ ID NO: 198 - DNA, 28 bp, synthetic construct
  source 1..28: mol_type = other DNA
cgaaattagaaacttgcgttcagtaaac

SEQ ID NO: 199 - DNA, 54 bp, synthetic construct
  source 1..54: mol_type = other DNA
ttttaagctttataattatccttagccttcgccactgtgcgcccagatagggtg

SEQ ID NO: 200 - DNA, 60 bp, synthetic construct
  source 1..60: mol_type = other DNA
cagattgtacaaatgtggtgataacagataagtcgccactggtaacttacctttctttgt

SEQ ID NO: 201 - DNA, 49 bp, synthetic construct
  source 1..49: mol_type = other DNA
tgaacgcaagtttctaatttcgattaaggctcgatagaggaaagtgtct

SEQ ID NO: 202 - DNA, 27 bp, synthetic construct
  source 1..27: mol_type = other DNA
ctgttatcaccacatttgtacaatctg

SEQ ID NO: 203 - DNA, 28 bp, synthetic construct
  source 1..28: mol_type = other DNA
ggataattataaagcttaaaaattacac

SEQ ID NO: 204 - DNA, 40 bp, synthetic construct
  source 1..40: mol_type = other DNA
gctagcgccattcgccattcggcgcgcccgtatctgtggg

SEQ ID NO: 205 - DNA, 52 bp, synthetic construct
  source 1..52: mol_type = other DNA
gatgctggtgcttctggtgctggtatgaatacatacgaacaaattaataaag

SEQ ID NO: 206 - DNA, 43 bp, synthetic construct
  source 1..43: mol_type = other DNA
ggaacttcgcgactcatagaaccaattagaatgaatatttccc

SEQ ID NO: 207 - DNA, 26 bp, synthetic construct
  source 1..26: mol_type = other DNA
ttctatgagtcgcgaagttcctattc

SEQ ID NO: 208 - DNA, 24 bp, synthetic construct
  source 1..24: mol_type = other DNA
accagcaccagaagcaccagcatc

SEQ ID NO: 209 - DNA, 18 bp, synthetic construct
  source 1..18: mol_type = other DNA
ggtgcttctggtgctggt

SEQ ID NO: 210 - DNA, 17 bp, synthetic construct
  source 1..17: mol_type = other DNA
ctgcccgccacacatgt

SEQ ID NO: 211 - DNA, 28 bp, synthetic construct
  source 1..28: mol_type = other DNA
cgaaattagaaacttgcgttcagtaaac

SEQ ID NO: 212 - DNA, 54 bp, synthetic construct
  source 1..54: mol_type = other DNA
ttttaagctttataattatccttagaagtcttcgctgtgcgcccagatagggtg

SEQ ID NO: 213 - DNA, 60 bp, synthetic construct
  source 1..60: mol_type = other DNA
cagattgtacaaatgtggtgataacagataagtcttcgcttataacttacctttctttgt

SEQ ID NO: 214 - DNA, 49 bp, synthetic construct
  source 1..49: mol_type = other DNA
tgaacgcaagtttctaatttcgattacttctcgatagaggaaagtgtct

SEQ ID NO: 215 - DNA, 27 bp, synthetic construct
  source 1..27: mol_type = other DNA
ctgttatcaccacatttgtacaatctg

SEQ ID NO: 216 - DNA, 28 bp, synthetic construct
  source 1..28: mol_type = other DNA
ggataattataaagcttaaaaattacac

SEQ ID NO: 217 - DNA, 36 bp, synthetic construct
  source 1..36: mol_type = other DNA
ctgattttcctgggccggcccaatgaataggtttac

SEQ ID NO: 218 - DNA, 42 bp, synthetic construct
  source 1..42: mol_type = other DNA
cttagggtaacaaaaaacaccgacgcgttatgagtaaacttg

SEQ ID NO: 219 - DNA, 22 bp, synthetic construct
  source 1..22: mol_type = other DNA
cggtgttttttgttaccctaag

SEQ ID NO: 220 - DNA, 20 bp, synthetic construct
  source 1..20: mol_type = other DNA
ggccggcccaggaaaatcag

SEQ ID NO: 221 - DNA, 46 bp, synthetic construct
  source 1..46: mol_type = other DNA
gatgctggtgcttctggtgctggtatggtatttgaaaaaattgata

SEQ ID NO: 222 - DNA, 43 bp, synthetic construct
  source 1..43: mol_type = other DNA
ggaacttcgcgactcatagaacttagggtaacaaaaaacaccg

SEQ ID NO: 223 - DNA, 26 bp, synthetic construct
  source 1..26: mol_type = other DNA
ttctatgagtcgcgaagttcctattc

SEQ ID NO: 224 - DNA, 24 bp, synthetic construct
  source 1..24: mol_type = other DNA
accagcaccagaagcaccagcatc

SEQ ID NO: 225 - DNA, 20 bp, synthetic construct
  source 1..20: mol_type = other DNA
atcgaagtcaacggagaatg

SEQ ID NO: 226 - DNA, 20 bp, synthetic construct
  source 1..20: mol_type = other DNA
ccgtcttcgagattcgttta

SEQ ID NO: 227 - DNA, 20 bp, synthetic construct
  source 1..20: mol_type = other DNA
gagctgacaccccgactgcc

SEQ ID NO: 228 - DNA, 20 bp, synthetic construct
  source 1..20: mol_type = other DNA
ccaggtctctttggagtcag

SEQ ID NO: 229 - DNA, 21 bp, synthetic construct
  source 1..21: mol_type = other DNA
gatcatcacagaggtccagca

SEQ ID NO: 230 - DNA, 23 bp, synthetic construct
  source 1..23: mol_type = other DNA
cttacgcgctcataggtcacatc

SEQ ID NO: 231 - DNA, 20 bp, synthetic construct
  source 1..20: mol_type = other DNA
acgagacggataggcagaga

SEQ ID NO: 232 - DNA, 20 bp, synthetic construct
  source 1..20: mol_type = other DNA
cacatgcaaggaagggaact

SEQ ID NO: 233 - DNA, 20 bp, synthetic construct
  source 1..20: mol_type = other DNA
tcacagcagcacatcaacaa

SEQ ID NO: 234 - DNA, 20 bp, synthetic construct
  source 1..20: mol_type = other DNA
tgtcctcatcctcgaaggtc

SEQ ID NO: 235 - DNA, 20 bp, synthetic construct
  source 1..20: mol_type = other DNA
ccggagaggagacttcacag

SEQ ID NO: 236 - DNA, 20 bp, synthetic construct
  source 1..20: mol_type = other DNA
tccacgatttcccagagaac

SEQ ID NO: 237 - DNA, 20 bp, synthetic construct
  source 1..20: mol_type = other DNA
ccctggactctccaccgcaa

SEQ ID NO: 238 - DNA, 20 bp, synthetic construct
  source 1..20: mol_type = other DNA
tccctccgcattgacacagc

SEQ ID NO: 239 - DNA, 20 bp, synthetic construct
  source 1..20: mol_type = other DNA
atggacagagccaatgggaa

SEQ ID NO: 240 - DNA, 20 bp, synthetic construct
  source 1..20: mol_type = other DNA
gtgtcctggtccaggcagag

SEQ ID NO: 241 - DNA, 20 bp, synthetic construct
  source 1..20: mol_type = other DNA
ctgacatcacatattggcat

SEQ ID NO: 242 - DNA, 21 bp, synthetic construct
  source 1..21: mol_type = other DNA
tctgaaccgaagcatgcatac

SEQ ID NO: 243 - DNA, 20 bp, synthetic construct
  source 1..20: mol_type = other DNA
acggcatggatctcaaagac

SEQ ID NO: 244 - DNA, 17 bp, synthetic construct
  source 1..17: mol_type = other DNA
gtgggtgagcacgtagt

SEQ ID NO: 245 - DNA, 20 bp, synthetic construct
  source 1..20: mol_type = other DNA
tgacgggggcccgcacaagc

SEQ ID NO: 246 - DNA, 20 bp, synthetic construct
  source 1..20: mol_type = other DNA
cacgagctgacgacaaccat

SEQ ID NO: 247 - DNA, 18 bp, synthetic construct
  source 1..18: mol_type = other DNA
gtgctgcagttaacgcaa

SEQ ID NO: 248 - DNA, 19 bp, synthetic construct
  source 1..19: mol_type = other DNA
gcgggacttaacccaacat

SEQ ID NO: 249 - DNA, 21 bp, synthetic construct
  source 1..21: mol_type = other DNA
ctgaatggcgaatggcgctag

SEQ ID NO: 250 - DNA, 20 bp, synthetic construct
  source 1..20: mol_type = other DNA
gcggccgcctacgtaggaga

SEQ ID NO: 251 - DNA, 22 bp, synthetic construct
  source 1..22: mol_type = other DNA
gtgtagtagcctgtgaaataag

SEQ ID NO: 252 - DNA, 54 bp, synthetic construct
  source 1..54: mol_type = other DNA
ctatttttatcaattttttcaaataccattaacacacctccttaaaaattacac

SEQ ID NO: 253 - DNA, 22 bp, synthetic construct
  source 1..22: mol_type = other DNA
ctcacgttaagggattttggtc

SEQ ID NO: 254 - DNA, 22 bp, synthetic construct
  source 1..22: mol_type = other DNA
gaccaaaatcccttaacgtgag

SEQ ID NO: 255 - DNA, 20 bp, synthetic construct
  source 1..20: mol_type = other DNA
gaatcaggggataacgcagg

SEQ ID NO: 256 - DNA, 20 bp, synthetic construct
  source 1..20: mol_type = other DNA
cctgcgttatcccctgattc

SEQ ID NO: 257 - DNA, 18 bp, synthetic construct
  source 1..18: mol_type = other DNA
gggcccgatcggtcttgc

SEQ ID NO: 258 - DNA, 18 bp, synthetic construct
  source 1..18: mol_type = other DNA
gcaagaccgatcgggccc

SEQ ID NO: 259 - DNA, 22 bp, synthetic construct
  source 1..22: mol_type = other DNA
cttatttcacaggctactacac

SEQ ID NO: 260 - DNA, 21 bp, synthetic construct
  source 1..21: mol_type = other DNA
ccgtaagttcccttatacgag

SEQ ID NO: 261 - DNA, 21 bp, synthetic construct
  source 1..21: mol_type = other DNA
ctcgtataagggaacttacgg

SEQ ID NO: 262 - DNA, 42 bp, synthetic construct
  source 1..42: mol_type = other DNA
cttatttcacaggctactacaccgaatttgtaaataaaccac

SEQ ID NO: 263 - DNA, 39 bp, synthetic construct
  source 1..39: mol_type = other DNA
gcaagaccgatcgggccccttaagtctaaaaattagggg

SEQ ID NO: 264 - DNA, 48 bp, synthetic construct
  source 1..48: mol_type = other DNA
cttatttcacaggctactacacgaaatgcaagtttctaactaacatac

SEQ ID NO: 265 - DNA, 43 bp, synthetic construct
  source 1..43: mol_type = other DNA
gcaagaccgatcgggcccgcattcacttcttttctatataaat

SEQ ID NO: 266 - DNA, 44 bp, synthetic construct
  source 1..44: mol_type = other DNA
cttatttcacaggctactacaccttaatcgcatttcatagattg

SEQ ID NO: 267 - DNA, 38 bp, synthetic construct
  source 1..38: mol_type = other DNA
gcaagaccgatcgggccccctgattctgtggataaccg

SEQ ID NO: 268 - DNA, 41 bp, synthetic construct
  source 1..41: mol_type = other DNA
cttatttcacaggctactacacgttcatttgatatgcctcc

SEQ ID NO: 269 - DNA, 38 bp, synthetic construct
  source 1..38: mol_type = other DNA
gcaagaccgatcgggcccctcacgttaagggattttgg

SEQ ID NO: 270 - DNA, 42 bp, synthetic construct
  source 1..42: mol_type = other DNA
cttatttcacaggctactacaccaggaaaatcagatccaacc

SEQ ID NO: 271 - DNA, 37 bp, synthetic construct
  source 1..37: mol_type = other DNA
gcaagaccgatcgggcccgcagcgaagatgttgtctg

SEQ ID NO: 272 - DNA, 43 bp, synthetic construct
  source 1..43: mol_type = other DNA
cttatttcacaggctactacacccttgtattgctattaatcgc

SEQ ID NO: 273 - DNA, 38 bp, synthetic construct
  source 1..38: mol_type = other DNA
gcaagaccgatcgggcccgaatggcgaatggcgctagc

SEQ ID NO: 274 - DNA, 40 bp, synthetic construct
  source 1..40: mol_type = other DNA
cttatttcacaggctactacaccaacttgcccactggccg

SEQ ID NO: 275 - DNA, 19 bp, synthetic construct
  source 1..19: mol_type = other DNA
gagaaactcttcgggatgc

SEQ ID NO: 276 - DNA, 37 bp, synthetic construct
  source 1..37: mol_type = other DNA
gcatcccgaagagtttctcgccagtgggcaagttgaa

SEQ ID NO: 277 - DNA, 37 bp, synthetic construct
  source 1..37: mol_type = other DNA
cgataccgtcgactcgggagtttaaacttagggtaac

SEQ ID NO: 278 - DNA, 40 bp, synthetic construct
  source 1..40: mol_type = other DNA
gcatcccgaagagtttctcctgcgctagcatgcctatttg

SEQ ID NO: 279 - DNA, 36 bp, synthetic construct
  source 1..36: mol_type = other DNA
cgataccgtcgactcggaaccgacgacaagctgacg

SEQ ID NO: 280 - DNA, 40 bp, synthetic construct
  source 1..40: mol_type = other DNA
cgaattcctgcagcccggggatcctggctcaggatgaacg

SEQ ID NO: 281 - DNA, 40 bp, synthetic construct
  source 1..40: mol_type = other DNA
gctctagaactagtggatcccttacggttaggcctgctac

SEQ ID NO: 282 - DNA, 40 bp, synthetic construct
  source 1..40: mol_type = other DNA
cgaattcctgcagcccgggcctaacacatgcaagtcgaac

SEQ ID NO: 283 - DNA, 40 bp, synthetic construct
  source 1..40: mol_type = other DNA
gctctagaactagtggatccccacgattactagcgattcc

SEQ ID NO: 284 - DNA, 39 bp, synthetic construct
  source 1..39: mol_type = other DNA
cgaattcctgcagcccgggagagtttgatcgtggctcag

SEQ ID NO: 285 - DNA, 37 bp, synthetic construct
  source 1..37: mol_type = other DNA
gctctagaactagtggatccgacgggcggtgtgtaca

SEQ ID NO: 286 - DNA, 19 bp, synthetic construct
  source 1..19: mol_type = other DNA
cgaattcctgcagcccggg

SEQ ID NO: 287 - DNA, 20 bp, synthetic construct
  source 1..20: mol_type = other DNA
gctctagaactagtggatcc

SEQ ID NO: 288-309 - skipped (22 entries)

SEQ ID NO: 310 - DNA, 4204 bp, synthetic construct
  source 1..4204: mol_type = other DNA
acgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccgcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaaccaataggccacatgcaactgtaaatgtttacgcgtcctcggtaccgcttcttccacaacagtctgcggttcctgtactatcacaggttcatcttctcccgcgaattcctgcagcccgggtggggatgcgttccattaggtagttggcggggtaacggcccaccaagcctacgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttatawgggaataaagttcggtacgtgtgggattttgtatgtaccatatgaataaggatcggctaactccgtgccagcagccgcggtaatacggaggatccgagcgttatccggatttattgggtttaaagggagcgtaggtggatcgttaagtcagttgtgaaagtttgcggctcaaccgtaaaattgcagttgatactggaggtcttgagtacagtagaggtaggcggaattcgtggtgtagcggtgaaatgcttagatatcacgaagaactccgattgcgaaggcagcttactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgctgaattggaaacagcttagccgcaaggcagatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcggagtctgcaacccgactccgtgaagctggattcgctagtaatcgcgcatcagccacggcgcggtgaatacgttcccgggccttgtacacaccgcccgtcaagccatgaaagccgggggtacctgaagtacgtggatccactagttctagagccgagtcgacggtatcgataagcttgatatcgaattcctgcagcccgggggatccactagttctagagcggccgccaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattg

SEQ ID NO: 311 - DNA, 2482 bp, Clostridium botulinum
  source 1..2482: mol_type = genomic DNA
gaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccagtgggcaagttg

SEQ ID NO: 312 - DNA, 1704 bp, Clostridium butyricum
  source 1..1704: mol_type = genomic DNA
gaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttg

SEQ ID NO: 313 - DNA, 3525 bp, Clostridium difficile
  source 1..3525: mol_type = genomic DNA
gaatggcgaatggcgctagcataaaaataagaagcctgcatttgcaggcttcttatttttatggcgcgcccgcccttaagtctaaaaattaggggagatgtaaggatttgggaaaaatagaagatgttataatcataaatatggtattcgtaggcttaaagtcaaaaaggaggtgaaatataaatagatttttagctaaattaagtaagaaataggaggagatttattgaacaaaaaattagaaaaaccatttgtatataagagagagtacgatttgactggatatgatgttgaaattttacaaaaatatgagttagaacaagcaatatatgtttatgttgggagtagttgtgcatataacatgagagctagaagtagtaaatggagataccatataagaacaaataataagtctatatgttgtaacattaaaaattttatacataacttggaattgttttataaaatggaattaaagttgtcagataatattattaatgataagctatactatagcaatatagcagagtttgaagaatttgaaacactagaaaaagctagagaggtagaaagtactattataagtcaatatcaatttttagattctataaatcacatgttaaaacaaaaaataattttattgagtaataaggatagtgtgttaaacataactaaaaatggaaatacaaattatttgaaagtaaaaaataaatacatagaaaaacataagaacaagccaataatgagataccatatcaactgtcaattcaatacagatggaagtgtcaaaagtattacacaggagtttgaaccaatattggaattaaacaaaaaaaataccctaagccgaccaagcagagtatttttaaaataatattttaagataacaacaaaatgagataatactactagacaatgacaactcaactaccaattgagtttatggagctaccaactccaatatcggtctaactgattaagtatctgtagttatataataatattgctatcaattttagcatcttaacaatattattatacatactaagctaaaattattcaatagttgtaaaagttgattagtcaataagtatatatttaatgtagtgttatctcttaaaaaaactagataaggagataataaatatatggaacaattagattcaaaatataagttgaaaaaatttctaatggcagtatttagagatggtataggacaaggaaataatcttattgataatgaatatgttagagtatttcaaaataataaaagtaatagtaaacaattagaactcggagaagaatttaaagaatatagtaaaacaactttttttaaaaatatagatgatatagtagaatttaccttcgcaaaaaatatttattatgaaaatacattttttaacctatgtactactgatggaaaagcaggaaccaatgaaaacttaataaatagatatgcattaggatttgattttgacaaaaaagaattaggacaaggttttaattataaagatataattaatttatttactaagataggattacattatcatatcctagttgatagtggaaatggattccatgtttatgtgctaattaataaaactaataacattaagttagtatcagaagttacaaatacattaataaataaattgggtgcagataaacaagcaaatttatctactcaagtattaagagtaccttatacatataatattaaaaatactactaaacaagtaaaaataatacaccaagacaaaaatatatatagatatgacatagaaaagttagctaaaaaatattgcaaagatgtaaaaacagtaggtaatactaatacaaaatatatattagatagtaagctaccaaattgtatagtagatattttaaaaaatggtagtaaagatggacataaaaacctagatttgcaaaaaatagttgtgactttaagattgaggaataaaagtttaagtcaagtaatatccgttgctagagaatggaactatatatcacaaaatagtctttcaaatagtgagctagaatatcaagtcaagtatatgtatgagaaacttaaaacggttaattttggttgtactggttgtgagtttaatagtgattgttggaataaaatagaatcagattttatatatagtgatgaagatactttgttcaatatgccacataagcactcaaaggatttgaaatataagaataggaaaggggttaaaataatgactggtaatcaattgtttatctataatgtgttacttaacaataaagatagagaattaaacatagacgatataatggagctgataacctataaacgtaagaagaaagttaaaaacattgttatgagtgaaaagacattaagagaaacattaaaagaacttcaacataatgattatattacaaaaacaaaaggtgttacaaagctaggaataaaagatacatacaatgtaaaagaagttagatgtaatatagataaacaatatactattagttactttgttaccatggcagtaatttggggaataatttcaactgaagaattaagattatatactcacatgagatataagcaagatttattggtcaaagatgataaaataaaaggaaatatattaagaattaatcaagaggaattagcaaaagatttaggagtaacacagcaaagaatttcaaatatgatagaatctttattagatactaaaattttagatgtatgggaaactaaaataaatgatagaggatttatgtactatacatatagattaaacaagtagatttttgataggattagaattgattttctagtcctatttttatgcaaaaaaactaattaataaaaatttcttttggtaaaataattgtacgagaattgcaaaaaaaaaatggcatcaaagtattgaaattaagccgttttaaaaatttcttttggtaaaataattctacatatatatgtagtatatatatatatgttttttagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatcctaattagtaggtgcttttttaaaacaagttaaaaatcaaaaatagtattagtaagcattggaaatgctagattctaaaatagaaaagtaaaaaattggtgcactatctaaacttatctatatcgctttttccgtcgtttggttctctagttacgatacaggggatatgcttatattgagttatagtactaatcagtgcttaatatagttaataaaattatagttaccatagtttagtaactatgatgtatgttagttagaaacttgcatttcggccggccagtgggcaagttg

SEQ ID NO: 314 - DNA, 957 bp, Bacillus subtilis
  source 1..957: mol_type = genomic DNA
gaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttg

SEQ ID NO: 315 - DNA, 1821 bp, synthetic construct
  source 1..1821: mol_type = other DNA
gaatggcgaatggcgctagcataaaaataagaagcctgcatttgcaggcttcttatttttatggcgcgcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctcctaaattcactttagataaaaatttaggaggcatatcaaatgaacggccggccagtgggcaagttg

SEQ ID NO: 316 - DNA, 3292 bp, Enterococcus faecalis
  source 1..3292: mol_type = genomic DNA
gaatggcgaatggcgctagcataaaaataagaagcctgcatttgcaggcttcttatttttatggcgcgccctcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaaataagcaacgatttcgtgcattattcacacgaagtcattgcttttttcttcttccatttctaaatccaatgttacttgttctgattctgtttctggttctggttctgttggctcatttgggattaaatccactactagcgttgagttagttaactttgcaatttgttctagtgtttttatggttggatctgattttcctgggccggccagtgggcaagttg

SEQ ID NO: 317 - DNA, 1691 bp, Lactococcus lactis
  source 1..1691: mol_type = genomic DNA
gaatggcgaatggcgctagcataaaaataagaagcctgcatttgcaggcttcttatttttatggcgcgccgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagccccctgacgaaagtcgaagggggtttttattttggtttgatgttgcgattaatagcaatacaaggccggccagtgggcaagttg

SEQ ID NO: 318 - DNA, 1691 bp, Bifidobacterium longum
  source 1..1691: mol_type = genomic DNA
gaatggcgaatggcgctagcataaaaataagaagcctgcatttgcaggcttcttatttttatggcgcgccgccgcgggcctcagcctgcggaacgcgcagcggacgccgacggctcagacggctcagaaacgtccgtgagtggcctccacgcggccgaacaggtcagggaggctcgcgcatacgtgagcggcgtggagaagcggctgaaggccgtccagcggcttttcgtgcaggatgtgctgggctgggttcagccgacgcttcgctgggctgaaatatctgacttggttcccgcgtatttgttcactgtacaaatacgatgtatgctgtagccatgtccgatgagtattcgcagccgacgcttgagctgtcgcgcacgttcgaaggctggtggctgcccgaacgcccgctgtgctgcgacgacgactactcccggctgcaccgcaggagccgcgccgacgcgctcaaatgcaagcacatcgaggcgaaccccgccgcgctggtgaacacgatcgtggtggacatcgacgacgcgaacgccaaggcgatggccctgtgggagcacgagggcatgcggccgaactggatcgcggagaacccggccaacgggcacgctcacgcgggctgggtgctcacctttccggtgcccagaaccgatctggcgcgtctcaagccgttgaagctcctgcacgccaccacggagggactgcgccgctcctgcgacggggacatgggctattcgggacttctgatgaagaaccccgagcatccggcgtgggcgtcggacatcatcgagtgggacacctacgacctggaacagctcgtgcagtcgctccaggaacacggggacatgccgcccgtcagctggaagcgcaccaagcgcgcccgcacgcaggggctgggacgcaactgcacgctcttcgacaaggcccgcacgctcgcctaccgctacgttgcggcggctgccgaccgttcggaggccagcagcgaggcattgcgcctatacgtgcgtcgcacctgccacgaactcaacgtctcgctgttccccgatccgctgcacgcgcgtgaggtcgaggacatcgccaagagcatccacaaatggatcgtcacccgcagccgcatgtggcgcgacggtgccattgccaacgcagccacattcatcgccatccaatccgcacgaggacacaaacacggtgagaacaaatatcagcaggtcatgaaggaggcactggaatggtaaggacgactttgaggaagaagcgcccggtgtctgcacgtgaattagctgaagcatacggcgtctccacgcgcaccattcagagctgggtggcaatgaagcgcgaggattggattgatgaacaagccgctatgcgcgaagcagtccgctcatatcacgatgacgagggccatacatggccgcagaccgccgagcatttcaacatgagccagggtgccgtgcgtcaacgctgctacagggctcgcaaggagcgcgaggacgaggcggcggagaaatcgaagcatctacccggcgagattccactgttcgactgacgctaaacgttgtcccaaacgcgaacgcagcacctccctcgccttgcggctttttcctcttccatcggccttcggcactcgggttgttgctccagcgccgcagggcgcgggaggctgcggccggccagtgggcaagttg

SEQ ID NO: 319 - DNA, 2433 bp, Clostridium perfringens
  source 1..2433: mol_type = genomic DNA
gaatggcgaatggcgctagcataaaaataagaagcctgcatttgcaggcttcttatttttatggcgcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgggccggccagtgggcaagttg